(19) United States
(12) Patent Application Publication
(10) Pub. No.: US 2025/0176881 A1
(43) Pub. Date: Jun. 5, 2025

(54) DEVICES FOR COLLECTING BIOLOGICAL SAMPLES

(71) Applicant: DRAWBRIDGE HEALTH, INC., Summerville, SC (US)

(72) Inventors: Brett L. Jordan, San Francisco, CA (US); Masao Drexel, Mountain View, CA (US); Dagmar L. Beyerlein, San Francisco, CA (US); Alicia Jackson, Menlo Park, CA (US); Kara Juneau, Palo Alto, CA (US); Rowan Chapman, Summerville, SC (US); Diana Maichin, Summerville, SC (US); Harry Glorikian, Summerville, SC (US)

(21) Appl. No.: 19/031,149

(22) Filed: Jan. 17, 2025

Related U.S. Application Data
(63) Continuation-in-part of application No. 17/681,648, filed on Feb. 25, 2022.
Application No. 17/681,648 is a continuation of application No. 16/685,954, filed on Nov. 15, 2019, now Pat. No. 11,298,060.
Application No. 16/685,954 is a continuation of application No. 16/104,846, filed on Aug. 17, 2018, now Pat. No. 10,638,963.
Application No. 16/104,846 is a continuation of application No. PCT/US2018/013223, filed on Jan. 10, 2018.
(60) Provisional application No. 62/468,906, filed on Mar. 8, 2017.
(60) Provisional application No. 62/444,764, filed on Jan. 10, 2017.

(51) Int. Cl. A61B 5/15; A61B 5/151; B01L 3/00; B01L 3/02; G01N 33/487; G01N 35/00

(52) U.S. Cl. CPC A61B 5/150099; A61B 5/150022; A61B 5/15016; A61B 5/150251; A61B 5/150305; A61B 5/150343; A61B 5/150427; A61B 5/150442; A61B 5/150977; A61B 5/150984; A61B 5/15113; A61B 5/15117; A61B 5/15142; B01L 3/0296; B01L 3/5023; B01L 3/508; G01N 33/487; G01N 35/00732; A61B 5/150236; A61B 5/150244; A61B 5/150503; A61B 5/150969; A61B 5/1513; B01L 2200/026; B01L 2200/18; B01L 2300/021; B01L 2300/041; B01L 2300/06; B01L 2300/0609; B01L 2300/0825; B01L 2300/0864; B01L 2400/0406; B01L 2400/049

(57) ABSTRACT

Disclosed herein are devices, apparatus, systems, methods and kits for collecting and storing a fluid sample from a subject. A device for collecting the fluid sample can include a housing comprising a recess having an opening, a vacuum chamber in the housing and in fluidic communication with the recess, and one or more piercing elements that are extendable through the opening to penetrate skin of the subject. The vacuum chamber can be configured for having a vacuum that draws the skin into the recess. The recess can be configured having a size or shape that enables an increased volume of the fluid sample to be accumulated in the skin drawn into the recess.

### CROSS-REFERENCE TO RELATED APPLICATIONS

This application is a continuation-in-part of U.S. patent application Ser. No. 17/681,648, filed Feb. 25, 2022, which is a continuation of U.S. patent application Ser. No. 16/685,954, filed Nov. 15, 2019, which is a continuation of U.S. patent application Ser. No. 16/104,846, filed Aug. 17, 2018, now U.S. Pat. No. 10,638,963, issued May 5, 2020, which is a continuation of International application number PCT/US2018/013223, filed Jan. 10, 2018, which claims the benefit of U.S. provisional patent application No. 62/468,906, filed Mar. 8, 2017, and U.S. provisional patent application Ser. No. 62/444,764, filed Jan. 10, 2017, which are herein incorporated by reference in their entirety.

### BACKGROUND

Body fluid collection, for example collection of blood samples for performing diagnostic tests, can be used to assess and inform the health of individuals. Early detection and reliable diagnosis can play a central role in making effective therapeutic decisions for treatment of diseases or managing certain physiological conditions. Detection can involve identification of disease-specific biomarkers in human body fluids that can indicate irregularities in cellular regulatory functions, pathological responses, or intervention to therapeutic drugs.

Many individuals, however, may not relish the process of having blood drawn from their bodies, possibly due to association with pain, cuts, bleeding, sharp objects, sight of blood, fear of infections, etc. Typically, venous blood collection of a subject is performed at external facilities such as hospitals, skilled nursing facilities, and outpatient environments such as primary care physician (PCP) & specialty hospital clinics, surgery centers, occupational health clinics, or physician offices. The blood collection process can be tedious and time consuming for individuals who have to visit those facilities for blood draw, and for healthcare personnel who can have to attend to multiple patient encounters within a single day.

Medical diagnostic tests can save lives; unfortunately they are not readily available to everyone. The growing costs of healthcare and availability of treatment can make it difficult for individuals around the world to receive adequate treatment. Having a simple blood panel done can require multiple visits to a medical doctor, first for blood collection then a follow up consultation or additional tests. The rising tangible costs of healthcare can be an additional deterrent; many patients cannot afford the time and cost of multiple visits to a doctor, and can put off tests until they experience symptoms that require medical treatment. New treatment paradigms can address the needs of the patient by reducing the time, cost and availability burdens. Improving patient access to diagnostics can improve the likelihood that tests will be conducted before symptoms appear which in turn can result in patients receiving treatment far earlier than previously possible. Early detection can facilitate early treatment, which in turn can ensure a better prognosis for the patient and an overall reduction in the cost of treatment.

Provided herein are improved solutions to enable a patient to collect, prepare, store, detect and analyze their own blood samples without the assistance of a medical practitioner or access to a medical facility.

Thus, a need exists for improved devices and methods that enable blood collection to be performed easily and conveniently by users, and that can decrease users' reliance on traditional healthcare facilities for blood draw.

### SUMMARY

The present disclosure addresses at least the above needs. Various embodiments of the present disclosure address the demand for devices and methods, that enable individuals to easily, conveniently, and reliably collect and store blood samples outside of traditional healthcare facilities, for example in their own homes, in remote locations, while traveling, etc. Individuals who have minimal to no medical training can use the disclosed devices and methods to efficiently collect and store blood on their own or with the help of others, without the need for trained healthcare personnel. The embodiments described herein can obviate the need for individuals to schedule, or make special or frequent trips to healthcare facilities for blood sample collection, which helps to free up the individuals' time and reduce patient load on healthcare resources. Nonetheless, it should be appreciated that the disclosed devices and methods are also suitable for use by healthcare or non-healthcare personnel in a variety of environments or applications, for example in personalized point-of-care (POC), Emergency Medical Services (EMS), ambulatory care, hospitals, clinics, emergency rooms, patient examination rooms, acute care patient rooms, field environments, nurse's offices in educational settings, occupational health clinics, surgery or operation rooms, etc.

Blood samples collected using the devices and methods described herein can be analyzed to determine a person's physiological state, for detecting diseases and also for monitoring the health conditions of the user. In some instances, individuals can rapidly evaluate their physiological status since blood samples can be quickly collected using the devices and methods described herein, and either (1) analyzed on the spot using for example immunoassays or (2) shipped promptly to a testing facility. The reduced lead-time for blood collection, analysis and quantification can be beneficial to many users, especially users who have certain physiological conditions/diseases that require constant and frequent blood sample collection/monitoring. Taking diabetes as an example, hemoglobin A1c (HbA1c) can make up 60% of all glycohemoglobins and can be used for monitoring glycemic control. The amount of HbA1c, as a percentage of total hemoglobin, can reflect the average blood glucose concentration in a patient's blood over the preceding 120 days. Generally it is recommended that diabetic patients test their HbA1c levels every three to six months. The glycemic recommendation for non-pregnant adults with diabetes can be <7.0%, while HbA1c levels of ≥8% can indicate that medical action can be required to control diabetic complications, including cognitive impairment and hypoglycemic vulnerability.

The various embodiments described herein are capable of drawing blood at increased flowrates and higher sample volumes beginning from time of skin incision, compared to traditional non-venous blood collection devices and method. The disclosed devices and methods can be used to collect blood samples of predefined volumes, for example through the use of custom matrices for sample collection, and absorbent pads for holding and metering out excess blood. Additionally, the blood collection devices and methods described herein are minimally invasive and permit lower levels of pain (or perception of pain) in a subject, which can help to improve the overall blood collection experience for the subject.

In one aspect, a method is disclosed. The method involves deploying a sample acquisition and stabilization system with a tag to a subject wherein the system includes a blood collection unit configured for collecting a blood sample from the subject, an optional separation module for separating one or more components from the collected blood sample, and a stabilization matrix configured for stabilizing the one or more components from the collected blood sample wherein the tag comprises a label associated with an assay. The method further involves collecting the deployed tagged sample acquisition and stabilization system, performing one or more assays on the one or more stabilized components based on the tag, and providing a report on the one or more assays performed. The method can further involve separating the stabilization matrix from the system prior to one of the aforementioned method steps. The tag can be on the stabilization matrix. Optionally, when the separation module is present, the tag can be on the optional separation module. The stabilization matrix can be configured to perform multiple assays. The method can further involve identifying stabilized bio-components within the blood sample based on the tag present in a deployed sample acquisition and stabilization system and from that identification determining a stabilized component-specific assay to perform. The sample acquisition and stabilization system can be labeled with an RFID transmitter, a color label, a barcode, or a second label that differs from the sample tag.

In another aspect, a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize at least one bio-component selected from the group consisting of: RNA, DNA, and protein. The method further involves performing one or more assays on the at least one stabilized bio-component in or on the sampling device at a location remote from one of the above-mentioned method steps. The one or more stabilization reagents can be disposed on or are integrated with a solid matrix and are in a substantially dry state. The solid matrix can be configured to substantially stabilize RNA for at least 11 days at 37 degrees Celsius. The assays performed in or on the device can include one or more of: PCR, DNA sequencing, RNA expression analysis, and RT-PCR.

In another aspect a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize one or more bio-components selected from the group consisting of: RNA, DNA, and protein. The method further involves removing from the sampling device the stabilization matrix with the one or more bio-components; and performing one or more assays on the stabilization matrix. The stabilization matrix can be removed from the sampling device at a location remote from where one of the above-mentioned steps is carried out.

In another aspect, a method is disclosed which involves receiving, under ambient conditions, a substantially dry solid matrix comprising one or more stabilization reagents that selectively stabilize one or more bio-components selected from the group consisting of: DNA, RNA and protein, and performing one or more assays directly off of the dry solid matrix for analyzing the one or more selectively stabilized bio-components. The stabilization reagent can include a solid substrate comprising melezitose under a substantially dry state with water content of less than 2%. The stabilization reagent can be coupled to a sample acquisition component which includes a solid matrix for extraction and storage of nucleic acids from the biological sample, wherein the solid matrix has an RNA Integrity Number (RIN) of at least 4.

In another aspect, a device for point-of-care or self-administered collection and stabilization of a bio-sample is disclosed and embodiments are illustrated herein. The device includes a sample acquisition component comprising a body and a plunger, wherein the body comprises a proximal end, a distal end, an outer body surface extending between the proximal and distal ends, a base comprising at least one aperture and attached to the distal end, and a lumen defined within the body surface, wherein the plunger is disposed within the lumen, wherein the plunger comprises a proximal end, a distal end, and at least one needle connected to either end, wherein the at least one needle is configured to pass through the at least one aperture in the base when the plunger is actuated. The device includes a sample stabilization component which includes a reagent and a substrate configured such that components of a bio-sample are extracted and stabilized as the bio-sample migrates through the matrix from a point of collection; wherein the sample stabilization component is disposed within the lumen of the body such that actuation of the plunger drives the bio-sample through the matrix to further facilitate separation of the bio-sample.

In another aspect, a kit for collecting and stabilizing a sample is disclosed. The kit includes a sample acquisition component with one or more piercing elements configured for penetrating skin to expose blood, and a sample stabilization component, wherein the sample stabilization component comprises a solid substrate comprising melezitose under a substantially dry state. The solid substrate can include one or more of a lysis reagent and a biomolecule stabilizing reagent. Optionally, the melezitose can be present at a concentration in a range of 10 to 30%. The solid substrate can be configured for preserving enzyme activity. The kit can further include a plasma separation component.

In another aspect, a kit for collecting and stabilizing a biological sample is disclosed. The kit includes a sample acquisition component with one or more piercing elements configured for penetrating skin to expose blood, and a sample stabilization component comprising a solid matrix for selectively stabilizing one or more of the following bio-components: RNA, DNA, and protein. The solid matrix can selectively stabilize RNA and RNA obtained from the solid matrix can have a RIN number greater than 4. The kit can further include a plasma separation component. RNA obtained from the solid matrix can have a RIN of at least 5 or of at least 6, or of at least 7, or have a RIN of greater than 7. The solid matrix can further include at least one protein denaturant present in the solid matrix in a dry state. The solid matrix can further include at least one reducing agent in the solid matrix in a dry state. The solid matrix can further include at least one UV protectant in the solid matrix in a dry state. The solid matrix can further include at least one buffer present in the solid matrix in a dry state. The buffer can be an acid-titrated buffer reagent that generates a pH in a range from 3 to 6. The solid matrix can be comprised of cellulose, cellulose acetate, glass fiber, or a combination thereof. The solid matrix can be a porous matrix. The solid matrix can be a non-dissolvable dry solid material. The solid matrix can be configured to provide an acidic pH upon hydration. The solid matrix can be configured to extract nucleic acids from a sample, and preserve the nucleic acids in a substantially dry state at ambient temperature. The sample acquisition component can be connected to the sample stabilization component.

In another aspect, a system for collecting and stabilizing a sample is disclosed. The system includes a sample acquisition component with one or more piercing elements configured for penetrating skin to expose blood, and a sample stabilization component coupled to the sample acquisition component, wherein the sample stabilization component comprises a solid substrate comprising melezitose under a substantially dry state with a water content of less than 2%. The solid substrate can include a lysis reagent, one or more biomolecule stabilizing reagents, or a combination thereof. The concentration of melezitose can be present in a range of 10 to 30%. The solid substrate can be configured for preserving enzyme activity.

In another aspect, a system for collecting and stabilizing a biological sample is disclosed. The system includes a sample acquisition component with one or more piercing elements configured for penetrating skin to expose blood, and a sample stabilization component coupled to the sample acquisition component comprising a solid matrix for extraction and storage of a nucleic acid from the biological sample, wherein RNA obtained from the solid matrix has a RIN of at least 4. RNA obtained from the solid matrix can have a RIN of at least 5, or of at least 6, or of at least 7, or can be greater than 7. The solid matrix can further include at least one protein denaturant present in the solid matrix in a dry state. The solid matrix can further include at least one reducing agent in the solid matrix in a dry state. The solid matrix can further include at least one UV protectant in the solid matrix in a dry state. The solid matrix can further include at least one buffer disposed on or impregnated within the solid matrix, wherein the solid matrix is substantially dry with a water content of less than 2%. The buffer can be an acid-titrated buffer reagent that generates a pH in a range from 3 to 6. The solid matrix can be comprised of cellulose, cellulose acetate, glass fiber, or a combination thereof. The solid matrix can also be a porous matrix. The solid matrix can be a non-dissolvable dry solid material. The solid matrix can be configured to provide an acidic pH upon hydration. The solid matrix can be configured to extract a nucleic acid from a sample, and preserve the nucleic acid in a substantially dry state at ambient temperature. The sample acquisition component can be connected to the sample stabilization component.

In another aspect, a system for collecting and stabilizing a biological sample is disclosed. The system includes a sample acquisition component with one or more piercing elements configured for penetrating skin to expose blood, and a sample separation component coupled to the sample acquisition component, wherein the sample acquisition component comprises two or more partially overlapping membranes configured such that one component of the biological sample is collected on a first membrane and the remainder of the sample is collected on a second membrane.

In another aspect, a method is disclosed which involves deploying a tagged sample acquisition and stabilization system to a subject wherein the system includes a blood collection unit configured for collecting a blood sample from the subject, an optional separation module for separating one or more components from the collected blood sample, and a stabilization matrix configured for stabilizing one or more components from the blood sample wherein the tagged sample acquisition and stabilization system comprises a label associated with an assay. The method further includes collecting a deployed tagged sample acquisition and stabilization system, performing one or more assays on the one or more stabilized components based on the tag, and providing a report on the one or more assays performed. The method can further involve separating the stabilization matrix from the system prior to one of the above-mentioned method steps. Optionally, the tag can be on the stabilization matrix. Optionally, when the separation module is present, the tag can be on the optional separation module. The stabilization matrix can be configured to perform multiple assays. The method can further involve identifying stabilized bio-components within the blood sample based on the tag present in a deployed sample acquisition and stabilization system and from that identification determining a stabilized component-specific assay to perform. The sample stabilization system can be labeled with an RFID transmitter, a color label, a barcode, or a second label that differs from the sample tag.

In another aspect, a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize at least one bio-component selected from the group consisting of: RNA, DNA, and protein. The method further involves performing one or more assays on the stabilized bio-components in or on the sampling device at a location remote from where the first method step is carried out. The one or more stabilization reagents can be disposed on or integrated with a solid matrix and can be in a substantially dry state. The solid matrix can be configured to substantially stabilize RNA for at least 11 days at 37 degrees Celsius. The assays can be performed in or on the device, and can include one or more of: PCR, DNA sequencing, RNA expression analysis, and RT-PCR.

In another aspect, a method for performing analysis on one or more bio-components from a bio-sample from a subject is disclosed. The method involves obtaining a sampling device which includes a sample acquisition component and a stabilization matrix comprising one or more stabilizing reagents configured to selectively stabilize the one or more bio-components selected from the group consisting of: RNA, DNA, protein, and a combination thereof. The method further involves removing from the sampling device the stabilization matrix with the one or more bio-components, and performing one or more assays on or directly off of the stabilization matrix. The stabilization matrix can be removed from the sampling device at a location remote from one of the above-mentioned method steps.

In another aspect, a method is disclosed which involves receiving, under ambient conditions, a substantially dry solid matrix comprising one or more stabilization reagents that selectively stabilize one or more bio-components selected from the group consisting of: DNA, RNA and protein, and performing one or more assays directly off of the dry solid matrix for analyzing the selectively stabilized bio-components. The stabilization reagent can include a solid substrate comprising melezitose under a substantially dry state with water content of less than 2%. The stabilization reagent can be coupled to the sample acquisition component comprising a solid matrix for extraction and storage of nucleic acids from the biological sample, wherein RNA obtained from the solid matrix has a RIN of at least 4.

In another aspect, a device for point-of-care or self-administered collection and stabilization of a sample is disclosed and embodiments are illustrated herein. The device includes a sample acquisition component comprising a body and a plunger, wherein the body comprises a proximal end, a distal end, an outer body surface extending between the proximal and distal ends, a base comprising at least one aperture and attached to the distal end, and a lumen defined within the body, wherein the plunger is disposed within the lumen, wherein the plunger comprises a proximal end, a distal end, and at least one needle connected to either end, wherein the at least one needle is configured to pass through the at least one aperture in the base when the plunger is actuated. The device includes a sample stabilization component comprising a reagent and a substrate configured such that components of a bio-sample are extracted and stabilized as the bio-sample migrates through the matrix from a point of collection, wherein the sample stabilization component is disposed within the lumen of the body, such that actuation of the plunger drives the bio-sample through the matrix to further facilitate separation of the bio-sample.

In another aspect, a kit for collecting and stabilizing a sample is disclosed. The kit includes a sample acquisition component with one or more piercing elements configured for penetrating skin to expose blood, and a sample stabilization component, wherein the sample stabilization component comprises a solid substrate comprising melezitose under a substantially dry state. The solid substrate can further include one or more of a lysis reagent and a biomolecule stabilizing reagent. The melezitose can be present at a concentration in a range of 10 to 30%. The solid substrate can be configured for preserving enzyme activity. The kit can further include a plasma separation component.

In one aspect, a method is disclosed. The method involves deploying a sample acquisition and stabilization system with a tag to a subject wherein the system includes a blood collection unit configured for collecting a blood sample from the subject, an optional separation module for separating one or more components from the collected blood sample, and a stabilization matrix configured for stabilizing the one or more components from the collected blood sample wherein the tag comprises a label associated with an assay. The method further involves collecting the deployed tagged sample acquisition and stabilization system, performing one or more assays on the one or more stabilized components based on the tag, and providing a report on the one or more assays performed. The method can further involve separating the stabilization matrix from the system prior to one of the aforementioned method steps. The tag can be on the stabilization matrix. Optionally, when the separation module is present, the tag can be on the optional separation module. The stabilization matrix can be configured to perform multiple assays. The method can further involve identifying stabilized bio-components within the blood sample based on the tag present in a deployed sample acquisition and stabilization system and from that identification determining a stabilized component-specific assay to perform. The sample acquisition and stabilization system can be labeled with an RFID transmitter, a color label, a barcode, or a second label that differs from the sample tag.

In another aspect, a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize at least one bio-component selected from the group consisting of: RNA, DNA, and protein. The method further involves performing one or more assays on the at least one stabilized bio-component in or on the sampling device at a location remote from one of the above-mentioned method steps. The one or more stabilization reagents can be disposed on or are integrated with a solid matrix and are in a substantially dry state. The solid matrix can be configured to substantially stabilize RNA for at least 11 days at 37 degrees Celsius. The assays performed in or on the device can include one or more of: PCR, DNA sequencing, RNA expression analysis, and RT-PCR.

In another aspect a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize one or more bio-components selected from the group consisting of: RNA, DNA, and protein. The method further involves removing from the sampling device the stabilization matrix with the one or more bio-components; and performing one or more assays on the stabilization matrix. The stabilization matrix can be removed from the sampling device at a location remote from where one of the above-mentioned steps is carried out.

In another aspect, a method is disclosed which involves receiving, under ambient conditions, a substantially dry solid matrix comprising one or more stabilization reagents that selectively stabilize one or more bio-components selected from the group consisting of: DNA, RNA and protein, and performing one or more assays directly off of the dry solid matrix for analyzing the one or more selectively stabilized bio-components. The stabilization reagent can include a solid substrate comprising melezitose under a substantially dry state with water content of less than 2%. The stabilization reagent can be coupled to a sample acquisition component which includes a solid matrix for extraction and storage of nucleic acids from the biological sample, wherein the solid matrix has an RNA Integrity Number (RIN) of at least 4.

In another aspect, a device for point-of-care or self-administered collection and stabilization of a bio-sample is disclosed and embodiments are illustrated herein. The device includes a sample acquisition component comprising a body and a plunger, wherein the body comprises a proximal end, a distal end, an outer body surface extending between the proximal and distal ends, a base comprising at least one aperture and attached to the distal end, and a lumen defined within the body surface, wherein the plunger is disposed within the lumen, wherein the plunger comprises a proximal end, a distal end, and at least one needle connected to either end, wherein the at least one needle is configured to pass through the at least one aperture in the base when the plunger is actuated. The device includes a sample stabilization component which includes a reagent and a substrate configured such that components of a bio-sample are extracted and stabilized as the bio-sample migrates through the matrix from a point of collection; wherein the sample stabilization component is disposed within the lumen of the body such that actuation of the plunger drives the bio-sample through the matrix to further facilitate separation of the bio-sample.

In another aspect, a kit for collecting and stabilizing a sample is disclosed. The kit includes a sample acquisition component with one or more piercing elements configured for penetrating skin to expose blood, and a sample stabilization component, wherein the sample stabilization component comprises a solid substrate comprising melezitose under a substantially dry state. The solid substrate can include one or more of a lysis reagent and a biomolecule stabilizing reagent. Optionally, the melezitose can be present at a concentration in a range of 10 to 30%. The solid substrate can be configured for preserving enzyme activity. The kit can further include a plasma separation component.

In another aspect, a kit for collecting and stabilizing a biological sample is disclosed. The kit includes a sample acquisition component with one or more piercing elements configured for penetrating skin to expose blood, and a sample stabilization component comprising a solid matrix for selectively stabilizing one or more of the following bio-components: RNA, DNA, and protein. The solid matrix can selectively stabilize RNA and RNA obtained from the solid matrix can have a RIN number greater than 4. The kit can further include a plasma separation component. RNA obtained from the solid matrix can have a RIN of at least 5 or of at least 6, or of at least 7, or have a RIN of greater than 7. The solid matrix can further include at least one protein denaturant present in the solid matrix in a dry state. The solid matrix can further include at least one reducing agent in the solid matrix in a dry state. The solid matrix can further include at least one UV protectant in the solid matrix in a dry state. The solid matrix can further include at least one buffer present in the solid matrix in a dry state. The buffer can be an acid-titrated buffer reagent that generates a pH in a range from 3 to 6. The solid matrix can be comprised of cellulose, cellulose acetate, glass fiber, or a combination thereof. The solid matrix can be a porous matrix. The solid matrix can be a non-dissolvable dry solid material. The solid matrix can be configured to provide an acidic pH upon hydration. The solid matrix can be configured to extract nucleic acids from a sample, and preserve the nucleic acids in a substantially dry state at ambient temperature. The sample acquisition component can be connected to the sample stabilization component.

In another aspect, a system for collecting and stabilizing a sample is disclosed. The system includes a sample acquisition component with one or more piercing elements configured for penetrating skin to expose blood, and a sample stabilization component coupled to the sample acquisition component, wherein the sample stabilization component comprises a solid substrate comprising melezitose under a substantially dry state with a water content of less than 2%. The solid substrate can include a lysis reagent, one or more biomolecule stabilizing reagents, or a combination thereof. The concentration of melezitose can be present in a range of 10 to 30%. The solid substrate can be configured for preserving enzyme activity.

In another aspect, a system for collecting and stabilizing a biological sample is disclosed. The system includes a sample acquisition component with one or more piercing elements configured for penetrating skin to expose blood, and a sample stabilization component coupled to the sample acquisition component comprising a solid matrix for extraction and storage of a nucleic acid from the biological sample, wherein RNA obtained from the solid matrix has a RIN of at least 4. RNA obtained from the solid matrix can have a RIN of at least 5, or of at least 6, or of at least 7, or can be greater than 7. The solid matrix can further include at least one protein denaturant present in the solid matrix in a dry state. The solid matrix can further include at least one reducing agent in the solid matrix in a dry state. The solid matrix can further include at least one UV protectant in the solid matrix in a dry state. The solid matrix can further include at least one buffer disposed on or impregnated within the solid matrix, wherein the solid matrix is substantially dry with a water content of less than 2%. The buffer can be an acid-titrated buffer reagent that generates a pH in a range from 3 to 6. The solid matrix can be comprised of cellulose, cellulose acetate, glass fiber, or a combination thereof. The solid matrix can also be a porous matrix. The solid matrix can be a non-dissolvable dry solid material. The solid matrix can be configured to provide an acidic pH upon hydration. The solid matrix can be configured to extract a nucleic acid from a sample, and preserve the nucleic acid in a substantially dry state at ambient temperature. The sample acquisition component can be connected to the sample stabilization component.

In another aspect, a system for collecting and stabilizing a biological sample is disclosed. The system includes a sample acquisition component with one or more piercing elements configured for penetrating skin to expose blood, and a sample separation component coupled to the sample acquisition component, wherein the sample acquisition component comprises two or more partially overlapping membranes configured such that one component of the biological sample is collected on a first membrane and the remainder of the sample is collected on a second membrane.

In another aspect, a method is disclosed which involves deploying a tagged sample acquisition and stabilization system to a subject wherein the system includes a blood collection unit configured for collecting a blood sample from the subject, an optional separation module for separating one or more components from the collected blood sample, and a stabilization matrix configured for stabilizing one or more components from the blood sample wherein the tagged sample acquisition and stabilization system comprises a label associated with an assay. The method further includes collecting a deployed tagged sample acquisition and stabilization system, performing one or more assays on the one or more stabilized components based on the tag, and providing a report on the one or more assays performed. The method can further involve separating the stabilization matrix from the system prior to one of the above-mentioned method steps. Optionally, the tag can be on the stabilization matrix. Optionally, when the separation module is present, the tag can be on the optional separation module. The stabilization matrix can be configured to perform multiple assays. The method can further involve identifying stabilized bio-components within the blood sample based on the tag present in a deployed sample acquisition and stabilization system and from that identification determining a stabilized component-specific assay to perform. The sample stabilization system can be labeled with an RFID transmitter, a color label, a barcode, or a second label that differs from the sample tag.

In another aspect, a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize at least one bio-component selected from the group consisting of: RNA, DNA, and protein. The method further involves performing one or more assays on the stabilized bio-components in or on the sampling device at a location remote from where the first method step is carried out. The one or more stabilization reagents can be disposed on or integrated with a solid matrix and can be in a substantially dry state. The solid matrix can be configured to substantially stabilize RNA for at least 11 days at 37 degrees Celsius. The assays can be performed in or on the device, and can include one or more of: PCR, DNA sequencing, RNA expression analysis, and RT-PCR.

In another aspect, a method for performing analysis on one or more bio-components from a bio-sample from a subject is disclosed. The method involves obtaining a sampling device which includes a sample acquisition component and a stabilization matrix comprising one or more stabilizing reagents configured to selectively stabilize the one or more bio-components selected from the group consisting of: RNA, DNA, protein, and a combination thereof. The method further involves removing from the sampling device the stabilization matrix with the one or more bio-components, and performing one or more assays on or directly off of the stabilization matrix. The stabilization matrix can be removed from the sampling device at a location remote from one of the above-mentioned method steps.

In another aspect, a method is disclosed which involves receiving, under ambient conditions, a substantially dry solid matrix comprising one or more stabilization reagents that selectively stabilize one or more bio-components selected from the group consisting of: DNA, RNA and protein, and performing one or more assays directly off of the dry solid matrix for analyzing the selectively stabilized bio-components. The stabilization reagent can include a solid substrate comprising melezitose under a substantially dry state with water content of less than 2%. The stabilization reagent can be coupled to the sample acquisition component comprising a solid matrix for extraction and storage of nucleic acids from the biological sample, wherein RNA obtained from the solid matrix has a RIN of at least 4.

In another aspect, a device for point-of-care or self-administered collection and stabilization of a sample is disclosed and embodiments are illustrated herein. The device includes a sample acquisition component comprising a body and a plunger, wherein the body comprises a proximal end, a distal end, an outer body surface extending between the proximal and distal ends, a base comprising at least one aperture and attached to the distal end, and a lumen defined within the body, wherein the plunger is disposed within the lumen, wherein the plunger comprises a proximal end, a distal end, and at least one needle connected to either end, wherein the at least one needle is configured to pass through the at least one aperture in the base when the plunger is actuated. The device includes a sample stabilization component comprising a reagent and a substrate configured such that components of a bio-sample are extracted and stabilized as the bio-sample migrates through the matrix from a point of collection, wherein the sample stabilization component is disposed within the lumen of the body, such that actuation of the plunger drives the bio-sample through the matrix to further facilitate separation of the bio-sample.

In another aspect, a kit for collecting and stabilizing a sample is disclosed. The kit includes a sample acquisition component with one or more piercing elements configured for penetrating skin to expose blood, and a sample stabilization component, wherein the sample stabilization component comprises a solid substrate comprising melezitose under a substantially dry state. The solid substrate can further include one or more of a lysis reagent and a biomolecule stabilizing reagent. The melezitose can be present at a concentration in a range of 10 to 30%. The solid substrate can be configured for preserving enzyme activity. The kit can further include a plasma separation component.

In one aspect, a method is disclosed. The method involves deploying a sample acquisition and stabilization system with a tag to a subject wherein the system includes a blood collection unit configured for collecting a blood sample from the subject, an optional separation module for separating one or more components from the collected blood sample, and a stabilization matrix configured for stabilizing the one or more components from the collected blood sample wherein the tag comprises a label associated with an assay. The method further involves collecting the deployed tagged sample acquisition and stabilization system, performing one or more assays on the one or more stabilized components based on the tag, and providing a report on the one or more assays performed. The method can further involve separating the stabilization matrix from the system prior to one of the aforementioned method steps. The tag can be on the stabilization matrix. Optionally, when the separation module is present, the tag can be on the optional separation module. The stabilization matrix can be configured to perform multiple assays. The method can further involve identifying stabilized bio-components within the blood sample based on the tag present in a deployed sample acquisition and stabilization system and from that identification determining a stabilized component-specific assay to perform. The sample acquisition and stabilization system can be labeled with an RFID transmitter, a color label, a barcode, or a second label that differs from the sample tag.

In another aspect, a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize at least one bio-component selected from the group consisting of: RNA, DNA, and protein. The method further involves performing one or more assays on the at least one stabilized bio-component in or on the sampling device at a location remote from one of the above-mentioned method steps. The one or more stabilization reagents can be disposed on or are integrated with a solid matrix and are in a substantially dry state. The solid matrix can be configured to substantially stabilize RNA for at least 11 days at 37 degrees Celsius. The assays performed in or on the device can include one or more of: PCR, DNA sequencing, RNA expression analysis, and RT-PCR.

In another aspect a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize one or more bio-components selected from the group consisting of: RNA, DNA, and protein. The method further involves removing from the sampling device the stabilization matrix with the one or more bio-components; and performing one or more assays on the stabilization matrix. The stabilization matrix can be removed from the sampling device at a location remote from where one of the above-mentioned steps is carried out.

In another aspect, a method is disclosed which involves receiving, under ambient conditions, a substantially dry solid matrix comprising one or more stabilization reagents that selectively stabilize one or more bio-components selected from the group consisting of: DNA, RNA and protein, and performing one or more assays directly off of the dry solid matrix for analyzing the one or more selectively stabilized bio-components. The stabilization reagent can include a solid substrate comprising melezitose under a substantially dry state with water content of less than 2%. The stabilization reagent can be coupled to a sample acquisition component which includes a solid matrix for extraction and storage of nucleic acids from the biological sample, wherein the solid matrix has an RNA Integrity Number (RIN) of at least 4.

In another aspect, a device for point-of-care or self-administered collection and stabilization of a bio-sample is disclosed and embodiments are illustrated herein. The device includes a sample acquisition component comprising a body and a plunger, wherein the body comprises a proximal end, a distal end, an outer body surface extending between the proximal and distal ends, a base comprising at least one aperture and attached to the distal end, and a lumen defined within the body surface, wherein the plunger is disposed within the lumen, wherein the plunger comprises a proximal end, a distal end, and at least one needle connected to either end, wherein the at least one needle is configured to pass through the at least one aperture in the base when the plunger is actuated. The device includes a sample stabilization component which includes a reagent and a substrate configured such that components of a bio-sample are extracted and stabilized as the bio-sample migrates through the matrix from a point of collection; wherein the sample stabilization component is disposed within the lumen of the body such that actuation of the plunger drives the bio-sample through the matrix to further facilitate separation of the bio-sample.

In another aspect, a kit for collecting and stabilizing a sample is disclosed. The kit includes a sample acquisition component with one or more piercing elements configured for penetrating skin to expose blood, and a sample stabilization component, wherein the sample stabilization component comprises a solid substrate comprising melezitose under a substantially dry state. The solid substrate can include one or more of a lysis reagent and a biomolecule stabilizing reagent. Optionally, the melezitose can be present at a concentration in a range of 10 to 30%. The solid substrate can be configured for preserving enzyme activity. The kit can further include a plasma separation component.

In another aspect, a kit for collecting and stabilizing a biological sample is disclosed. The kit includes a sample acquisition component with one or more piercing elements configured for penetrating skin to expose blood, and a sample stabilization component comprising a solid matrix for selectively stabilizing one or more of the following bio-components: RNA, DNA, and protein. The solid matrix can selectively stabilize RNA and RNA obtained from the solid matrix can have a RIN number greater than 4. The kit can further include a plasma separation component. RNA obtained from the solid matrix can have a RIN of at least 5 or of at least 6, or of at least 7, or have a RIN of greater than 7. The solid matrix can further include at least one protein denaturant present in the solid matrix in a dry state. The solid matrix can further include at least one reducing agent in the solid matrix in a dry state. The solid matrix can further include at least one UV protectant in the solid matrix in a dry state. The solid matrix can further include at least one buffer present in the solid matrix in a dry state. The buffer can be an acid-titrated buffer reagent that generates a pH in a range from 3 to 6. The solid matrix can be comprised of cellulose, cellulose acetate, glass fiber, or a combination thereof. The solid matrix can be a porous matrix. The solid matrix can be a non-dissolvable dry solid material. The solid matrix can be configured to provide an acidic pH upon hydration. The solid matrix can be configured to extract nucleic acids from a sample, and preserve the nucleic acids in a substantially dry state at ambient temperature. The sample acquisition component can be connected to the sample stabilization component.

In another aspect, a system for collecting and stabilizing a sample is disclosed. The system includes a sample acquisition component with one or more piercing elements configured for penetrating skin to expose blood, and a sample stabilization component coupled to the sample acquisition component, wherein the sample stabilization component comprises a solid substrate comprising melezitose under a substantially dry state with a water content of less than 2%. The solid substrate can include a lysis reagent, one or more biomolecule stabilizing reagents, or a combination thereof. The concentration of melezitose can be present in a range of 10 to 30%. The solid substrate can be configured for preserving enzyme activity.

In another aspect, a system for collecting and stabilizing a biological sample is disclosed. The system includes a sample acquisition component with one or more piercing elements configured for penetrating skin to expose blood, and a sample stabilization component coupled to the sample acquisition component comprising a solid matrix for extraction and storage of a nucleic acid from the biological sample, wherein RNA obtained from the solid matrix has a RIN of at least 4. RNA obtained from the solid matrix can have a RIN of at least 5, or of at least 6, or of at least 7, or can be greater than 7. The solid matrix can further include at least one protein denaturant present in the solid matrix in a dry state. The solid matrix can further include at least one reducing agent in the solid matrix in a dry state. The solid matrix can further include at least one UV protectant in the solid matrix in a dry state. The solid matrix can further include at least one buffer disposed on or impregnated within the solid matrix, wherein the solid matrix is substantially dry with a water content of less than 2%. The buffer can be an acid-titrated buffer reagent that generates a pH in a range from 3 to 6. The solid matrix can be comprised of cellulose, cellulose acetate, glass fiber, or a combination thereof. The solid matrix can also be a porous matrix. The solid matrix can be a non-dissolvable dry solid material. The solid matrix can be configured to provide an acidic pH upon hydration. The solid matrix can be configured to extract a nucleic acid from a sample, and preserve the nucleic acid in a substantially dry state at ambient temperature. The sample acquisition component can be connected to the sample stabilization component.

In another aspect, a system for collecting and stabilizing a biological sample is disclosed. The system includes a sample acquisition component with one or more piercing elements configured for penetrating skin to expose blood, and a sample separation component coupled to the sample acquisition component, wherein the sample acquisition component comprises two or more partially overlapping membranes configured such that one component of the biological sample is collected on a first membrane and the remainder of the sample is collected on a second membrane.

In another aspect, a method is disclosed which involves deploying a tagged sample acquisition and stabilization system to a subject wherein the system includes a blood collection unit configured for collecting a blood sample from the subject, an optional separation module for separating one or more components from the collected blood sample, and a stabilization matrix configured for stabilizing one or more components from the blood sample wherein the tagged sample acquisition and stabilization system comprises a label associated with an assay. The method further includes collecting a deployed tagged sample acquisition and stabilization system, performing one or more assays on the one or more stabilized components based on the tag, and providing a report on the one or more assays performed. The method can further involve separating the stabilization matrix from the system prior to one of the above-mentioned method steps. Optionally, the tag can be on the stabilization matrix. Optionally, when the separation module is present, the tag can be on the optional separation module. The stabilization matrix can be configured to perform multiple assays. The method can further involve identifying stabilized bio-components within the blood sample based on the tag present in a deployed sample acquisition and stabilization system and from that identification determining a stabilized component-specific assay to perform. The sample stabilization system can be labeled with an RFID transmitter, a color label, a barcode, or a second label that differs from the sample tag.

In another aspect, a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize at least one bio-component selected from the group consisting of: RNA, DNA, and protein. The method further involves performing one or more assays on the stabilized bio-components in or on the sampling device at a location remote from where the first method step is carried out. The one or more stabilization reagents can be disposed on or integrated with a solid matrix and can be in a substantially dry state. The solid matrix can be configured to substantially stabilize RNA for at least 11 days at 37 degrees Celsius. The assays can be performed in or on the device, and can include one or more of: PCR, DNA sequencing, RNA expression analysis, and RT-PCR.

In another aspect, a method for performing analysis on one or more bio-components from a bio-sample from a subject is disclosed. The method involves obtaining a sampling device which includes a sample acquisition component and a stabilization matrix comprising one or more stabilizing reagents configured to selectively stabilize the one or more bio-components selected from the group consisting of: RNA, DNA, protein, and a combination thereof. The method further involves removing from the sampling device the stabilization matrix with the one or more bio-components, and performing one or more assays on or directly off of the stabilization matrix. The stabilization matrix can be removed from the sampling device at a location remote from one of the above-mentioned method steps.

In another aspect, a method is disclosed which involves receiving, under ambient conditions, a substantially dry solid matrix comprising one or more stabilization reagents that selectively stabilize one or more bio-components selected from the group consisting of: DNA, RNA and protein, and performing one or more assays directly off of the dry solid matrix for analyzing the selectively stabilized bio-components. The stabilization reagent can include a solid substrate comprising melezitose under a substantially dry state with water content of less than 2%. The stabilization reagent can be coupled to the sample acquisition component comprising a solid matrix for extraction and storage of nucleic acids from the biological sample, wherein RNA obtained from the solid matrix has a RIN of at least 4.

In another aspect, a device for point-of-care or self-administered collection and stabilization of a sample is disclosed and embodiments are illustrated herein. The device includes a sample acquisition component comprising a body and a plunger, wherein the body comprises a proximal end, a distal end, an outer body surface extending between the proximal and distal ends, a base comprising at least one aperture and attached to the distal end, and a lumen defined within the body, wherein the plunger is disposed within the lumen, wherein the plunger comprises a proximal end, a distal end, and at least one needle connected to either end, wherein the at least one needle is configured to pass through the at least one aperture in the base when the plunger is actuated. The device includes a sample stabilization component comprising a reagent and a substrate configured such that components of a bio-sample are extracted and stabilized as the bio-sample migrates through the matrix from a point of collection, wherein the sample stabilization component is disposed within the lumen of the body, such that actuation of the plunger drives the bio-sample through the matrix to further facilitate separation of the bio-sample.

In another aspect, a kit for collecting and stabilizing a sample is disclosed. The kit includes a sample acquisition component with one or more piercing elements configured for penetrating skin to expose blood, and a sample stabilization component, wherein the sample stabilization component comprises a solid substrate comprising melezitose under a substantially dry state. The solid substrate can further include one or more of a lysis reagent and a biomolecule stabilizing reagent. The melezitose can be present at a concentration in a range of 10 to 30%. The solid substrate can be configured for preserving enzyme activity. The kit can further include a plasma separation component.

In some aspects, a handheld user-activable device or method disclosed herein can be configured or capable of collecting at least 150 μL of blood from a subject in less than 3 minutes beginning from time of incision or penetration of a skin portion of the subject.

In some aspects, a device for collecting fluid sample from a subject is provided. The device can comprise a recess and a pre-evacuated vacuum chamber located within the device. The recess can be configured to maintain contact with at least 5.0 cm² of a skin surface area of the subject under vacuum pressure, prior to and as the fluid sample is being collected from the skin of the subject.

In some aspects, a device for collecting fluid sample from a subject can comprise: a housing comprising a recess having an opening; a vacuum chamber in the housing in fluidic communication with the recess; and one or more piercing elements that are extendable through the opening to penetrate skin of the subject. The vacuum chamber can be configured for having a vacuum that draws the skin into the recess, and the recess can be configured having a size or shape that enables an increased volume of the fluid sample to be accumulated in the skin drawn into the recess.

In some aspects, a method for collecting a fluid sample from a subject can comprise: providing a device having a housing, said housing configured to support a vacuum chamber and a piercing module, the housing comprising a recess having an opening; placing the recess of the housing adjacent to skin of the subject; activating the vacuum in the vacuum chamber to draw the skin into the recess; accumulating an increased volume of the fluid sample in the skin drawn into the recess, wherein the recess is configured having a size or shape that enables the increased volume of the fluid sample to be accumulated; extending one or more piercing elements through the opening to penetrate the skin; and maintaining the device adjacent to the skin for a sufficient amount of time to draw the fluid sample into the device.

In some embodiments, the fluid sample can comprise blood from the subject. The recess can serve as a suction cavity for drawing the skin and increasing capillary pressure differential. The increased volume of the fluid sample can depend on a volume and/or surface area of the skin that is drawn into the recess. In some cases, the volume of the skin enclosed by the recess can range from about 0.4 cm³ to about 4.0 cm³. The surface area of the skin in contact with the recess can range from about 3.2 cm² to about 7.2 cm². The increased volume of the fluid sample can depend on a pressure of the vacuum in the vacuum chamber. The pressure of the vacuum in the vacuum chamber can range from about −4 psig to about −15 psig. The increased volume of the fluid sample in the skin drawn into the recess can be at least about 50 μL prior to the penetration of the skin. In some cases, the increased volume of the fluid sample in the skin drawn into the recess, an increased capillary pressure, and with aid of the vacuum, can permit the fluid sample to be drawn from the skin and collected at an average flowrate of at least 30 μL/min. In some cases, the fluid sample can be collected at an average flowrate of at least 100 μL/min. In some cases, the fluid sample can be collected at an average flowrate of at least 150 μL/min. In some cases, the average flowrate can be sustained at least until about 150-300 μL of the fluid sample has been collected. The size and/or shape of the recess can be configured to permit the skin to substantially conform to the recess. A gap between the skin and the recess can be negligible when the skin is drawn into the recess. A surface of the recess can be substantially in contact with the skin drawn into the recess. In some cases, a size of the recess can be at least two times a size of the opening within the recess. In some cases, the size of the opening within the recess can range from about 1.5 mm to about 6 mm, and the size of the recess at its outermost periphery can range from about 10 mm to about 60 mm. A surface area of the recess can be substantially greater than an area of the opening. In some cases, the surface area of the recess can be at least ten times the area of the opening. In some cases, the surface area of the recess can range from about 75 mm² to about 2900 mm², and the area of the opening can range from about 1.5 mm² to about 30 mm². In some cases, an area of the skin directly under the opening can be at least 1.5 times smaller than a total area of the skin drawn into the recess. In some cases, the area of the skin directly under the opening can be at least 5 times smaller than the total area of the skin drawn into the recess.

In some embodiments, the recess can comprise a concave cavity. In some cases, the concave cavity can have a volume ranging from about 1.0 cm³ to about 5.0 cm³. The recess can be in the shape of a spherical cap. In some cases, a base diameter of the spherical cap can range from about 10 mm to about 60 mm, and a height of the spherical cap can range from about 3 mm to about 30 mm. The spherical cap can be a hemisphere. The opening can be at an apex of the spherical-capped recess. In some embodiments, the recess can comprise one or more fillets configured to improve vacuum suction to the skin and reduce vacuum leak. The one or more fillets can extend continuously along a periphery of the recess. The one or more fillets of the recess can be configured to be in contact with the skin when the skin is drawn into the recess.

In some embodiments, a vacuum pressure of at least about −1 psig can be provided in order to draw the skin into and completely fill the recess. In some cases, the skin can be drawn into the recess by the vacuum and can completely fill the recess in less than 1 second. In some cases, the skin can be drawn into the recess by the vacuum and can completely fill the recess in no more than 5 seconds.

In some embodiments, (1) the size or shape of the recess or (2) a pressure of the vacuum can be configured to achieve a minimum capillary pressure in the skin drawn into the recess. In some cases, (1) the size or shape of the recess or (2) a pressure of the vacuum can be configured to achieve a minimum tension in the skin drawn into the recess. The device can be supported and held in place on the skin of the subject with the aid of an adhesive. The device can be supported and held in place on the skin of the subject with the aid of the vacuum. The device can be supported and held in place on the skin of the subject primarily with the aid of the vacuum. The device can be configured for use on an upper portion of the subject's arm. The device can be configured to remain in its position on the subject's arm independent of any movement or changes in orientation of the subject's arm.

In some embodiments, the device can be capable of collecting 250 μL of fluid sample from the subject in less than 1 minute 45 seconds. In some cases, the device can be capable of collecting at least 175 μL to 300 μL of fluid sample from the subject in less than 3 minutes. In some cases, the device can be capable of collecting at least 200 μL of fluid sample from the subject in less than 5 minutes. The device can be configured to collect the fluid sample at a rate that is dependent on the size or shape of the recess and/or vacuum pressure. The recess can be configured having a size and shape that enables an increased volume of the fluid sample to be accumulated in the skin drawn into the recess. The recess can be configured having a size and shape that enables the increased volume of the fluid sample to be accumulated. In some cases, (1) the size and shape of the recess and (2) a pressure of the vacuum can be configured to achieve a minimum capillary pressure in the skin drawn into the recess. In some cases, (1) the size and shape of the recess and (2) a pressure of the vacuum can be configured to achieve a minimum tension in the skin drawn into the recess. The device can be configured to collect the fluid sample at a rate that is dependent on the size and shape of the recess.

In some other aspects, a device for collecting a fluid sample from a subject is provided. The device can comprise: a housing comprising a piercing activator configured to activate one or more skin piercing elements, and a vacuum activator separate from the piercing activator and configured to activate an evacuated vacuum chamber prior to the activation of the one or more piercing elements by the piercing activator.

In some aspects, a method for collecting a fluid sample from a subject can comprise: placing a device packaged with an evacuated vacuum chamber and one or more piercing elements on skin area of the subject; activating the evacuated vacuum chamber to effectuate vacuum pressure on the skin area; piercing the skin area after vacuum activation; and maintaining the vacuum pressure during and after penetrating the skin area of the subject, in order to draw the fluid sample from the skin into device.

In some embodiments, the piercing activator and the vacuum activator can be two separate components. The vacuum activator can comprise a first input interface on the housing, and the piercing activator can comprise a second input interface on the housing. In some cases, at least one of the first input interface or the second input interface can comprise a button. In some alternative cases, the vacuum activator can comprise a first input interface and the piercing activator can comprise a second input interface, and at least one of the first input interface or the second input interface can be remote from the housing.

In some embodiments, the piercing activator can be configured to activate the one or more piercing elements after the skin is drawn into the recess. The piercing activator can be configured to activate the one or more piercing elements after the skin is drawn into the recess by the vacuum for a predetermined length of time. In some cases, the predetermined length of time can range from about 1 second to about 60 seconds. In some embodiments, the housing can comprise the pre-evacuated vacuum chamber, and the vacuum activator can be configured to activate the vacuum in the pre-evacuated vacuum chamber. In some cases, the piercing activator can be configured to activate the one or more piercing elements only after the vacuum has been activated. In some cases, the piercing activator can be locked and incapable of activating the one or more piercing elements prior to activation of the vacuum. The piercing activator can comprise a locking mechanism coupled to the vacuum activator. The locking mechanism can be configured such that the piercing activator is initially in a locked state. The vacuum activator can serve as a key for unlocking the piercing activator, and the piercing activator can be simultaneously unlocked when the vacuum activator is activated. The vacuum activator can be configured to activate the vacuum by establishing fluidic communication to the pre-evacuated vacuum chamber. For example, the vacuum activator can be configured to pierce a foil seal or open a valve to establish the fluidic communication to the pre-evacuated vacuum chamber.

In some embodiments, the vacuum activator can be located on the housing such that the vacuum activator is configured to be pressed in a first direction, and the piercing activator can be located on the housing such that the piercing activator is configured to be pressed in a second direction. In some cases, the first direction and the second direction can be substantially the same. Alternatively, the first direction and the second direction can be substantially different. In some cases, the first direction and the second direction can be substantially parallel to each other. In some cases, at least one of the first direction or the second direction does not extend toward the skin of the subject. For example, the second direction does not extend toward the skin of the subject. In some cases, at least one of the first direction or the second direction can extend substantially parallel to the skin of the subject. In some cases, the first direction and the second direction can both extend substantially parallel to the skin of the subject. In some cases, at least one of the first direction or the second direction can extend in a direction of gravitational force. In some cases, the first direction and the second direction can both extend in the direction of gravitational force. In some embodiments, the piercing activator and the vacuum activator can be located on a same side of the housing, and can be ergonomically accessible by the subject when the device is mounted onto an arm of the subject. For example, the piercing activator can be located on a cover of the housing, and the vacuum activator can be located on a base of the housing where the vacuum chamber is located. Alternatively, the piercing activator and the vacuum activator can be located on different sides of the housing, and can be ergonomically accessible by the subject when the device is mounted onto an arm of the subject.

In some further aspects, a method for collecting a fluid sample from a subject is provided. The method can comprise: with aid of a fluid acquisition device: piercing skin of the subject and delivering the fluid sample from the subject to a matrix disposed within a deposition chamber of the fluid acquisition device, wherein the delivery of the fluid sample is assisted or enhanced using (1) gravitational force, (2) vacuum force, (3) a pressure difference between capillary pressure and internal pressure of the device, and (4) wicking behavior of the fluid sample along the matrix.

In some aspects, a device for collecting a fluid sample from skin of a subject and delivering it to a deposition chamber is provided, wherein fluid flow from the skin to a matrix in the deposition chamber can be preferably enhanced by (1) gravitational force, (2) vacuum force, (3) a pressure differential between capillary pressure and internal pressure of the device, and (4) wicking behavior of the fluid sample along the matrix.

In some embodiments, the device can comprise an enclosure for holding one or more piercing elements, and the enclosure can be in fluidic communication with the deposition chamber. The deposition chamber and the enclosure can be initially at ambient pressure, prior to activation of a vacuum from a pre-evacuated vacuum chamber located onboard the device. In some cases, the deposition chamber, the vacuum chamber, and the enclosure can be configured to equalize to an internal pressure that is less than the ambient pressure after the vacuum has been activated. The internal pressure can be higher than the initial evacuated vacuum pressure of the vacuum chamber. In some cases, the internal pressure can be about −5.5 psig, and the sealed vacuum pressure can be about −12 psig. The internal pressure can be configured to draw the skin into a recess of the housing. The internal pressure can be configured to draw blood from capillary beds to the skin that is being drawn into the recess. A pressure differential can be created between capillary pressure and the internal pressure when the skin is penetrated by one or more piercing elements of the device. The internal pressure can increase as the fluid sample is drawn from the skin towards the deposition chamber and the enclosure. In some cases, the internal pressure in the enclosure can increase more rapidly compared to a collective internal pressure of the deposition chamber and the vacuum chamber. The internal pressure in the enclosure can increase substantially more than the collective internal pressure of the deposition chamber and the vacuum chamber. The substantially increased internal pressure of the enclosure can inhibit the flow of the fluid sample into the enclosure. The substantially increased internal pressure of the enclosure can result in preferential flow of the fluid sample towards the pressure of the enclosure can cause the flow of the fluid sample into the enclosure to slow or stop, while the fluid sample can continue to flow towards the deposition chamber under the influence of the pressure differential. In some cases, (1) a volume of the enclosure and (2) a collective volume of the deposition chamber and the vacuum chamber, can be configured such that minimal amounts of the fluid sample flows towards and into the enclosure. In some cases, a ratio of the volume of the enclosure to the collective volume of the deposition chamber and the vacuum chamber can range from about 1:5 to about 1:15. In some cases, the one or more piercing elements can be configured to penetrate the skin to generate cuts, and the pressure differential can enable deeper cuts and the cuts to be held open under tension. The pressure differential can be configured to increase the size of the cuts to enable a higher flowrate and volume of the fluid sample to be collected from the skin.

In some further aspects, a device for penetrating skin of a subject is provided. The device can comprise: one or more piercing elements supported by a piercing holder movable by two or more spring elements; a deployment spring positioned to deploy the one or more piercing elements through an opening in the device; and a retraction spring positioned to retract the one or more piercing elements back into the device, wherein a length of the one or more piercing elements is less than about 20 mm, and the depth of penetration of the one or more piercing elements is about 2 mm. In some cases, the length of the one or more piercing elements is about 12.7 mm.

In some aspects, a method for penetrating skin of a subject can comprise providing the aforementioned device; drawing the skin of the subject into a recess of the device; activating the deployment spring and deploying the one or more piercing elements through the opening in the device; penetrating the skin of the subject using the one or more piercing elements; and using the retraction spring to retract the one or more spring elements back into the device.

In some embodiments, two or more piercing elements can be supported by a holder in a random configuration. In some cases, the two or more piercing elements can have random orientations relative to each other. The two or more piercing elements can comprise beveled edges that are randomly oriented relative to each other. The beveled edges of the two or more piercing elements can be non-symmetrical to each other. The beveled edges of the two or more piercing elements can be at an acute or oblique angle relative to each other.

In some cases, two or more piercing elements can be supported by a holder in a predefined configuration. The two or more piercing elements can have predefined orientations relative to each other. The two or more piercing elements can comprise beveled edges that are oriented relative to each other in a predefined manner. The beveled edges of the two or more piercing elements can be symmetrical to each other.

In some embodiments, the piercing elements can comprise two or more lancets. Optionally, the piercing elements can comprise needles and/or microneedles. In some cases, two or more lancets can have a same bevel angle. Alternatively, two or more lancets can have different bevel angles. In some cases, the bevel angle(s) can range from about 10 degrees to about 60 degrees. In some cases, the two or more lancets can comprise beveled faces having a same bevel length. Alternatively, the two or more lancets can comprise beveled faces having different bevel lengths. In some cases, the bevel length(s) can range from about 2 mm to about 10 mm.

In some embodiments, two or more piercing elements can be configured to generate cuts on the skin that extend in different directions along the skin and that are non-parallel to each other.

In some embodiments, the deployment spring can be configured to move and cause the piercing elements to penetrate the skin of the subject at speeds ranging from about 0.5 m/s to about 2.0 m/s. The deployment spring can be configured to move and cause the piercing elements to penetrate the skin of the subject with a force ranging from about 1.3 N to about 24.0 N. A spring-force of the retraction spring can be less than a spring-force of the deployment spring. In some cases, the deployment spring can have a spring-rate of about 2625 N/m, and the retraction spring can have a spring-rate of about 175 N/m. The deployment spring can be configured to cause the one or more piercing elements to penetrate the skin to depths ranging from about 0.5 mm to about 3 mm. The retraction spring can be configured to retract the piercing elements from the skin of the subject at speeds ranging from about 0.1 m/s to about 1.0 m/s.

In some embodiments, the device can further comprise: a vacuum activator configured to activate a vacuum for drawing the skin into a recess of the device. In some cases, a piercing activator can be configured to activate the deployment spring only after the vacuum activator is activated.

In some further aspects, a device for monitoring fluid sample collection from a subject is provided. The device can comprise: a housing comprising a cartridge chamber; a cartridge operably coupled to the cartridge chamber; components for penetrating skin of the subject and drawing the fluid sample from the skin into the cartridge; and a flow meter on the housing that enables the subject or a user to monitor a progress of the fluid sample collection in real-time as the fluid sample is collected into the cartridge.

In some aspects, a method for monitoring fluid sample collection from a subject can comprise: providing (1) a housing comprising a cartridge chamber, (2) a cartridge operably coupled to the cartridge chamber, (3) components for penetrating skin of the subject and drawing the fluid sample from the skin into the cartridge, and (4) a flow meter on the housing; and monitoring, with aid of the flow meter, a progress of the fluid sample collection in real-time as the fluid sample is collected into the cartridge.

In some embodiments, the flow meter can be provided on a lid covering a base of the housing. The flow meter is not obscured by a cover of the housing. The flow meter can be in proximity to the cartridge chamber. The flow meter can be substantially aligned with a cartridge located within the cartridge chamber. In some embodiments, the flow meter can comprise a plurality of windows disposed parallel to a longitudinal axis of the cartridge. The plurality of windows can be made of an optically transparent material. The fluid sample can be visible through the windows and sequentially fills each window as the fluid sample is being collected into the cartridge. Each window can be indicative of a known amount of fluid sample that is collected. The fluid sample collection is complete when the fluid sample is visible in all of the windows. The plurality of windows can comprise three or more windows.

In some embodiments, the flow meter can comprise a single window disposed parallel to a longitudinal axis of the cartridge. The window can be made of an optically transparent material. The fluid sample can be visible through the window and continuously fills the window as the fluid sample is being collected into the cartridge. The fluid sample collection is complete when the fluid sample is visible throughout the window.

In some further aspects, a cartridge assembly is provided. The cartridge assembly can comprise: a cartridge for holding one or more matrices for storing a fluid sample thereon; a cartridge holder releasably coupled to the cartridge, wherein the cartridge assembly is releasably coupled to a device used for collecting the fluid sample.

In some embodiments, a device for collecting a fluid sample from a subject is provided. The device can comprise: a housing comprising a deposition chamber and a pre-evacuated vacuum chamber, wherein the deposition chamber is configured to receive and releasably couple to the cartridge assembly, and the deposition chamber is in fluidic communication with the vacuum chamber.

In some embodiments, a fluid sample collection kit can comprise the device and the cartridge assembly. In some embodiments, a fluid sample collection assembly can comprise the device and the cartridge assembly releasably coupled to said device. In some embodiments, an input port of the cartridge can be releasably coupled to and in fluidic communication with a channel of the device, and the fluid sample can be collected from penetrated skin of the subject and transported through the channel into the cartridge.

In some embodiments, a method for collecting a fluid sample from a subject can comprise: releasably coupling the cartridge assembly to the device; placing the device adjacent to skin of the subject; activating vacuum in the vacuum chamber to draw the skin into a recess of the housing; using one or more piercing elements of the device to penetrate the skin; maintaining the device adjacent to the skin for a sufficient amount of time to draw the fluid sample into the device and collect the fluid sample into the cartridge; and decoupling the cartridge assembly from the device after a certain amount of the fluid sample has been collected in the cartridge.

In some embodiments, the cartridge holder can be releasably coupled to the cartridge via a quick release mechanism. In some cases, the quick release mechanism can comprise one or more spring-clips on the cartridge holder. The cartridge assembly can be capable of being coupled to and detached from the deposition chamber without use of tools. The cartridge assembly can be capable of being coupled to and detached from the deposition chamber using no more than two motion steps. The cartridge assembly can be coupled to the deposition chamber prior to the collection of the fluid sample from the subject. The cartridge assembly can be decoupled from the deposition chamber after the fluid sample from the subject has been collected into the cartridge.

In some embodiments, the cartridge can comprise two or more matrices for collecting and storing the fluid sample thereon. The two or more matrices can be disposed in a configuration that permits the fluid sample to wick between and along the two or more matrices. For example, the two or more matrices can be disposed substantially parallel to each other. In some cases, the two or more matrices can be separated by a gap of about 0.5 mm. In some cases, at least one of the matrices can be capable of collecting at least 60 μL of fluid sample. In some cases, each of two or more matrices can be capable of collecting at least 60 μL of fluid sample.

In some embodiments, the cartridge can further comprise one or more absorbent pads configured to be in fluidic communication with the one or more matrices, wherein the one or more absorbent pads can be used to hold excess fluid sample. The one or more absorbent pads can aid in ensuring that a predefined volume of the fluid sample can be collected and maintained on the one or more matrices, regardless of an input volume of the fluid sample into the cartridge up to a predefined range. In some cases, the one or more matrices can include two matrices that are each configured to hold up to about 7 μL of the fluid sample. Each of the two matrices can be configured to hold and maintain about 75 μL of the fluid sample as the input volume of the fluid sample to the cartridge increases beyond 150 μL up to the predefined range. In some cases, the predefined range can be from about 150 μL to about 300 μL. In other cases, the predefined range can be greater than 300 μL. In some cases, the one or more absorbent pads can be capable of holding at least 100 μL of excess fluid sample.

In some embodiments, the cartridge holder can comprise a cartridge tab that is configured to be releasably coupled to a distal end of the deposition chamber. The cartridge tab can be configured such that the subject or a user is able to (1) support the cartridge assembly by holding the cartridge tab, (2) couple the cartridge assembly to the device by pushing the cartridge tab, and/or (3) decouple the cartridge assembly from the device by pulling the cartridge tab.

In some further aspects, a transportation sleeve is provided. The sleeve can comprise: an opening configured to couple to a cartridge tab included with the cartridge; and a dual support-release mechanism within the sleeve, wherein the dual support-release mechanism can comprise: (a) a retention element configured to engage with a corresponding mating feature on the cartridge and secure the cartridge within the sleeve, and (b) a release element configured to cause the spring-clips on the cartridge holder to release and thereby decouple the cartridge from the cartridge holder. The dual support-release mechanism can permit the cartridge holder to be removed from the opening of the sleeve while the cartridge is secured in place within the sleeve, without exposure of the strips to the ambient environment. In some cases, the transportation sleeve can further comprise a desiccant within the sleeve. In some cases, the sleeve can be sized and shaped to accommodate user or patient identity (ID) labels.

In some embodiments, a transportation assembly can comprise: the transportation sleeve, and the cartridge coupled to said transportation sleeve. In some cases, the cartridge tab can be configured to hermetically seal the opening of the sleeve.

In some embodiments, the cartridge can be oriented such that the flow of the fluid sample into the cartridge is further aided with gravity. In some cases, the cartridge can comprise a luer-type fitting that can engage with the device when the cartridge is inserted into the deposition chamber.

In some embodiments, the one or more matrices can comprise absorbent paper. In some cases, one or more of the matrices can comprise stabilization chemistry. In some cases, a first matrix can comprise a first stabilization chemistry and a second matrix can comprise a second stabilization chemistry different from the first stabilization chemistry. In some alternative cases, one or more of the matrices does not comprise stabilization chemistry.

Provided herein are medical systems, devices, and methods for sample collection and storage. The disclosed systems, devices, and methods can comprise structure features that facilitate sample collection (e.g. blood collection devices) as well as components for collecting blood sample on to substrate for storage and transport.

Any of the devices disclosed herein can rely on the generation of a vacuum to apply negative pressure to deform the skin of a subject and to apply local suction directly to the sample collection site, thereby facilitating sample flow and collection. Any of the devices disclosed herein can comprise a concave cavity that can be placed at the surface of the skin of the subject, this concave cavity can be configured to deliver vacuum (e.g. negative pressure, suction etc.) to the skin of the subject. Any of the devices disclosed herein can comprise an opening disposed at the apex of or other surface of the concave cavity, the inner diameter can be configured to allow a piercing element to pierce the skin of the subject; and a piercing element can be configured to pass through the inner diameter. Local suction can be applied to the sample collection site through the inner diameter.

In some embodiments, a vacuum can be configured to deform the skin of the subject using different mechanisms, for example the vacuum can be configured to draw the skin of the subject into the concave cavity. A concave cavity can be configured to constrain the surface of the skin against its entire or a portion of its concave surface of the subject at which point the piercing element can be configured to pierce the skin of the subject. An opening contiguous with a cylinder (e.g. a cylinder in fluid contact with a cartridge) can be configured to draw the blood from the subject into the device when the vacuum is applied to the skin of the subject and after an incision has been made in the skin of the subject.

Vacuum pressure can be generated using an evacuated vacuum chamber configured such that activation of the device pierces the evacuated vacuum chamber forming negative pressure that draws the blood from the subject through the opening and channels and into a cartridge and onto a solid matrix for sample storage vacuum pressures can be in the range of between 1-20 psi. The vacuum pressure can be about 5 psi. Vacuum chamber volume can be within a 10%-100% margin of twice the volume of the combined concave cavity, opening, channel and cartridge volume. Any of the devices disclosed herein can comprise a vacuum activation actuator configured to activate the vacuum upon actuation of the vacuum activation actuator. The vacuum activation actuator can comprise a button.

Any of the devices disclosed herein can be configured for drawing a specific volume (e.g. greater than 20 μL, greater than 40 μL, greater than 60 μL, greater than 80 μL, greater than 100 μL, greater than 150 μL, or greater than 200 μL) of blood (e.g. capillary blood) from a subject in defined period of time (e.g. less than 4 minutes), can have specific vacuum and device parameters. The structure of the concave cavity can have an impact on blood collection, for example the rate of blood sample collection can be dependent on the curvature and size of the concave cavity and the vacuum pressure.

To facilitate blood collection the surface area acted on by the vacuum can have specific parameters, for example the surface area of the skin under vacuum and in contact with the concave cavity can be within a 10% margin of 500 mm² and the opening in fluid contact with a cylinder (e.g. a cylinder in fluid contact with a cartridge) can have a diameter can be within a 10% margin of 8 mm². Any of the devices, systems and methods herein for collecting sample (e.g. blood samples) can be configured with a removable cartridge. The removable cartridge can be held in fluid communication with the cylinder (e.g. the cylinder in contact with the opening in the concave cavity). Any of the devices disclosed herein can comprise a visual metering window configured to permit visualization of the removable cartridge while the removable cartridge is in the device. Any of the devices disclosed herein can comprise a piercing module, wherein the piercing module comprises one or more piercing elements. The piercing elements can be actuated with a button. Before and after actuation, the piercing element can be withdrawn when the piercing element is in an unactivated state.

Also disclosed herein are cartridges configured to collect sample from the device and transfer it to solid substrate such that precise volumes of sample are collected on and metered by the absorbency of the solid substrate. For example, the standardized quantity of blood saturating each strip of the substrate can be within the range of 50-100 μL on a substrate with surface area within the range of 100-300 square millimeters. A cartridge can comprise a channel disposed between two strips of substrate configured for transferring a blood sample to the two strips of substrate. A cartridge can comprise a spacer disposed between a portion of each of the two strips of substrate. A spacer can be configured to adjust the space between the two strips of substrate depending on one or more conditions. Cartridges can be removable from the device, for example using methods to clip the cartridge into place. Cartridges can further comprise a wicking tail. A wicking tail can be configured for standardizing the quantity of blood saturated on the two strips of substrate.

Collecting standardized quantities of blood on substrate of specific surface area can be performed using various methods. Methods for applying blood to at least two solid supports can comprise the steps of providing a cartridge comprising at least two solid supports. The provided cartridge can comprise at least two solid supports are substantially the same size, such that a surface of each of the at least two solid supports face each other and the surface at the least two solid supports are substantially parallel to each other. The at least two solid supports can be separated by a defined distance (e.g. within 10% margin of 0.4 mm), and the cartridge can be configured so that a channel is formed between the two solid supports. Blood can be passing into the tunnel between the at least two solid supports, wherein the blood is absorbed to each of the at least two solid supports as it passes through the tunnel between the at least two solid supports. Solid supports used in these methods can comprise fixed dimensions (e.g. width between 3 mm and 10 mm and length between 3 mm and 26 mm). The cartridge used in the method can further comprise a wicking element configured for metering blood flow through the device.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

The present invention generally relates to medical systems, devices and methods, and more particularly relates to a system and methods for user-mediated collection, separation, and stabilization of a blood sample. Specifically, the herein disclosed system, device and methods provide for an integrated device comprising a sample acquisition component, a sample separation component and a blood stabilization component incorporated within an integrated device configured to collect blood from a subject upon actuation, and stabilize the blood for further processing within or outside the device.

These and other embodiments are described in further detail in the following description related to the appended drawing figures.

In one aspect, a method is disclosed. The method involves deploying a sample acquisition and stabilization system with a tag to a subject wherein the system includes a blood collection unit configured for collecting a blood sample from the subject, an optional separation module for separating one or more components from the collected blood sample, and a stabilization matrix configured for stabilizing the one or more components from the collected blood sample wherein the tag comprises a label associated with an assay. The method further involves collecting the deployed tagged sample acquisition and stabilization system, performing one or more assays on the one or more stabilized components based on the tag, and providing a report on the one or more assays performed. The method can further involve separating the stabilization matrix from the system prior to one of the aforementioned method steps. The tag can be on the stabilization matrix. Optionally, when the separation module is present, the tag can be on the optional separation module. The stabilization matrix can be configured to perform multiple assays. The method can further involve identifying stabilized bio-components within the blood sample based on the tag present in a deployed sample acquisition and stabilization system and from that identification determining a stabilized component-specific assay to perform. The sample acquisition and stabilization system can be labeled with an RFID transmitter, a color label, a barcode, or a second label that differs from the sample tag.

In another aspect, a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize at least one bio-component selected from the group consisting of: RNA, DNA, and protein. The method further involves performing one or more assays on the at least one stabilized bio-component in or on the sampling device at a location remote from one of the above-mentioned method steps. The one or more stabilization reagents can be disposed on or are integrated with a solid matrix and are in a substantially dry state. The solid matrix can be configured to substantially stabilize RNA for at least 11 days at 37 degrees Celsius. The assays performed in or on the device can include one or more of: PCR, DNA sequencing, RNA expression analysis, and RT-PCR.

In another aspect a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize one or more bio-components selected from the group consisting of: RNA, DNA, and protein. The method further involves removing from the sampling device the stabilization matrix with the one or more bio-components; and performing one or more assays on the stabilization matrix. The stabilization matrix can be removed from the sampling device at a location remote from where one of the above-mentioned steps is carried out.

In another aspect, a method is disclosed which involves receiving, under ambient conditions, a substantially dry solid matrix comprising one or more stabilization reagents that selectively stabilize one or more bio-components selected from the group consisting of: DNA, RNA and protein, and performing one or more assays directly off of the dry solid matrix for analyzing the one or more selectively stabilized bio-components. The stabilization reagent can include a solid substrate comprising melezitose under a substantially dry state with water content of less than 2%. The stabilization reagent can be coupled to a sample acquisition component which includes a solid matrix for extraction and storage of nucleic acids from the biological sample, wherein the solid matrix has an RNA Integrity Number (RIN) of at least 4.

In another aspect, a device for point-of-care or self-administered collection and stabilization of a bio-sample is disclosed and embodiments are illustrated herein. The device includes a sample acquisition component comprising a body and a plunger, wherein the body comprises a proximal end, a distal end, an outer body surface extending between the proximal and distal ends, a base comprising at least one aperture and attached to the distal end, and a lumen defined within the body surface, wherein the plunger is disposed within the lumen, wherein the plunger comprises a proximal end, a distal end, and at least one needle connected to either end, wherein the at least one needle is configured to pass through the at least one aperture in the base when the plunger is actuated. The device includes a sample stabilization component which includes a reagent and a substrate configured such that components of a bio-sample are extracted and stabilized as the bio-sample migrates through the matrix from a point of collection; wherein the sample stabilization component is disposed within the lumen of the body such that actuation of the plunger drives the bio-sample through the matrix to further facilitate separation of the bio-sample.

In another aspect, a kit for collecting and stabilizing a sample is disclosed. The kit includes a sample acquisition component with one or more piercing elements configured for penetrating skin to expose blood, and a sample stabilization component, wherein the sample stabilization component comprises a solid substrate comprising melezitose under a substantially dry state. The solid substrate can include one or more of a lysis reagent and a biomolecule stabilizing reagent. Optionally, the melezitose can be present at a concentration in a range of 10 to 30%. The solid substrate can be configured for preserving enzyme activity. The kit can further include a plasma separation component.

In another aspect, a kit for collecting and stabilizing a biological sample is disclosed. The kit includes a sample acquisition component with one or more piercing elements configured for penetrating skin to expose blood, and a sample stabilization component comprising a solid matrix for selectively stabilizing one or more of the following bio-components: RNA, DNA, and protein. The solid matrix can selectively stabilize RNA and RNA obtained from the solid matrix can have a RIN number greater than 4. The kit can further include a plasma separation component. RNA obtained from the solid matrix can have a RIN of at least 5 or of at least 6, or of at least 7, or have a RIN of greater than 7. The solid matrix can further include at least one protein denaturant present in the solid matrix in a dry state. The solid matrix can further include at least one reducing agent in the solid matrix in a dry state. The solid matrix can further include at least one UV protectant in the solid matrix in a dry state. The solid matrix can further include at least one buffer present in the solid matrix in a dry state. The buffer can be an acid-titrated buffer reagent that generates a pH in a range from 3 to 6. The solid matrix can be comprised of cellulose, cellulose acetate, glass fiber, or a combination thereof. The solid matrix can be a porous matrix. The solid matrix can be a non-dissolvable dry solid material. The solid matrix can be configured to provide an acidic pH upon hydration. The solid matrix can be configured to extract nucleic acids from a sample, and preserve the nucleic acids in a substantially dry state at ambient temperature. The sample acquisition component can be connected to the sample stabilization component.

In another aspect, a system for collecting and stabilizing a sample is disclosed. The system includes a sample acquisition component with one or more piercing elements configured for penetrating skin to expose blood, and a sample stabilization component coupled to the sample acquisition component, wherein the sample stabilization component comprises a solid substrate comprising melezitose under a substantially dry state with a water content of less than 2%. The solid substrate can include a lysis reagent, one or more biomolecule stabilizing reagents, or a combination thereof. The concentration of melezitose can be present in a range of 10 to 30%. The solid substrate can be configured for preserving enzyme activity.

In another aspect, a system for collecting and stabilizing a biological sample is disclosed. The system includes a sample acquisition component with one or more piercing elements configured for penetrating skin to expose blood, and a sample stabilization component coupled to the sample acquisition component comprising a solid matrix for extraction and storage of a nucleic acid from the biological sample, wherein RNA obtained from the solid matrix has a RIN of at least 4. RNA obtained from the solid matrix can have a RIN of at least 5, or of at least 6, or of at least 7, or can be greater than 7. The solid matrix can further include at least one protein denaturant present in the solid matrix in a dry state. The solid matrix can further include at least one reducing agent in the solid matrix in a dry state. The solid matrix can further include at least one UV protectant in the solid matrix in a dry state. The solid matrix can further include at least one buffer disposed on or impregnated within the solid matrix, wherein the solid matrix is substantially dry with a water content of less than 2%. The buffer can be an acid-titrated buffer reagent that generates a pH in a range from 3 to 6. The solid matrix can be comprised of cellulose, cellulose acetate, glass fiber, or a combination thereof. The solid matrix can also be a porous matrix. The solid matrix can be a non-dissolvable dry solid material. The solid matrix can be configured to provide an acidic pH upon hydration. The solid matrix can be configured to extract a nucleic acid from a sample, and preserve the nucleic acid in a substantially dry state at ambient temperature. The sample acquisition component can be connected to the sample stabilization component.

In another aspect, a system for collecting and stabilizing a biological sample is disclosed. The system includes a sample acquisition component with one or more piercing elements configured for penetrating skin to expose blood, and a sample separation component coupled to the sample acquisition component, wherein the sample acquisition component comprises two or more partially overlapping membranes configured such that one component of the biological sample is collected on a first membrane and the remainder of the sample is collected on a second membrane.

In another aspect, a method is disclosed which involves deploying a tagged sample acquisition and stabilization system to a subject wherein the system includes a blood collection unit configured for collecting a blood sample from the subject, an optional separation module for separating one or more components from the collected blood sample, and a stabilization matrix configured for stabilizing one or more components from the blood sample wherein the tagged sample acquisition and stabilization system comprises a label associated with an assay. The method further includes collecting a deployed tagged sample acquisition and stabilization system, performing one or more assays on the one or more stabilized components based on the tag, and providing a report on the one or more assays performed. The method can further involve separating the stabilization matrix from the system prior to one of the above-mentioned method steps. Optionally, the tag can be on the stabilization matrix. Optionally, when the separation module is present, the tag can be on the optional separation module. The stabilization matrix can be configured to perform multiple assays. The method can further involve identifying stabilized bio-components within the blood sample based on the tag present in a deployed sample acquisition and stabilization system and from that identification determining a stabilized component-specific assay to perform. The sample stabilization system can be labeled with an RFID transmitter, a color label, a barcode, or a second label that differs from the sample tag.

In another aspect, a method for performing analysis on one or more bio-components from a bio-sample from a subject is disclosed. The method involves obtaining a sampling device which includes a sample acquisition component and a stabilization matrix which includes one or more stabilizing reagents configured to selectively stabilize at least one bio-component selected from the group consisting of: RNA, DNA, and protein. The method further involves performing one or more assays on the stabilized bio-components in or on the sampling device at a location remote from where the first method step is carried out. The one or more stabilization reagents can be disposed on or integrated with a solid matrix and can be in a substantially dry state. The solid matrix can be configured to substantially stabilize RNA for at least 11 days at 37 degrees Celsius. The assays can be performed in or on the device, and can include one or more of: PCR, DNA sequencing, RNA expression analysis, and RT-PCR.

In another aspect, a method for performing analysis on one or more bio-components from a bio-sample from a subject is disclosed. The method involves obtaining a sampling device which includes a sample acquisition component and a stabilization matrix comprising one or more stabilizing reagents configured to selectively stabilize the one or more bio-components selected from the group consisting of: RNA, DNA, protein, and a combination thereof. The method further involves removing from the sampling device the stabilization matrix with the one or more bio-components, and performing one or more assays on or directly off of the stabilization matrix. The stabilization matrix can be removed from the sampling device at a location remote from one of the above-mentioned method steps.

In another aspect, a method is disclosed which involves receiving, under ambient conditions, a substantially dry solid matrix comprising one or more stabilization reagents that selectively stabilize one or more bio-components selected from the group consisting of: DNA, RNA and protein, and performing one or more assays directly off of the dry solid matrix for analyzing the selectively stabilized bio-components. The stabilization reagent can include a solid substrate comprising melezitose under a substantially dry state with water content of less than 2%. The stabilization reagent can be coupled to the sample acquisition component comprising a solid matrix for extraction and storage of nucleic acids from the biological sample, wherein RNA obtained from the solid matrix has a RIN of at least 4.

In another aspect, a device for point-of-care or self-administered collection and stabilization of a sample is disclosed and embodiments are illustrated herein. The device includes a sample acquisition component comprising a body and a plunger, wherein the body comprises a proximal end, a distal end, an outer body surface extending between the proximal and distal ends, a base comprising at least one aperture and attached to the distal end, and a lumen defined within the body, wherein the plunger is disposed within the lumen, wherein the plunger comprises a proximal end, a distal end, and at least one needle connected to either end, wherein the at least one needle is configured to pass through the at least one aperture in the base when the plunger is actuated. The device includes a sample stabilization component comprising a reagent and a substrate configured such that components of a bio-sample are extracted and stabilized as the bio-sample migrates through the matrix from a point of collection, wherein the sample stabilization component is disposed within the lumen of the body, such that actuation of the plunger drives the bio-sample through the matrix to further facilitate separation of the bio-sample.

In another aspect, a kit for collecting and stabilizing a sample is disclosed. The kit includes a sample acquisition component with one or more piercing elements configured for penetrating skin to expose blood, and a sample stabilization component, wherein the sample stabilization component comprises a solid substrate comprising melezitose under a substantially dry state. The solid substrate can further include one or more of a lysis reagent and a biomolecule stabilizing reagent. The melezitose can be present at a concentration in a range of 10 to 30%. The solid substrate can be configured for preserving enzyme activity. The kit can further include a plasma separation component.

The present disclosure addresses at least the above needs. Various embodiments of the present disclosure address the demand for devices and methods, that enable individuals to easily, conveniently, and reliably collect and store blood samples outside of traditional healthcare facilities, for example in their own homes, in remote locations, while traveling, etc. Individuals who have minimal to no medical training can use the disclosed devices and methods to efficiently collect and store blood on their own or with the help of others, without the need for trained healthcare personnel. The embodiments described herein can obviate the need for individuals to schedule, or make special or frequent trips to healthcare facilities for blood sample collection, which helps to free up the individuals' time and reduce patient load on healthcare resources. Nonetheless, it should be appreciated that the disclosed devices and methods are also suitable for use by healthcare or non-healthcare personnel in a variety of environments or applications, for example in personalized point-of-care (POC), Emergency Medical Services (EMS), ambulatory care, hospitals, clinics, emergency rooms, patient examination rooms, acute care patient rooms, field environments, nurse's offices in educational settings, occupational health clinics, surgery or operation rooms, etc.

Blood samples collected using the devices and methods described herein can be analyzed to determine a person's physiological state, for detecting diseases and also for monitoring the health conditions of the user. In some instances, individuals can rapidly evaluate their physiological status since blood samples can be quickly collected using the devices and methods described herein, and either (1) analyzed on the spot using for example immunoassays or (2) shipped promptly to a testing facility. The reduced lead-time for blood collection, analysis and quantification can be beneficial to many users, especially users who have certain physiological conditions/diseases that require constant and frequent blood sample collection/monitoring. Taking diabetes as an example, hemoglobin A1c (HbA1c) can make up 60% of all glycohemoglobins and can be used for monitoring glycemic control. The amount of HbA1c, as a percentage of total hemoglobin, can reflect the average blood glucose concentration in a patient's blood over the preceding 120 days. Generally it is recommended that diabetic patients test their HbA1c levels every three to six months. The glycemic recommendation for non-pregnant adults with diabetes can be <7.0%, while HbA1c levels of ≥8% can indicate that medical action can be required to control diabetic complications, including cognitive impairment and hypoglycemic vulnerability.

The various embodiments described herein are capable of drawing blood at increased flowrates and higher sample volumes beginning from time of skin incision, compared to traditional non-venous blood collection devices and method. The disclosed devices and methods can be used to collect blood samples of predefined volumes, for example through the use of custom matrices for sample collection, and absorbent pads for holding and metering out excess blood. Additionally, the blood collection devices and methods described herein are minimally invasive and permit lower levels of pain (or perception of pain) in a subject, which can help to improve the overall blood collection experience for the subject.

In some aspects, a handheld user-activable device or method disclosed herein can be configured or capable of collecting at least 150 μL of blood from a subject in less than 3 minutes beginning from time of incision or penetration of a skin portion of the subject.

In some aspects, a device for collecting fluid sample from a subject is provided. The device can comprise a recess and a pre-evacuated vacuum chamber located within the device. The recess can be configured to maintain contact with at least 5.0 cm² of a skin surface area of the subject under vacuum pressure, prior to and as the fluid sample is being collected from the skin of the subject.

In some aspects, a device for collecting fluid sample from a subject can comprise: a housing comprising a recess having an opening; a vacuum chamber in the housing in fluidic communication with the recess; and one or more piercing elements that are extendable through the opening to penetrate skin of the subject. The vacuum chamber can be configured for having a vacuum that draws the skin into the recess, and the recess can be configured having a size or shape that enables an increased volume of the fluid sample to be accumulated in the skin drawn into the recess.

In some aspects, a method for collecting a fluid sample from a subject can comprise: providing a device having a housing, said housing configured to support a vacuum chamber and a piercing module, the housing comprising a recess having an opening; placing the recess of the housing adjacent to skin of the subject; activating the vacuum in the vacuum chamber to draw the skin into the recess; accumulating an increased volume of the fluid sample in the skin drawn into the recess, wherein the recess is configured having a size or shape that enables the increased volume of the fluid sample to be accumulated; extending one or more piercing elements through the opening to penetrate the skin; and maintaining the device adjacent to the skin for a sufficient amount of time to draw the fluid sample into the device.

In some embodiments, the fluid sample can comprise blood from the subject. The recess can serve as a suction cavity for drawing the skin and increasing capillary pressure differential. The increased volume of the fluid sample can depend on a volume and/or surface area of the skin that is drawn into the recess. In some cases, the volume of the skin enclosed by the recess can range from about 0.4 cm³ to about 4.0 cm³. The surface area of the skin in contact with the recess can range from about 3.2 cm² to about 7.2 cm². The increased volume of the fluid sample can depend on a pressure of the vacuum in the vacuum chamber. The pressure of the vacuum in the vacuum chamber can range from about −4 psig to about −15 psig. The increased volume of the fluid sample in the skin drawn into the recess can be at least about 50 μL prior to the penetration of the skin. In some cases, the increased volume of the fluid sample in the skin drawn into the recess, an increased capillary pressure, and with aid of the vacuum, can permit the fluid sample to be drawn from the skin and collected at an average flowrate of at least 30 μL/min. In some cases, the fluid sample can be collected at an average flowrate of at least 100 μL/min. In some cases, the fluid sample can be collected at an average flowrate of at least 150 μL/min. In some cases, the average flowrate can be sustained at least until about 150-300 μL of the fluid sample has been collected. The size and/or shape of the recess can be configured to permit the skin to substantially conform to the recess. A gap between the skin and the recess can be negligible when the skin is drawn into the recess. A surface of the recess can be substantially in contact with the skin drawn into the recess. In some cases, a size of the recess can be at least two times a size of the opening within the recess. In some cases, the size of the opening within the recess can range from about 1.5 mm to about 6 mm, and the size of the recess at its outermost periphery can range from about 10 mm to about 60 mm. A surface area of the recess can be substantially greater than an area of the opening. In some cases, the surface area of the recess can be at least ten times the area of the opening. In some cases, the surface area of the recess can range from about 75 mm² to about 2900 mm², and the area of the opening can range from about 1.5 mm² to about 30 mm². In some cases, an area of the skin directly under the opening can be at least 1.5 times smaller than a total area of the skin drawn into the recess. In some cases, the area of the skin directly under the opening can be at least 5 times smaller than the total area of the skin drawn into the recess.

In some embodiments, the recess can comprise a concave cavity. In some cases, the concave cavity can have a volume ranging from about 1.0 cm³ to about 5.0 cm³. The recess can be in the shape of a spherical cap. In some cases, a base diameter of the spherical cap can range from about 10 mm to about 60 mm, and a height of the spherical cap can range from about 3 mm to about 30 mm. The spherical cap can be a hemisphere. The opening can be at an apex of the spherical-capped recess. In some embodiments, the recess can comprise one or more fillets configured to improve vacuum suction to the skin and reduce vacuum leak. The one or more fillets can extend continuously along a periphery of the recess. The one or more fillets of the recess can be configured to be in contact with the skin when the skin is drawn into the recess.

In some embodiments, a vacuum pressure of at least about −1 psig can be provided in order to draw the skin into and completely fill the recess. In some cases, the skin can be drawn into the recess by the vacuum and can completely fill the recess in less than 1 second. In some cases, the skin can be drawn into the recess by the vacuum and can completely fill the recess in no more than 5 seconds.

In some embodiments, (1) the size or shape of the recess or (2) a pressure of the vacuum can be configured to achieve a minimum capillary pressure in the skin drawn into the recess. In some cases, (1) the size or shape of the recess or (2) a pressure of the vacuum can be configured to achieve a minimum tension in the skin drawn into the recess. The device can be supported and held in place on the skin of the subject with the aid of an adhesive. The device can be supported and held in place on the skin of the subject with the aid of the vacuum. The device can be supported and held in place on the skin of the subject primarily with the aid of the vacuum. The device can be configured for use on an upper portion of the subject's arm. The device can be configured to remain in its position on the subject's arm independent of any movement or changes in orientation of the subject's arm.

In some embodiments, the device can be capable of collecting 250 μL of fluid sample from the subject in less than 1 minute 45 seconds. In some cases, the device can be capable of collecting at least 175 μL to 300 μL of fluid sample from the subject in less than 3 minutes. In some cases, the device can be capable of collecting at least 200 μL of fluid sample from the subject in less than 5 minutes. The device can be configured to collect the fluid sample at a rate that is dependent on the size or shape of the recess and/or vacuum pressure. The recess can be configured having a size and shape that enables an increased volume of the fluid sample to be accumulated in the skin drawn into the recess. The recess can be configured having a size and shape that enables the increased volume of the fluid sample to be accumulated. In some cases, (1) the size and shape of the recess and (2) a pressure of the vacuum can be configured to achieve a minimum capillary pressure in the skin drawn into the recess. In some cases, (1) the size and shape of the recess and (2) a pressure of the vacuum can be configured to achieve a minimum tension in the skin drawn into the recess. The device can be configured to collect the fluid sample at a rate that is dependent on the size and shape of the recess.

In some other aspects, a device for collecting a fluid sample from a subject is provided. The device can comprise: a housing comprising a piercing activator configured to activate one or more skin piercing elements, and a vacuum activator separate from the piercing activator and configured to activate an evacuated vacuum chamber prior to the activation of the one or more piercing elements by the piercing activator.

In some aspects, a method for collecting a fluid sample from a subject can comprise: placing a device packaged with an evacuated vacuum chamber and one or more piercing elements on skin area of the subject; activating the evacuated vacuum chamber to effectuate vacuum pressure on the skin area; piercing the skin area after vacuum activation; and maintaining the vacuum pressure during and after penetrating the skin area of the subject, in order to draw the fluid sample from the skin into device.

In some embodiments, the piercing activator and the vacuum activator can be two separate components. The vacuum activator can comprise a first input interface on the housing, and the piercing activator can comprise a second input interface on the housing. In some cases, at least one of the first input interface or the second input interface can comprise a button. In some alternative cases, the vacuum activator can comprise a first input interface and the piercing activator can comprise a second input interface, and at least one of the first input interface or the second input interface can be remote from the housing.

In some embodiments, the piercing activator can be configured to activate the one or more piercing elements after the skin is drawn into the recess. The piercing activator can be configured to activate the one or more piercing elements after the skin is drawn into the recess by the vacuum for a predetermined length of time. In some cases, the predetermined length of time can range from about 1 second to about 60 seconds. In some embodiments, the housing can comprise the pre-evacuated vacuum chamber, and the vacuum activator can be configured to activate the vacuum in the pre-evacuated vacuum chamber. In some cases, the piercing activator can be configured to activate the one or more piercing elements only after the vacuum has been activated. In some cases, the piercing activator can be locked and incapable of activating the one or more piercing elements prior to activation of the vacuum. The piercing activator can comprise a locking mechanism coupled to the vacuum activator. The locking mechanism can be configured such that the piercing activator is initially in a locked state. The vacuum activator can serve as a key for unlocking the piercing activator, and the piercing activator can be simultaneously unlocked when the vacuum activator is activated. The vacuum activator can be configured to activate the vacuum by establishing fluidic communication to the pre-evacuated vacuum chamber. For example, the vacuum activator can be configured to pierce a foil seal or open a valve to establish the fluidic communication to the pre-evacuated vacuum chamber.

In some embodiments, the vacuum activator can be located on the housing such that the vacuum activator is configured to be pressed in a first direction, and the piercing activator can be located on the housing such that the piercing activator is configured to be pressed in a second direction. In some cases, the first direction and the second direction can be substantially the same. Alternatively, the first direction and the second direction can be substantially different. In some cases, the first direction and the second direction can be substantially parallel to each other. In some cases, at least one of the first direction or the second direction does not extend toward the skin of the subject. For example, the second direction does not extend toward the skin of the subject. In some cases, at least one of the first direction or the second direction can extend substantially parallel to the skin of the subject. In some cases, the first direction and the second direction can both extend substantially parallel to the skin of the subject. In some cases, at least one of the first direction or the second direction can extend in a direction of gravitational force. In some cases, the first direction and the second direction can both extend in the direction of gravitational force. In some embodiments, the piercing activator and the vacuum activator can be located on a same side of the housing, and can be ergonomically accessible by the subject when the device is mounted onto an arm of the subject. For example, the piercing activator can be located on a cover of the housing, and the vacuum activator can be located on a base of the housing where the vacuum chamber is located. Alternatively, the piercing activator and the vacuum activator can be located on different sides of the housing, and can be ergonomically accessible by the subject when the device is mounted onto an arm of the subject.

In some further aspects, a method for collecting a fluid sample from a subject is provided. The method can comprise: with aid of a fluid acquisition device: piercing skin of the subject and delivering the fluid sample from the subject to a matrix disposed within a deposition chamber of the fluid acquisition device, wherein the delivery of the fluid sample is assisted or enhanced using (1) gravitational force, (2) vacuum force, (3) a pressure difference between capillary pressure and internal pressure of the device, and (4) wicking behavior of the fluid sample along the matrix.

In some aspects, a device for collecting a fluid sample from skin of a subject and delivering it to a deposition chamber is provided, wherein fluid flow from the skin to a matrix in the deposition chamber can be preferably enhanced by (1) gravitational force, (2) vacuum force, (3) a pressure differential between capillary pressure and internal pressure of the device, and (4) wicking behavior of the fluid sample along the matrix.

In some embodiments, the device can comprise an enclosure for holding one or more piercing elements, and the enclosure can be in fluidic communication with the deposition chamber. The deposition chamber and the enclosure can be initially at ambient pressure, prior to activation of a vacuum from a pre-evacuated vacuum chamber located onboard the device. In some cases, the deposition chamber, the vacuum chamber, and the enclosure can be configured to equalize to an internal pressure that is less than the ambient pressure after the vacuum has been activated. The internal pressure can be higher than the initial evacuated vacuum pressure of the vacuum chamber. In some cases, the internal pressure can be about −5.5 psig, and the sealed vacuum pressure can be about −12 psig. The internal pressure can be configured to draw the skin into a recess of the housing. The internal pressure can be configured to draw blood from capillary beds to the skin that is being drawn into the recess. A pressure differential can be created between capillary pressure and the internal pressure when the skin is penetrated by one or more piercing elements of the device. The internal pressure can increase as the fluid sample is drawn from the skin towards the deposition chamber and the enclosure. In some cases, the internal pressure in the enclosure can increase more rapidly compared to a collective internal pressure of the deposition chamber and the vacuum chamber. The internal pressure in the enclosure can increase substantially more than the collective internal pressure of the deposition chamber and the vacuum chamber. The substantially increased internal pressure of the enclosure can inhibit the flow of the fluid sample into the enclosure. The substantially increased internal pressure of the enclosure can result in preferential flow of the fluid sample towards the pressure of the enclosure can cause the flow of the fluid sample into the enclosure to slow or stop, while the fluid sample can continue to flow towards the deposition chamber under the influence of the pressure differential. In some cases, (1) a volume of the enclosure and (2) a collective volume of the deposition chamber and the vacuum chamber, can be configured such that minimal amounts of the fluid sample flows towards and into the enclosure. In some cases, a ratio of the volume of the enclosure to the collective volume of the deposition chamber and the vacuum chamber can range from about 1:5 to about 1:15. In some cases, the one or more piercing elements can be configured to penetrate the skin to generate cuts, and the pressure differential can enable deeper cuts and the cuts to be held open under tension. The pressure differential can be configured to increase the size of the cuts to enable a higher flowrate and volume of the fluid sample to be collected from the skin.

In some further aspects, a device for penetrating skin of a subject is provided. The device can comprise: one or more piercing elements supported by a piercing holder movable by two or more spring elements; a deployment spring positioned to deploy the one or more piercing elements through an opening in the device; and a retraction spring positioned to retract the one or more piercing elements back into the device, wherein a length of the one or more piercing elements is less than about 20 mm, and the depth of penetration of the one or more piercing elements is about 2 mm. In some cases, the length of the one or more piercing elements is about 12.7 mm.

In some aspects, a method for penetrating skin of a subject can comprise providing the aforementioned device; drawing the skin of the subject into a recess of the device; activating the deployment spring and deploying the one or more piercing elements through the opening in the device; penetrating the skin of the subject using the one or more piercing elements; and using the retraction spring to retract the one or more spring elements back into the device.

In some embodiments, two or more piercing elements can be supported by a holder in a random configuration. In some cases, the two or more piercing elements can have random orientations relative to each other. The two or more piercing elements can comprise beveled edges that are randomly oriented relative to each other. The beveled edges of the two or more piercing elements can be non-symmetrical to each other. The beveled edges of the two or more piercing elements can be at an acute or oblique angle relative to each other.

In some cases, two or more piercing elements can be supported by a holder in a predefined configuration. The two or more piercing elements can have predefined orientations relative to each other. The two or more piercing elements can comprise beveled edges that are oriented relative to each other in a predefined manner. The beveled edges of the two or more piercing elements can be symmetrical to each other.

In some embodiments, the piercing elements can comprise two or more lancets. Optionally, the piercing elements can comprise needles and/or microneedles. In some cases, two or more lancets can have a same bevel angle. Alternatively, two or more lancets can have different bevel angles. In some cases, the bevel angle(s) can range from about 10 degrees to about 60 degrees. In some cases, the two or more lancets can comprise beveled faces having a same bevel length. Alternatively, the two or more lancets can comprise beveled faces having different bevel lengths. In some cases, the bevel length(s) can range from about 2 mm to about 10 mm.

In some embodiments, two or more piercing elements can be configured to generate cuts on the skin that extend in different directions along the skin and that are non-parallel to each other.

In some embodiments, the deployment spring can be configured to move and cause the piercing elements to penetrate the skin of the subject at speeds ranging from about 0.5 m/s to about 2.0 m/s. The deployment spring can be configured to move and cause the piercing elements to penetrate the skin of the subject with a force ranging from about 1.3 N to about 24.0 N. A spring-force of the retraction spring can be less than a spring-force of the deployment spring. In some cases, the deployment spring can have a spring-rate of about 2625 N/m, and the retraction spring can have a spring-rate of about 175 N/m. The deployment spring can be configured to cause the one or more piercing elements to penetrate the skin to depths ranging from about 0.5 mm to about 3 mm. The retraction spring can be configured to retract the piercing elements from the skin of the subject at speeds ranging from about 0.1 m/s to about 1.0 m/s.

In some embodiments, the device can further comprise: a vacuum activator configured to activate a vacuum for drawing the skin into a recess of the device. In some cases, a piercing activator can be configured to activate the deployment spring only after the vacuum activator is activated.

In some further aspects, a device for monitoring fluid sample collection from a subject is provided. The device can comprise: a housing comprising a cartridge chamber; a cartridge operably coupled to the cartridge chamber; components for penetrating skin of the subject and drawing the fluid sample from the skin into the cartridge; and a flow meter on the housing that enables the subject or a user to monitor a progress of the fluid sample collection in real-time as the fluid sample is collected into the cartridge.

In some aspects, a method for monitoring fluid sample collection from a subject can comprise: providing (1) a housing comprising a cartridge chamber, (2) a cartridge operably coupled to the cartridge chamber, (3) components for penetrating skin of the subject and drawing the fluid sample from the skin into the cartridge, and (4) a flow meter on the housing; and monitoring, with aid of the flow meter, a progress of the fluid sample collection in real-time as the fluid sample is collected into the cartridge.

In some embodiments, the flow meter can be provided on a lid covering a base of the housing. The flow meter is not obscured by a cover of the housing. The flow meter can be in proximity to the cartridge chamber. The flow meter can be substantially aligned with a cartridge located within the cartridge chamber. In some embodiments, the flow meter can comprise a plurality of windows disposed parallel to a longitudinal axis of the cartridge. The plurality of windows can be made of an optically transparent material. The fluid sample can be visible through the windows and sequentially fills each window as the fluid sample is being collected into the cartridge. Each window can be indicative of a known amount of fluid sample that is collected. The fluid sample collection is complete when the fluid sample is visible in all of the windows. The plurality of windows can comprise three or more windows.

In some embodiments, the flow meter can comprise a single window disposed parallel to a longitudinal axis of the cartridge. The window can be made of an optically transparent material. The fluid sample can be visible through the window and continuously fills the window as the fluid sample is being collected into the cartridge. The fluid sample collection is complete when the fluid sample is visible throughout the window.

In some further aspects, a cartridge assembly is provided. The cartridge assembly can comprise: a cartridge for holding one or more matrices for storing a fluid sample thereon; a cartridge holder releasably coupled to the cartridge, wherein the cartridge assembly is releasably coupled to a device used for collecting the fluid sample.

In some embodiments, a device for collecting a fluid sample from a subject is provided. The device can comprise: a housing comprising a deposition chamber and a pre-evacuated vacuum chamber, wherein the deposition chamber is configured to receive and releasably couple to the cartridge assembly, and the deposition chamber is in fluidic communication with the vacuum chamber.

In some embodiments, a fluid sample collection kit can comprise the device and the cartridge assembly. In some embodiments, a fluid sample collection assembly can comprise the device and the cartridge assembly releasably coupled to said device. In some embodiments, an input port of the cartridge can be releasably coupled to and in fluidic communication with a channel of the device, and the fluid sample can be collected from penetrated skin of the subject and transported through the channel into the cartridge.

In some embodiments, a method for collecting a fluid sample from a subject can comprise: releasably coupling the cartridge assembly to the device; placing the device adjacent to skin of the subject; activating vacuum in the vacuum chamber to draw the skin into a recess of the housing; using one or more piercing elements of the device to penetrate the skin; maintaining the device adjacent to the skin for a sufficient amount of time to draw the fluid sample into the device and collect the fluid sample into the cartridge; and decoupling the cartridge assembly from the device after a certain amount of the fluid sample has been collected in the cartridge.

In some embodiments, the cartridge holder can be releasably coupled to the cartridge via a quick release mechanism. In some cases, the quick release mechanism can comprise one or more spring-clips on the cartridge holder. The cartridge assembly can be capable of being coupled to and detached from the deposition chamber without use of tools. The cartridge assembly can be capable of being coupled to and detached from the deposition chamber using no more than two motion steps. The cartridge assembly can be coupled to the deposition chamber prior to the collection of the fluid sample from the subject. The cartridge assembly can be decoupled from the deposition chamber after the fluid sample from the subject has been collected into the cartridge.

In some embodiments, the cartridge can comprise two or more matrices for collecting and storing the fluid sample thereon. The two or more matrices can be disposed in a configuration that permits the fluid sample to wick between and along the two or more matrices. For example, the two or more matrices can be disposed substantially parallel to each other. In some cases, the two or more matrices can be separated by a gap of about 0.5 mm. In some cases, at least one of the matrices can be capable of collecting at least 60 μL of fluid sample. In some cases, each of two or more matrices can be capable of collecting at least 60 μL of fluid sample.

In some embodiments, the cartridge can further comprise one or more absorbent pads configured to be in fluidic communication with the one or more matrices, wherein the one or more absorbent pads can be used to hold excess fluid sample. The one or more absorbent pads can aid in ensuring that a predefined volume of the fluid sample can be collected and maintained on the one or more matrices, regardless of an input volume of the fluid sample into the cartridge up to a predefined range. In some cases, the one or more matrices can include two matrices that are each configured to hold up to about 7 μL of the fluid sample. Each of the two matrices can be configured to hold and maintain about 75 μL of the fluid sample as the input volume of the fluid sample to the cartridge increases beyond 150 μL up to the predefined range. In some cases, the predefined range can be from about 150 μL to about 300 μL. In other cases, the predefined range can be greater than 300 μL. In some cases, the one or more absorbent pads can be capable of holding at least 100 μL of excess fluid sample.

In some embodiments, the cartridge holder can comprise a cartridge tab that is configured to be releasably coupled to a distal end of the deposition chamber. The cartridge tab can be configured such that the subject or a user is able to (1) support the cartridge assembly by holding the cartridge tab, (2) couple the cartridge assembly to the device by pushing the cartridge tab, and/or (3) decouple the cartridge assembly from the device by pulling the cartridge tab.

In some further aspects, a transportation sleeve is provided. The sleeve can comprise: an opening configured to couple to a cartridge tab included with the cartridge; and a dual support-release mechanism within the sleeve, wherein the dual support-release mechanism can comprise: (a) a retention element configured to engage with a corresponding mating feature on the cartridge and secure the cartridge within the sleeve, and (b) a release element configured to cause the spring-clips on the cartridge holder to release and thereby decouple the cartridge from the cartridge holder. The dual support-release mechanism can permit the cartridge holder to be removed from the opening of the sleeve while the cartridge is secured in place within the sleeve, without exposure of the strips to the ambient environment. In some cases, the transportation sleeve can further comprise a desiccant within the sleeve. In some cases, the sleeve can be sized and shaped to accommodate user or patient identity (ID) labels.

In some embodiments, a transportation assembly can comprise: the transportation sleeve, and the cartridge coupled to said transportation sleeve. In some cases, the cartridge tab can be configured to hermetically seal the opening of the sleeve.

In some embodiments, the cartridge can be oriented such that the flow of the fluid sample into the cartridge is further aided with gravity. In some cases, the cartridge can comprise a luer-type fitting that can engage with the device when the cartridge is inserted into the deposition chamber.

In some embodiments, the one or more matrices can comprise absorbent paper. In some cases, one or more of the matrices can comprise stabilization chemistry. In some cases, a first matrix can comprise a first stabilization chemistry and a second matrix can comprise a second stabilization chemistry different from the first stabilization chemistry. In some alternative cases, one or more of the matrices does not comprise stabilization chemistry.

Provided herein are medical systems, devices, and methods for sample collection and storage. The disclosed systems, devices, and methods can comprise structure features that facilitate sample collection (e.g. blood collection devices) as well as components for collecting blood sample on to substrate for storage and transport.

Any of the devices disclosed herein can rely on the generation of a vacuum to apply negative pressure to deform the skin of a subject and to apply local suction directly to the sample collection site, thereby facilitating sample flow and collection. Any of the devices disclosed herein can comprise a concave cavity that can be placed at the surface of the skin of the subject, this concave cavity can be configured to deliver vacuum (e.g. negative pressure, suction etc.) to the skin of the subject. Any of the devices disclosed herein can comprise an opening disposed at the apex of or other surface of the concave cavity, the inner diameter can be configured to allow a piercing element to pierce the skin of the subject; and a piercing element can be configured to pass through the inner diameter. Local suction can be applied to the sample collection site through the inner diameter.

In some embodiments, a vacuum can be configured to deform the skin of the subject using different mechanisms, for example the vacuum can be configured to draw the skin of the subject into the concave cavity. A concave cavity can be configured to constrain the surface of the skin against its entire or a portion of its concave surface of the subject at which point the piercing element can be configured to pierce the skin of the subject. An opening contiguous with a cylinder (e.g. a cylinder in fluid contact with a cartridge) can be configured to draw the blood from the subject into the device when the vacuum is applied to the skin of the subject and after an incision has been made in the skin of the subject.

Vacuum pressure can be generated using an evacuated vacuum chamber configured such that activation of the device pierces the evacuated vacuum chamber forming negative pressure that draws the blood from the subject through the opening and channels and into a cartridge and onto a solid matrix for sample storage vacuum pressures can be in the range of between 1-20 psi. The vacuum pressure can be about 5 psi. Vacuum chamber volume can be within a 10%-100% margin of twice the volume of the combined concave cavity, opening, channel and cartridge volume. Any of the devices disclosed herein can comprise a vacuum activation actuator configured to activate the vacuum upon actuation of the vacuum activation actuator. The vacuum activation actuator can comprise a button.

Any of the devices disclosed herein can be configured for drawing a specific volume (e.g. greater than 20 μL, greater than 40 μL, greater than 60 μL, greater than 80 μL, greater than 100 μL, greater than 150 μL, or greater than 200 μL) of blood (e.g. capillary blood) from a subject in defined period of time (e.g. less than 4 minutes), can have specific vacuum and device parameters. The structure of the concave cavity can have an impact on blood collection, for example the rate of blood sample collection can be dependent on the curvature and size of the concave cavity and the vacuum pressure.

To facilitate blood collection the surface area acted on by the vacuum can have specific parameters, for example the surface area of the skin under vacuum and in contact with the concave cavity can be within a 10% margin of 500 mm² and the opening in fluid contact with a cylinder (e.g. a cylinder in fluid contact with a cartridge) can have a diameter can be within a 10% margin of 8 mm². Any of the devices, systems and methods herein for collecting sample (e.g. blood samples) can be configured with a removable cartridge. The removable cartridge can be held in fluid communication with the cylinder (e.g. the cylinder in contact with the opening in the concave cavity). Any of the devices disclosed herein can comprise a visual metering window configured to permit visualization of the removable cartridge while the removable cartridge is in the device. Any of the devices disclosed herein can comprise a piercing module, wherein the piercing module comprises one or more piercing elements. The piercing elements can be actuated with a button. Before and after actuation, the piercing element can be withdrawn when the piercing element is in an unactivated state.

Also disclosed herein are cartridges configured to collect sample from the device and transfer it to solid substrate such that precise volumes of sample are collected on and metered by the absorbency of the solid substrate. For example, the standardized quantity of blood saturating each strip of the substrate can be within the range of 50-100 μL on a substrate with surface area within the range of 100-300 square millimeters. A cartridge can comprise a channel disposed between two strips of substrate configured for transferring a blood sample to the two strips of substrate. A cartridge can comprise a spacer disposed between a portion of each of the two strips of substrate. A spacer can be configured to adjust the space between the two strips of substrate depending on one or more conditions. Cartridges can be removable from the device, for example using methods to clip the cartridge into place. Cartridges can further comprise a wicking tail. A wicking tail can be configured for standardizing the quantity of blood saturated on the two strips of substrate.

Collecting standardized quantities of blood on substrate of specific surface area can be performed using various methods. Methods for applying blood to at least two solid supports can comprise the steps of providing a cartridge comprising at least two solid supports. The provided cartridge can comprise at least two solid supports are substantially the same size, such that a surface of each of the at least two solid supports face each other and the surface at the least two solid supports are substantially parallel to each other. The at least two solid supports can be separated by a defined distance (e.g. within 10% margin of 0.4 mm), and the cartridge can be configured so that a channel is formed between the two solid supports. Blood can be passing into the tunnel between the at least two solid supports, wherein the blood is absorbed to each of the at least two solid supports as it passes through the tunnel between the at least two solid supports. Solid supports used in these methods can comprise fixed dimensions (e.g. width between 3 mm and 10 mm and length between 3 mm and 26 mm). The cartridge used in the method can further comprise a wicking element configured for metering blood flow through the device.

In some aspects, a method is disclosed. In some embodiments, the method comprises: obtaining a device, comprising: (a) one or more piercing elements configured to penetrate skin of a subject to expose blood; (b) a plunger configured to drive the one or more piercing elements into the skin of the subject; and (c) a removable collection chamber for collecting the blood; applying the device to the skin of the subject; actuating the plunger to cause the one or more piercing elements to pierce the skin of the subject; creating a vacuum upon withdrawal of the one or more piercing elements from the skin; and collecting greater than 100 μL of the blood in the removable collection chamber, wherein the removable collection chamber is configured to be removed from the device after the blood is collected in the removable collection chamber.

In some embodiments, the method further comprises contacting the blood with a stabilizing reagent that preserves the blood for transport or storage. In some embodiments, the method further comprises contacting the blood with silica. In some embodiments, the method further comprises contacting the blood with one or more salts. In some embodiments, the method further comprises contacting the blood with one or more chelating agents. In some embodiments, the one or more chelating agents comprise ethylenediaminotetraacetic acid (EDTA).

In some embodiments, the method further comprises contacting the collected blood with a polymer.

In some embodiments, the one or more piercing elements comprise a microneedle. In some embodiments, the one or more piercing elements are configured to minimize physical pain to the subject. In some embodiments, the one or more piercing elements are configured to allow for penetration of the skin of the subject.

In some embodiments, the device comprises a body comprising a base comprising at least one aperture. In some embodiments, the one or more piercing elements are configured to pass through the at least one aperture in the base when the plunger is actuated. In some embodiments, the collecting comprises collecting greater than 500 μL of the blood. In some embodiments, the collecting comprises collecting greater than 750 μL of the blood. In some embodiments, the blood comprises one or more analytes. In some embodiments, the one or more analytes comprise nucleic acid.

In some embodiments, the method further comprises performing an assay for the one or more analytes. In some embodiments, the method further comprises performing an assay is at a location remote from a site of the collecting. In some embodiments, the method further comprises sequencing a nucleic acid from the blood or an amplified product thereof. In some embodiments, the method further comprises removing the removable collection chamber from the device following the collecting.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a perspective view of a sample acquisition device in accordance with some embodiments;

FIG. 1B shows a recess of the device for skin suction;

FIG. 1C shows a flow meter of the device for monitoring the progress of sample collection;

FIG. 1D shows a removable cartridge assembly for sample collection;

FIG. 2A shows a perspective view of a housing base assembly of the device;

FIG. 2B shows a perspective view of a housing cover of the device;

FIG. 2C shows another perspective view of the device;

FIG. 3A shows a side sectional view of the device prior to insertion of the cartridge assembly;

FIG. 3B shows a top view of the device of FIG. 3A;

FIG. 4A shows a side view of the device after insertion of the cartridge assembly;

FIG. 4B shows a top sectional view of the device of FIG. 4A;

FIG. 5A shows a side sectional view of the device placed on a subject's skin without vacuum activation;

FIG. 5B shows a schematic block diagram corresponding to the device of FIG. 5A;

FIG. 6A shows the subject's skin being drawn into the recess under vacuum pressure;

FIG. 6B shows a schematic block diagram corresponding to the device of FIG. 6A;

FIG. 7A shows a piercing activator of the device in a locked state;

FIG. 7B shows the piercing activator of the device in an unlocked state;

FIG. 8A shows the subject's skin being penetrated by piercing elements after the piercing elements have been deployed;

FIG. 8B shows a schematic block diagram corresponding to the device of FIG. 8A;

FIG. 9A shows blood being drawn from the cuts on the skin after the piercing elements have been retracted;

FIG. 9B shows a schematic block diagram corresponding to the device of FIG. 9A;

FIG. 10A shows the preferential and enhanced flow of blood from the cuts towards the cartridge in the deposition chamber of the device;

FIG. 10B shows a schematic block diagram corresponding to the device of FIG. 10A;

FIGS. 11A and 11B show schematic block diagrams of a sample acquisition device prior to insertion of a cartridge assembly;

FIGS. 12A and 12B show schematic block diagrams of the device after insertion of the cartridge assembly;

FIGS. 13A and 13B show the device of FIGS. 12A/12B being placed on a subject's skin;

FIGS. 14A and 14B show the equalization of pressures and the subject's skin being drawn into the recess, upon piercing the foil separating the vacuum chamber and the deposition chamber;

FIGS. 15A and 15B show the subject's skin being completely drawn into the recess by negative pressure;

FIG. 16A shows the deployment of the piercing elements and penetration of the subject's skin in the recess;

FIG. 16B shows the subject's skin being penetrated and the retraction of the piercing elements;

FIG. 16C shows the initial flow of blood from the cuts on the skin;

FIG. 16D shows the blood being drawn towards the cartridge in the deposition chamber with aid of the vacuum, pressure differentials, and gravitational force;

FIG. 16E shows the preferential flow of blood towards the deposition chamber, and the wicking of blood along matrices in the cartridge;

FIG. 16F shows the blood absorbed onto the matrices, and completion of the blood collection;

FIGS. 17A, 18A, and 19A show schematic block diagrams of blood flow along the matrices in the cartridge at different stages of the blood collection;

FIGS. 17B, 18B, and 19B illustrate a flow meter indicating the progress of the blood collection in accordance with some embodiments;

FIGS. 17C, 18C, and 19C illustrate a flow meter indicating the progress of the blood collection in accordance with some other embodiments;

FIG. 20A shows a top view of a device with the flow meter indicating that the blood collection has been completed;

FIG. 20B is a schematic block diagram corresponding to the device of FIG. 20A prior to removal of the filled cartridge assembly;

FIG. 21A shows a top view of the device with the filled cartridge assembly removed;

FIG. 21B is a schematic block diagram corresponding to the device of FIG. 21A with the filled cartridge assembly removed;

FIG. 22A shows a perspective view of a transportation sleeve;

FIG. 22B shows a top view of the transportation sleeve and a filled cartridge assembly prior to its insertion into the sleeve;

FIG. 22C shows the filled cartridge assembly inserted into the transportation sleeve;

FIG. 23 shows an exploded view of the transportation sleeve and cartridge assembly;

FIG. 24A shows a side sectional view of the transportation sleeve with cartridge assembly inserted therein;

FIG. 24B shows a side sectional view with the cartridge holder removed, leaving the cartridge within the transportation sleeve;

FIGS. 25A and 25B show an exemplary procedure of collecting blood samples from a subject using a sample acquisition device, and packaging of the blood samples for shipment;

FIG. 26 shows an exploded view of certain components of the sample acquisition device;

FIG. 27A shows different views of a piercing elements;

FIG. 27B shows the piercing elements being supported by a holder;

FIG. 28A shows different views of a deployment spring;

FIG. 28B shows different views of a retraction spring;

FIG. 29 shows different views of two matrices separated by spacers with an absorbent pad on one end;

FIG. 30 shows examples of different types of recesses that can be used in a sample acquisition device;

FIG. 31A, FIG. 31B, FIG. 31C, and FIG. 31D illustrates features that can be included in a device or device for collecting a blood sample;

FIG. 32A, FIG. 32B, FIG. 32C, and FIG. 32D illustrate front, side, and back views of a device that can be used to collect a sample of defined volume and store it on a removable stabilization matrix;

FIG. 33A, FIG. 33B, and FIG. 33C illustrate features of a device that can be used to enhance sample collection;

FIG. 34A, FIG. 34B, FIG. 34C, and FIG. 34D illustrate an embodiment of a device for collecting a blood sample from a subject, and means for storing the sample in a removable cartridge;

FIG. 35 illustrates an inside view of a removable cartridge that can be used with any of the disclosed sample collection devices (e.g., sample collection devices illustrated in FIGS. 31A-31D, FIGS. 32A-32D, FIGS. 33A-33C, and FIGS. 34A-34D);

FIG. 36A and FIG. 36B illustrate an exemplary orientation of a device or device configured to use one or more mechanisms (e.g. gravity, capillary action, global vacuum, and local suction) collect and deposit a blood sample on a solid matrix for storage;

FIG. 37A, FIG. 37B, FIG. 37C, and FIG. 37D illustrate a modular design of an device with components configured for generating a vacuum, lancing a subject's skin, collecting, metering and stabilizing a blood sample from the subject, and storing the collected sample;

FIG. 38A illustrates external features of an exemplary low profile embodiment of a device provided herein;

FIG. 38B illustrates internal workings of a device provided herein in an exemplary starting position, when the device is not activated;

FIG. 38C illustrates internal workings of a device provided herein once the button is depressed (1), and the blade holder is released (2);

FIG. 38D illustrates internal workings of a device provided herein at an end of a travel path of a blade holder, where the collection arm is released by a latch that makes contact with the blade holder at the end of the travel path;

FIG. 38E shows a side view of a device provided herein, providing a view of a release mechanism for a blood collection arm;

FIG. 38F illustrates a released blood collection arm creating a seal around cuts created by blades shown in FIG. 38E;

FIG. 39A, FIG. 39B, FIG. 39C, FIG. 39D, and FIG. 39E illustrate top, bottom, side and internal views of an exemplary low profile blood collection device;

FIG. 40A, FIG. 40B, FIG. 40C, and FIG. 40D illustrate various views of a blood collection device and components thereof configured for lancing a subject using vertical cutting, and extracting a sample from the subject using a syringe;

FIG. 41A illustrates a safety mechanism that can be used to prevent inadvertent blade deployment of a blood sample collection device for collecting a sample that relies on vertical cutting using a rotatable blade;

FIG. 41B illustrates a mechanism for collecting a sample using a blood collection device that relies on vertical cutting using a rotatable blade;

FIG. 42A, FIG. 42B, and FIG. 42C illustrates a device and mechanism for collecting a sample using a spring loaded blade rotatable blade to perform vertical cutting;

FIG. 43A and FIG. 43B illustrates a device for applying global vacuum and local suction to collect an appropriate amount of sample within a desired period of time (e.g. at a rate that falls within a desired range);

FIG. 44A and FIG. 44B illustrate two views of a device for simultaneously lancing a subject and forming a seal;

FIG. 45A, FIG. 45B, and FIG. 45C illustrate an exemplary vacuum chamber that can be used with any of the devices and methods disclosed herein;

FIG. 46A, FIG. 46B, and FIG. 46C illustrate an exemplary chamber for collecting, metering, storing and stabilizing a sample, and mechanisms for driving sample through the chamber and onto the solid matrices for storing the sample;

FIG. 47 illustrates the components of a system configured for collecting a blood sample using a sample collection device and a removable sample storage cartridge;

FIG. 48 illustrates steps that a subject or clinician might take to collect and provide a sample to a facility for analysis;

FIG. 49 illustrates steps that a lab (e.g. a CLIA certified lab or other facility) might perform to prepare a sample for analysis;

FIG. 50A, FIG. 50B, and FIG. 50C illustrate a visual metering window permitting a user to view draw progress (FIG. 50A illustrates visually tracking by the health care provider (HCP) as the stabilization matrix strip fills. When the final window fills, the draw is complete. FIG. 50B illustrates a wicking pad capturing excess blood. FIG. 50C illustrates varying levels of blood deposition on matrix strips.);

FIG. 51 illustrates the percentage of HbA1c in blood samples of five different volumes (30 μL, 45 μL, 60 μL, 75 μL, 100 μL) drawn from two different donors;

FIG. 52 illustrates a flow chart of a clinical trial to access precision of blood tests done using blood drawn with devices disclosed herein compared to venipunctures;

FIG. 53 illustrates principles of operation and use flow of a device;

FIG. 54 illustrates HbA1c as a percent of total hemoglobin (Y-axis) for various experimental conditions (X-axis)(For each condition, the average of the replicate measurements is plotted as a black bar and sample measurements are shown as open circles. The dotted lines delineate ±6% relative error around the Day-0 average measurement.);

FIG. 55 illustrates HbA1c as a percent of total hemoglobin (Y-axis) for various experimental conditions (X-axis)(Four individual, Day-0, liquid whole-blood, replicates for each donor are plotted as circles. Two technical replicates for each dried blood spot (DBS)-strip are averaged and the resulting DBS-strip averages are also plotted as circles. For each experimental condition, the average of all measurements is plotted as a black bar. Dotted lines delineate±% relative error around the donor-specific, Day-0, average measurements.); and

FIG. 56 illustrates an exemplary procedure to collect and store blood using a device described herein.

FIG. 57A, FIG. 57B, and FIG. 57C illustrate different sample acquisition components and piercing elements.

FIG. 58 illustrates an embodiment of a sample acquisition component.

FIG. 59 is a flow diagram depicting a method for extracting a blood sample using a tourniquet.

FIG. 60A and FIG. 60B illustrate an embodiment of the separation component.

FIG. 61 illustrates another embodiment of the sample separation component.

FIG. 62 illustrates a sample stabilization component with a stabilization matrix.

FIG. 63A, FIG. 63B, FIG. 63C, FIG. 63D, FIG. 63E, FIG. 63F, FIG. 63G, AND FIG. 63H are a non-limiting list of tests that can be conducted on the sample.

### DETAILED DESCRIPTION

Reference will now be made in detail to exemplary embodiments of the disclosure, examples of which are illustrated in the accompanying drawings. Wherever possible, the same reference numbers will be used throughout the drawings and disclosure to refer to the same or like parts.

### I. General

Provided herein are devices, methods, and kits for collecting a fluid sample, e.g., from a subject's body. The fluid sample can be, for example, blood drawn from penetrated skin of the subject. The devices disclosed herein can be handheld and user-activable, and suitable for use outside of traditional healthcare facilities, for example in homes, in remote locations, while a subject is traveling, etc. The devices can be portable and easy to use, and allow individuals to efficiently and reliably collect their own blood samples, without relying on trained healthcare personnel, and without requiring the individual to have any prior blood draw training experience. The devices and methods described herein can be minimally invasive and permit lower levels of pain (or perception of pain) in a subject relative to use of other devices and methods, which can help to improve the overall blood draw experience for the subject. Kits can be provided with detailed instructions that guide users on how the devices can be used for blood sample collection and storage. Optionally in any of the embodiments disclosed herein, the kits can include transportation sleeves and pouches for shipping/transportation of cartridges to testing facilities. A cartridge can be configured to support one or more matrices configured to hold at least a predefined volume of collected blood.

Notably, the sample acquisition devices and methods disclosed herein can enhance collection of a fluid sample (e.g., blood) from the subject. The disclosed sample acquisition devices and methods can be capable of drawing blood at increased flowrates and higher sample volumes beginning from time of skin incision, compared to currently available non-venous blood collection devices and methods. According to various embodiments of the present disclosure, an average collection flowrate and collected sample volume can be increased with aid of a number of features, e.g., a recess that is configured or optimally designed for skin suction, vacuum, pressure differentials, aid of gravitational force, wicking or capillary effects, as described in further detail herein. Additionally, the embodiments disclosed herein are advantageous over currently available non-venous blood collection devices and methods, in that the disclosed devices and methods can permit stabilization of controlled volumes of blood samples to be deposited on one more matrix strips. Further advantages of the disclosed embodiments can include ease of sample removal from a sample acquisition device, and the packaging of the removed sample for subsequent transportation to testing facilities.

Samples, e.g., blood samples, collected using the sample acquisition devices and methods described herein can be analyzed to determine a person's physiological state, for detecting diseases and also for monitoring a health condition of the user. Individuals can rapidly evaluate their physiological status, since samples, e.g., blood samples can be quickly collected using the disclosed devices and methods, and the samples, e.g., blood samples can be either (1) analyzed on the spot using, for example, immunoassays or (2) shipped promptly to a testing facility. The reduced lead-time for blood collection, analysis and quantification can be beneficial to many users, e.g., users who have certain physiological conditions/diseases that require constant and frequent blood sample collection/monitoring.

Various aspects of the devices, methods, and kits described herein can be applied to any of the particular applications set forth herein and for any other types of fluid sample devices, in addition to blood collection devices. The devices, methods, and kits can be used in any system that requires a fluid sample to be drawn from the subject's body. The devices, methods, and kits described herein can be applied as a standalone apparatus or method, or as part of a medical system in a healthcare environment. It shall be understood that different aspects of the devices, methods, and kits described herein can be appreciated individually, collectively, or in combination with each other.

### II. Sample Acquisition Devices

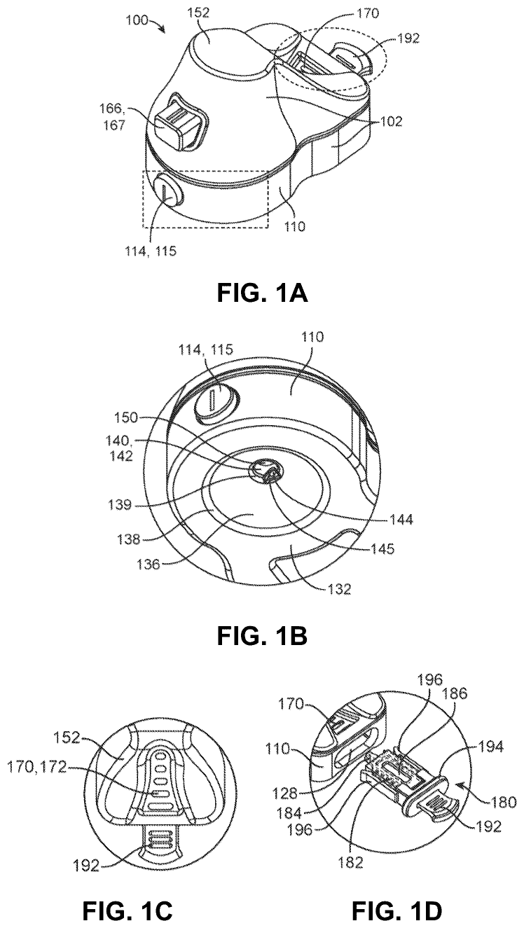

FIGS. 1A-1D illustrate sample acquisition device 100 in accordance with some embodiments. A sample acquisition device as described herein can refer to any apparatus, device or system that is designed, configured, or used for collecting, storing, and/or stabilizing a fluid sample, e.g., a fluid sample drawn from a subject. In various aspects, the sample is a biological sample. Non-limiting examples of biological samples suitable for use with the devices of the disclosure can include whole blood, blood serum, blood plasma, and the like.

The devices herein can be used in a variety of environments and applications including an individual's own home, remote locations, on-site or while traveling, personalized healthcare, point-of-care (POC), hospitals, clinics, emergency rooms, patient examination rooms, acute care patient rooms, ambulatory care, pediatrics, field environments, nurse's offices in educational settings, occupational health clinics, surgery or operation rooms.

In some of the embodiments described herein, a sample acquisition device is preferably used to collect and store a sample, e.g., blood, drawn from a subject. A subject as described herein can be an individual, a user, an end user, a patient, and the like. A subject can be an animal, preferably a primate or a non-primate. A subject can be a male or female. A subject can be pregnant, suspected of being pregnant, or planning to become pregnant. A subject can be ovulating. A subject can have a condition, e.g., cancer, autoimmune disease, or diabetes. A human can be an infant, child, teenager, adult, or elderly person. In certain embodiments, the mammal is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100, or over 12 years old, over 16 years old, over 18 years old, or over 21 years old.

The sample acquisition devices herein can be easily and conveniently used by a subject to draw a sample, e.g., blood sample, without the help or aid of others. Optionally in some cases, the device can be used by a third party to collect blood from a subject. A third party can include, for example a family member of the subject, trained medical professionals such as physicians and nurses, Emergency Medical Technicians (EMTs), clinicians, laboratory technicians, untrained medical personnel, etc. Optionally in any of the embodiments disclosed herein, a third party can be a non-living entity, e.g. a robot.

The device can be designed such that it is minimally invasive and generates a low level of pain (or reduced perception of pain) in the users. For example, the device can include a low number (e.g. one or two) piercing elements, instead of an array of multiple (three, four, five or more) needles or microneedles for penetrating the skin. Optionally, a device need not be pre-packaged with one or more piercing elements. For example, a variety of piercing elements can be operably and releasably coupled to the device, and/or interchanged onto the device e.g., after each use. In some alternate cases, a device can be operated without using piercing elements. For example, a subject's skin can have one or more pre-existing cuts, and the device can be placed over the one or more pre-existing cuts to draw blood using skin suction and vacuum pressure.

The device can be portable, disposable and designed for use in a single patient encounter. Optionally in any of the embodiments disclosed herein, the device can be re-usable. For example, a device can be used more than once, for example twice, three, four, five, five, six, seven, eight, nine, ten or more times. Optionally in any of the embodiments disclosed herein, a single device can be used in multiple patient encounters, either with a same subject or with a plurality of different subjects. The device can be of a form factor and ergonomically designed to facilitate the sample collection process. Sample collection, treatment and storage can be performed on a single device. In some cases, sample collection, treatment and storage can be performed using multiple components or devices (e.g., a piercing module and a vacuum module can be provided as separate devices that are operably connected or coupled together via one or more channels).

In some embodiments, a sample acquisition device can be configured or capable of collecting at least 150 μL of blood from a subject within a time window beginning from time of incision or penetration of a skin portion of the subject. The time window can be less than 5 minutes, preferably less than 3 minutes. In some embodiments, the time window can be under 2 minutes. Optionally in any of the embodiments disclosed herein, the time window can be under one minute. The device is capable of collecting a larger volume of blood at higher average flowrates compared to currently available non-venous collection devices.

In some other embodiments, a sample acquisition device can be configured to collect smaller amounts of blood (e.g. less than 150 μL, 140 μL, 130 μL, 120 μL, 110 μL, 100 μL, 90 μL, 80 μL, 70 μL, 60 μL, 50 μL, 40 μL, 30 μL, or 25 μL) of blood from a subject within a time window beginning from time of incision or penetration of a skin portion of the subject. The time window can be less than 5 minutes, preferably less than 3 minutes. In some embodiments, the time window can be under 2 minutes. Optionally in any of the embodiments disclosed herein, the time window can be under one minute.

FIGS. 1A, 1B, 1C, and 1D illustrate different views of an exemplary sample acquisition device 100. Specifically, FIG. 1A shows an overall perspective view of the device. The device can include a housing 102. The housing can include a housing base 110 and a housing cover 152 operably coupled to each other. The housing base can encompass a vacuum chamber and a deposition chamber as described further herein.

Optionally in any of the embodiments disclosed herein, a housing can be provided separately from the components of the device, and the housing need not be part of or integrated with the components. For example, a vacuum chamber, deposition chamber, cartridge chamber, and/or cartridge assembly as described elsewhere herein can be operably coupled to a separately provided housing. A recess as described herein can be provided on a portion of the housing. A housing can include a casing, enclosure, shell, box, and the like. A housing can include one or more hollow chambers, cavities or recesses. The housing may be formed having any shape and/or size. The housing can be configured to support one or more components as described elsewhere herein. Additionally or optionally, one or more of the components can serve or function as the housing. The housing can be integrated with one or more of the components herein, or one or more of the components can be integrated with or into the housing. The housing can be configured for mounting onto a surface such as, for example, skin of a subject. Optionally in any of the embodiments disclosed herein, a bracket or strap can be provided that allows the housing to be mounted to a surface.

The device can include a vacuum activator 114. The vacuum activator can include a button 115 located on the housing base. In some cases, the device does not have a vacuum activator or need not have a vacuum activator (e.g., the device can be configured to automatically configured to provide a vacuum upon sensing contact to an appropriate surface, without requiring a user to manually or semi-manually activate a vacuum activator). The device can further include a piercing activator 166. The piercing activator can include a button 167 located on the housing cover. In some cases, the device does not have a piercing activator or need not have a piercing activator (e.g., the device can be used to draw blood from skin that has already been penetrated or pre-cut by other discrete stand-alone piercing elements). The piercing activator can be preferably activated after the vacuum activator has been activated. In some cases, the piercing activator can be activated independently of the vacuum activator or vacuum state of the device. In some embodiments, the piercing activator can be locked prior to use of the device, and the piercing activator can be activated only after the vacuum activator has been activated. In some cases, the vacuum activator is locked prior to use of the device, and the vacuum activator can be activated only after the piercing activator has been activated. The piercing activator (e.g., button 115) and vacuum activator (e.g., button 167) can be located on the same side or face of the housing. Alternatively, the piercing activator (e.g., button 115) and vacuum activator (e.g., button 167) can be located on different sides or faces of the housing. The device 100 or any of the devices herein can further include a cartridge assembly 180. Such cartridge assembly can be releasably coupled to the device and detached from the device. As shown in FIG. 1A, a cartridge tab 192 of the cartridge assembly can protrude from an edge of the device. Optionally in any of the embodiments disclosed herein, the cartridge tab and the piercing activator/vacuum activator (e.g., buttons 115/167) can be located on different sides (e.g. opposite ends) of the housing. Additional details about the vacuum activator and the piercing activator are described herein.

FIG. 1B shows a bottom perspective view of the device, in particular a recess 136 provided on the housing base 110. The recess can be a concave cavity. The recess can have a cup-like shape. Optionally in any of the embodiments disclosed herein, the recess can have a substantially hemispherical shape. The housing base can be configured to be placed and releasably attached onto a portion of a subject's body, for example on the subject's upper arm. A portion of a subject's skin can be drawn into the recess with aid of vacuum pressure, e.g., as described elsewhere herein. The recess can be configured having a shape and/or size that enables an increased volume of a fluid sample (e.g., blood) to be collected from a subject. The housing base can include a planar portion 132 to be placed on the subject's skin. The planar portion can surround the periphery of the recess. The planar portion of the housing base can have any shape. Optionally in any of the embodiments disclosed herein, the planar portion can include an annular ring-like shape. An adhesive (not shown) can be placed on the planar portion of the housing base to promote adhesion of the device to the subject's skin, and to create an airtight hermetic seal after the device has been placed onto the skin. Optionally in any of the embodiments disclosed herein, a fillet 138 can be provided between the periphery of the recess and the planar portion of the housing base. The fillet can improve vacuum suction to the skin and reduce leaks. As shown in FIG. 1B, the recess can include an opening 140. The opening can be located anywhere in the recess. For example, the opening can be located at an innermost portion of the recess. Optionally in any of the embodiments disclosed herein, a fillet 139 can be provided at the periphery of the opening. Additional details about the recess, the opening, and suction of skin into the recess are described herein.

The opening 140 can be an opening of a lumen 142. The lumen can include a port 144 leading to a deposition chamber (not shown) located in the housing base. Optionally in any of the embodiments disclosed herein, the lumen can include a cutout 145, and the port 144 can be provided within or proximal to the cutout. The cutout 145 can help to reduce or prevent occlusion of the port 144 by a subject's skin when the skin is drawn into the recess of the housing base. Keeping access to the port 144 open (e.g. by not having the port occluded or blocked by skin) can help to ensure that blood drawn from a subject's skin is able to flow through the port 144 into the deposition chamber. The lumen can further include a port 150 leading to an enclosure for holding one or more piercing elements (not shown). The one or more piercing elements can be configured to extend out of the opening to penetrate the subject's skin when (or after) the skin is drawn into the recess by vacuum pressure. The one or more piercing elements can be subsequently retracted back into the housing after penetrating the skin. Additional details about the one or more piercing elements and their actuation are described herein.

Blood can be drawn from cuts made on the skin. The blood can flow from the cuts through the port 144 towards a cartridge (not shown) located in a deposition chamber in the housing base. The flowrate and volume of the blood collection can be enhanced (e.g. increased) with aid of the vacuum, pressure differentials, gravitational force, and wicking/capillary effects, e.g., as described in detail elsewhere herein. The cartridge can include one or more matrices for collecting and storing a predefined volume of the blood. Additional details about the enhanced fluid collection are described in various parts of the Specification, for example in Section II Part G.

FIG. 1C shows a flow meter 170 of the device. The flow meter can include one or more optically transparent windows 172. The flow meter can be substantially aligned with the cartridge (specifically the matrices in the cartridge) when the cartridge assembly is inserted into the device. The flow meter can allow the subject or another user to monitor a progress of the sample (e.g., blood) collection in real-time as the sample is being collected into the cartridge. In some embodiments, the flow meter can be provided on a lid of the housing base. For example, the flow meter can be formed as part of the lid. The lid can be an intervening layer between the housing base and the housing cover. The lid can cover the housing base, and seal a vacuum chamber in the housing base. In some embodiments, the lid can be ultrasonically welded to the housing base. The lid can provide an airtight hermetic seal. Additional details about the flow meter are described, e.g., in Section II Part F of the Specification.

FIG. 1D shows a cartridge assembly 180 that can be releasably coupled to the device. The cartridge assembly can be part of the device, and can be decoupled from the device. The cartridge assembly can be inserted into a deposition chamber (or cartridge chamber) of the housing base via an opening 128. The cartridge assembly can include a cartridge 182 and a cartridge holder 190. The cartridge holder is configured to support the cartridge. The cartridge holder can include a cartridge tab 192, a seal/gasket 194, and spring clips 196. A subject or user can handle or hold the cartridge assembly using the cartridge tab. For example, the subject can insert the cartridge assembly into the deposition chamber (cartridge chamber) of the device by pushing in the cartridge tab. After the sample collection has been completed, the subject can remove the cartridge assembly from the deposition chamber (cartridge chamber) of the device by pulling the cartridge tab. The subject can also hold the cartridge assembly by the cartridge tab to avoid contamination to the cartridge and/or sample. The seal/gasket 194 can hermetically seal the deposition chamber (cartridge chamber) once the cartridge assembly is properly inserted into the device. The spring clips 196 allow the cartridge to be held in place by the cartridge holder.

The cartridge can be configured to support one or more matrices 186 on which the fluid sample (e.g., blood) is collected. In some embodiments, the cartridge can support two or more matrices. The two or more matrices can separated by one or more spacers. The cartridge can include a cartridge port 184 and a channel (not shown) leading to the matrices. The cartridge can be configured to support one or more absorbent pads (not shown) for holding excess fluid. The absorbent pads help to ensure that a predefined volume of fluid can be collected on each of the matrices. Additional details about the cartridge assembly are described, e.g., in Section II Part C of the Specification.

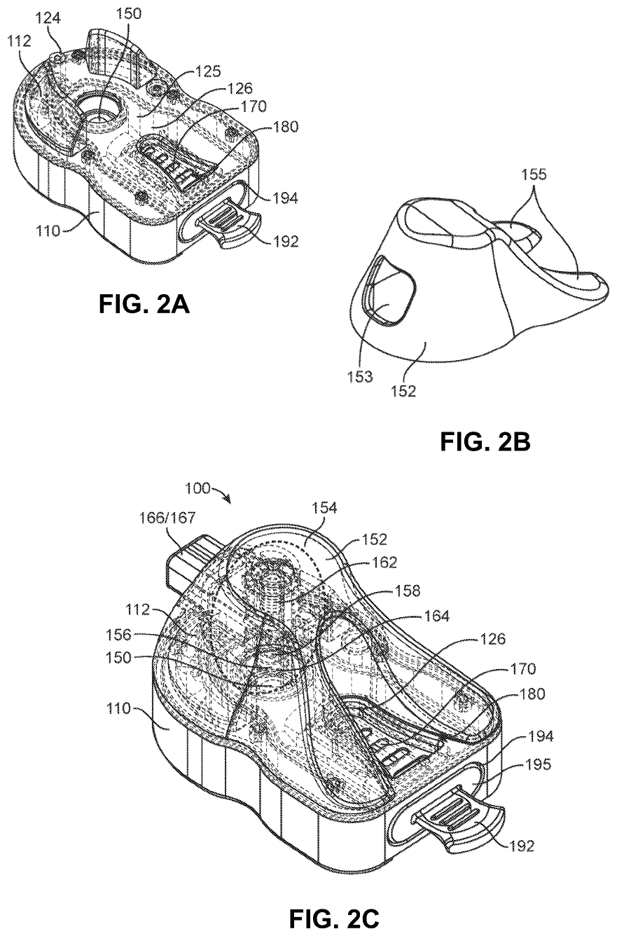

The housing base 110 and the housing cover 152 can each be separately provided, and coupled together to form the housing. For example, FIG. 2A shows a perspective view of the housing base 110 with a lid 124 covering/sealing the housing base. The housing base can include a vacuum chamber 112 and a deposition chamber 126. The vacuum chamber and the deposition chamber can be separated by one or more walls 125. The walls can be substantially impermeable to fluids (e.g. gases and liquids). The lid 124 can hermetically seal the vacuum chamber and the deposition chamber. The lid can include the flow meter 170. The deposition chamber can also serve as a cartridge chamber, and can be interchangeably referred to as such herein. A cartridge assembly 180 is shown inserted into the deposition chamber (or cartridge chamber). The seal/gasket 194 can hermetically seal the deposition chamber once the cartridge assembly is fully inserted into the deposition chamber. FIG. 2B shows a perspective view of the housing cover 152. The housing cover can include a through-hole 153 through which the button 167 of the piercing activator 166 can be inserted. The housing cover can include wings 155 having a U or V-like shape to prevent obscuring the flow meter on the lid of the housing base. Accordingly, the housing cover can be shaped in a manner that allows a subject or another user to view the flow meter and monitor the progress of the fluid sample collection. The housing cover can have sufficient vertical (Z-height) clearance to permit placement of a piercing module 154 therein. The piercing module can comprise one or more piercing elements that are configured to extend and retract through the opening of the recess. FIG. 2C shows a perspective view of the assembled device 100 whereby the housing cover and the housing base are coupled together. Exemplary means of attachment of the housing cover to the housing base can include snapfits, ultrasonic welding, nuts and bolts, rivets, screws, nails, locks, latches, wires, joints, soldering, welding, gluing and the like. In some alternative embodiments, the housing base and housing cover can be monolithically and collectively formed as a single component.

The housing of the device can be formed having any shape and/or size. The housing or any components thereof can be formed using any number of techniques known in the art such as injection molding, blow molding, three-dimensional (3D) printing, etc. The housing can include materials suitable for healthcare applications (e.g., the housing material is compatible for use with biological materials), depending on the particular application. For example, components of the housing can include or be fabricated from materials such as cellophane, vinyl, acetate, polyethylene acrylic, butyl rubber, ethylene-vinyl acetate, natural rubber, a nitrile, silicone rubber, a styrene block copolymer, a vinyl ether, or a tackifier. Optionally in any of the embodiments disclosed herein, the device can include antimicrobial and/or antiseptic materials, for example sodium bicarbonate; hydrogen peroxide; benzalkonium chloride; chlorohexidine; hexachlorophene; iodine compounds; and combinations thereof.

Optionally in any of the embodiments disclosed herein, one or more components of the device can include or can be fabricated from materials such as polyvinyl chloride, polyvinylidene chloride, low density polyethylene, linear low density polyethylene, polyisobutene, poly [ethylene-vinylacetate]copolymer, lightweight aluminum foil and combinations thereof, stainless steel alloys, commercially pure titanium, titanium alloys, silver alloys, copper alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, partially resorbable materials, such as, for example, composites of metals and calcium-housing based ceramics, composites of PEEK and calcium housing based ceramics, composites of PEEK with resorbable polymers, totally resorbable materials, such as, for example, calcium housing based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

The housing of the device can comprise acrylobutadiene styrene (ABS), polypropylene (PP), polystyrene (PS), polycarbonate (PC), polysulfone (PS), polyphenyl sulfone (PPSU), polymethyl methacrylate (acrylic)(PMMA), polyethylene (PE), ultra high molecular weight polyethylene (UHMWPE), lower density polyethylene (LPDE), polyamide (PA), liquid crystal polymer (LCP), polyaryl amide (PARA), polyphenyl sufide (PPS), polyether etherketone (PEEK), polyvinyl chloride (PVC), polyethylene terephthalate (PET), polytetra flouroethylene (PTFE), polyaryletherketone (PAEK), polyphenyl sulfone (PPSU), or a combination thereof. In some embodiments, a device disclosed herein can comprise polypropylene, polycarbonate, glass filled polycarbonate, a low permeability copolyester (e.g. Eastman MN211), polyisoprene rubber, and/or TPE injection moldable seals.

Various components of the device can have material composites, including one or more of the above materials, to achieve various desired characteristics such as strength, rigidity, elasticity, compliance, biomechanical performance, durability and/or radiolucency preference. One or more of the components of the device can comprise antimicrobial and/or antiseptic materials. The components of the device, individually or collectively, can also be fabricated from a heterogeneous material such as a combination of two or more of the above-described materials. The components of the device can be monolithically formed or integrally connected.

The device can be ergonomically designed such that a subject or user is able to hold the device comfortably with one hand or both hands. The device can have a compact form factor that makes it highly portable (e.g. easy to be carried around in a user's bag or purse). Exemplary dimensions (e.g. length, width and height) of the device can be given as follows. In some embodiments, the length is about 1.5 inches, about 2.0 inches, about 2.5 inches, about 3.0 inches, or about 3.5 inches. The length can be between about 2.0 inches and about 3.0 inches. The length can be between about 1.5 inches and about 3.5 inches. In some embodiments, the width is about 1.25 inches, about 1.5 inches, about 1.75 inches, about 2.0 inches, or about 2.25 inches. The width can be between about 1.5 inches and about 2.0 inches. The width can be between about 1.25 inches and about 2.25 inches. In some embodiments, the height is about 1.25 inches, about 1.5 inches, about 1.65 inches, about 2.0 inches, or about 2.25 inches. The height can be between about 1.5 inches and about 2.0 inches. The height can be between about 1.25 inches and about 2.25 inches. The length by width by height can be about 2.5 inches by about 1.75 inches by about 1.65 inches.

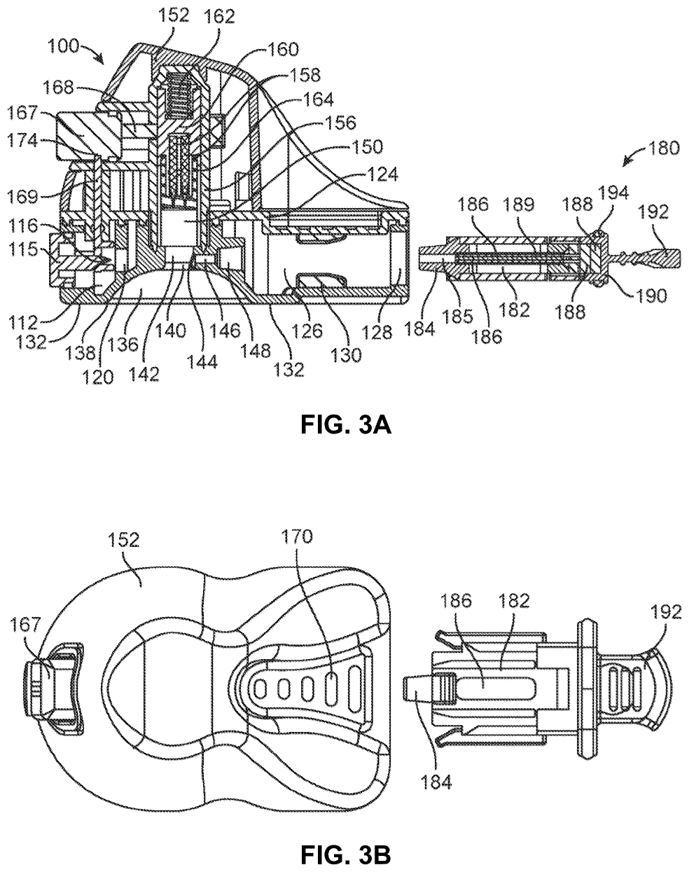

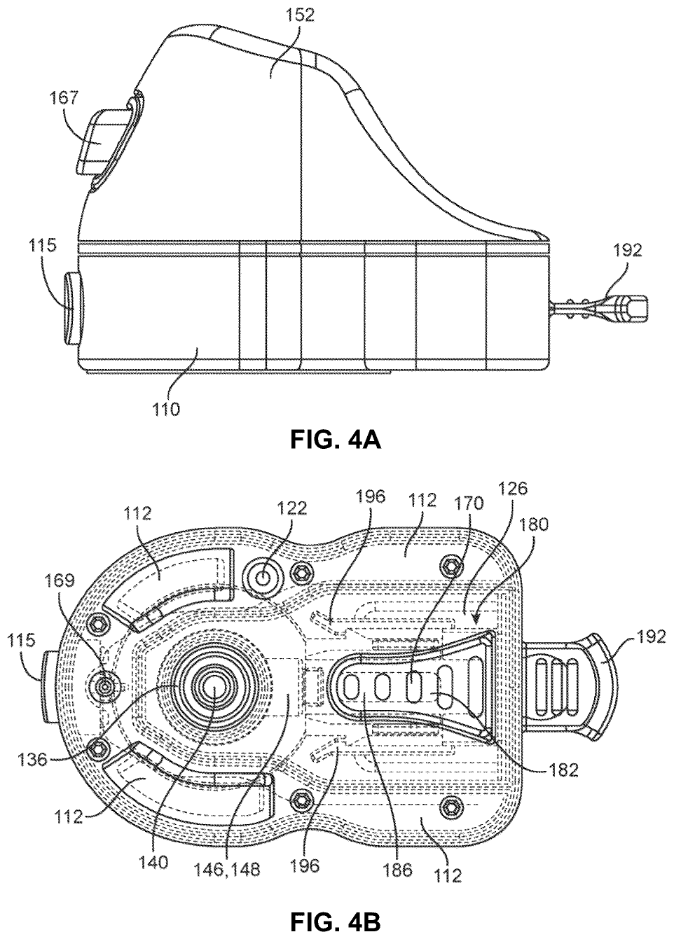

FIG. 3A shows a side sectional view of the device 100 prior to insertion of the cartridge assembly 180 into the device, and FIG. 3B shows a corresponding top view. FIG. 4A shows a side view of the device with the cartridge assembly inserted therein, and FIG. 4B shows a corresponding top sectional view. Various features of the device 100 and the cartridge assembly 180 are next described in detail with reference to the above figures and other relevant figures.

### A. Recess for Skin Suction

Referring to FIGS. 1B and 3A, the housing base 102 of the device can include the recess 136. The recess can be provided on a portion (e.g. bottom surface) of the housing base. The recess can be formed as a sunken cavity or trench on the housing base. In some cases, the recess can be formed as a molded extrusion into the housing base. The recess can be shaped like a cup and configured to provide a skin “cupping” effect with aid of vacuum pressure. The recess can be sized and/or shaped to receive a portion of a surface, e.g., subject's skin therein, and to permit the surface, e.g., skin to substantially conform to the recess under application of vacuum pressure. A surface of the recess can be substantially in contact with the skin drawn into the recess. A gap between the skin and the recess can be negligible when the skin is drawn into the recess. The recess can serve as a suction cavity for drawing the skin therein and for increasing capillary pressure differential. The recess can be configured having a size and/or shape that enables an increased volume of blood to be accumulated in the skin drawn into the recess. The increased volume of the fluid sample can depend in part on a volume and/or surface area of the skin that is drawn into the recess.

In some alternative embodiments, the device can be configured to draw other types of objects (e.g. objects that are not skin or skin surfaces) into the recess under vacuum, and to further draw a fluid sample from those objects. Examples of those other types of objects can include sponges, clothes, fabrics, paper, porous materials, organic produce such as fruits or vegetables, or any solid materials that are holding (or capable of holding) fluid samples therein or thereon. Additional non-limiting examples of biological samples suitable for use with the devices of the disclosure can include sweat, tears, urine, saliva, feces, vaginal secretions, semen, interstitial fluid, mucus, sebum, crevicular fluid, aqueous humour, vitreous humour, bile, breast milk, cerebrospinal fluid, cerumen, enolymph, perilymph, gastric juice, peritoneal fluid, vomit, and the like. In some embodiments, a fluid sample can be a solid sample that has been modified with a liquid medium. In some instances, a biological sample can be obtained from a subject in a hospital, laboratory, clinical or medical laboratory.

The recess can be configured to maintain contact with a skin surface area of the subject under vacuum pressure, prior to and as blood is being collected from penetrated skin of the subject. In some embodiments, the skin surface area of the subject in contact with the recess can be at least 3 cm², 4 cm², 5 cm², 6 cm², 7 cm², 8 cm², 9 cm², or 10 cm², or any value therebetween. In some preferred embodiments, at least 5 cm² of the skin surface area of the subject can be in full contact with the surface of the recess when the skin is drawn into the recess under vacuum pressure. In some embodiments, the volume of the skin enclosed within the recess can be at least about 1.0 cm³, 1.1 cm³, 1.3 cm³, 1.4 cm³, 1.4 cm³, 1.5 cm³, 1.6 cm³, 1.7 cm³, 1.8 cm³, 1.9 cm³, 2.0 cm³, 2.1 cm³, 2.2 cm³, 2.3 cm³, 2.4 cm³, 2.5 cm³, 2.6 cm³, 2.8 cm³, 2.9 cm³, 3.0 cm³, or any value therebetween. In some embodiments, at least 1.8 cm³ of the subject's skin can be enclosed within the recess when the skin is drawn into the recess under vacuum pressure. In some embodiments, the volume of the enclosed within the recess can be substantially the same as an inner volume of the recess.

Optionally in any of the embodiments disclosed herein, the housing base of the device can have more than one recess, e.g. 2, 3, 4, 5, 6, 7, 8, 9, 10 or more recesses. The recesses can be connected to one another, for example by one or more channels. Alternatively, the recesses need not be connected to one another. The recesses can be in fluidic communication with one or more of the vacuum chambers and deposition chambers described elsewhere herein. The plurality of recesses can be configured to permit suction to occur on multiple portions of a surface (e.g. skin surface). In some cases, the plurality of recesses can enable blood to be drawn from different portions of a user's skin (that is drawn into the plurality of recesses).

The recess can be formed having any shape, design, depth, surface area, and/or size. The recess can have any convenient shape, such as a curved shape, hemispherical, spherical cap, square, circle, cuboid, trapezoidal, disc, etc. The recess can be symmetrical, for example a hemisphere. Alternatively, the recess can have an irregular shape and need not be symmetrical. The recess can have rounded corners or edges. Additional examples of possible shapes or designs include but are not limited to: mathematical shapes, two-dimensional geometric shapes, multi-dimensional geometric shapes, curves, polygons, polyhedral, polytopes, minimal surfaces, ruled surfaces, non-orientable surfaces, quadrics, pseudospherical surfaces, algebraic surfaces, riemann surfaces, geometric shapes, and so forth. Optionally in any of the embodiments disclosed herein, the recess can have a substantially circular or elliptical shape. The surface of the recess can be smooth. In some embodiments, the recess can be configured to have a shape and/or size that can reduce or eliminate bruising on the skin when the skin is drawn into the recess by vacuum pressure. Optionally, the surface of the recess can take on a variety of alternative surface configurations. For example, in some cases, the surface of the recess can contain raised or depressed regions.

Referring to FIG. 1B, the recess can comprise a concave cavity. The concave cavity can enclose an interior volume of at least about 1.0 cm³, 1.1 cm³, 1.3 cm³, 1.4 cm³, 1.4 cm³, 1.5 cm³, 1.6 cm³, 1.7 cm³, 1.8 cm³, 1.9 cm³, 2.0 cm³, 2.1 cm³, 2.2 cm³, 2.3 cm³, 2.4 cm³, 2.5 cm³, 2.6 cm³, 2.8 cm³, 2.9 cm³, 3.0 cm³, or any value therebetween. In some embodiments, the concave cavity can preferably enclose an interior volume of about 1.85 cm³.

The recess can have a depth ranging from about 2 mm to about 30 mm, or preferably at least deep enough such that a skin portion of the subject is drawn into and completely fills the recess under vacuum pressure. The depth can be a height of the recess. The depth can be measured relative to an innermost portion of the recess. In some other embodiments, the recess can have a depth that is less than 2 mm or greater than 10 mm.

The recess can have a rigid surface (e.g. a rigid concave surface) that does not deform when skin of a subject is drawn into the recess under vacuum pressure. Alternatively, the recess can have a flexible surface (e.g. a flexible concave surface). For example, the bottom of the recess can include an elastic material such as an elastomer. The elastic material can be configured to conform to the skin when the skin is drawn into the recess. The elastic material can compress or press against the skin when the skin is drawn into the recess. The compression can help to improve the contact area between the skin and the recess. Increased contact area can allow the skin to completely fill the recess with reduced gaps or creases inbetween. This can help to ensure that the skin is sufficiently taut (under tension) prior to penetration of the skin for blood collection. Holding the skin taut can enable deeper cuts to be made in the skin. Furthermore, holding the skin taut can also hold the cuts open better compared to loose skin.

As shown in FIGS. 1B and 3A, the recess can be in the shape of a spherical cap. The spherical cap can be, for example a hemisphere or part of a hemisphere. In some embodiments, a housing base diameter of the spherical cap can range from about 10 mm to about 60 mm, preferably about 25 mm. A height of the spherical cap can range from about 2 mm to about 30 mm, preferably about 6 mm. A volume of a hemisphere formed by the concave surface can be equivalent to, or about half, or about a quarter, of a volume of a vacuum chamber in the device.

Referring to FIGS. 1B and 3A, the recess can include the opening 140. The opening can be located at an innermost portion of the recess. For example, the opening can be located at an apex of the spherical-shaped recess. The opening can be formed having any shape and/or size. In some embodiments, the opening can have a substantially circular or elliptical shape. In some cases, the recess can have more than one opening, e.g., 1, 2, 3, 4, 5, 6, 7, 8, 9, 10, 25, 50, 100, 1000, or more openings. One or more of the openings can be in fluidic communication with a vacuum source and/or an enclosure holding one or more piercing elements. In some cases, one or more openings, e.g., connected to a vacuum source, can be found throughout the surface of the recess, or at least 10%, 25%, or 50% of the surface of the recess. Optionally in any of the embodiments disclosed herein, a plurality of openings can be distributed across the surface of the recess, for example in a manner similar to a showerhead. A vacuum can be applied via the plurality of openings to draw a subject's skin into the recess. In some cases, one or more of the plurality of the openings can be further configured to permit one or more piercing elements to extend and retract therethrough, and to pierce the skin that is drawn into the recess.

The opening 140 can provide access to/from the lumen 142. The device can include one or more piercing elements that are configured to extend through the lumen and out of the opening into the recess, to penetrate skin that is drawn into the recess under vacuum pressure. The penetration of the skin can permit blood to be drawn from the subject, e.g., as described in detail elsewhere herein. The lumen can include two or more ports. For example, the lumen can include a first port 144 leading to the deposition chamber 126 located in the housing base, and a second port 150 leading to an enclosure 156 located in the housing cover. A piercing module 154 comprising one or more piercing elements 158 can be provided in the enclosure 156.

A size of the recess 136 can be substantially greater than a size of the opening 140. For example, a size of the recess can be at least twice a size of the opening. In some embodiments, the size (e.g. diameter) of the opening can range from about 1.5 mm to about 6 mm, and the size (e.g. base diameter or width) of the recess can range from about 10 mm to about 60 mm. In some preferred embodiments, a diameter of the opening can be about 5 mm, and a base diameter of the recess can be about 25 mm.

In some embodiments, a ratio of the size (e.g. diameter) of the opening to the size (e.g. base diameter) of the recess can be about 1:2, about 1:3, about 1:4, about 1:5, about 1:6, about 1:7, about 1:8, about 1:9, about 1:10, about 1:20, about 1:25, about 1:50, or about 1:100, or any ratios therebetween. In some embodiments, a ratio of the size (e.g. diameter) of the opening to the size (e.g. base diameter) of the recess can be about 1:2 to about 1:10, or from about 1:5 to about 1:50, or from about 1:10 to about 1:100. The aforementioned ratio can be also less than about 1:2, 1:3, 1:4, 1:5, 1:10, 1:15, 1:20; 1:25; 1:30; 1:50, or 1:100. In some embodiments, the ratio of the size (e.g. diameter) of the opening to the size (e.g. base diameter) of the recess can be preferably at least about 1:5.

A surface area of the recess 136 can be substantially greater than an area of the opening 140. The surface area of the recess can be associated with the interior of the recess (excluding the opening), and can be measured across a 3D (e.g. a concave hemispherical) plane. The area of the opening can be measured across a substantially 2D or quasi-2D plane defined by the opening. In some embodiments, the surface area of the recess can be at least five times, six times, seven times, eight times, nine times, ten times, or twenty times the area of the opening. In some embodiments, the surface area of the recess can range from about 75 mm² to about 2900 mm², and the area of the opening can range from about 1.5 mm² to about 30 mm². In some embodiments, the area of the opening can preferably be about 0.2 cm², and the surface area of the recess can preferably be about 5.2 cm².

In some embodiments, an area of the skin directly under the opening 140 can be at least 1.5 times smaller than a total area of the skin drawn into the recess 136. In some embodiments, the area of the skin directly under the opening can be preferably at least 5 times smaller than the total area of the skin drawn into the recess.

Referring to FIG. 1B, the planar portion 132 of the housing base can be configured to be placed onto the skin (e.g., on the upper arm) of the subject. The planar portion can be provided surrounding the recess. An adhesive (not shown) can be placed on the planar portion of the housing base. The adhesive can create an airtight hermetic seal on the skin that prevents air from the ambient environment from entering the recess after the device is placed onto the subject's skin. The seal can also prevent fluids (e.g., blood, gas, etc.) from escaping out of the recess into the ambient environment after the device is placed onto the subject's skin. An appropriate biocompatible adhesive material or gasket material can be placed on the planar portion on the housing base, to promote adhesion of the device onto the subject's skin for improved contact. Any suitable adhesive can be used. The adhesive can be a hydrogel, an acrylic, a polyurethane gel, a hydrocolloid, or a silicone gel.

The adhesive can be a hydrogel. Optionally in any of the embodiments disclosed herein, the hydrogel can comprise a synthetic polymer, a natural polymer, a derivative thereof, or a combination thereof. Examples of synthetic polymers include, but are not limited to poly(acrylic acid), poly(vinyl alcohol)(PVA), poly(vinyl pyrrolidone)(PVP), poly(ethylene glycol)(PEG), and polyacrylamide. Examples of natural polymers include, but are not limited to alginate, cellulose, chitin, chitosan, dextran, hyaluronic acid, pectin, starch, xanthan gum, collagen, silk, keratin, elastin, resilin, gelatin, and agar. The hydrogel can comprise a derivatized polyacrylamide polymer.

In some embodiments, the adhesive can be a 3-layer laminate comprising of (1) hydrogel for applying to the skin side), (2) Tyvek™, and (3) a secondary adhesive for bonding to the planar portion of the housing base of the device.

In some embodiments, the adhesive can be pre-attached to the planar portion on the housing base of the device 100. The device can comprise a protective film or backing covering the adhesive on the planar portion. The protective film can be removed prior to use of the device and placement of the device on the subject's skin. In another embodiment, an adhesive in the form of a gel, a hydrogel, a paste, or a cream can be applied to skin of the subject or to the planar portion on the housing base of the device, prior to placement of the device on the subject's skin. The adhesive can then be placed in contact with the subject's skin for a predetermined amount of time (e.g., on the order of several seconds to several minutes) in order to form an adhesion layer between the skin and device. The adhesive can be a pressure-sensitive adhesive or a heat-sensitive adhesive. In some embodiments, the adhesive can be hypoallergenic.

In some embodiments, the adhesive can be a peelable adhesive, and can have a shape and size corresponding to the planar portion on the housing base of the device. In the example shown in FIG. 1B, the planar portion on the housing base can be in the shape of an annular ring, although any shape can be contemplated. Accordingly, the peelable adhesive can be provided as an annular ring corresponding to the planar portion on the housing base.

In some embodiments, a fillet 138 can be provided at an interface between the planar portion and the recess. For example, the fillet 138 can extend continuously along a periphery of the recess adjoining the planar portion of the housing base. The fillet can be configured having a radius or curvature that can help to improve vacuum suction to the skin and to reduce vacuum leak. For example, the fillet of the recess can conform to and be substantially in contact with the skin of the subject when the skin is drawn into the recess. In some embodiments, a fillet 139 can be provided along the periphery of the opening, for example as shown in FIGS. 1B and 3A. The use of fillets can also eliminate sharp edges and reduce unwanted cuts or bruises to the skin when the skin is drawn into the recess under vacuum pressure.

Optionally in any of the embodiments disclosed herein, the recess can be coated or sprayed with a copper, silver, titanium or other metal, coating, or any other antimicrobial material, anti-viral material, surfactants or agents that are designed to reduce microorganisms, disease, virus, cellular, bacteria, or airborne or surface particulates from clinging onto the surface and/or edges of the recess. Optionally in any of the embodiments disclosed herein, one or more walls of the recess can be impregnated with an antimicrobial material. For example, the antimicrobial material can be integrally formed with the recess of the housing to help control the bacterial level present on or within the recess.

### B. Vacuum Chamber and Deposition Chamber

The device can include a vacuum chamber 112 and a deposition chamber 126, for example as shown in FIGS. 2A, 2C, 3A and 4B. The vacuum chamber and the deposition chamber can be provided in the housing (e.g. integrated into the housing base). Optionally, the vacuum chamber and the deposition chamber can be operably coupled to a separately provided housing. The vacuum chamber can be configured to be in fluidic communication with the recess and the deposition chamber. The vacuum chamber and the deposition chamber can be part of the housing base. The vacuum chamber and the deposition chamber can be located in different sections (e.g. compartments) of the housing base, and provided having various shapes or configurations. For example, in some embodiments, the vacuum chamber can be shaped like a horse-shoe surrounding the deposition chamber, as shown in FIGS. 2A and 4B. The vacuum chamber and the deposition chamber can be separated by one or more walls 125. The walls can be substantially impermeable to fluids (e.g. gases and liquids) and can prevent leaks between the chambers. The walls can be made materials having very low permeability values. For example, polypropylene can have a permeability coefficient of 9×10⁻¹¹ (cm³ cm)/(sec·cm²·cm·Hg) to oxygen, and 4.5×10⁻¹¹ (cm³ cm)/(sec·cm²·cm·Hg) to air. As an example, PETG can have a permeability coefficient of 1.5×10⁻¹¹ (cm³ cm)/(sec·cm²·cm·Hg) to oxygen, and 7.5×10⁻¹² (cm³ cm)/(sec·cm²·cm·Hg) to air. In some alternative cases, the vacuum chamber and the deposition chamber need not be separated, e.g., by walls. For example, the vacuum chamber and the deposition chamber can be the same chamber in a device as packaged. The combined vacuum chamber and the deposition chamber can be a monolithic chamber. A chamber can have more than one function or purpose, for example 2, 3, 4, 5, 6, 7, 8, 9, 10 or more functions or purposes. For example, in some cases, a vacuum chamber can also serve the function of a deposition chamber. Likewise, in some cases, a deposition chamber can also serve the function of a vacuum chamber.

The deposition chamber can be interchangeably referred to as a cartridge chamber, since the deposition chamber can be configured to receive a cartridge assembly 180 therein. Blood can be collected from the subject, and transported from the recess into the deposition chamber for collection and storage onto a cartridge 182. In some cases, the device comprises more than one vacuum chamber, e.g., 2, 3, 4, 5, 6, 7, 8, 9, 10, or more vacuum chambers (each vacuum chamber can be connected to a different recess or the same recess), and/or more than one deposition chamber, e.g., 2, 3, 4, 5, 6, 7, 8, 9, 10, or more deposition chambers (each deposition chamber can be connected to the same vacuum chamber or a different vacuum chamber). Any number of vacuum chambers and/or deposition chambers can be contemplated for depending on design applications and needs.

The housing base can include a lid 124 that covers and hermetically seals the vacuum chamber. The lid can serve as a vacuum chamber lid. The lid can also cover the deposition chamber or a portion thereof. The vacuum chamber can be an evacuated chamber, and can be referred to interchangeably as such. Referring to FIG. 4B, the vacuum chamber can include a self-sealing septum 122 through which air can be drawn out of the vacuum chamber. A vacuum state can be generated in the vacuum chamber, for example by inserting a distal end of a syringe through the septum 122, and using the syringe to draw air out of the vacuum chamber. The distal end of the syringe can comprise a needle that is inserted through the septum into the vacuum chamber. The septum can be made of any appropriate airtight flexible or elastomeric material. In some embodiments, the septum can be made of polyisoprene. The septum can be in a naturally sealed state, and can revert to its sealed state when the needle is removed from the septum.

In some other embodiments, a mechanical device such as a vacuum pump can be used to evacuate the vacuum chamber (e.g., before or after packaging). The mechanical device can include components such as pistons, motors, blowers, pressure regulators, and the like. In some cases, non-mechanical means, such as chemicals or other reactants, can be introduced to the vacuum chamber and can undergo reaction to decrease pressure within the vacuum chamber (e.g., create a vacuum state).

The housing base can include a separation interface 120 that separates the vacuum chamber from the deposition chamber. The separation interface can be, for example a foil. In some embodiments, the separation interface can be a multi-layer foil laminate. The separation interface can include any materials or means that can serve as a fluidic barrier between the vacuum chamber and the deposition chamber. The separation interface can be “opened” to enable fluidic communication between the vacuum chamber and the deposition chamber. Other non-limiting examples of a separation interface include diaphragms, caps, seals, lids, membranes, valves, and the like. The separation interface can be bonded to the housing base using any of the attachment means described herein. The separation interface can include any suitable polymer or composite material that can be pierced by a sharp object. The separation interface can be impermeable or semipermeable to gas or liquids. For example, suitable materials for use in the separation interface can include polymer thin films, polyethylene, latex, etc.

The separation interface, e.g., foil, can help to maintain the vacuum pressure in the vacuum chamber, and the pressure difference between the vacuum chamber and the deposition chamber. Piercing the separation interface, e.g., foil, can result in pressure equalization between the vacuum chamber and the deposition chamber, and create a pressure differential (negative pressure) that (1) draws the skin into the recess and (2) further draws blood from skin of the subject after the skin has been penetrated. In some embodiments, a vacuum pressure of at least about −1 psig to −2 psig is provided, in order to draw the surface, e.g., skin into the recess and completely fill the recess. In some embodiments, the skin is drawn into the recess by the vacuum and completely fills the recess in less than 2 seconds, preferably less than 1 second. In some embodiments, the skin is drawn into the recess by the vacuum and completely fills the recess in no more than 5 seconds.

In some cases, the vacuum chamber and the deposition chamber need not be separated, i.e., the vacuum chamber and the deposition chamber can be the same chamber, or can collectively constitute a same chamber. In those cases, the combined vacuum chamber/deposition chamber can be separated from an opening of the recess by a separation interface, e.g., foil. As an example, the separation interface can be provided at or proximal to the opening of the recess, and can be used to establish fluidic communication between the recess and the combined vacuum chamber/deposition chamber.

As previously described, the recess of the device can be configured having a size and/or shape that enables higher average flowrate, and an increased volume of blood to be accumulated and collected. The collection flowrate can be dependent on the shape and/or size of the recess. For example, the recess shown in FIG. 1B can aid in enhancing the flowrate of blood collected from the subject.

The increased volume and flowrate of the blood collection can also depend on a starting or initial vacuum pressure of the vacuum chamber. The starting or initial vacuum pressure can correspond to the pressure of the vacuum chamber post evacuation. In some embodiments, the initial vacuum pressure of the vacuum chamber can range from about −4 psig to about −15 psig, preferably about −8 psig to about −12 psig. In some preferred embodiments, the initial vacuum pressure of the vacuum chamber can be about −12 psig. In some other embodiments, the initial vacuum pressure of the vacuum chamber can be less than −15 psig, for example −16 psig, −17 psig, −18 psig, −19 psig, −20 psig, −21 psig, −22 psig, −23 psig, −24 psig or lower.

The vacuum chamber can have a volume V1 ranging from about 3 cm³ to about 30 cm³. The deposition chamber can have a volume V2 ranging from about 1 cm³ to about 20 cm³. In some embodiments, the volume V1 of the vacuum chamber is preferably about 10 cm³, and the volume V2 of the deposition chamber is preferably about 6 cm³.” The volumes of the vacuum chamber and the deposition chamber can be designed such that the pressure in both chambers equalizes to a desired value when the separation interface, e.g., foil, separating the two chambers is pierced. For example, the vacuum chamber can have an initial starting vacuum pressure of about −12 psig, and the ratio of V1 to V2 can be configured such that the equalized pressure in both chambers is about −4 psig after the foil is pierced. Any ratio of V1:V2 can be contemplated, for example 1:1, 1:2, 1:3 and so forth.

In some embodiments, the increased volume of the blood in the skin drawn into the recess is at least about 20 μL, 30 μL, 40 μL, 50 μL, 60 μL, or 70 μL prior to the penetration of the skin. Higher flowrates and blood sample collection volumes can be achieved in part due to the increased volume of blood in the skin drawn into the recess, increased capillary pressure, and with aid of the vacuum pressure. In some embodiments, the device is capable of drawing blood from penetrated skin and collecting the blood at a flowrate of at least about 30 μL/min. In some embodiments, the device can be capable of drawing blood from penetrated skin and collecting the blood at a flowrate of more than 600 μL/min. Generally, the device is capable of drawing blood from penetrated skin and collecting the blood at an average flowrate of at least about 100 μL/min, 125 μL/min, 150 μL/min, or any values or ranges therebetween. In some embodiments, the device can sustain the aforementioned average flowrate(s) at least until a substantial amount of blood has been collected (e.g. ranging from about 150 μL to about 1000 μL of blood, or in some cases more than 1 mL of blood). In some embodiments, the device is capable of collecting about 250 uL of fluid sample from the subject in less than 1 min 45 secs. In some cases, the device is capable of collecting at least 175 uL to 300 uL of fluid sample from the subject in less than 2 mins. In some cases, the device is capable of collecting at least 200 μL of fluid sample from the subject in less than 4 minutes.

In some other embodiments, the device 100 can be configured to collect smaller amounts of blood (e.g. less than 150 uL, 140 uL, 130 μL, 120 uL, 110 uL, 100 uL, 90 uL, 80 μL, 70 uL, 60 uL, 50 uL, 40 uL, 30 uL, or 25 uL) of blood from a subject within a time window beginning from time of incision or penetration of a skin portion of the subject. The time window can be less than 5 minutes, preferably less than 3 minutes. In some embodiments, the time window can be under 2 minutes. In some embodiments, the time window can be under one minute.

In some embodiments, (1) the size and/or shape of the recess and/or (2) the vacuum pressure can be configured to achieve a minimum capillary pressure in the skin drawn into the recess. Similarly, (1) the size and/or shape of the recess and/or (2) the vacuum pressure can be configured to achieve a minimum tension in the skin drawn into the recess. As an example, the tension of the skin can be about 0.8 lbs/force at a vacuum pressure of about −1 psig.

An area of skin under vacuum when the device is applied to the skin can be about 100 to about 1000 mm², or about 100, 200, 300, 400, 500, 600, 700, 800, or 900 mm². An area of skin under the opening can be about 0.1 mm² to about 20 mm², or about 2, 4, 6, 8, 10, 12, 14, 16, 18, or 20 mm². An area of skin under vacuum when the device is applied to the skin can be at least 100, 200, 300, 400, 500, 600, 700, 800, or 900 mm², or less than 100, 200, 300, 400, 500, 600, 700, 800, or 900 mm², or about 100 to about 900 mm², or about 200 to 800 mm²

In some embodiments, an area of skin under vacuum is an area of skin encompassed by the area of the concave cavity at the housing base of the device. In some embodiments, an area of skin under vacuum is an area skin under the opening. In some embodiments, an area of the skin under the opening is at least 5 times smaller than an area of skin under vacuum when the device is applied to the skin. In some embodiments, an area of skin under the opening is about 5, about 10, about 20, about 30, about 40, about 50, about 60, about 70, about 80, about 90, about 100 times, about 200, about 300, about 400, about 500, about 600, about 700, about 800, about 900, about 1000, about 2000, about 3000, about 4000, about 5000, about 6000, about 7000, about 8000, about 9000, or about 10,000 times smaller than an area of skin under the vacuum. An area of skin under the opening can be less than 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 times smaller than an area of skin under the vacuum.

### C. Piercing Module

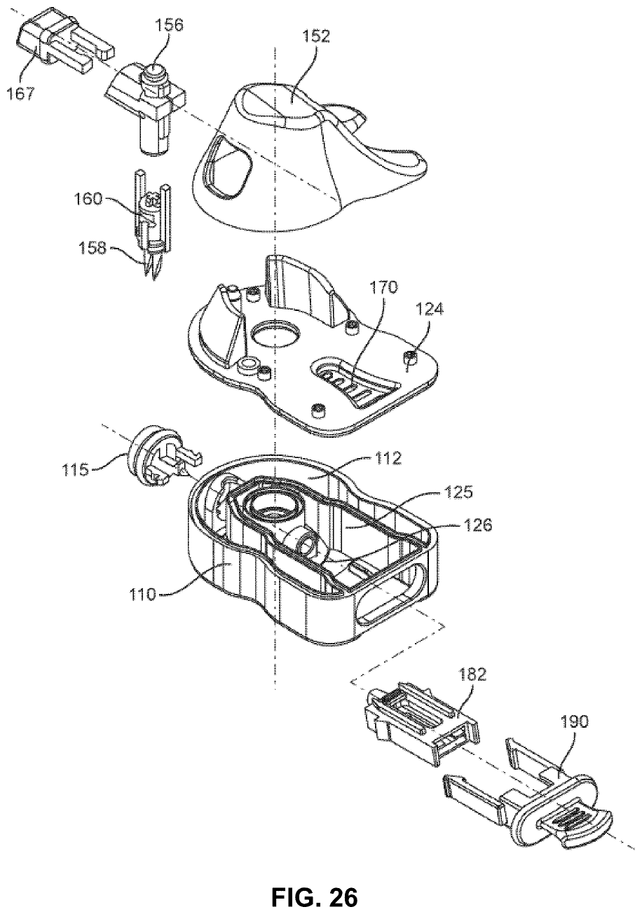

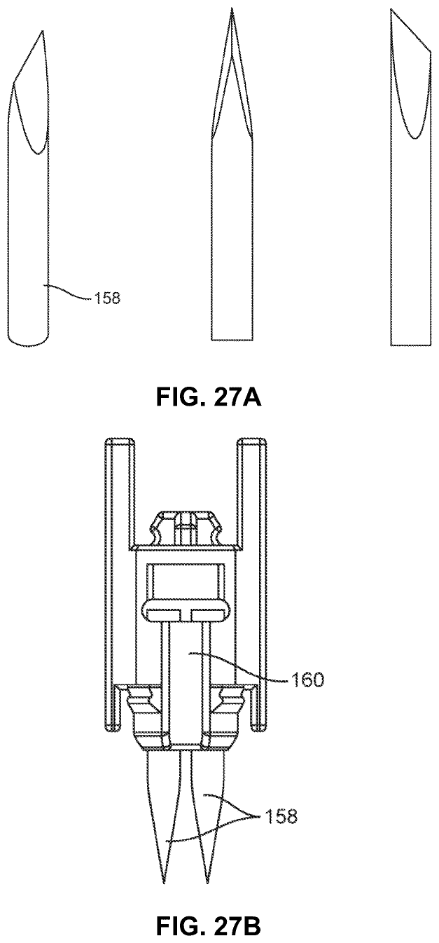

The device can include a piercing module 154 for penetrating skin of the subject when the skin is drawn into the recess under vacuum pressure. In some alternative cases, the device need not comprise a piercing module. The piercing module 154 can be provided in an enclosure 156. The enclosure can be located within the housing cover 152. The enclosure can be provided as a separate component that is coupled to the housing cover (see, e.g. FIG. 26). The piercing module can include one or more piercing elements 158 supported by a holder 160, for example as shown in FIGS. 27A and 27B. The piercing elements can include lancets, lances, blades, needles, microneedles, surgical knives, sharps, rods, and the like. Any number of piercing elements (e.g., at least 1, 2, 3, 4, 5, 6, 7, 8, 9, 10 or more piercing elements) can be contemplated. In some embodiments, the piercing elements can preferably comprise two lancets.

The piercing elements can comprise tempered steel, high carbon steel, or stainless steel. Examples of stainless steel include, but are not limited to 304 stainless steel, 316 stainless steel, 420 stainless steel, and 440 stainless steel. In some embodiments, the piercing elements can be coated with a surface finish. The surface finish can increase lubricity during a skin cut. The surface finish can also improve sharpness or penetration ability of the piercing elements. In some embodiments, the surface finish can be a zirconium nitride coating or a titanium nitride coating.

The piercing elements can be made of a biocompatible plastic or a biocompatible metal. The biocompatible plastic can include a number of suitable types of polymeric materials including, but not limited to, thermosets, elastomers, or other polymeric materials. Further, suitable biocompatible metals can include, for example, stainless steel, titanium, etc. Additionally or optionally, the piercing elements can be formed from various composite materials. The piercing elements can be manufactured using a number of suitable production processes. For example, the piercing elements can be fabricated using known metal processing techniques, such as casting or forging, or for the case of polymeric materials, any suitable polymer processing system can be used, including, for example, injection molding. A piercing element can have a sharp, pointed end that can be used to pierce a user's skin in order to collect blood.

The piercing module can further comprise one or more actuation elements (e.g., spring elements) for actuating the holder and moving the piercing elements. Other non-limiting examples of actuation elements can include magnets, electromagnets, pneumatic actuators, hydraulic actuators, motors (e.g. brushless motors, direct current (DC) brush motors, rotational motors, servo motors, direct-drive rotational motors, DC torque motors, linear solenoids stepper motors, ultrasonic motors, geared motors, speed-reduced motors, or piggybacked motor combinations), gears, cams, linear drives, belts, pulleys, conveyors, and the like. Non-limiting examples of spring elements can include a variety of suitable spring types, e.g., nested compression springs, buckling columns, conical springs, variable-pitch springs, snap-rings, double torsion springs, wire forms, limited-travel extension springs, braided-wire springs, etc. Further, the actuation elements (e.g., spring elements) can be made from any of a number of metals, plastics, or composite materials.

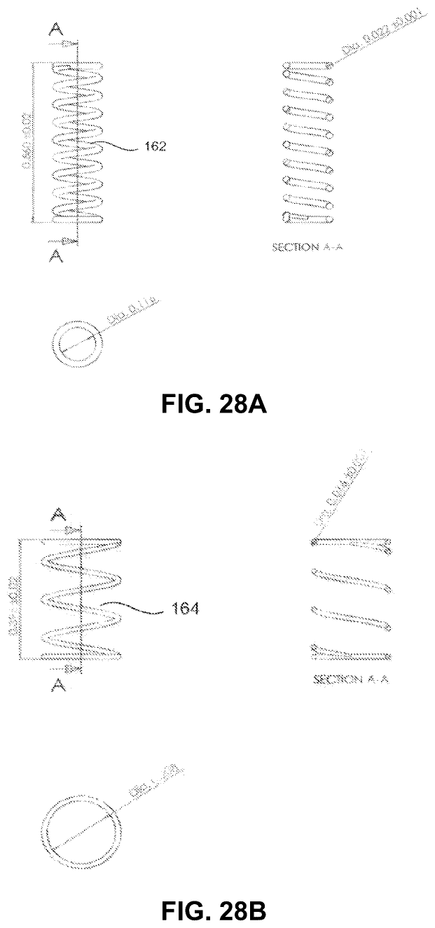

In some embodiments, the spring elements can include a deployment spring 162 positioned to deploy the one or more piercing elements through the opening of the recess, to penetrate the skin of the subject. An example of a deployment spring is shown in FIG. 28A. In some embodiments, the deployment spring can be configured to move and cause the piercing elements to penetrate the skin at speeds ranging from about 0.5 m/s to about 1.5 m/s, preferably about 1 m/s, and with a force ranging from about 1.3N to about 18N. The deployment spring can be configured to cause the one or more piercing elements to penetrate the skin to depths ranging from about 0.5 mm to about 3 mm.

The spring elements can further include a retraction spring 164 positioned to retract the one or more piercing elements through the opening back into the device, after the skin of the subject has been penetrated. An example of a retraction spring is shown in FIG. 28B. The retraction spring can be configured to retract the piercing elements from the skin of the subject at a speed of about 0.2 m/s. A spring-force of the retraction spring can be less than a spring-rate of the deployment spring. In some embodiments, the deployment spring can have a spring-rate of about 2625 N/m, and the retraction spring can have a spring-force of about 175 N/m.

A piercing element can have a length of about 1.0 mm to about 40.0 mm, or about 1.0 mm, about 1.5 mm, about 2.0 mm, about 4.0 mm, about 6.0 mm, about 8.0 mm, about 10.0 mm, about 15.0 mm, about 20.0 mm, about 25.0 mm, about 30.0 mm, about 35.0 mm, about 40.0 mm; a width of about 0.01 mm to about 3.0 mm, or about 0.01 mm, about 0.05 mm, about 0.1 mm, about 0.5 mm, about 1.0 mm, about 1.5 mm, about 2.0 mm, about 2.5 mm, about 3.0 mm. The length of a piercing element can be measured along a longitudinal direction, for example as shown by length/in FIG. 27A.

Each of the one or more piercing elements can be configured to pierce the skin of the subject to a depth of about 1.0 mm to about 25.0 mm, or about 1.0 mm, 1.5 mm, 2.0 mm, 3.0 mm, 4.0 mm, 5.0 mm, about 6.0 mm, about 7.0 mm, about 8.0 mm, about 9.0 mm, about 10.0 mm, about 15.0 mm, about 20.0 mm or about 25 mm. In some embodiments, a penetration depth of the one or more piercing elements can be preferably about 2 mm into the skin of the subject.

In some embodiments, the piercing elements can include lancets, and a length/of the lancet can be preferably less than about 13 mm. This length can be relatively shorter than currently commercially available lancets, and the shorter length of the lancets in the embodiments described herein can help to reduce the form factor of the device, as well as the type of spring and spring forces for actuating those lancets. For example, a shorter spring with lower spring-rate is needed to actuate a shorter lancet, as compared to longer lancets which tend to require longer springs and higher spring-rates. Shorter springs and lancets can help to reduce the size of the piercing module, which leads to a corresponding reduction in the size of the housing cover and the overall size of the device.

In some embodiments, two or more piercing elements can be supported by the holder in a random configuration. For example, two or more piercing elements can have random orientations relative to each other. The two or more piercing elements can comprise beveled edges that are randomly oriented relative to each other. The beveled edges of the two or more piercing elements can be non-symmetrical to each other. For example, the beveled edges of the two or more piercing elements can be at an acute or oblique angle relative to each other. Accordingly, the two or more piercing elements in the above configuration can be configured to generate cuts on the skin that extend in different directions along the skin, and that are non-parallel to each other.

In some alternative embodiments, two or more piercing elements can be supported by the holder in a predefined configuration. The two or more piercing elements can have predefined orientations relative to each other. For example, the two or more piercing elements can comprise beveled edges that are oriented relative to each other in a predefined manner. The beveled edges of the two or more piercing elements can be symmetrical to each other.

In some embodiments, the piercing elements can include two or more lancets. The lancets can have a same bevel angle, or different bevel angles. An example of a lancet and a bevel angle is shown in FIG. 27A. The bevel angle(s) can range from about 10 degrees to about 60 degrees. In some embodiments, the bevel angle of the lancets can be preferably about 42 degrees. The two or more lancets can have a same bevel length. Alternatively, the two or more lancets can have different bevel lengths. The bevel length of a lancet as described herein can refer to a length of the sharp beveled or slanted edge of the lancet, as shown by l′in FIG. 27A. In some embodiments, the bevel length of a lancet can range from about 1.6 mm to about 2.2 mm.

A method for penetrating the skin of a subject using the device 100 can be provided as follows. The method can include (1) placing the device onto the skin of the subject, (2) drawing skin into the recess of the device using vacuum, (3) activating an actuation element (e.g., a deployment spring) and deploying the one or more piercing elements through the opening in the device; (4) penetrating the skin of the subject using the one or more piercing elements; and (5) using another actuation element (e.g., a retraction spring) to retract the one or more piercing elements back into the device.

### D. Vacuum Activator and Piercing Activator

The device can include a vacuum activator 114 configured to activate the (evacuated) vacuum chamber, which generates a vacuum pressure that can draw the skin into the recess and subsequently facilitate collection of blood from the penetrated skin. The device can also include a piercing activator 166 configured to activate the deployment spring, for actuating the piercing elements. The vacuum activator can be separate from the piercing activator. For example, the vacuum activator and the piercing activator can be two separate discrete components of the device. In some alternative embodiments (not shown), the vacuum activator and the piercing activator can be integrated together as a single component that can be used to simultaneously or sequentially activate the vacuum and the piercing elements.

The vacuum activator can include a first input interface, and the piercing activator can include a second input interface. The first and second input interfaces can be located on different parts of the housing. Examples of suitable input interfaces can include buttons, knobs, finger triggers, dials, touchscreens, keyboards, mice, or joysticks. In some embodiments, at least one of the first input interface or the second input interface can comprise a button. For example, the vacuum activator can include a button 115 located on the housing base 110, and the piercing activator can include a button 167 located on the housing cover 152. In some embodiments, the vacuum activator and the piercing activator can be located on a same side of the housing, and the buttons 115/167 can be ergonomically accessible by the subject when the device is mounted onto an arm of the subject. The buttons can have distinct or different shapes and/or sizes, and can be ergonomically located for ease of use (e.g. easy identification by the user and well placed locations for simple activation).

In some alternative embodiments (not shown), at least one of the first or second input interfaces can be remote from the housing of the device. For example, one or both of the first and second input interfaces can be located on a user terminal (e.g. a mobile device or remote controller) that is connected with the device 100 via one or more wired or wireless communication channels.

Examples of wireless communication channels can include Bluetooth®, WiFi, Near Field Communication (NFC), 3G, and/or 4G networks. Signals for activating the vacuum and/or the piercing elements can be transmitted remotely from the user terminal to the device 100 over the one or more communication channels.

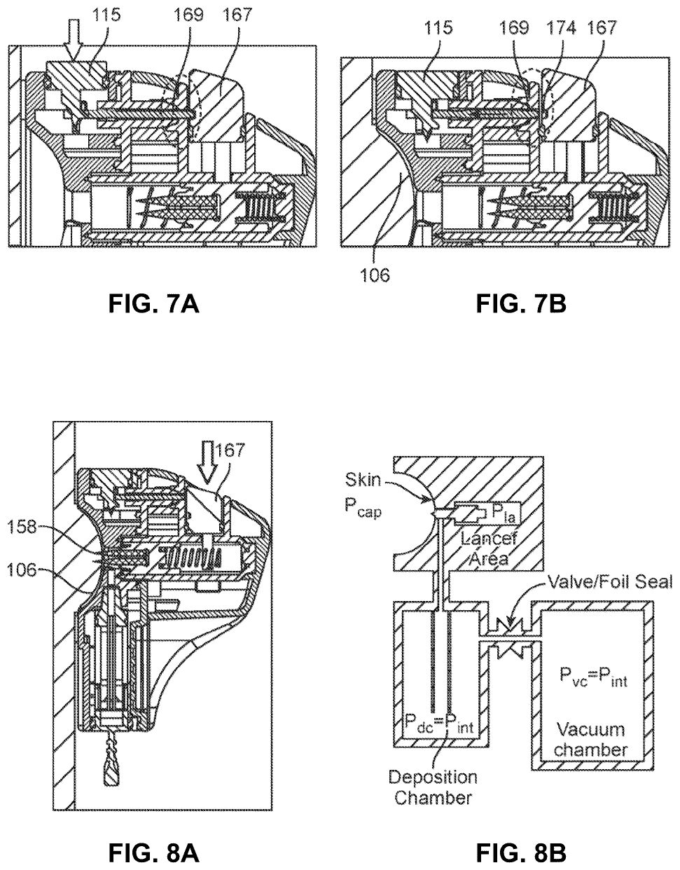

In some embodiments, the vacuum activator can be activated first, followed by the piercing activator. In other words, vacuum pressure can be activated prior to activation of the piercing elements. In certain embodiments, the piercing activator can be activated only after the vacuum activator and vacuum have been activated. For example, the piercing activator can be initially in a locked state, and incapable of activating the one or more piercing elements prior to activation of the vacuum. The piercing activator can be unlocked only after the vacuum activator has been activated. The above effect can be achieved by providing a locking mechanism that couples the piercing activator to the vacuum activator. The locking mechanism can be configured such that the piercing activator is initially in the locked state. The vacuum activator can function as a key for unlocking the piercing activator, and the piercing activator can be simultaneously unlocked when the vacuum activator is activated. Referring to FIGS. 7A and 7B, the locking mechanism can include a locking pin 169 coupled to the button 115 of the vacuum activator. Prior to use of the device for sample collection, the locking pin can be engaged in a slot or hole 174 located on the button 167 of the piercing activator, which prevents the button 167 from being pressed down by a user. Accordingly, the piercing activator is incapable of being activated when the button 167 is in the locked position. When the user presses the button 115, the locking pin 169 retracts in the direction shown in FIG. 7B and disengages from the slot 174, thus unlocking the button 167. Pressing the button 115 also pierces the foil 120 separating the vacuum chamber and the deposition chamber which causes the vacuum to be activated. Specifically, the chambers equalize to a negative pressure which draws the subject's skin into the recess. The user can then press down the unlocked button 167 to activate the piercing elements 158 for penetrating the subject's skin that is drawn into the recess.

In some embodiments, the piercing activator can be configured to activate the one or more piercing elements after the skin is drawn into the recess. The piercing activator can be configured to activate the one or more piercing elements after the skin is drawn into the recess by the vacuum for a predetermined length of time. The predetermined length of time can range, for example from about 1 second to about 60 seconds.

The vacuum activator can be configured to activate the vacuum by piercing the foil, which establishes fluidic communication between the vacuum chamber, deposition chamber, and the recess, and introduces negative pressure in the recess and the deposition chamber.

In some embodiments (not shown), the foil can be replaced by a valve, and the vacuum activator can be configured to open the valve to establish the fluidic communication. A valve can be a flow control valve having a binary open and closed position. Alternatively, a flow control valve can be a proportional valve that can control the flow rate of the air that flows between the vacuum chamber and the deposition chamber. For example, a proportional valve can have a wide open configuration that can permit a greater rate of flow than a partially open configuration that can permit a lesser rate of flow. Optionally, regulating, throttling, metering or needle valves can be used. Return or non-return valves can be used. A valve can have any number of ports. For example, a two-port valve can be used. Alternatively, a three-port, four-port or other type of valve can be used in alternative configurations. Any description herein of valves can apply to any other type of flow control mechanism. The flow control mechanisms can be any type of binary flow control mechanism (e.g., containing only an open and closed position) or variable flow control mechanism (e.g., which can include degrees of open and closed positions).

In some embodiments, the vacuum activator can be located on the housing such that the button 115 is configured to be pressed in a first direction when the device is mounted onto the subject's arm. The piercing activator can be located on the housing such that the button 167 is configured to be pressed in a second direction when the device is mounted onto the subject's arm. In some embodiments, the first direction and the second direction can be substantially the same. The first direction and the second direction can be substantially parallel to each other. In some embodiments, the first direction and the second direction can be substantially different, e.g. orthogonal or oblique to each other.

In some embodiments, at least one of the first direction or the second direction does not extend toward the skin of the subject. For example, the second direction may not extend toward the skin of the subject. At least one of the first direction or the second direction can extend substantially parallel to the skin of the subject. In some embodiments, the first direction and the second direction can both extend substantially parallel to the skin of the subject. At least one of the first direction or the second direction can extend in a direction of gravitational force. In some embodiments, the first direction and the second direction can both extend in the direction of gravitational force.

It is noted that pressing the button 167 of the piercing activator (which activates the piercing elements) in a direction away from the skin, for example downwards as opposed to against the skin, can be advantageous in reducing the perception of fear and pain associated with skin penetration. By locating the piercing activator and the button 167 on the housing in the configuration as shown, the overall user experience with the device can be improved.

In some alternative embodiments (not shown), the vacuum activator can be configured to generate one or more visual, audio, tactile, and/or message signals to indicate the status of the vacuum to a user. The signals can indicate to the user, for example that (1) the vacuum has been activated, (2) the pressure(s) within the different chamber(s), (3) the vacuum post internal pressure equalization, (4) that the piercing activator is next ready for activation, and the like. The visual signals can be generated using visible markers that are viewable to the naked eye. A visible marker can include an image, shape, symbol, letter, number, bar code (e.g., 1D, 2D, or 3D barcode), quick response (QR) code, or any other type of visually distinguishable feature. A visible marker can include an arrangement or sequence of lights that can be distinguishable from one another. For examples, lights of various configurations can flash on or off. Any light source can be used, including but not limited to, light emitting diodes (LEDs), OLEDs, lasers, plasma, or any other type of light source. The visible markers can be provided in black and white or in different colors. The visible markers can be substantially flat, raised, indented, or have any texture. In some instances, the visible markers can emit heat or other IR spectrum radiation, UV radiation, radiation along the electromagnetic spectrum.

The audio signals can include vibrations or sounds of different frequencies, pitches, harmonics, ranges, or patterns of sounds that can be detected by the user. For example, the sounds can include words, or musical tones. The vibrations/sounds can be discernible by the human ear. The vibrations/sounds can be used to indicate the status of the vacuum. For example, a first vibration/sound can be generated when the vacuum is properly activated, and a second vibration/sound different from the first can be generated if the vacuum is improperly activated or below a minimum internal pressure differential.

In some alternative embodiments (not shown), the piercing activator can be configured to generate one or more visual, audio, tactile, and/or message signals to a user. Such signals can be useful, for example in preparing the user's state of mind for an impending penetration of the skin by one or more piercing elements. Such signals can be used to distract the user prior to, during and/after the cuts on the skin are made. For example, lights and/or music emitted by the device can be used to attract the user's attention, which can potentially help to reduce the pain level (or perception of pain) during and after the cuts are made.

Optionally in any of the embodiments disclosed herein, the vacuum activation can be semi-automatic or fully automatic. In some embodiments, the device need not require manual vacuum activation. For example, the device can be configured to automatically apply the vacuum upon sensing or detecting that the device has been placed on a surface (e.g., on a subject's skin), or that the recess of the device is properly placed over the surface. Optionally in any of the embodiments disclosed herein, activation of the piercing elements can be semi-automatic or fully automatic. For example, the piercing elements can be automatically activated to penetrate the surface (e.g., a subject's skin) upon sensing or detecting that the surface is drawn into the recess of the device, and/or that the surface is in proximity to the opening (e.g., 140) of the recess. The above sensing or detection (for the vacuum activation and/or piercing activation) can be enabled using any variety or number of sensors. The sensors can be included with the device (e.g., onboard the device) or remote from the device. Non-limiting examples of sensors that can be used with any of the embodiments herein include proximity sensors, tactile sensors, acoustic sensors, motion sensors, pressure sensors, interferometric sensors, inertial sensors, thermal sensors, image sensors, and the like. In some cases, if the vacuum activation and/or piercing activation is configured to be semi-automatic or fully automatic, the buttons for the piercing activator and/or piercing activator can be optionally included (or omitted) from the device.

### E. Cartridge Assembly

As previously described, the deposition chamber of the device can also function as a cartridge chamber, and these two terms can be interchangeably used herein. The cartridge chamber can be configured to receive a cartridge assembly. The cartridge assembly can include a cartridge configured hold one or more matrices for storing a fluid sample (e.g., blood) thereon, and a cartridge holder. The cartridge holder can be releasably coupled to the cartridge using for example spring-clips. The cartridge assembly can be configured to releasably couple to the device 100 used for collecting blood from the subject. The cartridge holder can include a cartridge tab that is configured to be releasably coupled to a distal end of the cartridge chamber. The cartridge tab can be designed such that the subject or a user is able to (1) support the cartridge assembly by holding the cartridge tab, (2) couple the cartridge assembly to the device by pushing in the cartridge tab, and/or (3) decouple the cartridge assembly from the device by pulling the cartridge tab.

Referring to FIG. 3A, the cartridge can include a cartridge port 184 that is configured to be releasably coupled to an output port 148 in the deposition chamber 126. Fluidic communication can be established between a channel 146 of the device and a channel 185 of the cartridge when the ports 148 and 184 are coupled to each other. As shown in FIG. 3A, the channel 146 can extend towards the port 144 which is adjacent to the opening 140 of the recess 136. Blood can be drawn from the penetrated skin of the subject, and transported through the channels 146 and 185 into the cartridge with aid of vacuum, pressure differentials, and gravitational force.

The cartridge chamber can include cartridge guides 130 for guiding and holding the cartridge inside the cartridge chamber. The cartridge assembly can be releasably coupled to the cartridge chamber via a quick release mechanism. A quick release coupling mechanism can enable a user to rapidly mechanically couple (attach) and/or decouple (remove) the cartridge assembly from the cartridge chamber with a short sequence of simple motions (e.g., rotating or twisting motions; sliding motions; depressing a button, switch, or plunger, etc.). For example, a quick release coupling mechanism can require no more than one, two, three, or four user motions to perform a coupling and/or decoupling action. In some instances, a quick release coupling mechanism can be coupled and/or decoupled manually by a user without the use of tools. In some embodiments, the quick release coupling mechanism can include a luer-type fitting that mechanically engages with the cartridge when the cartridge assembly is inserted into the cartridge chamber.

The cartridge assembly can be coupled to the cartridge chamber prior to the collection of blood from the subject, and decoupled from the cartridge chamber after blood from the subject has been collected into the cartridge. The cartridge can include one or more matrices for collecting, storing, and/or stabilizing the collected blood sample. The matrices can be provided in strip form (as strips). A strip as used herein can refer to a solid matrix that is sized to maximize blood collection volume while still fitting into commonly used containers (e.g., a 3 ml BD vacutainer, deep well plate or 2 ml Eppendorf tube). A matrix as used herein can be interchangeably referred to herein as a matrix strip, a strip, a solid matrix, a solid matrix strip, and the like. A solid matrix can be configured to meter out, collect and stabilize fixed volumes of blood or plasma (e.g., greater than 25 μL, greater than 50 μL, greater than 75 μL, greater than 100 μL, greater than 125 μL, greater than 150 μL, greater than 175 μL, greater than 200 μL, or greater than 500 μL of blood or plasma). The cartridge assembly can be configured to hold any number of matrices (e.g., 1, 2, 3, 4, 5, 6, 7, 8, 9, 10 or more strips) and in various configurations.

The matrices can also enable lateral transport/flow of the blood. Non-limiting examples of the matrices can include absorbent paper strips, or a membrane polymer such as nitrocellulose, polyvinylidene fluoride, nylon, Fusion 5™, or polyethersulfone. In some embodiments, the matrices can comprise cellulose housing based paper (e.g. Whatman™ 903 or 226 paper), paper treated with chemicals or reagents for stabilizing the sample or one or more components of the sample (e.g., RNA stabilization matrix or Protein Stabilization Matrix). In some embodiments, the matrix comprises a cellulose filter paper. Any suitable commercially available filter paper can be used. Examples of commercially available filter paper include, but are not limited to, filter paper from Whatman®, such as 903 sample collection cards and fast transit analysis (FTA®) card. In some embodiments, the matrix can comprise a nitrocellulose filter paper. In some embodiments, the matrix does not comprise glass fiber filter paper.

The collection of the fluid sample can be aided by the natural wicking or capillary action associated with the matrix, which can enhance and accelerate the absorption or collection of the fluid sample onto the matrix. For a matrix having a surface area within the range of 100-300 square millimeters, a standardized quantity of blood saturating the matrix can be within a range of about 50-100 μL. In some embodiments, the quantity of blood absorbed by each matrix is about 30 to about 100 μL. In some embodiments, the quantity of blood absorbed by each matrix is about 67 to about 82 μL. In some embodiments, the quantity of blood absorbed by each matrix is 30 μL. In some embodiments, the quantity of blood absorbed by each matrix is about 45 μL. In some embodiments, the quantity of blood absorbed by each matrix is about 60 uL. In some embodiments, the quantity of blood absorbed by each matrix is about 75 μL. In some embodiments, the quantity of blood absorbed by each matrix is about 100 μL. In some cases, the matrices can be composed of a material comprising a plurality of capillary beds such that, when contacted with a fluid sample, the fluid sample is transported laterally across the matrices. The fluid sample fluid can flow along a flow path from a proximal end to a distal end of the matrices, for example by wicking or capillarity.

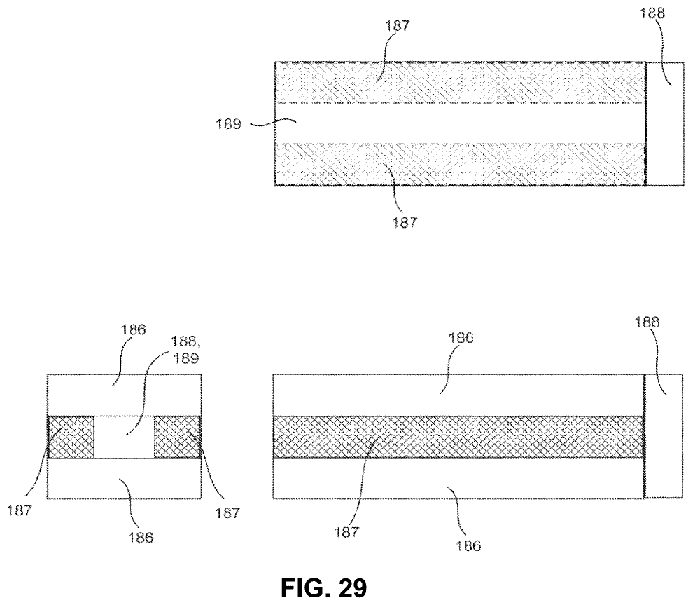

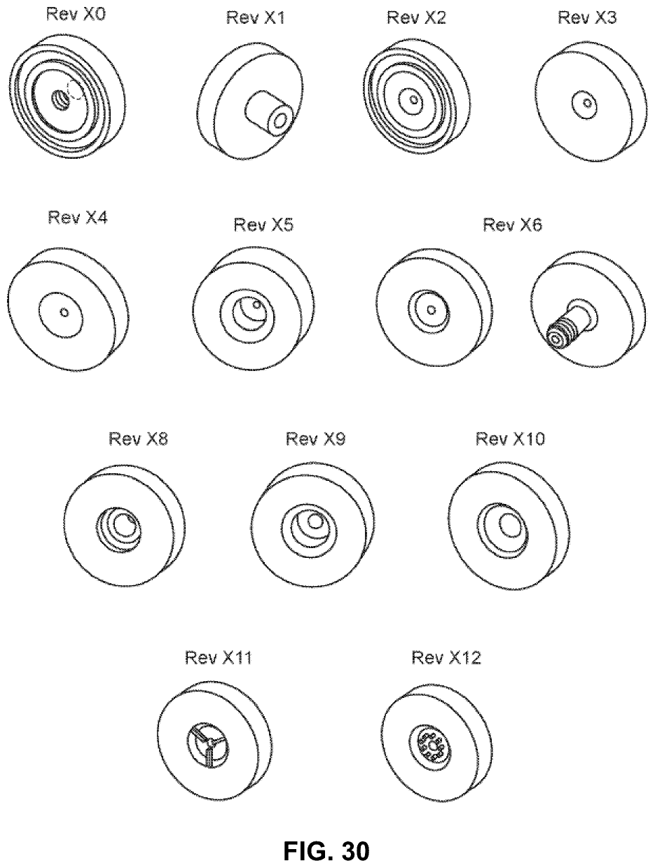

In some embodiments, two or more matrices are disposed in a configuration within the cartridge that permits the blood to wick between and flow along the matrices. The two or more matrices can be disposed substantially parallel to each other. The two or more matrices can be separated by spacers. The spacers can be made of an appropriate biocompatible material. Two or more spacers can be placed between two matrices to form a channel through the blood can flow via capillary action and wicking. In the example of FIGS. 3A and 29, the two matrices 186 can be separated by a pair of spacers 187. The spacers can be positioned on opposing lengths of the matrices to form a channel 189 through the blood can flow via capillary action and wicking. In some embodiments, the two or more matrices can be separated by a gap of about 0.5 mm (i.e. the spacers can have a thickness of about 0.5 mm). Any gap size may be contemplated. The spacers between the matrices can be adjustable and removable, depending on other relevant aspect (e.g. the needs and application of the sample being collected, stability of the analyte, rate of absorption requirements etc). The spacers can comprise a range of widths and coatings. Exemplary widths include widths in the millimeter to centimeter range (e.g., greater than 2 mm, greater than 4 mm, greater than 6 mm, greater than 8 mm, greater than 10 mm, greater than 0.2 cm, greater than 0.4 cm etc.). In further embodiments, the spacers can be coated with materials including hydrophobic coatings, hydrophilic coatings, antimicrobial coatings, coatings that bind to one or more components of a sample, coatings for binding to or inhibiting enzymes that can degrade or otherwise impact the quality of one or more analytes on the sample.

In some embodiments, at least one of the matrices is capable of collecting at least 60 μL of blood. In some cases, each of the two or more matrices is capable of collecting at least 60 μL of blood. The volume of blood collected can depend on the number of the matrices in the cartridge. For example, providing two matrices each with 60 μL holding capacity can yield a total blood sample volume of about 120 μL.

Referring to FIGS. 3A and 29, the cartridge assembly 180 can include one or more absorbent pads 188 for holding excess fluid sample (e.g. excess blood flowing beyond the matrices). The absorbent pads can serve as a wicking tail that can be used to absorb excess sample, and standardize or meter the volume of blood deposited on the saturated matrices. The absorbent pads can be placed at a distal end of the channel 189 opposite to the cartridge port 184, and can be placed in contact with end portions of the matrices 186. The absorbent pads can be supported or held in place by the cartridge holder 190. For example, the absorbent pads can be placed in a slot in the cartridge holder. The absorbent pads can be configured to absorb excess sample overflow. Each absorbent pad can be capable of holding at least about 10 μL of excess fluid sample. In some cases, each absorbent pad can be capable of holding at least about 20 μL, 30 μL, 40 μL, 50 μL, 60 μL, 70 μL, 80 μL, 90 μL, 100 μL, or more than 100 μL of excess fluid sample. The absorbent pads can be used to enable controlled metering of the matrices. The absorbent pads and their ability to contain the blood beyond the saturation volume of the matrices can enable consistent volumes of blood on the matrices, independent of varying input volumes to the device and cartridge. The absorbent pads can be configured (e.g., composition adjusted) so that the absorbent pads can be used as a means to control the volume of the sample absorbed on the matrices.

The cartridge assembly can comprise self-metering capability which can be advantageous for collecting a predefined volume of blood on the matrix strips for each individual, regardless of varying input volumes of blood flow to the cartridge for different individuals. The variations in input blood volume can occur since capillary pressures and blood flow can often vary from individual to individual (e.g., due to age, gender, health, etc.). The design of the cartridge assembly can ensure that matrix strips consistently contain a target blood volume independent of the volume of the blood that enters the cartridge (within or up to a predefined range). In the example of FIG. 29, the two matrix strips (e.g. 186) are in contact with one or more absorbent pads (e.g. 188) at the ends opposite the inlet port (e.g. 184) of the cartridge. As blood enters the cartridge via the port 184 during the draw, the matrix strips gradually saturate and during this time, the volume contained within the strips can increase (e.g. linearly) with the volume of blood that enters the cartridge. In some embodiments, once the matrix strips are saturated at ˜75 μL, the excess blood can wick onto the absorbent pad(s). By using the absorbent pads to absorb excess blood, the blood contained within the two matrix strips can be maintained at about 75 μL on each strip, even if (or as) the input volume of blood flowing into the cartridge increases beyond 150 μL. The volume of blood on the matrix strips can be metered/maintained unless or until the absorbent pads saturate with blood. In some embodiments, any input volume of blood between ˜150 μL and 300 μL to the cartridge can still result in the same volume (˜75 μL) of blood contained on each of the two matrix strips, with aid of the absorbent pad(s). In some embodiments, a range of blood volume collected on the matrix strips can be increased or decreased, for example by adding one or more additional absorbent pads, increasing or decreasing the strip size/saturation level, etc.

The collection of blood on the matrix strips can occur in phases. For example, during an initial phase, while the input volume of blood to the cartridge is between 0-150 μL, the two strips are filling but have not yet saturated, and the blood volume on each of the two strips increases gradually from 0-75 μL. During a subsequent phase, as the input volume of blood to the cartridge increases beyond 150 μL (e.g. 150 μL-300 μL), the strips are saturated at constant blood volume of ˜75 μL per strip, with excess blood flowing into the absorbent pads. The above-described passive metering mechanism can be advantageous in maintaining a predefined blood volume (e.g. 75 μL per strip) with varying blood input volumes within a target range.

It should be appreciated that the cartridge can include any number of matrix strips. The matrix strips can have the same saturation volumes or have different saturation volumes. The cartridge can also include any number of absorbent pads. The number of absorbent pads may or may not be the same as the number of matrix strips. The saturation volumes for the absorbent pads can be the same or different. The cartridge can be designed such that the matrix strips and absorbent pads have a self-metering capability as described above. For example, the sample volumes collected on the matrix strips can increase until the matrix strips reach their saturation volumes. After the matrix strips are saturated, any excess fluid is collected the absorbent pads. Accordingly, controlled well-defined volumes of the sample can be collected on the matrix strips, even though the input volume to the cartridge can and often exceeds the total saturation volumes of the matrix strips.

The use of the matrices with absorbent pads can facilitate accurate and precise sample collection. Two or more matrices can be stacked or arranged in ways that facilitate blood collection, distribution, precision and reproducible volumes of sample or analyte per surface area of each matrix. In some embodiments, the matrices can have different compositions or purposes. For example, a first matrix(es) can be used to separate cells from a cell free component and collect the cell free component on one matrix, and a second matrix(es) can be used collect raw unseparated sample. In some embodiments, the absorbent pads can be used as or incorporated into an indicator or be visible through a viewing window (of a flow meter) to inform a user that the collection procedure is complete.

In some embodiments, a method for collecting a fluid sample (e.g., blood) from a subject can be provided. The method can include: (1) releasably coupling the cartridge assembly to a device (e.g. device 100); (2) placing the device adjacent to skin of the subject; (3) activating vacuum in the pre-evacuated vacuum chamber to draw the skin into a recess of the housing; (4) using one or more piercing elements of the device to penetrate the skin; (5) maintaining the device adjacent to the skin for a sufficient amount of time to draw the fluid sample into the device and collect the fluid sample into the cartridge; and (6) decoupling the cartridge from the device after a certain amount of the fluid sample has been collected in the cartridge.

In some embodiments, one or more of the matrices can be designed and fabricated on a substrate. The substrate can be rigid or flexible. Examples of suitable substrates can include silicon, glass, printed circuit boards, polyurethane, polycarbonate, polyamide, polyimide, and the like.

The cartridges described herein generally depict fluid samples stored on solid matrices. However, this should not be taken to limit the devices disclosed herein. For example, the devices can include cartridges or means for collecting, treating, stabilizing and storing sample in either a liquid or a solid state. In some embodiments (not shown), the cartridge can include a vessel for storing liquid sample. The vessel can be used in conjunction with one or more matrices. Alternatively, the vessel can be used in place of matrices. Any number of vessels for storing liquid sample can be contemplated.

In some embodiments, the device disclosed herein can have multiple vacuum chambers (e.g. 2, 3, 4, 5 or more vacuum chambers) and multiple piercing modules (e.g., 2, 3, 4, 5 or more piercing modules). The device can be resuable and can be used to collect multiple samples in multiple cartridges. For example, a first vacuum chamber and a first piercing module can be activated to fill a first cartridge, a second vacuum chamber and a second piercing module can be activated to fill a second cartridge, a third vacuum chamber and a third piercing module can be activated to fill a third cartridge, and so forth. In some embodiments, a same vacuum chamber and piercing module can be used to fill a plurality of different cartridges, either within a same sample procedure or multiple procedures performed at different points in time.

### F. Flow Meter

In some embodiments, the device can include a flow meter 170 on the housing. The flow meter can be interchangeably referred to herein as a metering window (or metering windows). The flow meter can enable a subject or a user to monitor a progress of the fluid sample collection (e.g. blood sample collection) in real-time as the fluid sample is collected into the cartridge. For example, the subject or user can rely on the flow meter to determine whether the fluid sample collection is complete or near completion. In some embodiments, the flow meter can be provided on the housing base 110. For example, the flow meter can be a part of, or integrated into the lid 124 of the housing base. The flow meter can be in proximity to the deposition chamber 126 (or cartridge chamber). The flow meter can be located directly above the deposition chamber (or cartridge chamber). The flow meter can be substantially aligned with the cartridge 182 when the cartridge assembly is inserted into the cartridge chamber, for example as shown in FIGS. 3B, 4B, 20A, and 20B.

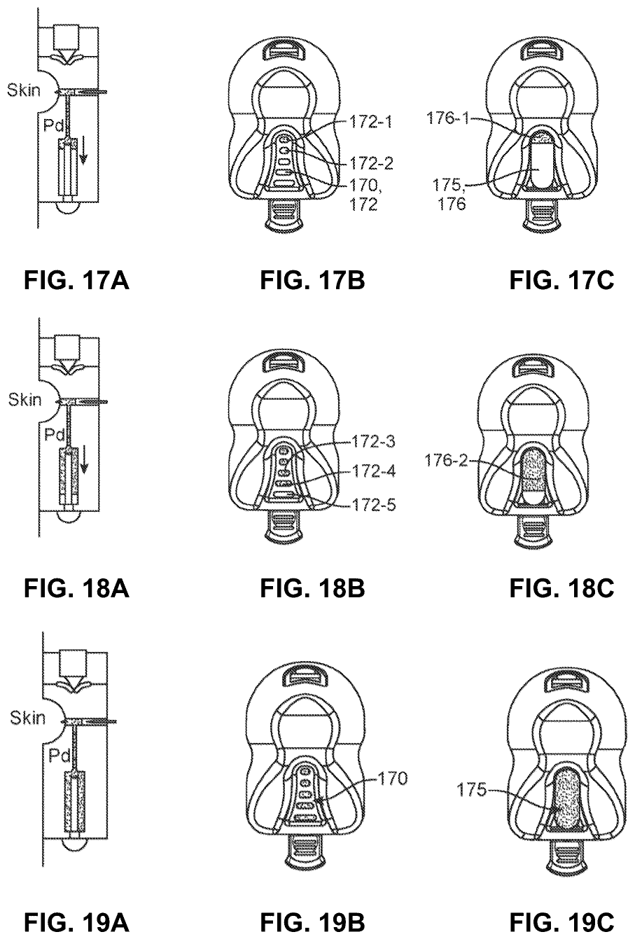

In some embodiments, the flow meter 170 can include a plurality of windows 172 disposed parallel to a longitudinal axis of the cartridge chamber. The plurality of windows can include three, four, five or more windows. In the example of FIGS. 17B, 18B, and 19B, the flow meter 170 can include windows 172-1, 172-2, 172-3, 172-4, and 172-5. The windows can line up with the matrices 186 of the cartridge when the cartridge assembly is inserted into the cartridge chamber. The windows can be made of an optically transparent material that allows the subject or user to see the underlying matrices in the cartridge. The fluid sample that is collected on the matrices can be visible through the windows. The fluid sample and the matrices of the cartridge can have different colors, preferably highly contrasting colors to permit easy viewing of the flow of the fluid sample along the matrices. The color of the fluid sample (e.g. red color for blood) can sequentially fill each window as the fluid sample is being collected on the matrices in the cartridge. Each window can be indicative of a known amount of fluid sample that is collected. For example, in FIG. 17B, the window 172-1 can have a visible color that indicates to the user that the matrices are about 20% filled. In FIG. 18B, the windows 172-1, 172-2, 172-3, and 172-4 can have a visible color that indicates to the user that the matrices are about 80% filled. In FIG. 19B, all of the windows 172-1, 172-2, 172-3, 172-4, and 172-5 can have a visible color that indicates to the user that the matrices are 100% filled. Accordingly, the user is able to determine that the sample collection is complete when the color of the fluid sample is visible in all of the windows.

FIGS. 17C, 18C, and 19C show a flow meter 175 in accordance with some other embodiments. The flow meter 175 can consist of a single window 176 disposed parallel to a longitudinal axis of the cartridge chamber. The single window can line up with the matrices 186 of the cartridge when the cartridge assembly is inserted into the cartridge chamber. The single window can be made of an optically transparent material. The fluid sample can be visible through the single window. The color of the fluid sample (e.g. red color for blood) can continuously fill the window as the fluid sample is being collected in the cartridge. In some embodiments, the window can include one or more markers that indicate a known amount of fluid sample that is collected. A user can be able to determine that the fluid sample collection is complete when the color of the fluid sample is visible throughout the entire window.

In some alternative embodiments (not shown), the flow meter can include one or more visible markers. The visible markers can replace the windows of the flow meter, or can be used in conjunction with the metering windows. The visible markers can be viewable to the naked eye. A visible marker can include an image, shape, symbol, letter, number, bar code (e.g., 1D, 2D, or 3D barcode), quick response (QR) code, or any other type of visually distinguishable feature. A visible marker can include an arrangement or sequence of lights that can be distinguishable from one another. For examples, lights of various configurations can flash on or off. Any light source can be used, including but not limited to, light emitting diodes (LEDs), OLEDs, lasers, plasma, or any other type of light source. The visible markers can be provided in black and white or in different colors. The visible markers can be substantially flat, raised, indented, or have any texture.

In some instances, the visible markers can emit heat or other IR spectrum radiation, UV radiation, radiation along the electromagnetic spectrum. In another example, the device or flow meter can emit vibrations or sounds of different frequencies, pitches, harmonics, ranges, or patterns of sounds that can be detected by the user. For example, the sounds can include words, or musical tones. The vibrations/sounds can be discernible by the human ear. The vibrations/sounds can be used to indicate a progress of the fluid sample collection process. For example, a first vibration/sound can be generated when the fluid sample starts flowing onto the matrices, and a second vibration/sound different from the first can be generated when the fluid sample has completely filled the matrices.

In some embodiments, the flow meter can be used to detect (e.g. enable the subject or a user to view) a feature, colorimetric change, display of a symbol, masking of a symbol, or other means of indicating the progress of the fluid sample collection, and to indicate that the fluid sample collection has been completed.

In some embodiments, one or more graphical user interfaces (GUIs) can be provided on the device. The GUIs can complement the use of the flow meter. In some embodiments, the function of the flow meter can be incorporated into the GUIs. The GUIs can be rendered on a display screen on the device. A GUI is a type of interface that allows users to interact with electronic devices through graphical icons and visual indicators such as secondary notation, as opposed to text-housing based interfaces, typed command labels or text navigation. The actions in a GUI can be performed through direct manipulation of the graphical elements. In addition to computers, GUIs can be found in hand-held devices such as MP3 players, portable media players, gaming devices and smaller household, office and industry equipment. The GUIs can be provided in a software, a software application, etc. The GUIs can be provided through a mobile application. The GUIs can be rendered through an application (e.g., via an application programming interface (API) executed on the device). The GUIs can allow a user to visually monitor the progress of the sample collection. In some embodiments, the GUIs can allow a user to monitor levels of analytes of interest in the collected sample.

In some embodiments, the device can be capable of transmitting data to a remote server or mobile devices. The data can include for example, user details/information, the date/time/location at which the sample is collected from the subject, the amount/volume of sample collected, time taken to complete the sample collection, maximum/minimum/average flowrates during sample collection, position of the subject's arm during sample collection, whether any errors or unexpected events occurred during the sample collection, etc. In some cases, the data can be transmitted to a mobile device (e.g., a cell phone, a tablet), a computer, a cloud application or any combination thereof. The data can be transmitted by any means for transmitting data, including, but not limited to, downloading the data from the system (e.g., USB, RS-232 serial, or other industry standard communications protocol) and wireless transmission (e.g., Bluetooth®, ANT+, NFC, or other similar industry standard). The information can be displayed as a report. The report can be displayed on a screen of the device or a computer. The report can be transmitted to a healthcare provider or a caregiver. In some instances, the data can be downloaded to an electronic health record. Optionally, the data can comprise or be part of an electronic health record. For example, the data can be uploaded to an electronic health record of a user of the devices and methods described herein. In some cases, the data can be transmitted to a mobile device and displayed for a user on a mobile application.

### G. Sample Collection

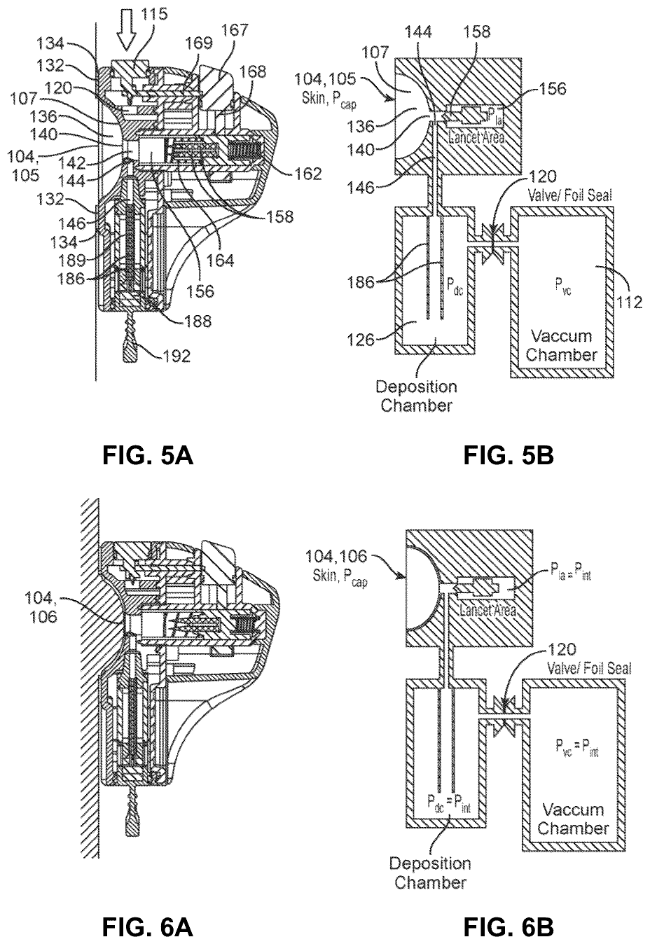

Next, exemplary methods of use of the devices herein for sample collection are described with detail with reference to various figures. Referring to FIG. 5A, the device 100 having cartridge assembly 180 can be placed on a subject's skin 104 (e.g. on the subject's upper arm). The subject's skin can be initially in a free state 105 (i.e. the skin is not under tension or drawn into the recess by vacuum pressure). The planar portion 132 of the housing base 110 can be in contact with the subject's skin, and attached to the skin with aid of an adhesive 134 as described elsewhere herein. The device can be configured for use in the orientation as shown in FIG. 5A, with the channels 146 and 189 and matrices 186 substantially aligned in the direction of gravity to aid sample flow.

FIG. 5B shows a schematic block diagram corresponding to FIG. 5A, and depicts the different chambers and enclosure. Referring to FIG. 5A, the device 100 can include the (1) deposition chamber 126, (2) vacuum chamber 112, (3) enclosure 156 for holding the piercing module 154, and (4) a cavity 107 enclosed between the skin and the surface of the recess. The vacuum chamber and the deposition chamber can be separated by the foil 120. The deposition chamber can be in fluidic communication with the cavity 107 and the enclosure 156 via channel 146. Prior to vacuum activation, the pressures within the deposition chamber 126 (Pac), enclosure 156 (Pₗₐ), and channel 146 can be at atmospheric pressure (or ambient pressure). The pressure P_{vc} within the vacuum chamber 112 can be at its maintained pressure which is below atmospheric while the separation interface 120 is closed (e.g. when the foil is intact). In some embodiments, the pressure P_{vc} can be about −12 psig prior to breaking of the foil 120. The capillary blood pressure within the skin (P_{cap}) is at a pressure greater than atmospheric. In some embodiments (not shown), the separation interface 120 can include a valve which can be opened to establish fluidic communication between the vacuum chamber and the deposition chamber. In some cases, the foil can be replaced by the valve, or used in conjunction with the valve.

Referring to FIGS. 6A and 6B, the vacuum in the vacuum chamber 112 can be activated by opening the separation interface 120, for example by breaking the foil (or in some cases, opening a valve). The vacuum activator can comprise a sharp protrusion 116 coupled to the button 115. The vacuum can be activated by pressing the button 115 downwards (FIG. 5A), which causes the protrusion 116 to break the foil (FIG. 6A). Subsequently, the pressure within the vacuum chamber, deposition, chamber, the enclosure and the internal channel equalizes at a pressure (Pᵢₙₜ) which is below atmospheric but greater than the initial pressure of the vacuum chamber. In some embodiments, the equalized pressure can be about −4 psig. This negative gauge pressure can draw the skin into the recess 136 and dras blood to that region within the capillary beds. This action can result in an increase in the capillary blood pressure within the skin which is now under tension within the recess.

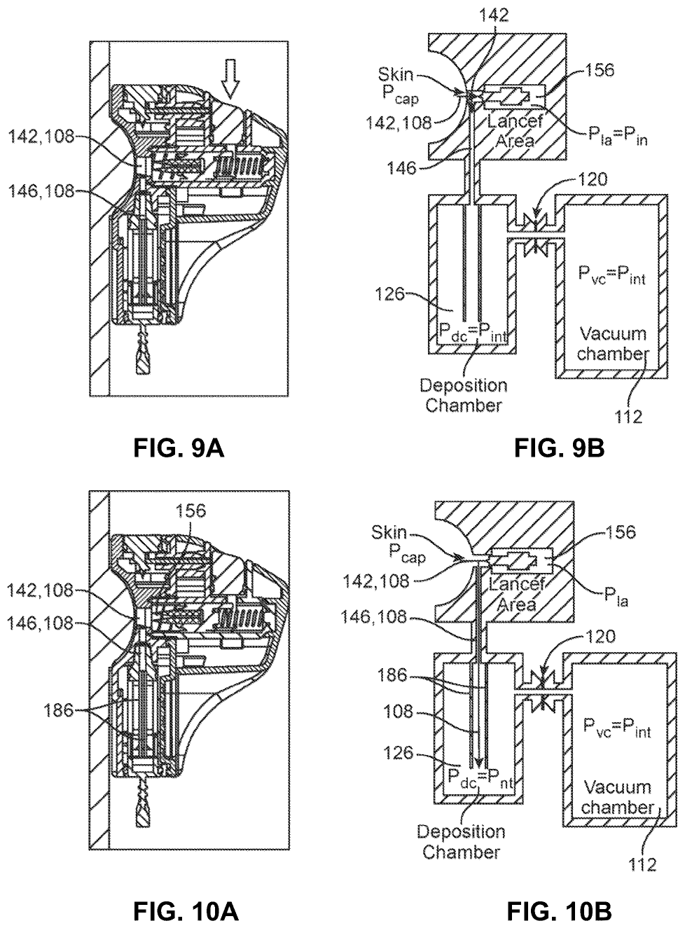

As previously described, activation of the vacuum can release the lock on the button 167 of the piercing activator. Referring to FIGS. 8A and 8B, when the button 167 is pressed downwards, the deployment spring 162 (which can be initially in a compressed state) is deployed, and extends the piercing elements 158 towards the opening 140 to penetrate the skin at the opening. In some embodiments (not shown), the deployment spring can be initially in an uncompressed state, and compressed by one or more actuating elements in preparation for deployment of the piercing elements. Referring to FIGS. 9A and 9B, the piercing elements are retracted from the skin by the retraction spring 164 after the skin has been penetrated. The initial flow of blood is driven by the pressure differential between the capillary blood pressure (P_{cap}) and the internal pressure of the device (Pᵢₙₜ). As previously mentioned, the internal pressure can be about −4 psig, and the capillary blood pressure is greater than atmospheric. Initially, a small amount of blood can travel towards and into the enclosure 156 while blood can also enter the channel 146 guiding it towards the deposition chamber 126.

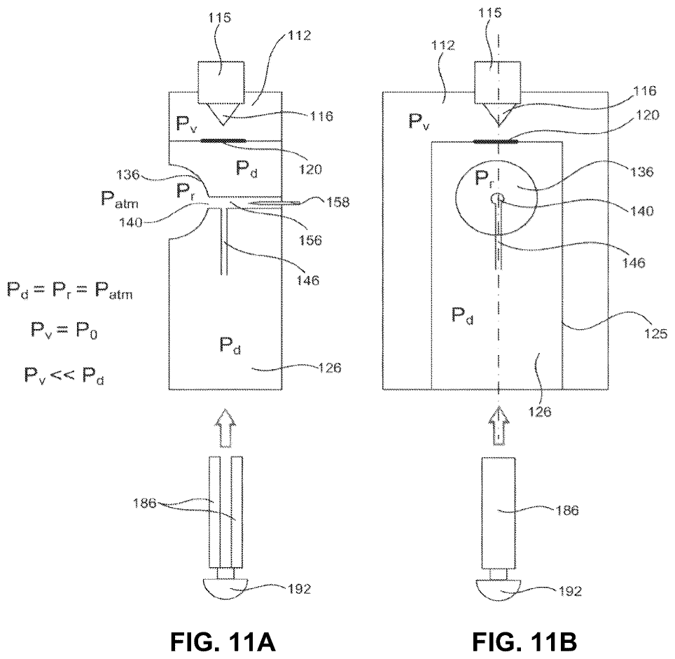

Referring to FIGS. 10A, 10B, and 11, the flow of blood can quickly reach a “steady state.” As blood enters the device, the volume of the blood present naturally causes the internal pressure to increase due to its negative gauge internal pressure. The volume Vₗₐ of the enclosure 156 can be substantially smaller than the combined volume V_{dc+vc} of the deposition chamber 126 and vacuum chamber 112. In some embodiments, a ratio of Vₗₐ to V_{dc+vc} can be about 1:10. The enclosure 156 can have an internal pressure P_{int_la}, and the deposition chamber 126 and vacuum chamber 112 can collectively have an internal pressure P_{int_dc+vc}. Due to the substantially smaller internal volume of the enclosure, the internal pressure P_{int_la} within the enclosure increases with the presence of blood much more rapidly than the internal pressure P_{int_dc+vc} within the deposition chamber and vacuum chamber which increases a very small amount. The internal pressure buildup in the enclosure causes the flow of blood into the enclosure to slow or stop, while the blood continues to be drawn into the deposition chamber by the pressure differential between the internal pressures P_{int_la} and P_{int_dc+vc} and the capillary blood pressure (P_{cap}). Blood flow towards the deposition chamber can further be aided by gravitational force, and by capillary action along the channels 146 of the device and the channel 189 of the cartridge. The blood flow can be further aided by wicking along the matrices 186 as the blood flows through the channel 189 of the cartridge.

The preferential flow of blood towards the deposition chamber 126 allows more blood to be collected in the deposition chamber. Minimal blood flowing into the enclosure 156 can also help to reduce wastage of blood, since blood in the enclosure is not collected and used. Accordingly, the above-described device configurations can help to increase the flowrate and volume of blood collected in the deposition chamber.

FIGS. 11A through 16F are schematic block diagrams showing the same operating principles as the embodiments described in FIGS. 5A through 10B. The schematic block diagrams are simplified generalized views of the device and the cartridge assembly, to show the change in pressures between the chambers and the flow of fluids. As such, some of the elements can be omitted in the interest of clarity. Like reference numerals refer to like elements throughout.

FIG. 11A shows a side sectional view of the device prior to insertion of the cartridge assembly, and FIG. 11B shows the corresponding front view. The cartridge assembly can include the matrices 186 and the cartridge tab 192. The device can include the (1) vacuum chamber 116, (2) deposition chamber 126, (3) recess 136, (4) enclosure 158 for the piercing element 158, and (5) channel 146 leading to the deposition chamber. The deposition chamber and the vacuum chamber can be separated by the foil 120. As shown in the FIG. 11B, the vacuum chamber can surround the deposition chamber in a U-like shape, and the two chambers can be separated by one or more walls 125. The pressures in the vacuum chamber, deposition chamber, and recess can be given by Pᵥ, P_{d}, and Pᵣ, respectively. Initially, P_{d} and Pᵣ can be at atmospheric pressure (Pₐₜₘ). The pressure Pᵥ within the vacuum chamber can be at a pre-evacuated vacuum pressure (P₀) which is below atmospheric while the foil 120 is closed (i.e. the foil is intact). Initially, P₀ can be substantially less than P_{d}. In some embodiments, P₀ can be about −12 psig. In some embodiments (not shown), the foil 120 can be replaced by a valve which can be opened to establish fluidic communication between the vacuum chamber and the deposition chamber.

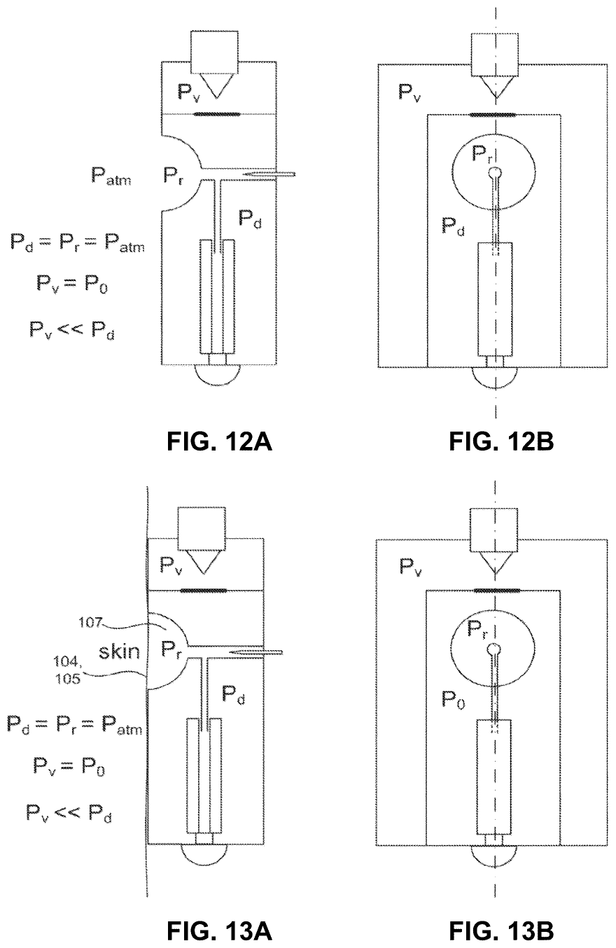

FIGS. 12A and 12B show the cartridge assembly inserted into the deposition chamber. Next, the device can be placed onto a subject's skin 104, as shown in FIG. 13A. The skin can be initially in a free state 105 (i.e. not under tension due to vacuum suction). A cavity 107 can be enclosed between the skin 104 and the surface of the recess 136. The initial pressures within the chambers and various compartments can remain the same since there is no fluidic communication causing any pressure changes.

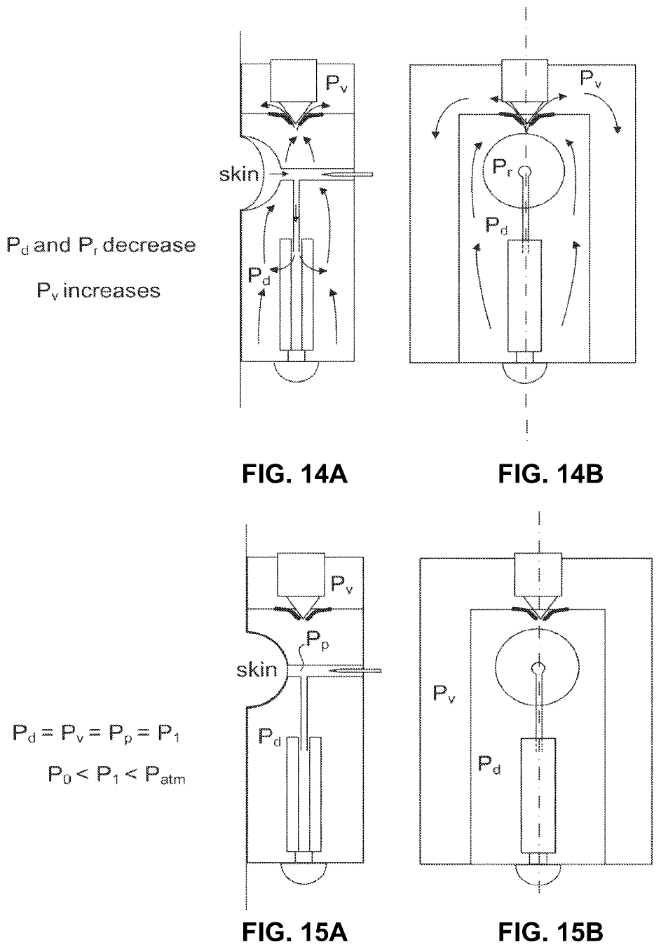

Referring to FIGS. 14A and 14B, the vacuum in the vacuum chamber 112 can be activated by breaking the foil 120 (or in some cases, opening a valve). The vacuum activator can comprise a sharp protrusion 116 coupled to the button 115. The vacuum can be activated by pressing the button 115 downwards, which can causes the protrusion 116 to break the foil. Air from the deposition chamber 126, cavity 107, enclosure 156, and channel 146 can be drawn into the vacuum chamber to equalize the pressures, as shown in FIGS. 14A and 14B. As a result, Pa and Pᵣ will decrease while Pᵥ increases. At the same time, the skin can be drawn into the recess by the pressure differentials.

Referring to FIGS. 15A and 15B, the skin can be completely drawn into the recess. The pressure Pₚ in the enclosure 156, Pᵥ and P_{d}, and the pressure in the channel 146 equalize at a pressure P₁, whereby P₀<P₁<Pₐₜₘ. In some embodiments, P₁ can be about −4 psig. This negative gauge pressure can draw and holds the skin in the recess 136, and draws blood to the skin region within the capillary beds. This can result in an increase in the capillary blood pressure P_{c} within the skin which can now be under tension.

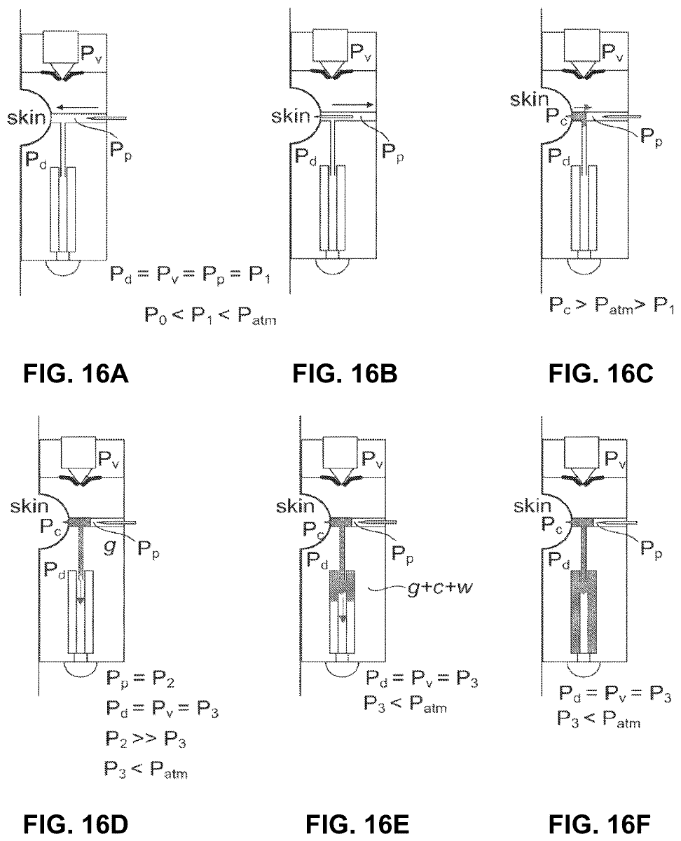

Next, referring to FIGS. 16A and 16B, the piercing element 158 can be deployed and penetrate the skin at the opening 140 of the recess, and retracted from the skin as shown in FIG. 16C. The initial flow of blood can be driven by the pressure differential between P_{c} and Pᵢₙₜ, whereby P_{c}>Pₐₜₘ>P₁. Initially, a small amount of blood can travel towards and into the enclosure 156 while blood also enters the channel 146 guiding it towards the deposition chamber 126, as shown in FIG. 16C.

The volume Vₗₐ of the enclosure 156 can be substantially smaller than the combined volume V_{dc+vc} of the deposition chamber 126 and vacuum chamber 112. In some embodiments, a ratio of Vₗₐ to V_{dc+vc} can be about 1:10. As blood flows into the enclosure and towards the deposition chamber, the pressure Pₚ of the enclosure increases to P₂, and the pressures P_{d} and Pᵥ of the deposition chamber and the vacuum chamber can increase to P₃. However, P₂ can be substantially greater than P₃ since Vₗₐ can be substantially smaller than V_{dc+vc}. In other words, the pressure in the enclosure 156 increases much more rapidly than the pressure within the deposition chamber and vacuum chamber which increases by a very small amount. The internal pressure buildup in the enclosure causes the flow of blood into the enclosure to slow or stop, while the blood continues to be drawn into the deposition chamber by the pressure differential between the internal pressures P_{int_la} and P_{int_dc+vc} and the capillary blood pressure P_{cap}. Accordingly, the flow of blood reaches a “steady state” in which the blood is drawn only towards the deposition chamber. Blood flow towards the deposition chamber can be further aided by gravitational force g, and by capillary action c along the channels 146 of the device and the channel 189 of the cartridge. The blood flow can be further aided by wicking w along the matrices 186 as the blood flows through the channel 189 of the cartridge.

As previously described, the preferential flow of blood towards the deposition chamber 126 can allow more blood to be collected in the deposition chamber. Minimal blood flowing into the enclosure 156 can also help to reduce wastage of blood, since in some cases blood in the enclosure is not collected and used. Accordingly, the above-described device configurations can help to increase the flowrate and volume of blood collected in the deposition chamber.

### III. Packaging and Transportation of Cartridge Post Sample Collection

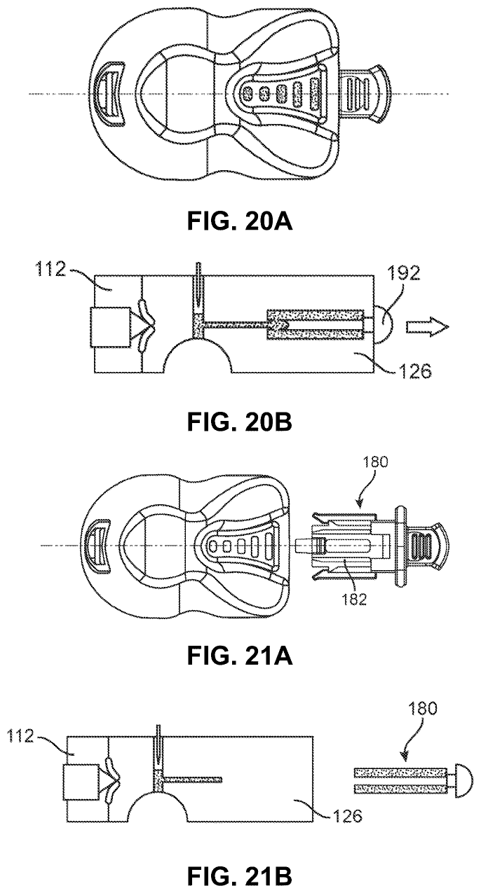

As previously described with reference to FIGS. 17A-19A, 17B-19B, and 17C-19C, the use of flow meters on the device can allow a user to monitor the progress of the sample collection, and to know when the sample collection has been completed. FIG. 20A shows a top view of the device with a completely filled cartridge, and FIG. 21A shows a top view with the filled cartridge removed from the device. The cartridge assembly can be removed from the deposition chamber of the device by pulling the cartridge tab. The filled cartridge can be subsequently packaged and transported to an external facility for further processing. For example, the sample can be treated, stabilized and stored. In any of the embodiments described herein, the devices can be configured to collect, treat, and store the sample. Samples drawn by the device can be stored in liquid or solid form. The sample can undergo optional treatment before being stored. Storage can occur on the device, off the device, or in a removable container, vessel, compartment, or cartridge within the device.

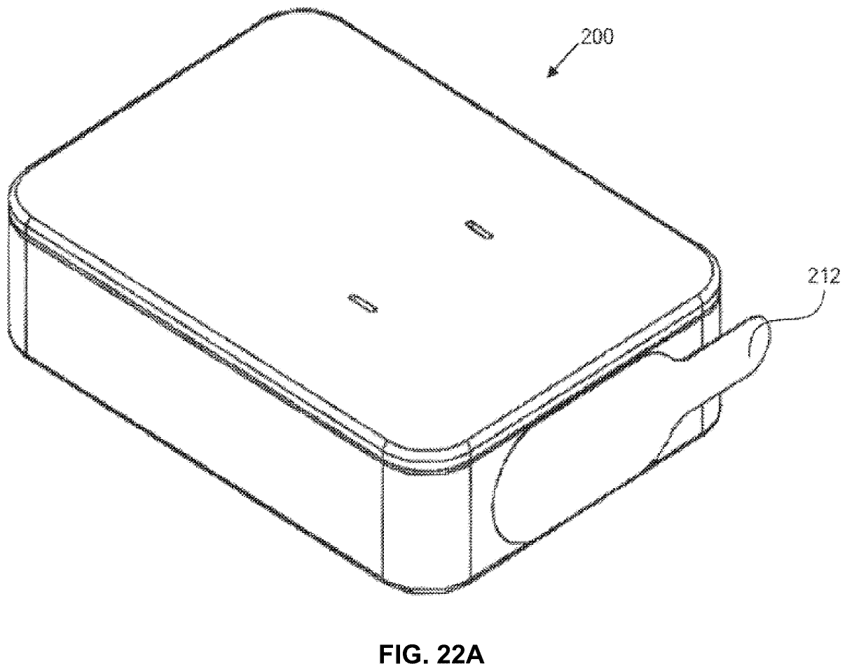

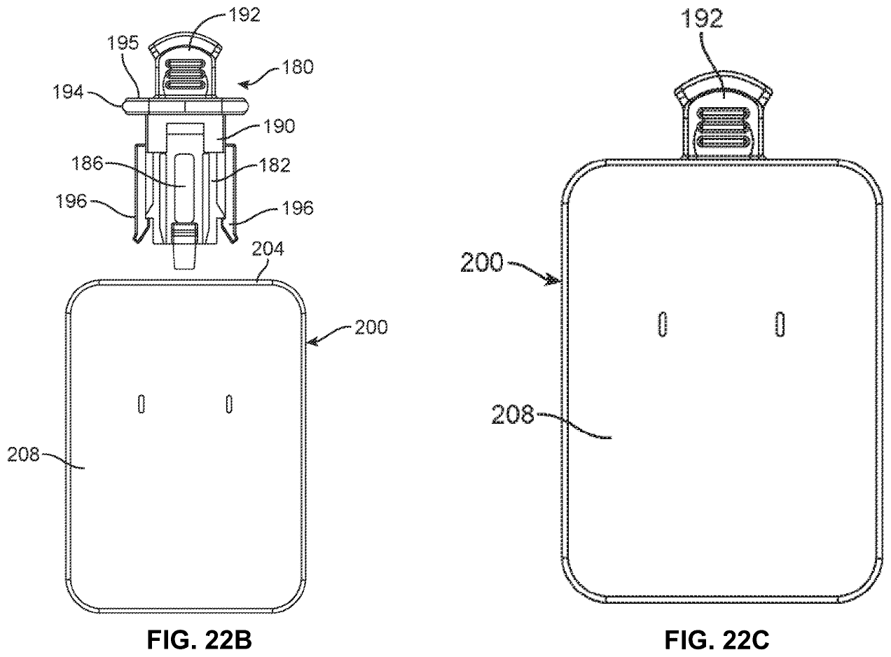

FIG. 22A shows a perspective view of a transportation sleeve 200 that can be used for packaging of a filled cartridge or samples within the cartridge. The sleeve can include a hollow interior for storing the filled cartridge or samples during shipment/transportation. The sleeve can include an opening for receiving the cartridge. In some embodiments, the sleeve can include a cover 212 for covering the opening prior to use of the sleeve. The cover 212 can be, for example a peel foil that can be attached to the opening via an adhesive, and peeled off by a user prior to use of the sleeve. A dessicant (not shown) can be disposed within the sleeve, and used for keeping the samples dry. The peel foil can help to protect the interior of the sleeve from moisture and contamination prior to use.

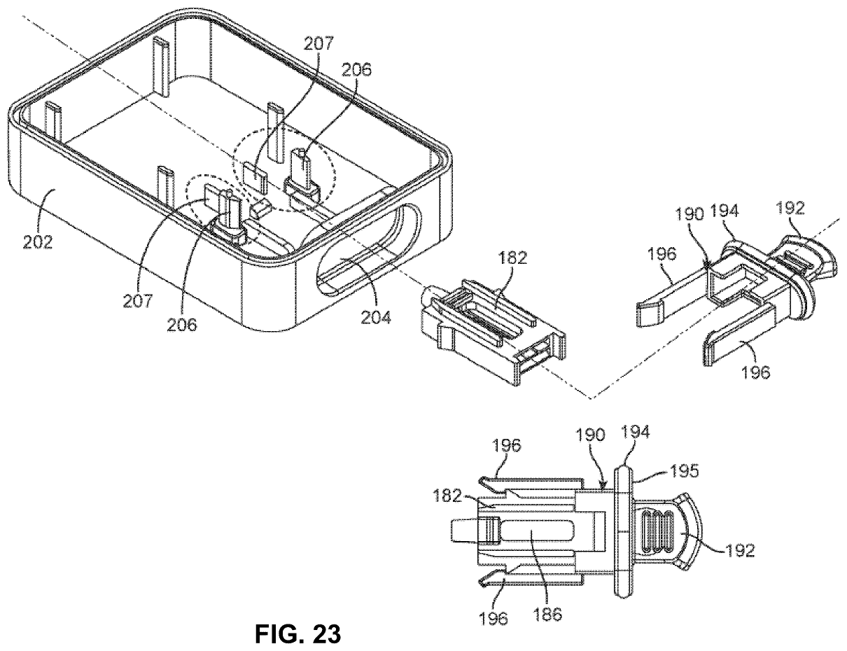

FIG. 22B shows a top view of the transportation sleeve and a filled cartridge assembly prior to its insertion into the sleeve. FIG. 22C shows the filled cartridge assembly inserted into the transportation sleeve, with the cartridge tab 192 extending from an edge of the sleeve. FIG. 23 shows an exploded view of the transportation sleeve and cartridge assembly. Referring to the above figures, the sleeve can include a sleeve base 202 and a sleeve lid 208 configured to be operably coupled to each other. The sleeve base can include an opening 204 for receiving the cartridge assembly. The opening can be configured to couple to the cartridge holder (e.g. proximal to the cartridge tab). The sleeve can include a dual support-release mechanism comprising (a) a retention element configured to engage with a corresponding mating feature on the cartridge and secure the cartridge within the sleeve, and (b) a release element configured to cause the spring-clips on the cartridge holder to release and thereby decouple the cartridge from the cartridge holder. In some embodiments, the dual support-release mechanism can be implemented using a plurality of posts 206 and 207.

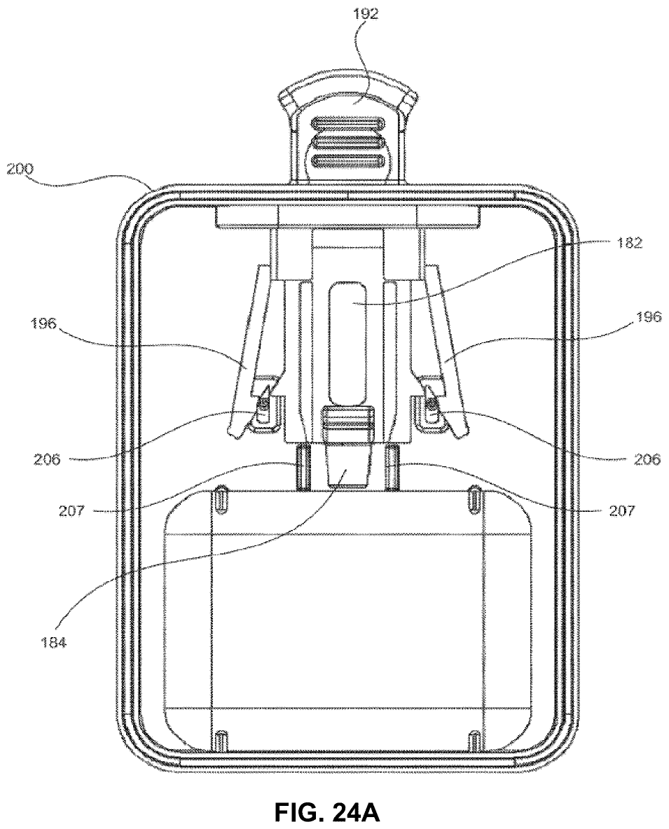

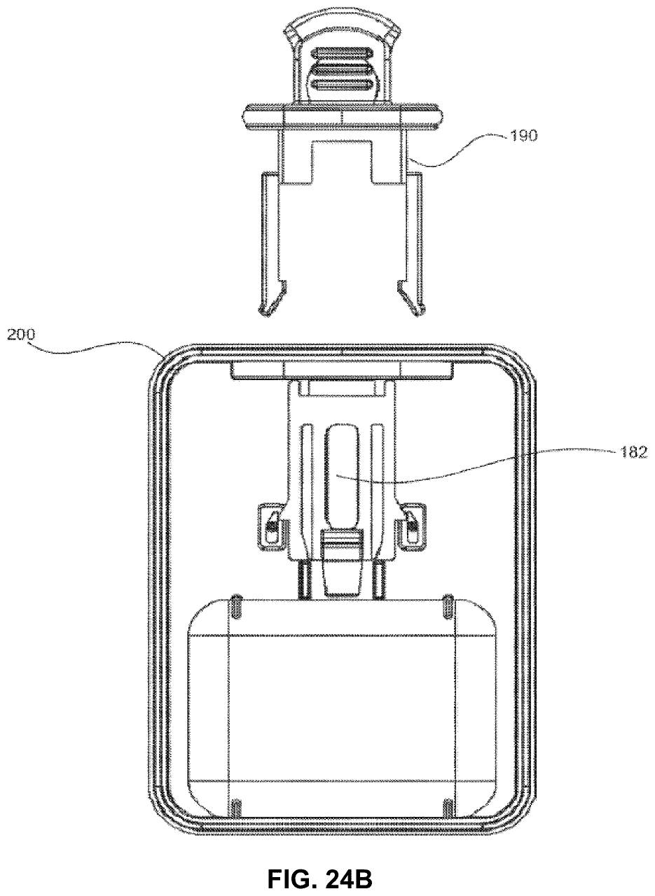

FIG. 24A shows a side sectional view of the transportation sleeve with the cartridge assembly inserted therein. FIG. 24B shows a side sectional view with the cartridge holder removed, leaving the cartridge within the transportation sleeve. As shown in the above figures, the cartridge assembly is inserted into the opening 204 of the sleeve 200 by pushing the cartridge tab 192 until a rear portion of the cartridge holder and the seal/gasket 194 comes into contact with and seals the opening 204. The posts 206 can be configured to engage and release the spring clips 196 on the cartridge holder when the cartridge assembly is properly inserted into the sleeve. The release of the spring clips decouples the cartridge from the cartridge holder. The posts 207 can serve as stoppers, and come into contact with a portion of the cartridge adjacent to the cartridge port 184. As shown in FIG. 24B, the cartridge holder can be subsequently removed from the sleeve, leaving the cartridge held in place by posts 206 and 207 within the sleeve. As described above, the post 206 and 207 can provide the dual support-release mechanism. The decoupling of the cartridge from the cartridge holder via the dual support-release mechanism can permit the cartridge holder to be removed from the opening of the sleeve while the cartridge is secured in place within the sleeve, without exposure of the strips to the ambient environment.

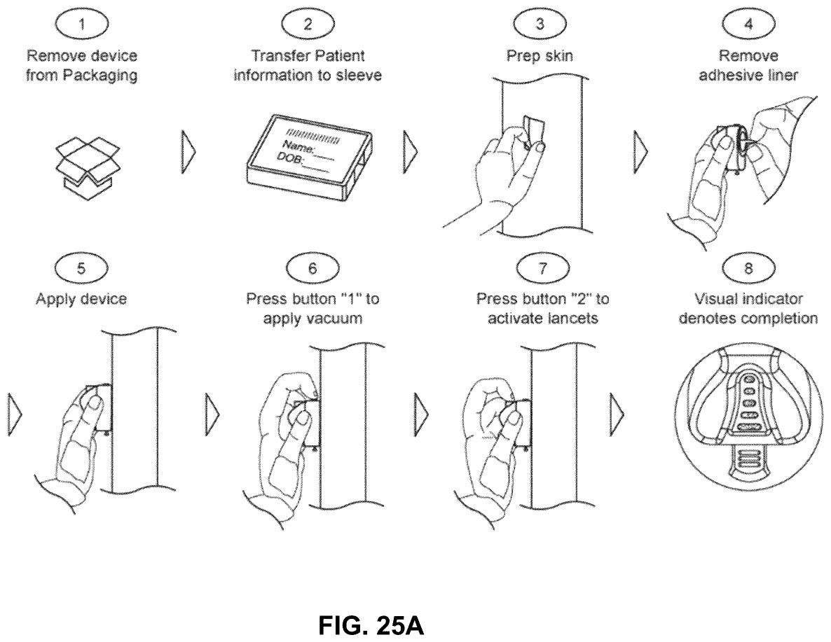

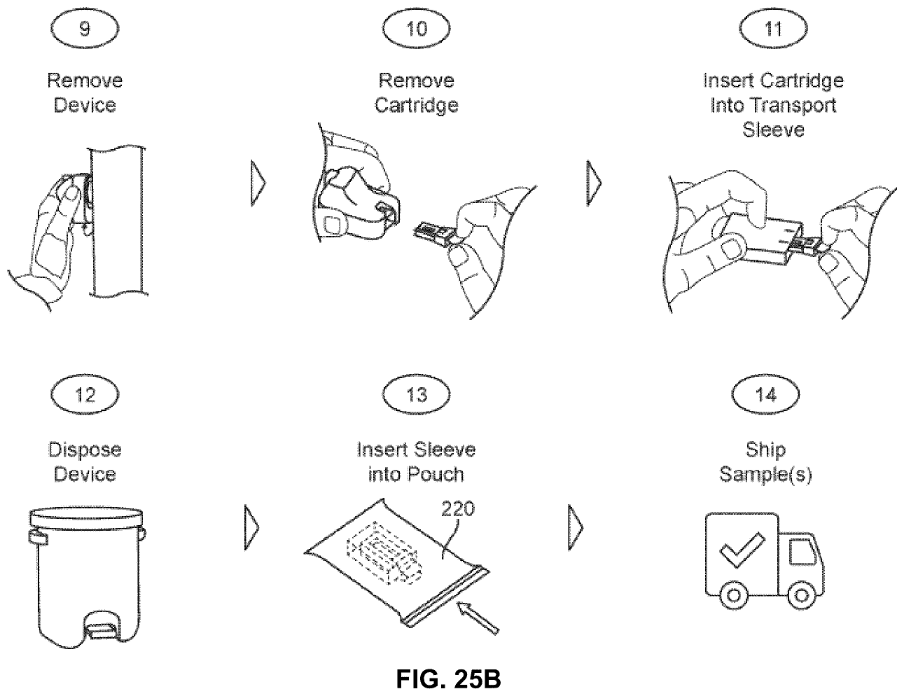

In some embodiments, additional treatment and/or stabilization of the sample on the matrices 186 can take place within the transportation sleeve following the release of the cartridge from the cartridge holder. In some embodiments, a desiccant can be provided within the sleeve for drying the sample on the matrices. In some embodiments, the sleeve can be placed in a carrier pouch 220 and shipped for further processing (see e.g., steps 13 and 14 of FIG. 25B).

FIGS. 25A and 25B illustrate exemplary procedures to collect and store blood samples using any of the devices described herein (e.g. device 100). Referring to FIG. 25A, the device can first be removed from its packaging (step 1). A subject or another user (e.g. a healthcare personnel) can record the patient's information on a sleeve label (step 2). An alcohol swab is then used to clean the skin on the patient's upper arm where the device will be applied (step 3). Next, an adhesive liner is removed from the planar portion on the housing base of the device to reveal a hydrogel adhesive (step 4). Next, the device is placed and adhered to the patient's skin with the hydrogel adhesive (step 5). The button labeled “1” on the device is pressed to activate the vacuum to drawn the patient's skin into the recess (step 6). The button labeled “2” on the device is next pressed to activate one or more piercing elements to penetrate the patient's skin at the opening of the recess (step 7). Blood is absorbed by one or more matrices in the cartridge of the device. As blood is absorbed, the flow meter on the device can indicate the progress of the blood collection, and indicate when the matrices are full (step 8). Once the matrices are full, the device is removed (step 9). The cartridge is removed from the device (step 10) and inserted into a transportation sleeve (step 11). The device is no longer needed and can be disposed appropriately in a sharps container (step 12). The sleeve can be placed into a pouch (step 13) which is used to ship the sample to a lab for processing (step 14).

### IV. Additional Embodiments

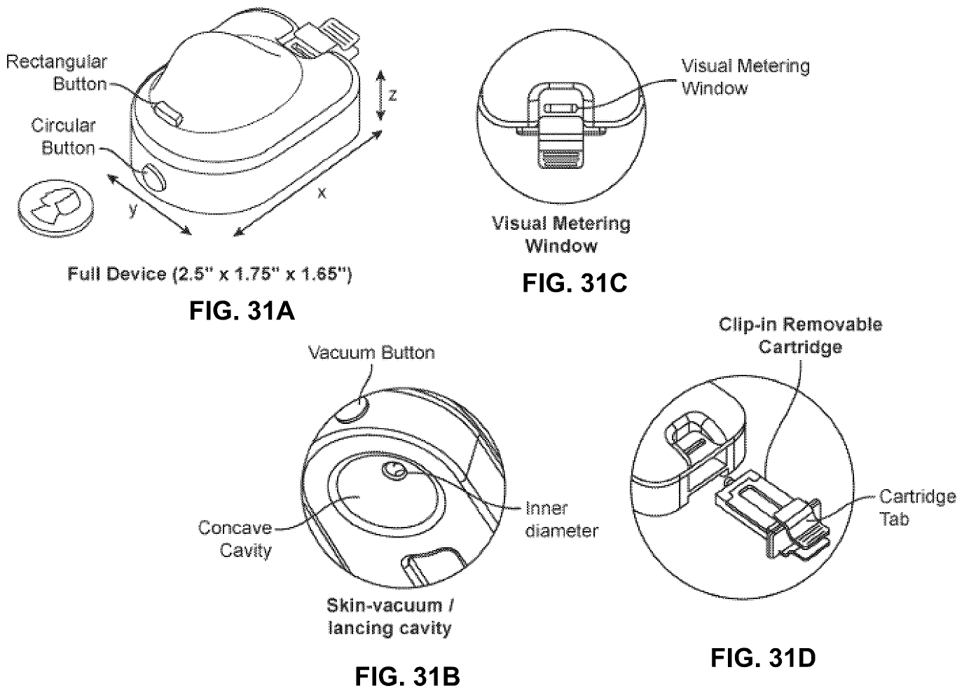

Provided herein are devices, methods, and kits for collecting blood from a subject. Devices, methods, and kits provided herein can permit application of a vacuum to skin of a subject, followed by piercing of the skin of the subject under vacuum (e.g., with one or more blades). Application of the vacuum can enhance blood flow to a region of skin under vacuum and can increase the rate and volume of blood collection in the device. The vacuum can be generated using a cupping action via, e.g., a rigid concave surface or flexible concave surface, e.g., a concave cavity (see, e.g., FIGS. 31A-31D). A volume of a hemisphere formed by the concave surface can be equivalent to, or about half, or about a quarter, of a volume of a vacuum chamber in the device. The concave cavity can comprise an opening with an inner diameter, and the concave cavity can comprise a diameter at a base of the device.

Any of the devices provided herein can comprise one or more piercing elements, e.g., blades. The one or more piercing elements, e.g., blades, can be configured to pass through the opening of the device and pierce the skin of a subject. Each of one or more blades can comprise a length of about 1 mm to about 10 mm, or about 1 mm, 1.5 mm, 2 mm, 4 mm, 6 mm, 8 mm, 10 mm, a width of about 0.01 to about 2 mm, or about 0.01 mm, 0.05 mm, 0.1 mm, 0.5 mm, 1 mm, 2 mm, and a depth of about 1 to about 20 mm, or about 1, 5, 10, 15, or 20 mm. The devices can comprise one or more piercing elements, e.g., at least 1, 2, 3, 4, 5, 6, or 7 piercing elements (e.g., lancets, needles, or blades).

A method for collecting blood from a subject is provided herein, the method comprising applying a vacuum to skin of a subject using a device; after applying the vacuum, piercing the skin of the subject under which the vacuum is applied, wherein the device is used to pierce the skin of the subject, thereby generating an incision in the skin under which the vacuum is applied; and collecting the blood from the incision under the vacuum, wherein the collecting occurs in the device. The vacuum can deform skin, enhance perfusion and draw blood from the smaller incision area. The vacuum can be a global vacuum. A local vacuum can also be used, but the skin deformation and perfusion can be much less.

In some embodiments, the subject has diabetes. In some embodiments, collecting blood from a subject further comprises stabilizing a component or analytes of interest from the blood. In some embodiments, the analyte of interest is hemoglobin A1c (HbA1c).

The device can be configured with user friendly features. FIG. 31A, FIG. 31B, FIG. 31C, and FIG. 31D illustrate features that can be integrated into devices disclosed in the present application. Such features can include single or multiple (e.g., 2, 3, 4, 5) actuators or activators (e.g., which can include buttons) for device activation, with positions that can be readily activatable by the user given the shape of the device and location of the actuator. Actuators or activators can have distinct shapes, sizes, and locations configured (e.g. positioned or structured on the device) for ease of use (e.g. easy identification by the user and well placed locations for simple activation). An example of a device with actuators or activators for performing one or more user direct actions is shown in FIG. 31A, wherein two buttons are shown each with an easily identified shape and comfortable to use location. The circular button shown in FIG. 31A can be used for activating a vacuum and the rectangular button can activate a piercing element (e.g., lancet) for piercing the skin. In some cases, a single actuator or activator can be used to activate a vacuum and a piercing element. The device can comprise a lancet activation actuator configured to activate the lancet upon actuation of the lancet activation actuator. The lancet activation actuator can comprise a button.

Features, e.g., user friendly features, can comprise mechanisms for expediting blood collection by enhancing a rate or means of collecting a sample, thus reducing the time it takes to collect a sample. One such feature is illustrated in FIG. 31B, which depicts a device with a skin-vacuum and lancing cavity for reducing the amount of time required to collect a sample. The skin-vacuum and lancing cavity can comprise a concave cavity into which the skin of the subject can be drawn (e.g., under negative pressure), and an opening comprising an inner opening through which a one or more piercing elements (e.g., lancets), e.g., 1, 2, 3, 4, 5, 6, 7, 8, 9, or 10 piercing elements, can exit and pierce skin so that a blood sample can be drawn from the subject. In some embodiments the device can comprise a vacuum actuator (e.g., button) for activating the vacuum.

FIG. 31C shows additional features. For example a device for collecting a blood sample can comprise a visual metering window that can allow a user to monitor sample collection and determine when the sample collection is complete. When the sample collection is complete, a visual metering window can be used to detect (e.g., visualize) a feature, colorimetric change, display of a symbol, masking of a symbol, or other means of indicating that collection is complete. Further user friendly features can comprise a removable cartridge (e.g., clip-in removable cartridge) for collecting and transporting a blood sample, as shown in FIG. 31D. A removable cartridge (e.g., clip-in removable cartridge) can comprise a cartridge tab for releasing and removing the cartridge. In some embodiments a removable cartridge (e.g., clip-in removable cartridge) can comprise a solid matrix for collecting, storing, and/or stabilizing a collected sample, and the removable cartridge can facilitate easy transport (e.g. transport at room temperature), and transport without the need for subsequent sample preparation or stabilization procedures.

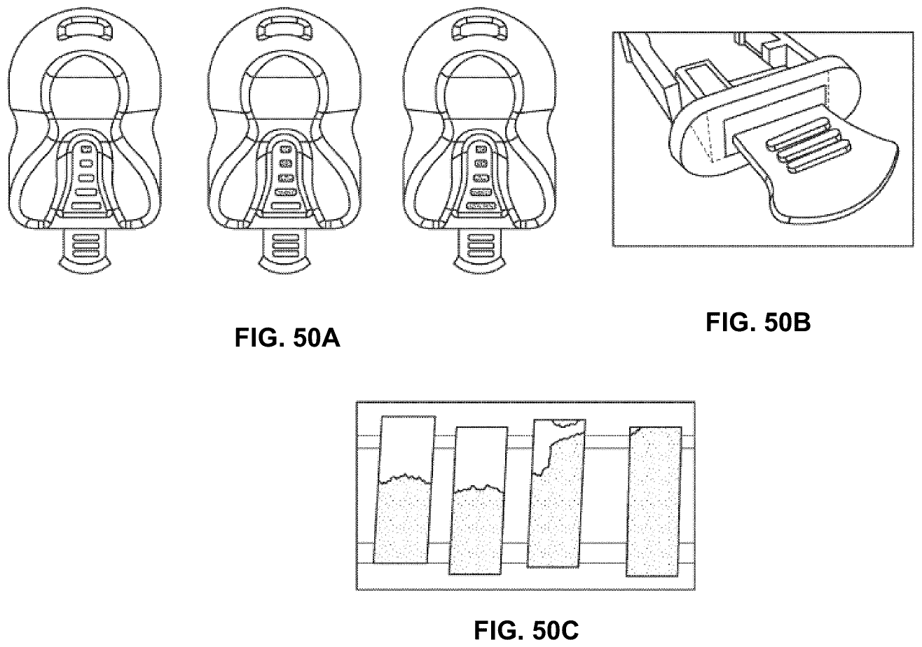

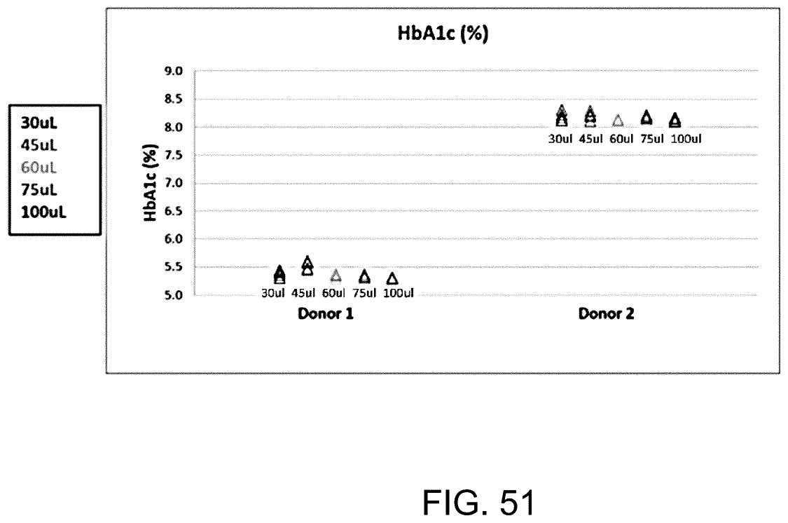

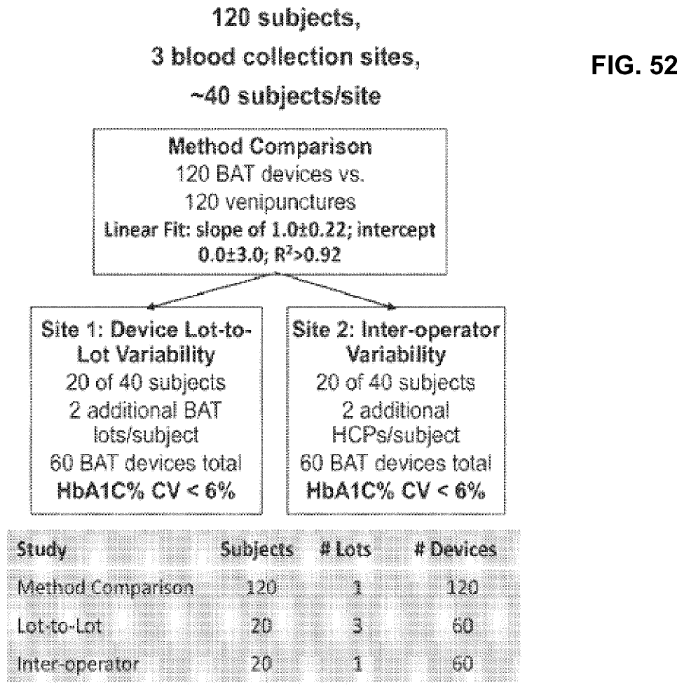

FIG. 50A shows an additional embodiment of the visual metering window and illustrates how blood absorption on the matrix strips can appear. In some embodiments a wicking pad captures excess blood unable to be absorbed by the matrix strips (FIG. 50B). Blood absorption on the matrix strips is illustrated in FIG. 50C.

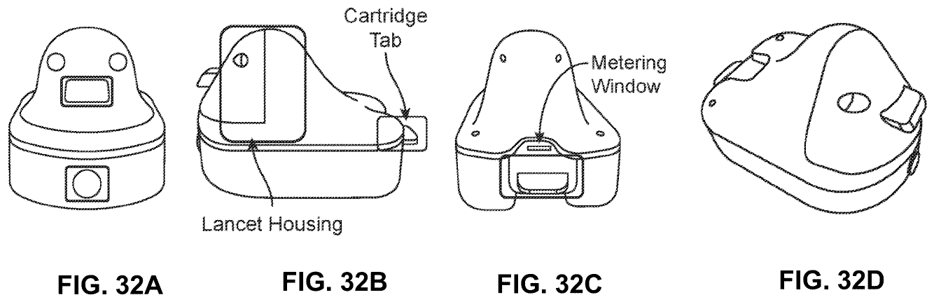

FIG. 32A, FIG. 32B, FIG. 32C, and FIG. 32D illustrate an integrated device with several of the user features described in FIG. 31A, FIG. 31B, FIG. 31C, and FIG. 31D. FIG. 32A illustrates a front view of a device with a dual button configuration. In some embodiments one button can be responsible for activating a vacuum and a second button can be responsible for activating a piercing (e.g., lancet piercing) mechanism; for example, the lower round button or vacuum button can be configured to cause a vacuum (negative gauge pressure) to be applied to the skin, and the upper rectangular button or lancet button can be configured to activate a vertical lancing mechanism to pierce the skin. In alternate embodiments buttons can be activated using a variety of methods; for example the buttons can be activated separately, in a specific sequence or order, or the two buttons can be combined into one button so that only a single button is needed to activate the collection mechanisms on the device. Buttons can perform different functions, and have different shapes, sizes, colors, or locations that support the function of each button. FIG. 32B illustrates a side view of the device depicted in FIG. 32A. FIG. 32B illustrates a device with a lancet housing, the lancet housing in this embodiment comprises a raised area for houses the lancing mechanism. Also depicted is a removable cartridge for storing a solid matrix, with a cartridge tab for removing the removable matrix cartridge. FIG. 32C depicts an alternate view of the device illustrated in FIGS. 32A and 32B. Illustrated features include the rear cartridge lid closure and cartridge tab, as well a visual metering widow configured to alert the user when the draw is complete. FIG. 32D illustrates a side perspective view of the device illustrated in FIG. 32A, FIG. 32B, and FIG. 32C.

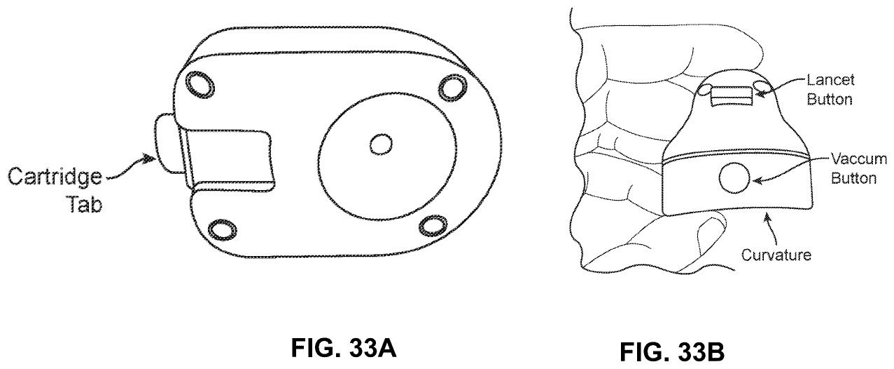

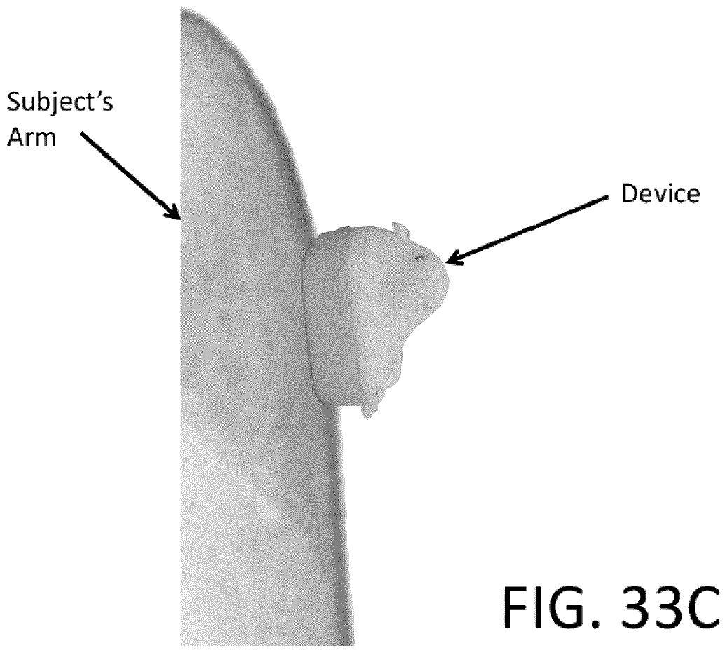

FIG. 33A depicts the bottom view of a device for collecting a blood sample, the depicted bottom region is the site of the device configured for making contact with the skin of the subject. As shown, the bottom of the device can comprise a concave cavity, for example a concave hemispherical cavity as shown here, although other shapes can also be used. The concave cavity in this embodiment forms a hemispherical cup disposed within the bottom of the device. The cupped skin area can be substantially larger than the lanced area. In some embodiments the ratio of the cupped skin to the lanced area can be greater than 20:1, greater than 30:1, greater than 40:1, greater than 50:1, greater than 60:1, greater than 70:1, greater than 80:1, greater than 90:1, or greater than 100:1. In some embodiments the cupped area can be within 20% margin of 500 mm² and the lanced area can be within a 20% margin of 8 mm². The lanced area can comprise a hole in the center of the concave cavity from which lancets can protrude; this area can additionally act as a vacuum channel and as part of the blood path to the deposition cartridge. The lancets or other piercing element can be held in a cylinder shaped lancet actuator. The lancet actuator can have a diameter of 1-10 mm (e.g. 1, 2, 3, 4, 5, 6, 7, 9, 10 mm). The area of the lancet actuator can be between 5 and 100 mm² (e.g. 5, 10, 13.2, 15, 20, 40, 60, 80, 100 mm²). The lancets or blades held by the lancet actuator can generate an incision area of between 1 and 20 mm² (e.g. 1, 3, 5, 9, 11, 15, 17, 20 mm²).

Any of the sample acquisition devices herein can also be referred to as the “device,” The housing, outer housing, upper housing, lower housing, or lancet housing of the device can comprise acrylobutadiene styrene (ABS), polypropylene (PP), polystyrene (PS), polycarbon (PC), polysulfone (PS), polyphenyl sulfone (PPSU), polymethyl methacrylate (acrylic)(PMMA), polyethylene (PE), ultra high molecular weight polyethylene (UHMWPE), lower density polyethylene (LPDE), polyamide (PA), liquid crystal polymer (LCP), polyaryl amide (PARA), polyphenyl sufide (PPS), polyether etherketone (PEEK), polyvinyl chloride (PVC), polyethylene terephthalate (PET), polytetra flouroethylene (PTFE), polyaryletherketone (PAEK), polyphenyl sulfone (PPSU), or a combination thereof. In some embodiments, the outer housing comprises polypropylene.

After the device is placed on the skin of the subject and the device is activated, a vacuum or pressure differential can form between the surface of the skin as well as components disposed within the device. Skin can be pulled into the cavity by the pressure differential and can be constrained by the walls of the cavity. At some point after the vacuum is formed between the device and the skin, a piercing element (e.g., a lancet) can be activated to pierce the skin. As such, the vacuum “cupping” can be configured to enhance blood flow to the lanced area and also aspirate blood from the opening collection site, through the device and into a collection cartridge.

A side view of the device depicted in FIG. 33A is illustrated in FIG. 33B. In some embodiments the bottom of the device can comprise a curved base. Slight curvature at the base of the device can allow the device to better conform to the patient's anatomy (e.g., arm, e.g., upper arm) and can guide orientation of the device. In some embodiments, the device described herein is used to draw blood from the arm. In some embodiments, the device is not used to draw blood from the fingertip. In some embodiments, the device is not used to draw blood from a neonate.

Collection of the sample can comprise steps and components configured for piercing (e.g., lancing) the subject's skin and providing or creating a vacuum to facilitate extraction of the sample. In some instances a vacuum can be provided before lancing of the skin; in other instances the vacuum can be provided after lancing of the subject's skin, and in still other instances the vacuum can be provided simultaneously with lancing the subject's skin. FIG. 33A and FIG. 33B illustrate features of a device that can facilitate efficient blood collection using application of a vacuum to the skin of the subject. The vacuum can operate as a means of deforming the skin, and this action coupled with lancing of the deformed skin can facilitate sample collection. In further instances the device can be configured to perform one or more additional processing steps (e.g. treatment, stabilization, and storage of the collected sample).

FIGS. 33A and 33B illustrates an embodiment of a device for collecting a sample using global vacuum and local suction. Methods for using the device can comprise multiple steps. For example, a device as depicted in FIG. 33A and FIG. 33B can be placed on the arm of a subject using the orientation illustrated in FIG. 33C. The global vacuum cavity can be placed in contact with the skin, and a seal can be created with an adhesive material or gasket material placed on the foot of the device (e.g. in curved surface of the device show in FIG. 33B). Vacuum can be applied with the press of a button or other mechanism. Thereafter, lancing can be applied, for example utilizing a spring loaded plunging mechanism which cause two (can be more or fewer) lancets to penetrate the skin and retract. Lancing can be performed by a single blade or multiple blades (e.g. two or more, three or more, four or more, five or more, or ten or more blades). Blades can have various tip sized and shapes (e.g. slanted, triangular, circular, pointed, blunt, serrated). In instances where more than one blade is present, blades can be configured or arranged into patters with different shapes or orientations (e.g. ring, star, hash, square, rectangular etc.)

After sample is collected additional processing steps can be performed on the sample. Once blood is collected using a sample acquisition device, the sample can be treated, stabilized and stored. In some embodiments collection devices, e.g. devices disclosed in the present application, can be configured to collect, treat, and store the sample. Sample drawn by the device can be stored in liquid or solid form. The sample can undergo optional treatment before being stored. Storage can occur on the device, off the device, or in a removable container, vessel, compartment, or cartridge within the device.

A sample acquisition device can be configured to collect, treat, stabilize, and store a collected sample. Additional processing (e.g. treatment, stabilization, and storage) can comprise steps or methods and device components configured for concentrating the sample, adjusting or metering the flow of the sample, exposing the sample to one or more reagents, and depositing the sample on a solid substrate or matrix. Methods for using a sample acquisition device can include steps to perform one or more of the following processes: collection, treatment, stabilization, and storage of the sample. Collection, treatment, stabilization, and storage can be performed within a single device. Treatment can comprise filtration of the sample to separate components or analytes of interest. In some embodiments, the collected sample can be collected, treated, and stabilized prior to transfer to a removable cartridge for storage. In other embodiments, one or more steps comprising collecting, treating, and stabilizing, can occur on a removable cartridge.

In some embodiments, single action (e.g. activation using a button) can activate alternate processing steps including sample treatment, stabilization, and storage. Additional processing steps can be performed on the device in response to single action, or in some instances two or more user actions can be necessary to move the sample through one or more different processes (e.g. collection, treatment, stabilization, and storage). User actions can comprise pressing a single button, pressing multiple buttons, pressing two or more buttons at the same time, and pressing two or more buttons in a prescribed sequence (e.g. based on a prescribed sequence to perform a set of treatment steps desired by the user.)

Sample collected on a device can undergo a treatment step prior to being deposited on a solid substrate. A cartridge containing the two or more deposition strips can be maintained in a near vertical orientation to reduce deposition speed and increase sample deposition consistency. Vacuum can be released by the user and device can be removed when a visual (or other) metering mark is observed. The sample cartridge containing the two or more solid matrix strips can be removed from the device.

In some embodiments, solid matrix strips can be sized to maximize blood collection volume while still fitting into commonly used containers (e.g. a 3 ml BD vacutainer, deep well plate or 2 ml Eppendorf tube). Solid matrix can be configured to meter out, collect and stabilize fixed volumes of blood or plasma (e.g. greater than 25 μL, 50 μL, greater than 75 μL, greater than 100 μL, greater than 125 μL, greater than 150 μL, greater than 175 μL, greater than 200 μL, or greater than 500 μL of blood or plasma). A solid matrix can comprise cellulose based paper (e.g. Whatman™ 903 or 226 paper), paper treated with chemicals or reagents for stabilizing the sample or one or more components of the sample (e.g. RNA stabilization matrix or Protein Stabilization Matrix). In some embodiments, the solid matrix comprises a cellulose filter paper. In some embodiments, any suitable commercially available filter paper is used. Examples of commercially available filter paper include, but are not limited to, filter paper from Whatman®, such as 903 sample collection cards and fast transit analysis (FTA®) card. In some embodiments, the solid matrix comprises a nitrocellulose filter paper. In some embodiments, the solid matrix does not comprise glass fiber filter paper.

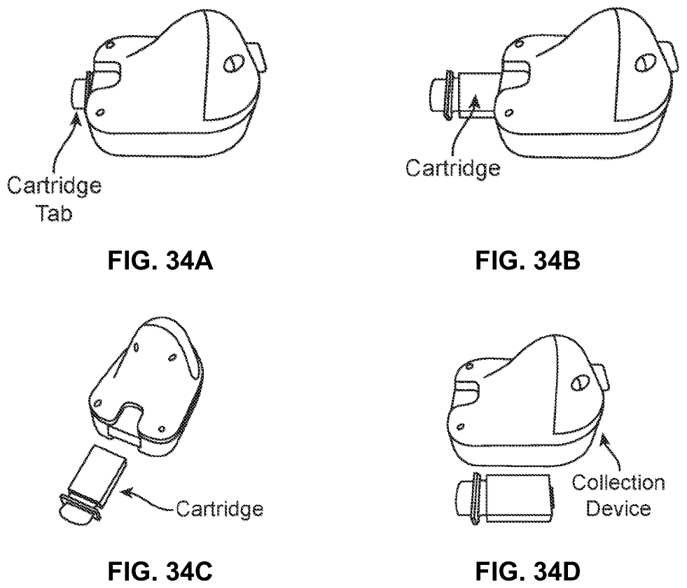

Sample acquisition devices (e.g. the devices depicted in FIGS. 31A-D, FIGS. 32A-D, and FIGS. 33A-C) can comprise a removable cartridge or enclosure for storing a liquid sample or solid matrix for removing the sample once it has been collected. FIG. 34A, FIG. 34B, FIG. 34C, and FIG. 34D illustrate steps for removing a removable cartridge from an exemplary devices configured with a removable cartridge (e.g. the devices depicted in FIGS. 31A-D, FIGS. 32A-D, and FIGS. 33A-C). A device can come with a cartridge pre-loaded in the device, as shown in FIG. 34A, or a device can come without the cartridge such that a cartridge can be acquired separately and installed into the device by the user prior to sample collection. The device illustrated in FIG. 34A is shown with the cartridge loaded in the device and with the cartridge tab projecting from the back of the device. After a draw is complete the cartridge can be removed as shown. The cartridge can comprise one or more solid matrix strips, or a vessel for storing liquid sample. Alternatively, the cartridge can be empty. In some cases, the cartridge can include liquid handling reagents. In some embodiments, the cartridge/device interface can contain a seal (e.g. gasket or other type) to maintain internal pressure during the draw period.

FIG. 34B illustrates the partially removed cartridge. Removal of the cartridge can be performed using the cartridge tab shown in FIG. 34A. In FIG. 34C, the cartridge depicted in FIG. 34B has been completely removed and is placed at the back of the collection device in an orientation by which it was removed. FIG. 34D illustrates the fully removed cartridge placed parallel with the collection device to illustrate the positioning of the cartridge within the device. Once removed, a cartridge can be placed in a secondary vessel with desiccant to dry the sample. In instances where the cartridge comprises strips of solid matrix for storing the sample, the strips can be removed with an extraction tool or other mechanism prior to analysis.

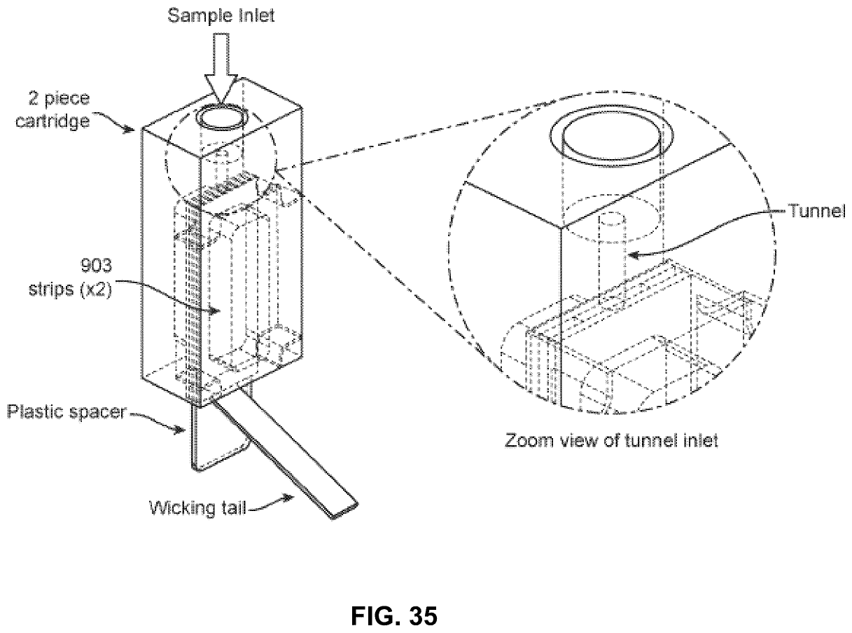

A cartridge, for example the cartridge illustrated in FIGS. 34A-D, can comprise multiple components for facilitating accurate and precise sample collection. FIG. 35 illustrates a cross sectional and zoom-in of a cartridge embodiment that can be used in any of the devices disclosed herein. In some instances a cartridge can comprise one or more solid matrices for collecting a blood sample. In embodiments where two or more solid matrices are included in the sample, the matrices can be stacked or arranged in ways that facilitate blood collection, distribution, precision and reproducible volumes of sample or analyte per surface area of solid substrate. In instances where two or more solid matrices are include, the matrices can have different compositions or purposes; for example one matrix can separate cells from a cell free component and collect the cell free component on one matrix, and a second matrix or other matrices can collect raw unseparated sample.

An exemplary sample storage cartridge is depicted in FIG. 35. The cartridge can comprises two pieces, a top piece and a bottom piece which can be merged to form internal chambers. Sample can move through the opening in the concave cavity of the device and into the cylinder shaped sample inlet into a tunnel inlet before entering a chamber. The chamber can comprise solid matrix strips for absorbing the sample and a spacer (e.g., plastic spacer) to separate the two solid matrix strips. The spacer (e.g., plastic spacer) between the two strips can be adjustable and removable, depending on other relevant aspect (e.g. the needs and application of the sample being collected, stability of the analyte, rate of absorption requirements etc). The spacer (e.g., plastic spacer) can comprise a range of widths and coatings. Exemplary widths include widths in the millimeter to centimeter range (e.g. greater than 2 mm, greater than 4 mm, greater than 6 mm, greater than 8 mm, greater than 10 mm, greater than 0.2 cm, greater than 0.4 cm etc.). In further embodiments, the spacer (e.g., plastic spacer) can be coated with materials including hydrophobic coatings, hydrophilic coatings, antimicrobial coatings, coatings that bind to one or more components of a sample, coatings for binding to or inhibiting enzymes that can degrade or otherwise impact the quality of one or more analytes on the sample.

As shown in FIG. 35, after moving thought the sample chamber, excess sample can move out of the storage cartridge through a wicking tail. The wicking tail can be configured to absorb excess sample overflow. The wicking tail can be configured (e.g. composition adjusted) so that the wicking tail can be used as a means to control the volume of the sample absorbed on the solid matrix strips. In further embodiments, the wicking tail can be used as or incorporated into an indicator or be visible through a viewing window configured for informing a user that the collection procedure is complete. The cartridges illustrated in FIG. 35 depict sample stored on a solid matrix; however, this should not be taken to limit the devices disclosed herein-devices can comprise cartridges or means for collecting, treating, stabilizing and storing sample in either a liquid or a solid state.

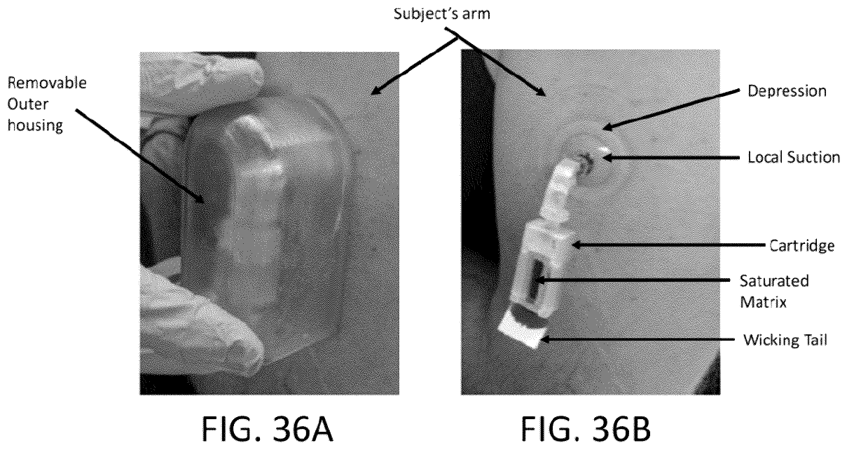

FIG. 36A and FIG. 36B illustrate an exemplary device configured with a sample storage cartridge similar to the cartridge illustrated in FIG. 35. FIG. 36A shows a removable outer housing configured for applying global vacuum to the sample collection site located on a subject's arm. In some embodiments the global vacuum can be applied through a concave cavity for deforming the skin prior to lancing. FIG. 36B illustrates exemplary local suction and blood collection components of the device depicted in FIGS. 36A and 36B. A depression is apparent on the arm of the subject, indicating that global suction was applied. Local suction, through a suction cup, is provided on the surface of the skin around the location where the skin of the subject was lanced. The sample is showing moving from the lanced site into a cartridge comprising a saturated matrix and a wicking tail. The wicking tail can be used to absorb excess sample and standardize or meter the volume of blood deposited on the saturated matrix.

In some embodiments, solid matrices, for example solid matrices included in a cartridge, can be sized to maximize blood collection volume while still fitting into commonly used containers (e.g. a 3 ml BD vacutainer, deep well plate or 2 ml Eppendorf tube). The cartridge can include one solid matrix, two solid matrices, three solid matrices, four solid matrices, or more than four solid matrices. In some embodiments, the cartridge includes two solid matrices. Solid matrix can be configured to meter out, collect and stabilizes fixed volumes of blood or plasma (e.g. greater than 50 μL, greater than 75 μL, greater than 100 μL, greater than 125 μL, greater than 150 μL, greater than 175 μL, greater than 200 μL, or greater than 500 μL of blood or plasma). In some embodiments, the cartridge comprises two solid matrices, wherein each solid matrix stabilizes 75 μL of blood for a total of 150 μL of blood. A solid matrix can comprise cellulose based paper (e.g. Whatman™ 903 paper), paper treated with chemicals or reagents for stabilizing the sample or one or more components of the sample (e.g. RNA stabilization matrix or Protein Stabilization Matrix).

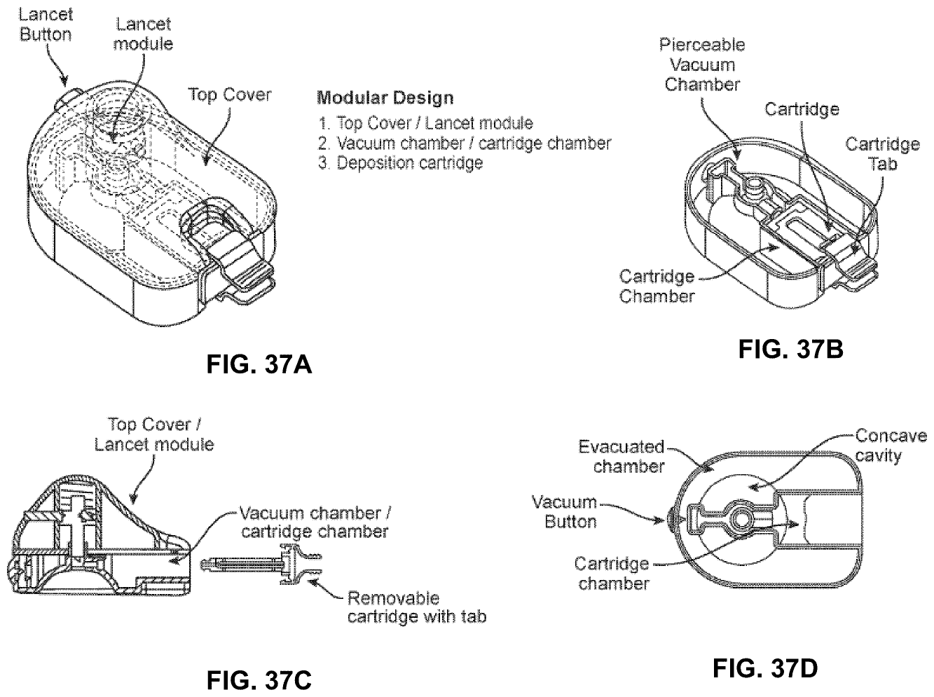

Devices for collecting a blood sample can be modular, with two or more compartments for performing specific actions or functions on the device. An exemplary modular device is depicted in FIG. 37A, FIG. 37B, FIG. 37C, and FIG. 37D. FIG. 37A illustrates the top view of a modular sample acquisition device (e.g. similar to the devices depicted in FIGS. 31A-D, FIGS. 32A-D, and FIGS. 33A-C, and FIGS. 34A-D). Disposed within the top cover of the device illustrated in the FIG. 37A, is a lancet module and a lancet button for activating the lancet module. FIG. 37B illustrates the vacuum chamber and cartridge chamber disposed within the lower portion or “foot” of the device. This module comprises a pierceable vacuum chamber and a cartridge chamber, within the cartridge chamber is a cartridge. Projecting out of the backside of the device is a cartridge tab, which can be used to remove the cartridge as illustrated in FIGS. 34A-D. FIG. 37C illustrates a cross section of the device. The cross section displays the top cover and lancet module also shown in FIG. 37A, and in the bottom of the device the vacuum chamber/cartridge chamber shown in FIG. 37B. Also shown in FIG. 37C is a side view of the removable cartridge with the cartridge tabs, the cartridge is removed from the device and positioned to the side of the cartridge chamber where the cartridge can be inserted or from where the cartridge can be removed. FIG. 37D illustrates a top view of the device in a top down view showing components present in FIG. 37B. FIG. 37D shows the vacuum button, with a sharp end for piercing the evacuated chamber, piercing the evacuated chamber can form suction pulling the sample through the opening of the concave cavity, into the sample inlet of the cartridge and onto the solid matrix strips in the cartridge.

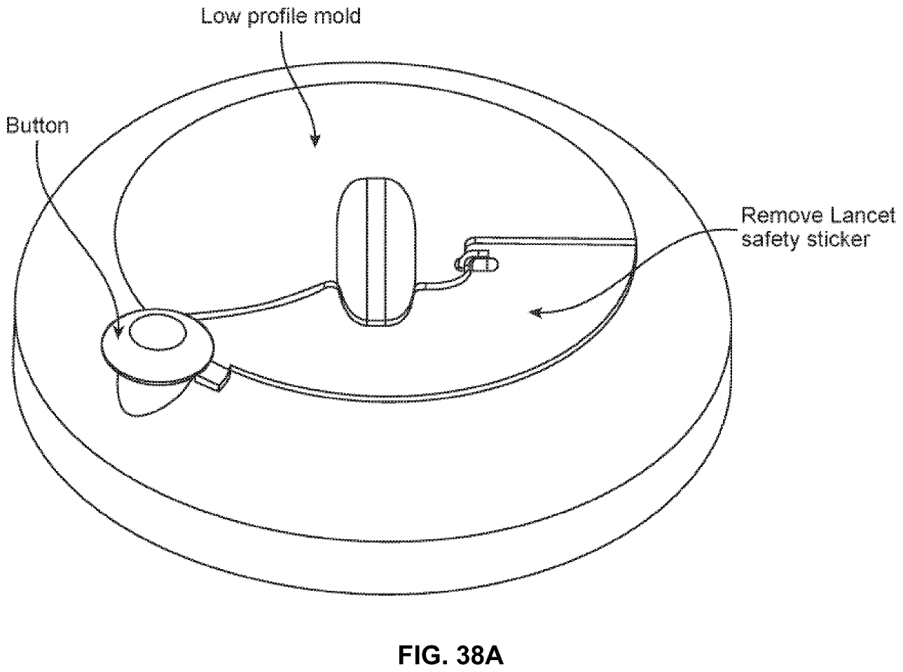

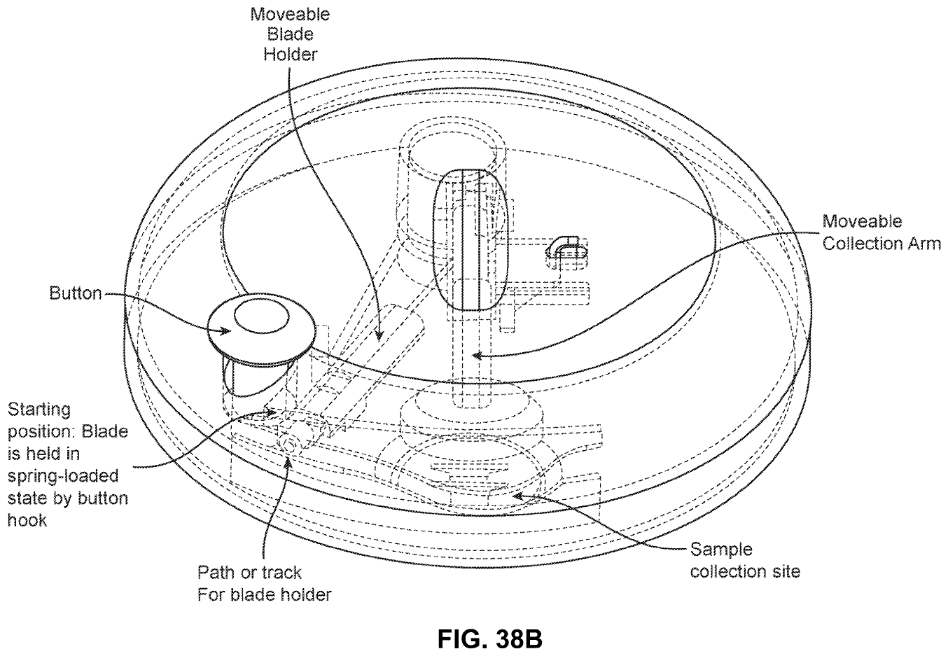

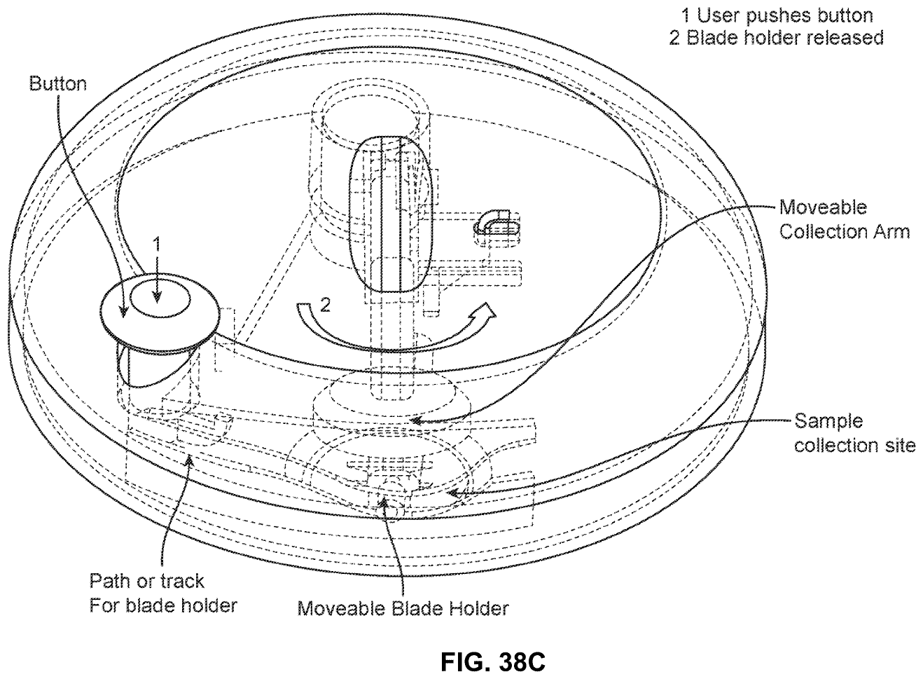

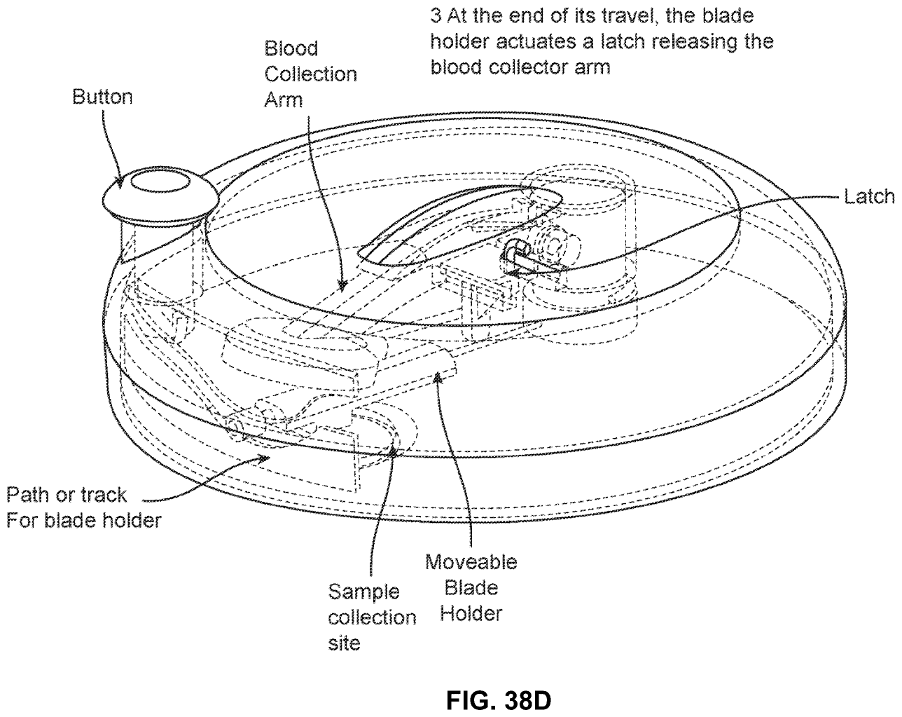

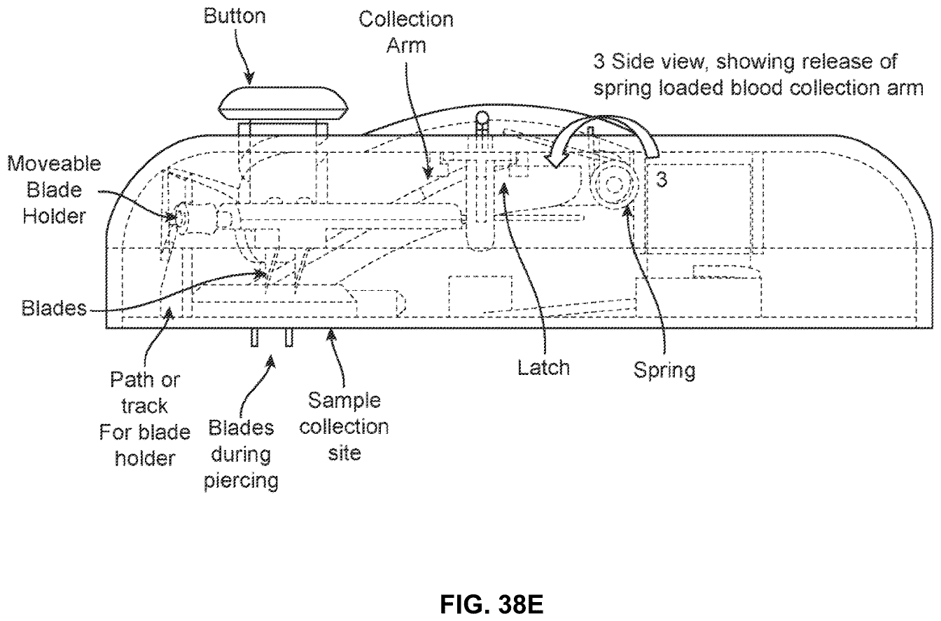

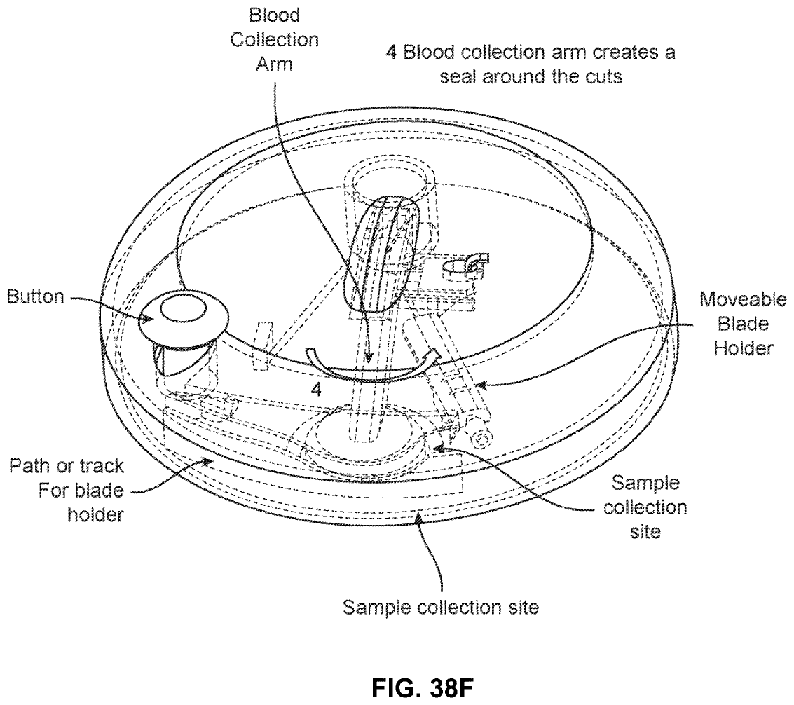

FIGS. 38A-38F shows various views of an exemplary embodiment of a device configured for single activation piercing and collection of a blood sample from a patient. As shown in FIG. 38A the device can comprise a low profile mold with a removable lancet safety sticker, and a button for single activation of the device. FIG. 38B illustrates the internal workings of the device in an exemplary starting position. In the starting position, a moveable blade holder is held in a spring-loaded state by a button hook that releases the blade holder when the button depressed. The device also comprises a path or track for the released blade holder to move along once the blade holder is released. Also illustrated in FIG. 38B is the sample collection site and the moveable collection arm. FIG. 38C illustrates the internal workings of the device once the button is depressed (1), and the blade holder is released (2). When the button is depressed the moveable blade holder is released, and moves down the path or track. At this point in device activation, the moveable collection arm is still in the initial position, disposed above the sample collection site with sufficient space for the moveable blade holder to move between the moveable blade holder and the sample collection site. FIG. 38D illustrates the internal working of the device. Once released, the button actuated moveable blade holder rotates through the device by moving along the path or track. At the end of the path or track it actuates a latch, thereby releasing the blood collection arm. FIG. 38E shows a side view of the device, illustrating the movable blade holder reaching the end of the path or track where the removable blade holder releases a latch that activates the spring loaded blood collection arm resulting in release of the spring loaded collection arm (3). The blood collection arm is released over the sample collection site. Also depicted in FIG. 38E are the blades and an exemplary depth of the blades at a depth by which the blades are configured to project through the bottom of the device and into the subjects skin. The depth of the blades is established by the shape and height of the track or path on which the moveable blade holder travels. FIG. 38F illustrates the formation of a seal by the released blood collection arm over the sample collection site (4). The exemplary device illustrated in FIGS. 38A-38F comprises four steps for activation; first a single button is depressed causing a blade holder to be released and the blade holder moves down the depicted track or path. Along the path, the blades held by the blade holder pierce the skin, and at the end of the blade holder track or path the blade holder activates a latch that releases a spring loaded collection arm over the sample collection site. The collection arm forms a seal with the sample collection site, and the collection arm can draw the blood from the subject. In some instances, the device illustrated in FIGS. 38A-38F can comprise an evacuated chamber or onboard vacuum for creating suction.

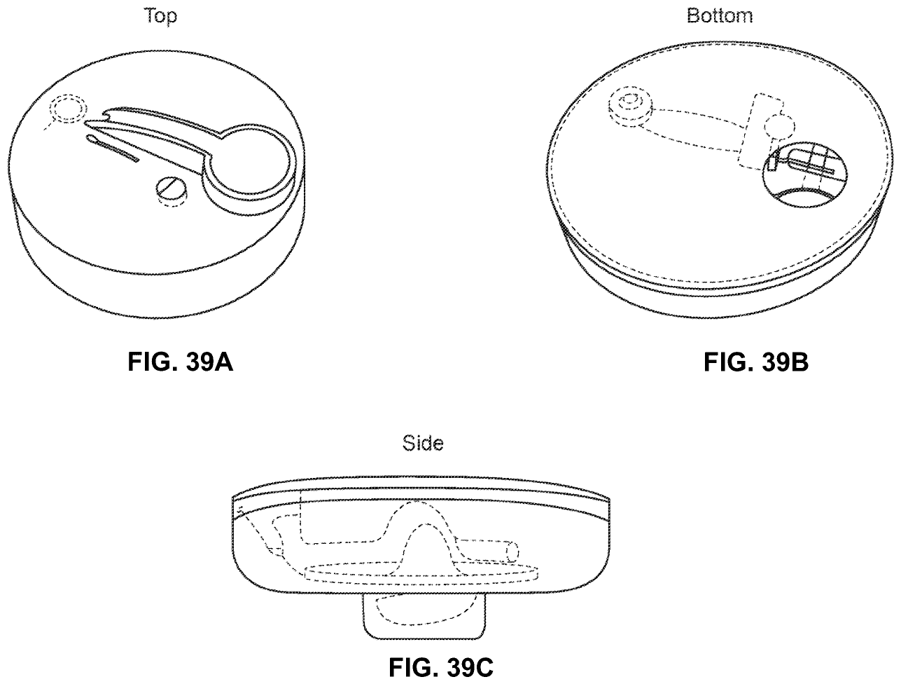

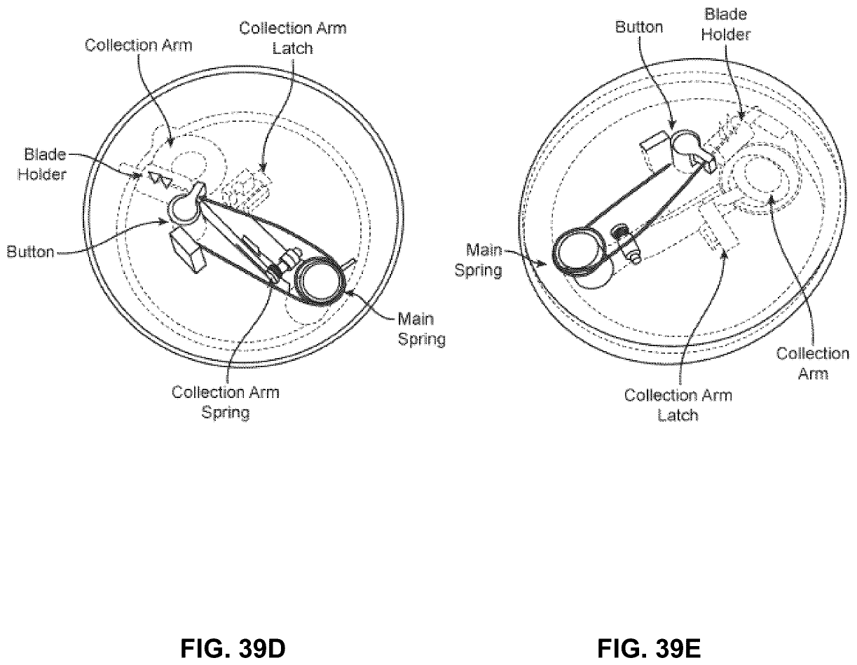

An alternate embodiment of a low profile sample acquisition device is shown in FIGS. 39A-39E. A top view (FIG. 39A), bottom view (FIG. 39B), and side view (FIG. 39C) of an exemplary low profile sample acquisition device, along with internal views of the device (FIG. 39D and FIG. 39E), illustrate the button, blade holder with two blades, collection arm, collection arm main spring and release spring, as well as the latch that releases the collection arm. In this embodiment (FIG. 39D and FIG. 39E), the button can be depressed by the user, causing the blade holder and installed blades to rotate, driven by the main spring, and during the rotation pierce the subject's skin, at the end of the rotation the blade holder can activate the collection arm latch that releases the collection arm spring causing the collection arm to release bringing it in contact with the skin of the subject. The collection arm can create contact with the skin, and can be configured to provide suction or vacuum to extract blood sample.

The devices illustrated in FIGS. 38A-38F and FIGS. 39A-39E, and any devices disclosed in the present application, can comprise a single or multiple blades for piercing a subjects skin; for example one or more, two or more, three or more, four or more, five or more, or ten or more blades. Blades can be configured in different shapes or orientations, for example a ring shape, a star shape, a hash shape, square shapes, rectangular shapes, etc.

The devices illustrated in FIGS. 38A-38F and FIGS. 39A-39E, and any devices disclosed in the present application, can be configured to collect, treat, and store the sample. The sample drawn by the device can be stored in liquid or solid form. The sample can undergo optional treatment before being stored. Storage can occur on the device, off the device, or in a removable container, vessel, compartment, or cartridge within the device.

The devices illustrated in FIGS. 38A-38F and FIGS. 39A-39E, and any devices disclosed in the present application can be configured to collect, treat, stabilize, and store a collected sample. A device can be configured to perform one or more of the following processes: collection, treatment, stabilization, and storage of the sample. Collection, treatment, stabilization, and storage can be performed within a single device. Treatment can comprise filtration of the sample to separate components or analytes of interest. Treatment can also comprise exposure to buffers or reagents for stabilizing the sample. In some embodiments the device can be configured to concentrate one or more components of the sample.

In some instances, one or more of the processes (e.g. collection, treatment, stabilization, and storage of the sample) can be performed on the device in response to singe activation of the device by the user. In other instances two or more user actions can need to be performed to move the sample through one or more different processes (e.g. collection, treatment, stabilization, and storage). User actions can comprise pressing a single button, pressing multiple buttons, pressing two or more buttons at the same time, and pressing two or more buttons in a prescribed sequence (e.g. based on a prescribed sequence to perform a set of treatment steps desired by the user.)

Collection of the sample can comprise steps and components configured for lancing the subject's skin and providing or creating a vacuum to extract the sample. In some instances a vacuum can be provided before lancing of the skin, in other instances the vacuum can be provided after lancing of the subject's skin, in further instances the vacuum can be provided simultaneously with lancing the subject's skin.

Treatment of the device can comprise concentrating the sample, adjusting or metering the flow of the sample, exposing the sample to one or more reagents, and depositing the sample on a solid substrate or matrix. Embodiments the device can comprise a removable cartridge or enclosure for storing a liquid sample or solid matrix for removing the sample once it has been collected. A solid matrix can comprise cellulose based paper (e.g. Whatman™ 903 paper), paper treated with chemicals or reagents for stabilizing the sample or one or more components of the sample (e.g. RNA stabilization matrix or Protein Stabilization Matrix).

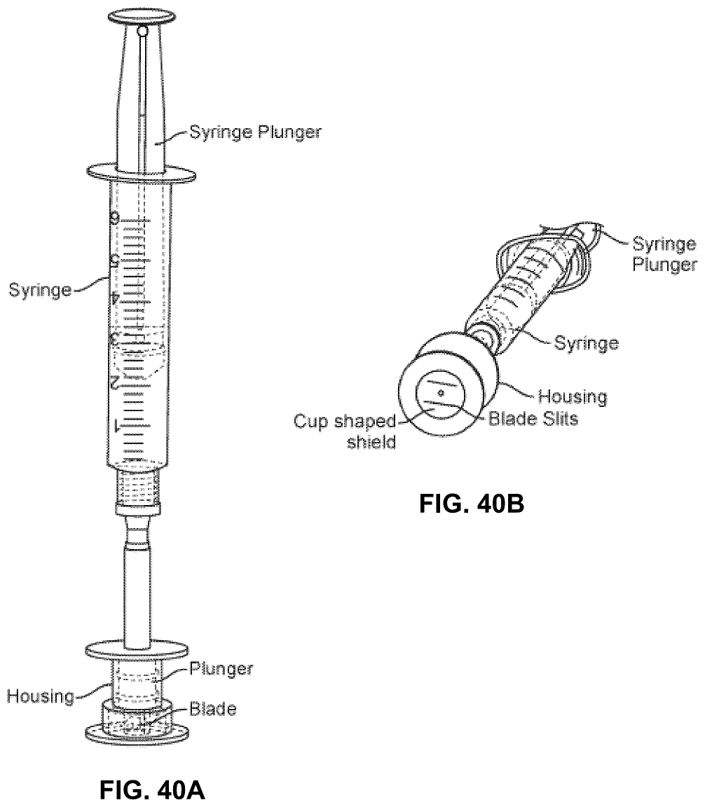

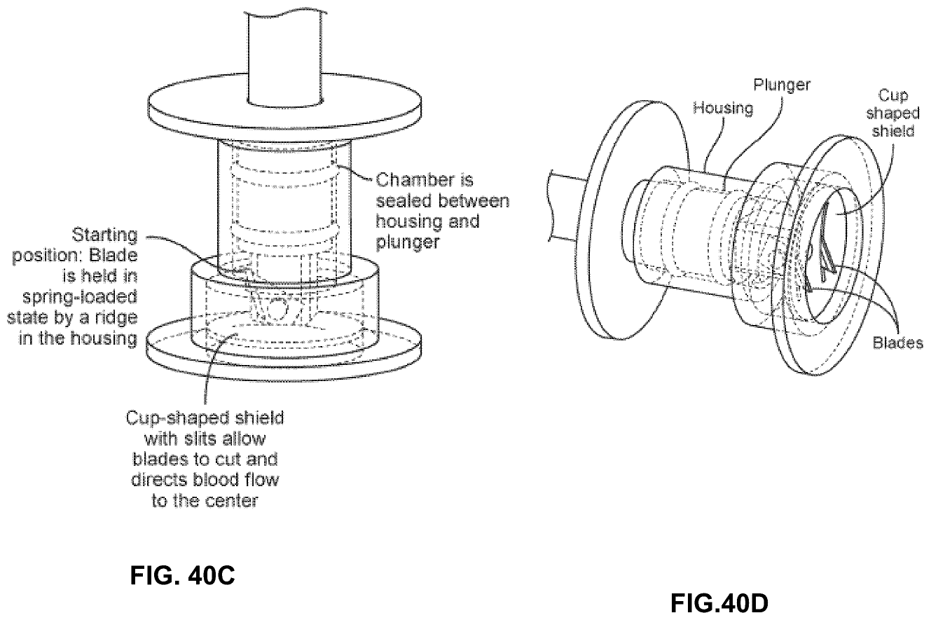

Devices for collecting a blood sample from a subject can also rely on a vertically oriented device, as shown in FIGS. 40A-40D, FIGS. 41A-41B, and FIGS. 42A-42C.

FIG. 40A illustrates an exemplary embodiment of a sample acquisition device with a vertical cutting modality. The device can comprise a syringe with syringe plunger, connected to a housing comprising a plunger and a blade. The device can comprise a housing within which a plunger and blade or disposed. The housing can be oriented towards the skin of the subject with the syringe and syringe plunger oriented away from the subject. FIG. 40B shows the same device positioned on its side to illustrate the cup shaped shield with slits for the blades. FIG. 40C illustrates a side view of the housing portion of the device in the starting position. The view illustrates the presence of a chamber sealed between the housing and a plunder disposed within the housing. The blade is held in a spring-loaded state by a ridge disposed within the housing. On the bottom of the device, a cup-shaped shield with slits allows blades to move through the cup-shaped shield to pierce the skin of the subject and, through micro-channels cut into the cup-shaped shield, direct blood flow to the center of the cup. FIG. 40D is a side view of the device with a view of the housing and the plunder disposed within. Also visible on the bottom of the device is the cup-shaped shield with blades projecting through the slits of the cup-shaped shield, showing how cutting of the skin of the subject is performed.

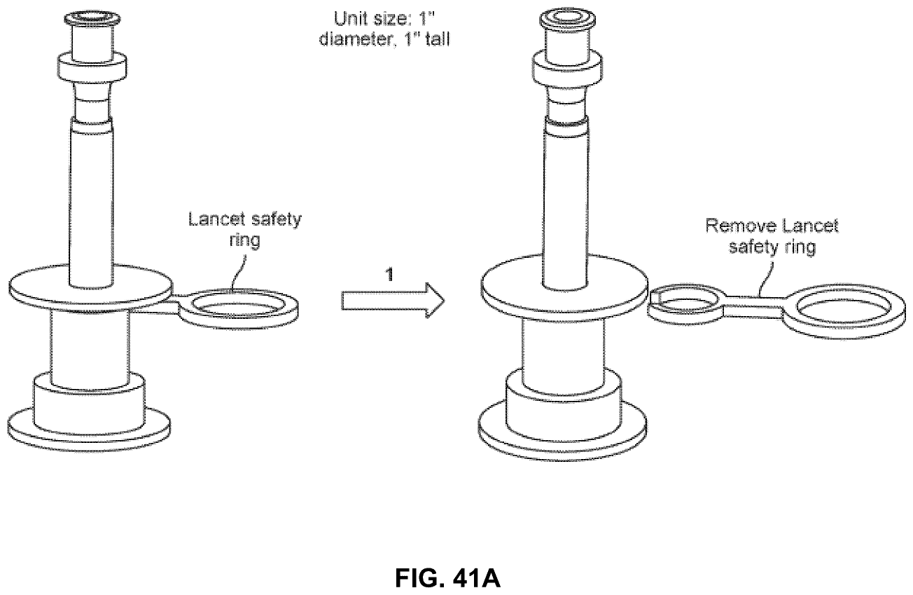

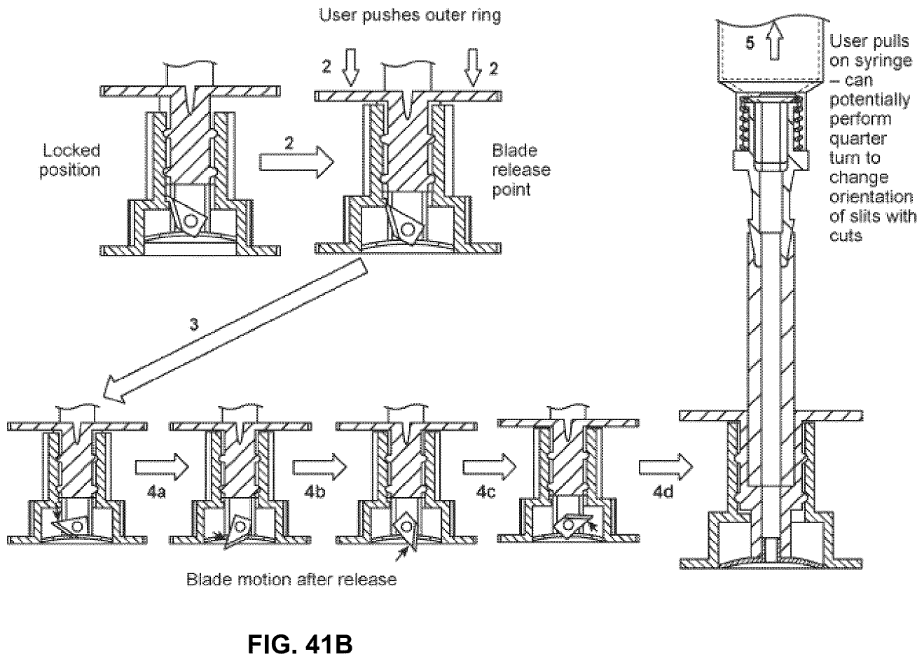

Methods for using the device illustrated in FIGS. 40A-40D, are illustrated in FIG. 41A and FIG. 41B. As shown in FIG. 41A, a lancet safety ring is then removed from the device (1). At this point, the device is still in the locked position with the blade resting on a ridge within the device (See FIG. 41B). Then a user pushes down on the outer ring (2), depressing an internal spring and releasing the blade (3). The blade then rotates (4a-4d) cutting the skin of the user and exposing blood that moves into the cup-shaped shield. Finally, the user pulls on the syringe plunger (5) to create negative pressure and draw the sample through the micro channels and slits in the bottom of the cup-shaped shield, and into the syringe.

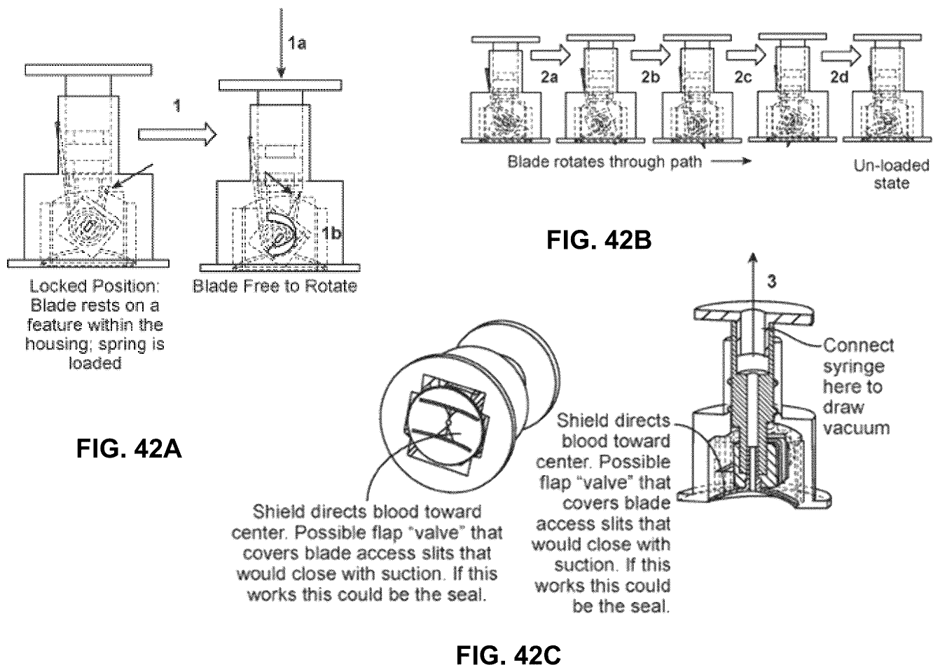

FIG. 42A, FIG. 42B, and FIG. 42C illustrate a device with a vertical rotational cutter similar to those illustrated in FIGS. 40A-40D, and FIGS. 41A-41B, however a wound spring mechanism is used to control a blade holder and thus drive rotation of the blade in the device. FIG. 42A illustrates the blade and spring, with the blade in the locked position-resting on a features within the housing, with the spring loaded. A force is applied to the top of the device to depress the spring (1a), and the blade is free to rotate (1b). As shown in FIG. 42B, the blade rotates through the path (2a-2d) during which it cuts the skin of the subject, until it reaches an unloaded state. The cut skin of the subject releases blood sample which moves through the shield which directs blood follow towards the center, as shown in FIG. 42C (side view). A possible flap “valve” can be included that covers the blade access slits and form a seal to close the suction. Finally, as shown in FIG. 42C, the syringe can be retracted and the sample can be draw into a sample storage compartment disposed within the device.

Sample acquisition devices (e.g. devices illustrated in FIGS. 40A-40D, FIGS. 41A-41B, and FIGS. 42A-42C, can comprise a single or multiple blades for piercing a subjects skin; for example one or more, two or more, three or more, four or more, five or more, or ten or more blades. Blades can be configured in different shapes or orientations, for example a ring shape, a star shape, a hash shape, square shapes, rectangular shapes, etc.

Sample acquisition devices (e.g. devices illustrated in FIGS. 40A-40D, FIGS. 41A-41B, and FIGS. 42A-42C, can be configured to collect, treat, and store the sample. The sample drawn by the device can be stored in liquid or solid form. The sample can undergo optional treatment before being stored. Storage can occur on the device, off the device, or in a removable container, vessel, compartment, or cartridge within the device.

Sample acquisition devices (e.g. devices illustrated in FIGS. 40A-40D, FIGS. 41A-41B, and FIGS. 42A-42C, can be configured to collect, treat, stabilize, and store a collected sample. A device can be configured to perform one or more of the following processes: collection, treatment, stabilization, and storage of the sample. Collection, treatment, stabilization, and storage can be performed within a single device. Treatment can comprise filtration of the sample to separate components or analytes of interest. In some instances one or more of the processes (e.g. collection, treatment, stabilization, and storage of the sample) can be performed on the device in response to singe activation of the device by the user. In other instances two or more user actions can need to be performed to move the sample through one or more different processes (e.g. collection, treatment, stabilization, and storage). User actions can comprise pressing a single button, pressing multiple buttons, pressing two or more buttons at the same time, and pressing two or more buttons in a prescribed sequence (e.g. based on a prescribed sequence to perform a set of treatment steps desired by the user.)

Collection of the sample can comprise steps and components configured for lancing the subject's skin and providing or creating a vacuum or suction to extract the sample. In some instances a vacuum or suction can be provided before lancing of the skin, in other instances the vacuum or suction can be provided after lancing of the subject's skin, in further instances the vacuum can be provided simultaneously with lancing the subject's skin.

Treatment of the device can comprise concentrating the sample, adjusting or metering the flow of the sample, exposing the sample to one or more reagents, and depositing the sample on a solid substrate or matrix. Embodiments the device can comprise a removable cartridge or enclosure for storing a liquid sample or solid matrix for removing the sample once it has been collected. A solid matrix can comprise cellulose based paper (e.g. Whatman™ 903 paper), paper treated with chemicals or reagents for stabilizing the sample or one or more components of the sample (e.g. RNA stabilization matrix or Protein Stabilization Matrix).

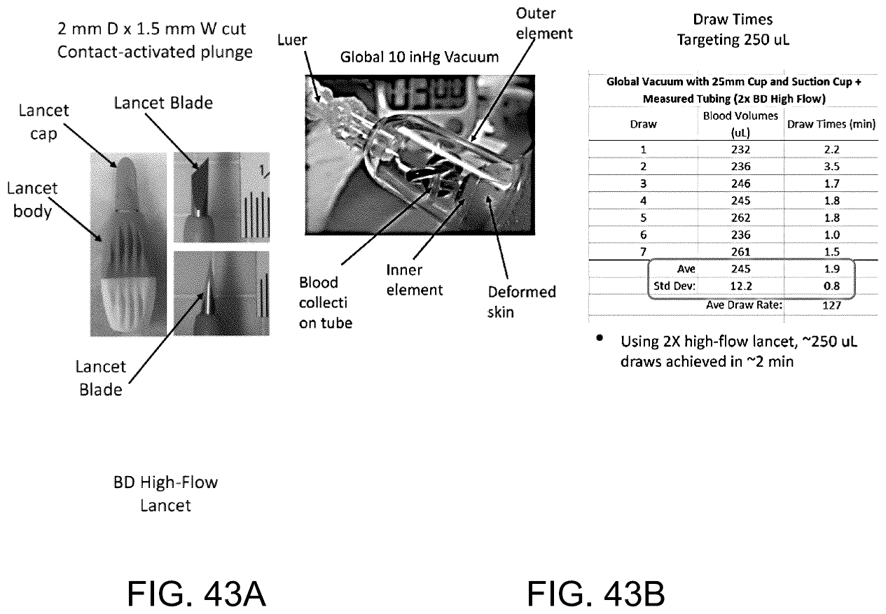

FIG. 43B illustrates a device configured for applying global vacuum and local suction to collect a sample. Lancet blades can be used to pierce the skin of a subject prior to applying the device to collect the sample. Lancets can comprise high flow or low flow. After lancing, a device for applying global vacuum and local suction is applied to the location of the cut. The device, as shown in FIG. 43B, can comprise two nested components, an outer element for applying global vacuum to deform the skin and an inner element for providing local suction. Connected to the inner element is a tube with a luer adaptor at the end of it, suction is provided through the luer adaptor, enabling sample to be drawn into the collection tube. The suction provided through the luer adaptor is used to both deform the skin and to extract the sample.

The method and device for collecting a blood sample, as illustrated in FIG. 43B is configured to collect a targeted volume of blood in under 5 minutes. Examples of blood volumes and corresponding collection times for seven samples are presented in Table 1. The average blood volume drawn was 245 μL+/−12.2 μL in an average of 1.9 minutes+/−0.8 minutes. The average rate for blood collection was 127 μL per minutes. Blood samples collected methods and devices comprising global vacuum and local suction can cover the range of greater than 50 μL per minute, greater than 75 μL per minute, greater than 100 μL per minute, greater than 125 μL per minute, greater than 150 μL per minute, greater than 175 μL per minute and greater than 200 μL per minute. Examples of the pressure generate by the global vacuum can include greater than 5inHg, greater than 8inHg, greater than 10inHg, greater than 12 inHb, and any pressures or ranges of pressures sufficient to pull skin into chamber of the outer element and create overall vacuum on skin in the tube.

Mechanisms that incorporate global vacuum and local suction can increase the rate of sample collection over methods that do not have global vacuum and local suction. Table 1 below illustrates draw times for global vacuum and local suction device illustrated in FIG. 43B. Global vacuum and local suction can comprise any method or device configured for, under negative pressure, sucking or deforming skin into a larger cavity and drawing blood sample out of the surface of the sample. In mechanisms that rely on global vacuum and local suction there can be two or more contacts; for example the outer element (e.g. the bell shaped cup) and the inner element (e.g. inner local suction cup). These nested elements can be configured such that the ratio of the effected surface areas (e.g. ratio generated by the surface area of the global vacuum area divided by the local suction area) are present at a particular ratio. The ratio can be configured to deform the skin and then disrupt the site above the incision to facilitate extraction of the sample.

TABLE 1
Draw times for Global Vacuum Local Suction Blood Collection Method
Global Vacuum with 25 mm Cup and Suction Cup +
Measured Tubing (2X Becton Dickinson (BD) High Flow lancets)

| Draw | Blood Volumes (uL) | Draw Times (min) |
|---|---|---|
| 1 | 232 | 2.2 |
| 2 | 236 | 3.5 |
| 3 | 246 | 1.7 |
| 4 | 245 | 1.8 |
| 5 | 262 | 1.8 |
| 6 | 236 | 1.0 |
| 7 | 261 | 1.5 |
| Ave | 245 | 1.9 |
| Std Dev: | 12.2 | 0.8 |
| | Ave Draw Rate: | 127 |

The devices illustrated in FIGS. 43A and 43B, and any sample acquisition devices disclosed in the present application, can comprise a single or multiple blades for piercing a subjects skin; for example one or more, two or more, three or more, four or more, five or more, or ten or more blades. Blades can be configured in different shapes or orientations, for example a ring shape, a star shape, a hash shape, square shapes, rectangular shapes, etc.

The devices illustrated in FIGS. 43A and 43B, and any sample acquisition devices disclosed in the present application, can be configured to collect, treat, and store the sample. The sample drawn by the device can be stored in liquid or solid form. The sample can undergo optional treatment before being stored. Storage can occur on the device, off the device, or in a removable container, vessel, compartment, or cartridge within the device.

The devices illustrated in FIGS. 43A and 43B, and any sample acquisition devices disclosed in the present application can be configured to collect, treat, stabilize, and store a collected sample. A device can be configured to perform one or more of the following processes: collection, treatment, stabilization, and storage of the sample. Collection, treatment, stabilization, and storage can be performed within a single device. Treatment can comprise filtration of the sample to separate components or analytes of interest.

In some instances one or more of the processes (e.g. collection, treatment, stabilization, and storage of the sample) can be performed on the device in response to singe activation of the device by the user. In other instances two or more user actions can need to be performed to move the sample through one or more different processes (e.g. collection, treatment, stabilization, and storage). User actions can comprise pressing a single button, pressing multiple buttons, pressing two or more buttons at the same time, and pressing two or more buttons in a prescribed sequence (e.g. based on a prescribed sequence to perform a set of treatment steps desired by the user.)

The device can be adhered to the skin of a patient with an adhesive. In some embodiments, any suitable adhesive is used. The adhesive can be a hydrogel, an acrylic, a polyurethane gel, a hydrocolloid, or a silicone gel.

The adhesive can be a hydrogel. In some embodiments, the hydrogel comprises a synthetic polymer, a natural polymer, a derivative thereof, or a combination thereof. Examples of synthetic polymers include, but are not limited to poly(acrylic acid), poly(vinyl alcohol)(PVA), poly(vinyl pyrrolidone)(PVP), poly(ethylene glycol)(PEG), and polyacrylamide. Examples of natural polymers include, but are not limited to alginate, cellulose, chitin, chitosan, dextran, hyaluronic acid, pectin, starch, xanthan gum, collagen, silk, keratin, elastin, resilin, gelatin, and agar. The hydrogel can comprise a derivatized polyacrylamide polymer.

In some embodiments, the adhesive comes attached to the device. The device can comprise a protective film or backing covering the adhesive on the base of the device, wherein prior to use the protective film is removed. In another embodiment, an adhesive in the form of a gel, a hydrogel, a paste, or a cream is applied to skin of the subject or the base of the device prior in order to adhere the skin to the device. The adhesive can be in contact with the patient for less than about 10 minutes. In some embodiments, the adhesive is a pressure-sensitive adhesive. In some embodiments, the adhesive is hypoallergenic.

Collection of the sample can comprise steps and components configured for lancing the subject's skin and providing or creating a vacuum to extract the sample. In some instances a vacuum can be provided before lancing of the skin, in other instances the vacuum can be provided after lancing of the subject's skin, in further instances the vacuum can be provided simultaneously with lancing the subject's skin.

Treatment of the device can comprise concentrating the sample, adjusting or metering the flow of the sample, exposing the sample to one or more reagents, and depositing the sample on a solid substrate or matrix. Embodiments the device can comprise a removable cartridge or enclosure for storing a liquid sample or solid matrix for removing the sample once it has been collected. A solid matrix can comprise cellulose based paper (e.g. Whatman™ 903 paper), paper treated with chemicals or reagents for stabilizing the sample or one or more components of the sample (e.g. RNA stabilization matrix or Protein Stabilization Matrix).

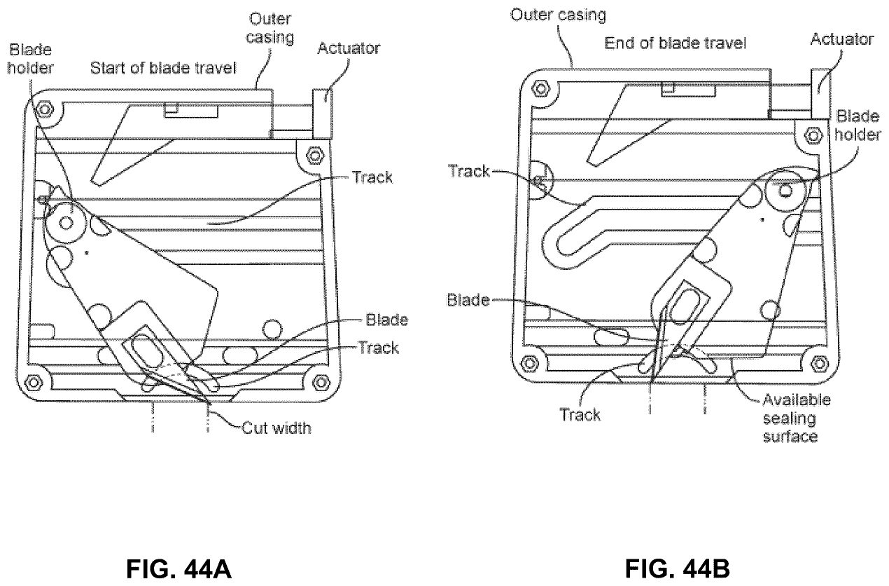

FIGS. 44A and 44B illustrate a device configured for horizontal cutting, with simultaneous seal formation. The device can comprise a square shaped outer casing. A blade holder can be installed on a track, and the blade can be arranged to move on a semi-circular track. When the actuator is depressed, the blade moves along the semi-circular track, cutting an elastomeric material (e.g. polyurethane) and creating a seal between an adhesive (e.g. hydrogel) circle or donut shaped material disposed from the base of the device. In this embodiment the activation of the actuator triggers the blade which cuts the elastomeric material forming a seal, while simultaneously lancing the subject's skin. FIG. 44A shows the blade before it is actuated, and FIG. 44B shows the blade after it has cut the elastomeric material and formed a seal with the subject's skin.

The devices illustrated in FIGS. 44A-44B, and any sample acquisition devices disclosed in the present application, can comprise a single or multiple blades for piercing a subjects skin; for example one or more, two or more, three or more, four or more, five or more, or ten or more blades. Blades can be configured in different shapes or orientations, for example a ring shape, a star shape, a hash shape, square shapes, rectangular shapes, etc.

The devices illustrated in FIGS. 44A-44B, and any sample acquisition devices disclosed in the present application, can be configured to collect, treat, and store the sample. The sample drawn by the device can be stored in liquid or solid form. The sample can undergo optional treatment before being stored. Storage can occur on the device, off the device, or in a removable container, vessel, compartment, or cartridge within the device.

The devices illustrated in FIGS. 44A-44B, and any sample acquisition devices disclosed in the present application can be configured to collect, treat, stabilize, and store a collected sample. A device can be configured to perform one or more of the following processes: collection, treatment, stabilization, and storage of the sample. Collection, treatment, stabilization, and storage can be performed within a single device. Treatment can comprise filtration of the sample to separate components or analytes of interest.

In some instances, one or more of the processes (e.g. collection, treatment, stabilization, and storage of the sample) can be performed on the device in response to singe activation of the device by the user. In other instances two or more user actions can need to be performed to move the sample through one or more different processes (e.g. collection, treatment, stabilization, and storage). User actions can comprise pressing a single button, pressing multiple buttons, pressing two or more buttons at the same time, and pressing two or more buttons in a prescribed sequence (e.g. based on a prescribed sequence to perform a set of treatment steps desired by the user.)

Collection of the sample can comprise steps and components configured for lancing the subject's skin and providing or creating a vacuum to extract the sample. In some instances a vacuum can be provided before lancing of the skin, in other instances the vacuum can be provided after lancing of the subject's skin, in further instances the vacuum can be provided simultaneously with lancing the subject's skin.

Treatment of the device can comprise concentrating the sample, adjusting or metering the flow of the sample, exposing the sample to one or more reagents, and depositing the sample on a solid substrate or matrix. Embodiments the device can comprise a removable cartridge or enclosure for storing a liquid sample or solid matrix for removing the sample once it has been collected. A solid matrix can comprise cellulose based paper (e.g. Whatman™ 903 paper), paper treated with chemicals or reagents for stabilizing the sample or one or more components of the sample (e.g. RNA or DNA).

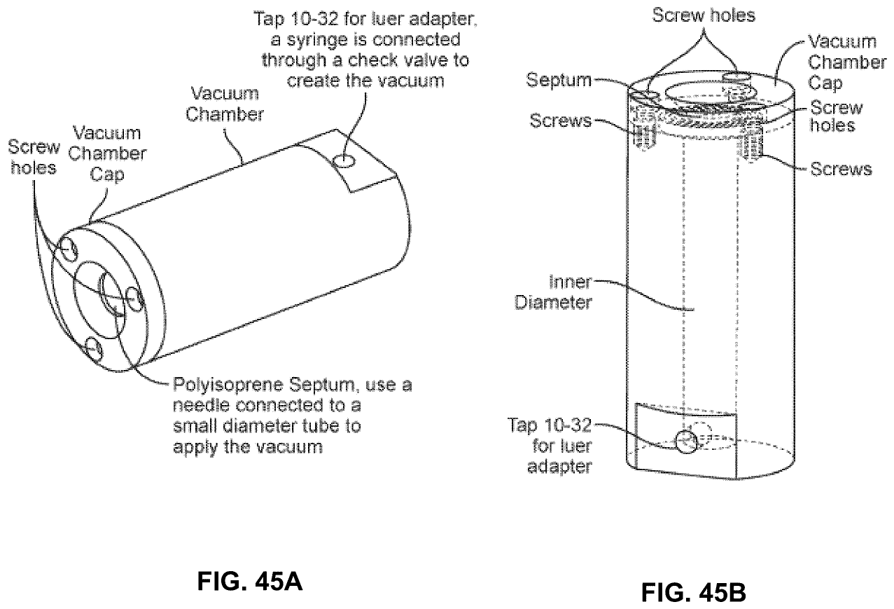

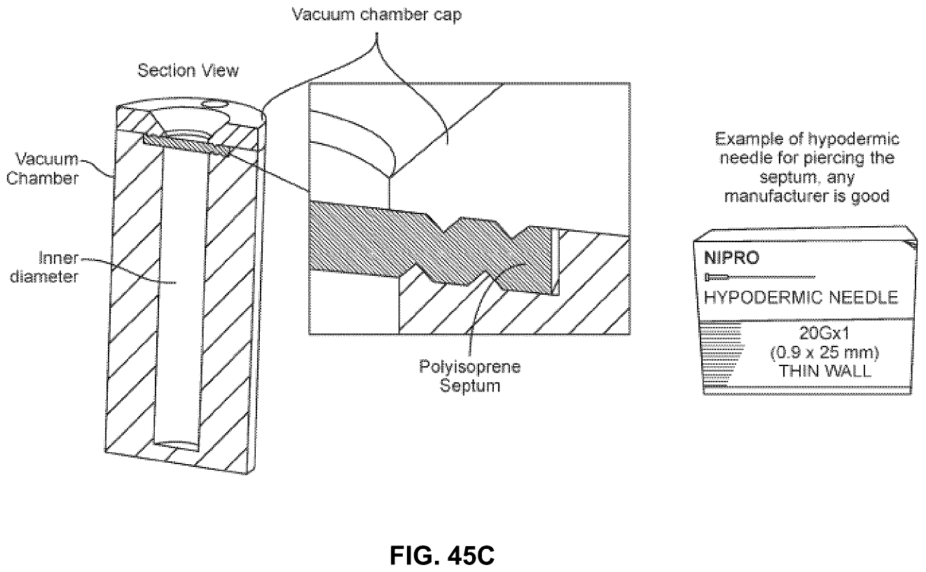

Any of the embodiments disclosed in the present application can comprise a vacuum chamber. Vacuum chambers can vary in size, shape, pressure, and can have structural variations as well as a variety of mechanisms for generating the vacuum. A vacuum chamber can come pre-charged using an onboard evacuated chamber (e.g. a chamber installed on the device using a membrane that when penetrated generates negative pressure in contiguous enclosures), or generated through user action by way of a syringe or other means of generating negative pressure. The vacuum chamber (e.g. evacuated chamber) can seal on one end with foil or elastomer (e.g. polyisoprene) on the other end, such that piercing the foil or septum allows the vacuum to generate within the device. Vacuum chamber sizes can vary, for example the vacuum chamber can be greater than 2 mL, greater than 4 mL, greater than 6 mL, greater than 8 mL, or greater than 10 mL in volume. One embodiment of a vacuum chamber is illustrated in FIGS. 45A-45C. FIGS. 45A and 45B illustrates a side view of a vacuum chamber that could be used in the disclosed devices. The vacuum chamber can comprise a Polyisoprene septum with a needle connected to a small diameter tube to apply the vacuum. The chamber can also comprise, on the opposite side, a luer adaptor so that a syringe can be connected through a check valve to create the vacuum. The vacuum chamber can comprise an opening, a vacuum chamber cap, and one or more screw holes with screws for holding the cap in place. FIG. 45C shows a side view of a vacuum chamber, as well as a zoom-in view of the groves that hold the septum in place and illustration of the type of needles that can be used with the vacuum chamber.

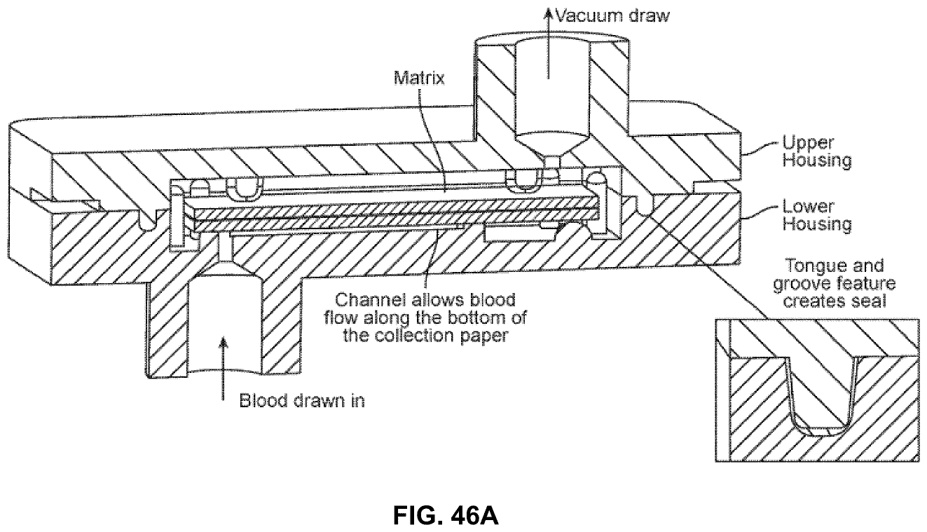

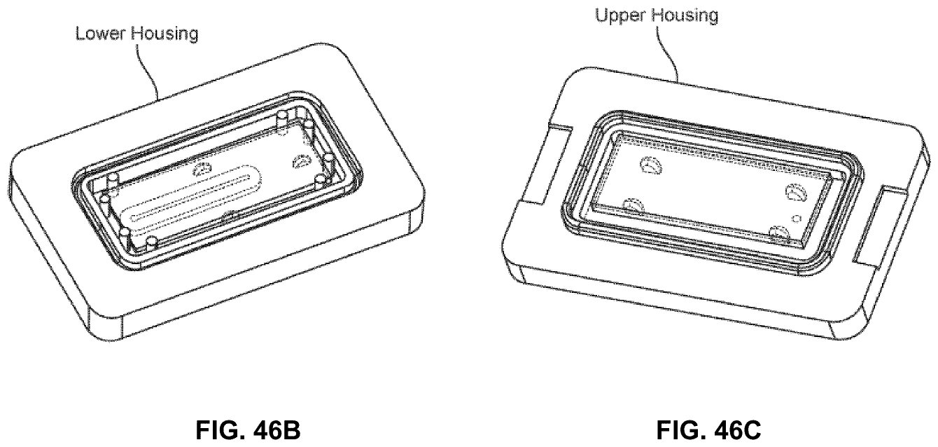

Once a device lances the skin of a subject and the blood sample is drawn into the device, the sample can be optionally treated then stored on a sample collection matrix. The storage and sample treatment methods can comprise treating the sample to fix the volume, uniformity, or concentration of the sample deposited on sample collection matrix. Methods and devices for collecting and storing the sample on the matrix can comprise a cartridge or compartment that can be removed from the device. An exemplary cartridge or compartment for depositing and storing the collected sample is illustrated in FIGS. 46A-46C.

FIG. 46A, FIG. 46B, and FIG. 46C illustrate a sample collection matrix for collecting and storing sample on a stabilization matrix. As shown in FIG. 46A, the sample collection matrix can comprise an inlet where the blood sample is drawn into a channel within the device that allows blood to flow along the bottom of the solid matrix. A vacuum draw is present on the other side of the device to draw the sample into the solid matrix. The sample collection housing can comprise a upper housing and a lower housing (as shown in FIGS. 46B and 46C) with the matric and channel moving sample within the housing, disposed between the upper and lower housing. A tongue and groove feature can create a seal between the upper and lower housing.

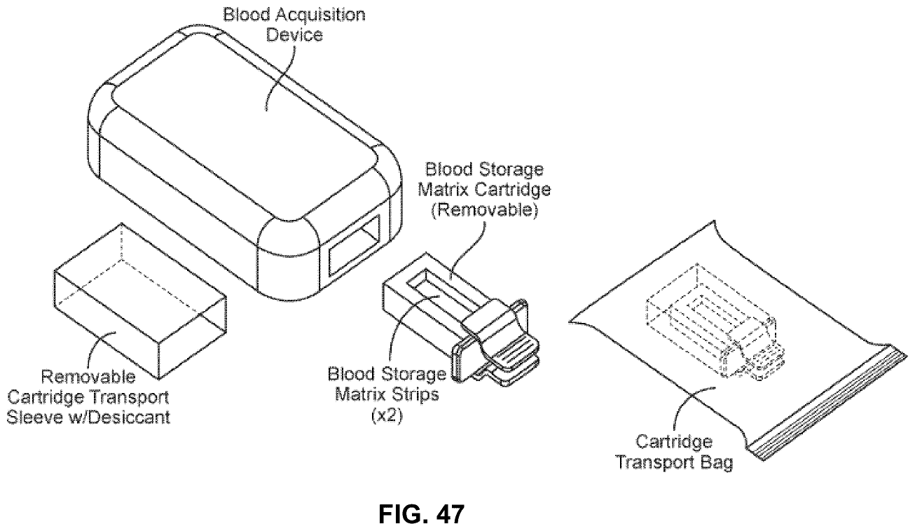

FIG. 47 illustrates components of a device or kit for collecting a sample from a subject. The kit can comprise a sample collection device, a removable cartridge transport sleeve with desiccant (with or without a barcode or label), a removable blood storage matrix cartridge, blood storage matrix strips, and a cartridge transport bag.

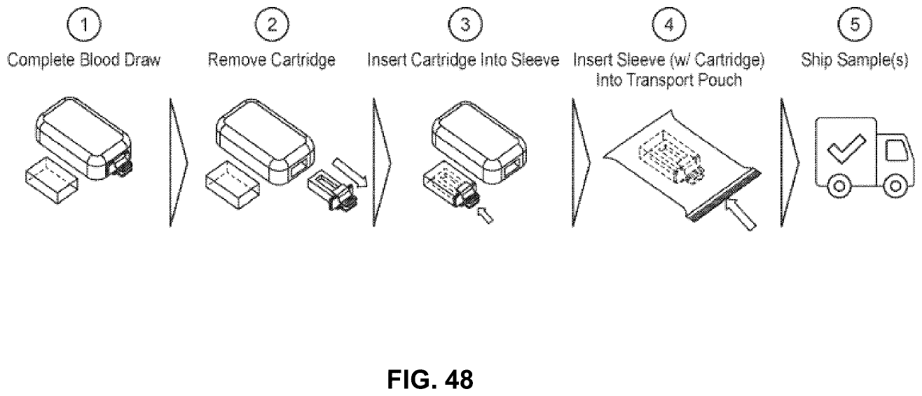

FIG. 48 illustrates methods by which a user can acquire a sample using the kit. The kit can be obtained and the user can insert the cartridge into the device. Steps executed by the user can comprise using the device to collect a sample, remove the cartridge once sample collection is complete, place the removable cartridge into the transportation sleeve with desiccant, and place it in the cartridge transport bag. Multiple samples can be taken by the user, and then the user can ship the sample(s) to a facility for analysis.

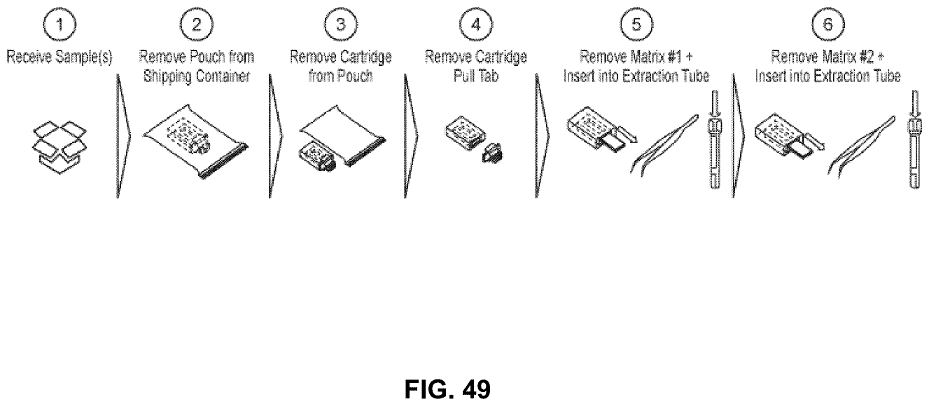

FIG. 49 illustrates exemplary method steps that a laboratory can perform upon receipt of a shipping container containing the sample(s). The sample pouch can be removed from the shipping container, the sample cartridge can be removed from the sample pouch, and the pull tab can then be removed from the cartridge. Matrix #1 can be removed from the cartridge and placed in an extraction tube, then matrix #2 can be removed from the cartridge and placed in an extraction tube. The extraction tube matrix #2 is placed in can be a different extraction tube from the extraction tube matrix #1 is placed in. The extraction tube matrix #2 is placed in can be the same extraction tube from the extraction tube matrix #1 is placed in. The extraction tube can be a microfuge tube. From there any number of tests or analyses can be performed on the sample.

The devices, systems, and methods disclosed herein can stabilize sample on a matrix (e.g. blood storage matrix, sample collection matrix, matrix, sample stabilization matrix, stabilization matrix (e.g. RNA Stabilization Matrix, Protein Stabilization Matrix), solid matrix, solid substrate, solid support matrix, or solid support). The matrix can be integrated into the device, or external to the device. In some embodiments the matrix can be incorporated into a cartridge for removal (e.g. after sample collection). In some embodiments the matrix can matrix comprise a planar dimensional that is at least 176 mm². A matrix can be prepared according to the methods of U.S. Pat. Nos. 9,040,675, 9,040,679, 9,044,738, or U.S. Pat. No. 9,480,966 of which are all herein incorporated by reference in their entirety.

In some embodiments, a system, a method, or a device can comprise a high surface area matrix that selectively stabilizes nucleic acids or proteins. In some instances the matrix can be configured to comprise a planar sheet with total dimensional area (length multiplied by width) greater than 176 mm².

The matrix can be configured to selectively stabilize sample preparation reagents comprising protein and/or nucleic acids. The matrix can be configured to stabilize protein and nucleic acids can comprise an oligosaccharide (e.g. a trisaccharide) under a substantially dry state. The oligosaccharide or trisaccharide can be selected from a group comprising: melezitose, raffinose, maltotriulose, isomaltotriose, nigerotriose, maltotriose, ketose, cyclodextrin, trehalose or combinations thereof. In some embodiments the matrix can comprise melezitose. In further embodiments the melezitose can be under a substantially dry state. In some embodiments, melezitose under a substantially dry state can have less than 2% of water content. In the matrix, the concentration of the melezitose can be in range of about 10% to about 30% weight percent by mass (e.g. calculates as the mass of the solute divided by the mass of the solution where the solution comprises both the solute and the solvent together. The concentration of melezitose can be 15% weight percent by mass. The melezitose can be impregnated in the matrix. In some embodiments, the impregnated melezitose concentration in the matrix results from immersing the matrix in a melezitose solution comprising between about 10 to about 30%. In some other embodiments, 15% melezitose is impregnated into the matrix in a dried state. The matrix can be passively coated or covalently-modified with melezitose. In other embodiments the melezitose can be applied to the surface of the matrix (e.g. with dipping, spraying, brushing etc.). In some other embodiments, the matrix can be coated with a 15% solution of melezitose. In some embodiments the matrix can matrix comprise a planar dimensional with a surface area that is at least 176 mm². In some embodiments the melezitose can be present at greater than 0.01 ng/mm², greater than 0.05 ng/mm², greater than 0.1 ng/mm², greater than 0.5 ng/mm², greater than 1 ng/mm², greater than 5 ng/mm², greater than 0.01 μg/mm², greater than 0.05 μg/mm², greater than 0.1 μg/mm², greater than 1 μg/mm², greater than 5 μg/mm², greater than 0.01 mg/mm², greater than 0.05 mg/mm², greater than 0.1 mg/mm², greater than 1 mg/mm², greater than 5 mg/mm², greater than 10 mg/mm², greater than 50 mg/mm², greater than 1g/mm², greater than 5g/mm², or greater than 10g/mm². The matrix can comprise additional components to stabilize protein and/or nucleic acids, including various stabilization molecules. A non-limiting example of a stabilization molecule is validamycin. In some embodiments the matrix can comprise 31-ETF (e.g. cellulose based matrix) and melezitose.

The matrix can comprise a buffer reagent. A buffer reagent can be impregnated into the matrix. Buffers can stabilize sample preparation reagents and/or various sample components. The matrix can further include at least one buffer disposed on or impregnated within the matrix, wherein the matrix can be substantially dry with a water content of less than 2%. The buffer can be an acid-titrated buffer reagent that generates a pH in a range from about 3 to about 6, or about 2 to about 7. The matrix can contain any one of the following: 2-Amino-2-hydroxymethyl-propane-1,3-diol (Tris), 2-(N-morpholino) ethanesulfonic acid (MES), 3-(N-morpholino) propanesulfonic acid (MOPS), citrate buffers, 4-(2-hydroxyethyl)-1-piperazineethanesulfonic acid (HEPES), phosphate buffers or combinations thereof, or Tris-Hydrochloride (TrisHCl). The matrix can be configured to yield a solution upon rehydration comprising about 20 to about 70 mM Tris-HCl and about 5 to about 30 mM MgCl₂. The amount of various dehydrated buffer reagents impregnated into a matrix can be configured for stabilizing sample preparation reagent(s).

The matrix can comprise a reagent or compound that minimizes nuclease activity, e.g., a nuclease inhibitor. Examples of nuclease inhibitors include RNase inhibitor, compounds able to alter pH such as mineral acids or bases such as HCl, NaOH, HNO₃, KOH, H₂SO₄, or combinations thereof; denaturants including urea, guanidine hydrochloride, guanidinium thiocyanate, a one metal thiocyanate salt that is not guanidinium thiocyanate (GuSCN) beta-mercaptoethanol, dithiothreitol; inorganic salts including lithium bromide, potassium thiocyanate, sodium iodide, or detergents including sodium dodecyl sulfate (SDS).

The matrix can comprise a reagent or compound that minimizes or inhibits protease activity, e.g., a protease inhibitor. A protease inhibitor can be synthetic or naturally-occurring (e.g., a naturally-occurring peptide or protein). Examples of protease inhibitors include aprotinin, bestatin, chymostatin, leupeptin, alpha-2-macroglobulin, pepstatin, phenylmethanesulfonyl fluoride, N-ethylmaleimide, ethylenediaminetetraacetid acid, antithrombin, or combinations thereof. In one example, protease inhibitors enhance the stability of the proteins by inhibiting proteases or peptidases in a sample.

The matrix can comprise one or more free radical scavengers. The matrix can comprise a UV protectant or a free-radical trap. Exemplary UV protectants include hydroquinone monomethyl ether (MEHQ), hydroquinone (HQ), toluhydroquinone (THQ), and ascorbic acid. In certain aspects, the free-radical trap can be MEHQ. The matrix can also comprise oxygen scavengers, e.g. ferrous carbonate and metal halides. Other oxygen scavengers can include ascorbate, sodium hydrogen carbonate and citrus.

The matrix can comprise a cell lysis reagent. Cell lysis reagents can include guanidinium thiocyanate, guanidinium hydrochloride, sodium thiocyanate, potassium thiocyanate, arginine, sodium dodecyl sulfate (SDS), urea or a combination thereof. Cell lysis reagents can include detergents, wherein exemplary detergents can be categorized as ionic detergents, non-ionic detergents, or zwitterionic detergents. The ionic detergents can comprise anionic detergent such as, sodium dodecylsulphate (SDS) or cationic detergent, such as ethyl trimethyl ammonium bromide. Examples of non-ionic detergent for cell lysis include TritonX-100, NP-40, Brij 35, Tween 20, Octyl glucoside, Octyl thioglucoside or digitonin. Some zwitterionic detergents can comprise 3-[(3-Cholamidopropyl)dimethylammonio]-1-propanesulfonate (CHAPS) and 3-[(3-Cholamidopropyl)dimethylammonio]-2-hydroxy-1-propanesulfonate (CHAPSO). The cell lysis reagent can comprise a thiocyanate salt. One or more embodiments of the solid support matrix comprises a thiocyanate salt impregnated in a dry state. Exemplary thiocyanate salts include, but are not limited to, guanidinium thiocyanate, sodium thiocyanate, potassium thiocyanate or combinations thereof. In some other embodiments, the cell lysis reagent is selected from guanidinium thiocyanate, sodium thiocyanate, sodium dodecyl sulfate (SDS) or combinations thereof.

A solid support matrix can comprise a reducing agent. Reducing agents can include dithiothreitol (DTT), 2-mercaptoethanol (2-ME), tris(2-carboxyethyl) phosphine (TCEP) and combinations thereof. Reducing agents can further comprise oxygen scavengers. Oxygen scavengers or reducing agents can comprise ferrous carbonate and metal halides. A solid support matrix can comprise a chelating agent. Chelating agents can include ethylenediaminetetraacetic acid (EDTA), citric acid, ethylene glycol tetraacetic acid (EGTA), or combinations thereof. The solid support matrix can be configured to provide an acidic pH upon hydration and/or preserve nucleic acids in a substantially dry state at ambient temperature. The solid support matrix can be configured to provide a pH between about 2 and about 7 upon hydration. The solid matrix can be configured to provide a pH between about 3 and about 6 upon hydration.

In some embodiments, a sample can be filtered or separated before being deposited on a matrix. Liquid sample can collect or pool into a collection chamber, after the collection chamber or in lieu of a collection chamber the sample can optionally be absorbed through one or more particles, materials, structures or filters with optimized porosity and absorptivity for drawing the sample into the device. Materials for drawing the sample into the devices herein can consist of any absorptive or adsorptive surfaces, or materials with modified surfaces; optional materials including but not limited to paper-based media, gels, beads, membranes, matrices including polymer based matrices, or any combination thereof.

In some embodiments, the device or cartridge can comprise a sample separation unit comprising one or more substrates, membranes, or filters for separating sample components. The sample separation unit can be integrated within the sample stabilization component, or it can be attached to or separate from the sample stabilization component. In some embodiments, sample separation can occur as an intermediate step between sample acquisition and transfer of sample to the matrix. In some instances sample separation and stabilization can occur in one step without the need for user intervention. Sample separation can further occur sequentially or simultaneously with sample stabilization.

In some embodiments, sample acquisition and stabilization can require user action to proceed between one or more phases of the sample collection, optional separation, and stabilization process. A device can require user action to activate sample acquisition, and move sample between separation, stabilization, and storage. Alternatively, user action can be required to initiate sample acquisition as well as one or more additional steps of the sample collection, separation or stabilization process. User action can include any number of actions, including pushing a button, tapping, shaking, rupture of internal parts, turning or rotating components of the device, forcing sample through one or more chambers and any number of other mechanisms. Movement through the phases can occur in tandem with sample collection, or can occur after sample collection. Anytime during or prior to the processing phases the entire sample or components of the sample can be exposed to any number of techniques or treatment strategies for pre-treatment of cells of biological components of the sample; potential treatment includes but is not limited to treatment with reagents, detergents, evaporative techniques, mechanical stress or any combination thereof.

In some embodiments, the devices described herein are configured to draw capillary blood.

In some embodiments, the devices disclosed herein are designed to be used once and then discarded. Resterilization or reuse can compromise the structural integrity of the device or increase the risk of contamination or infection leading to device failure, cross-infection, or patient injury, illness, or death.

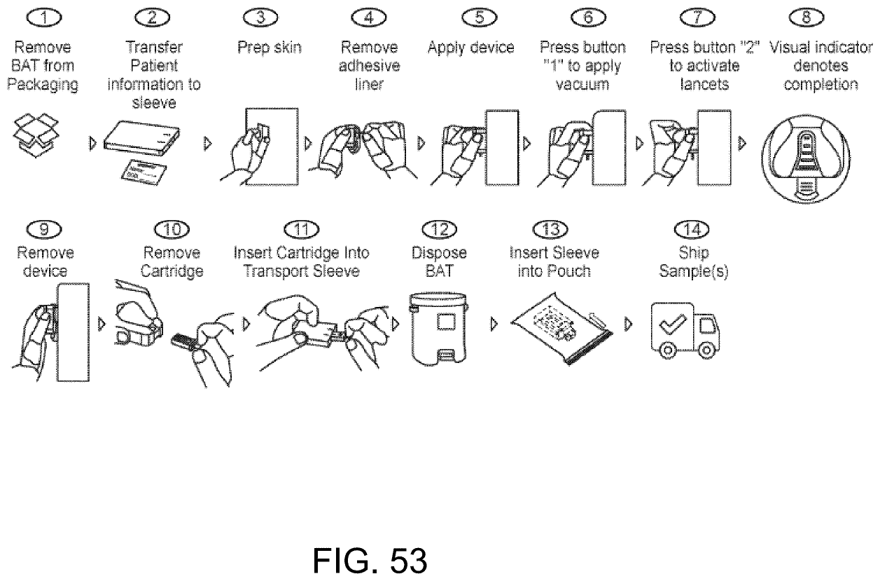

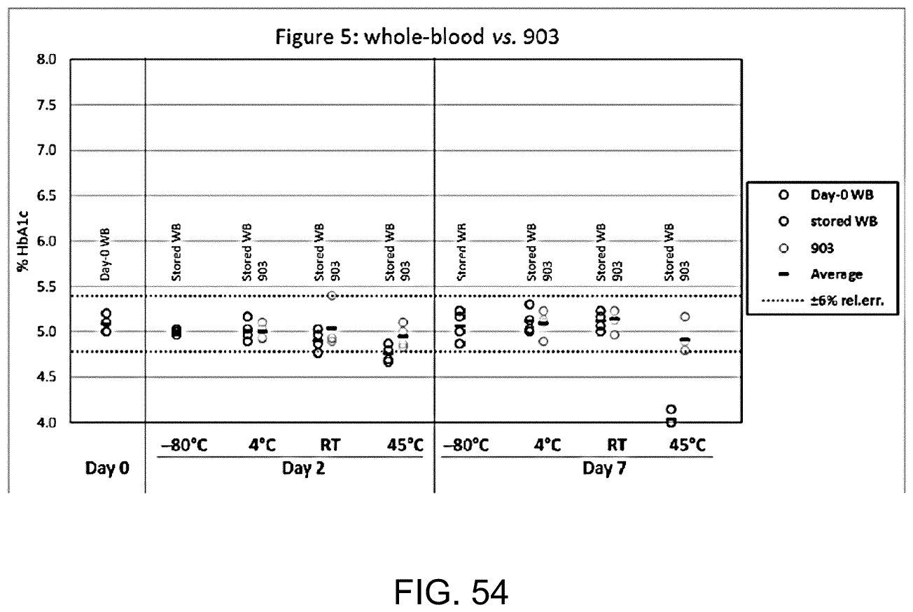

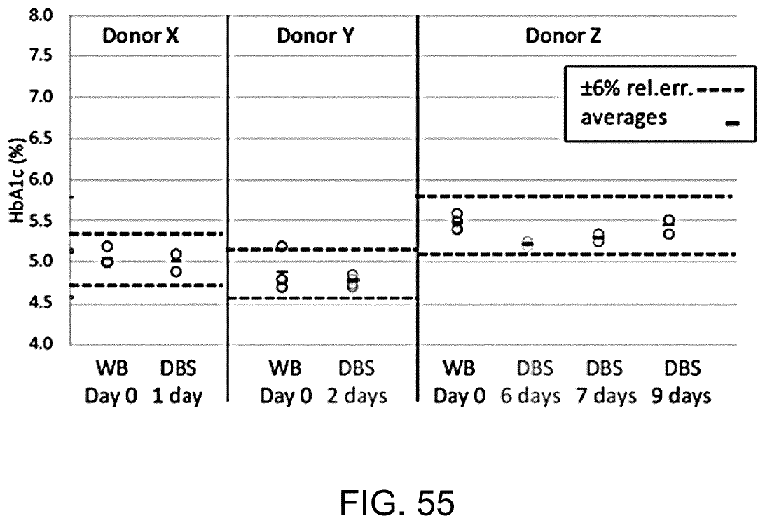

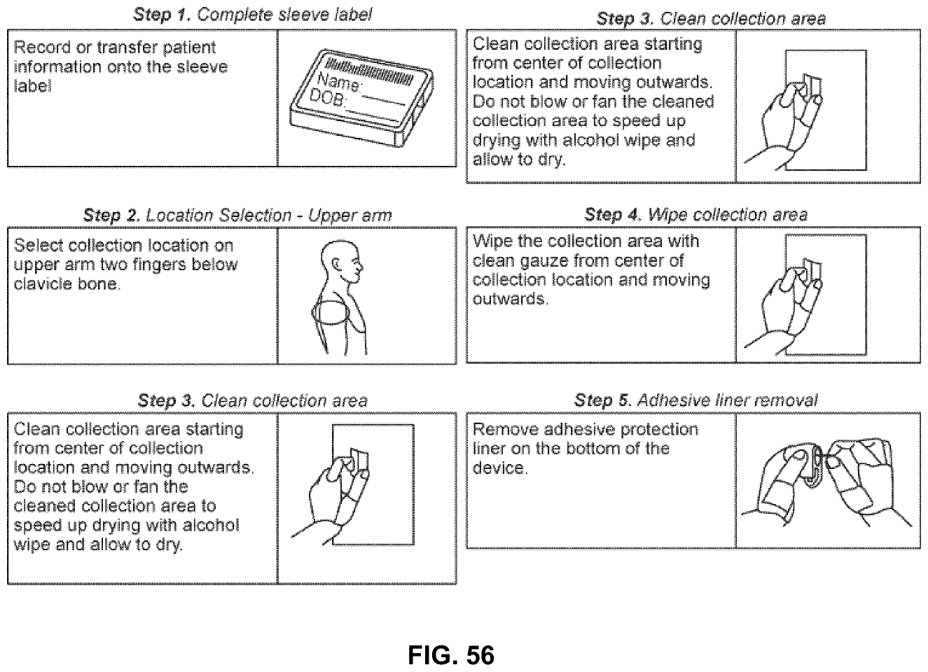

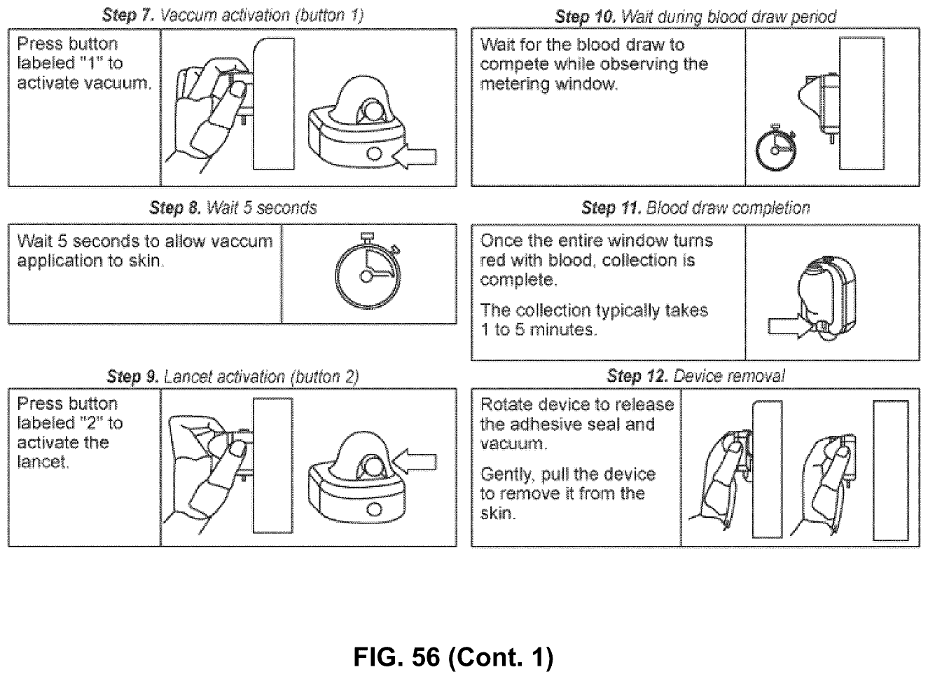

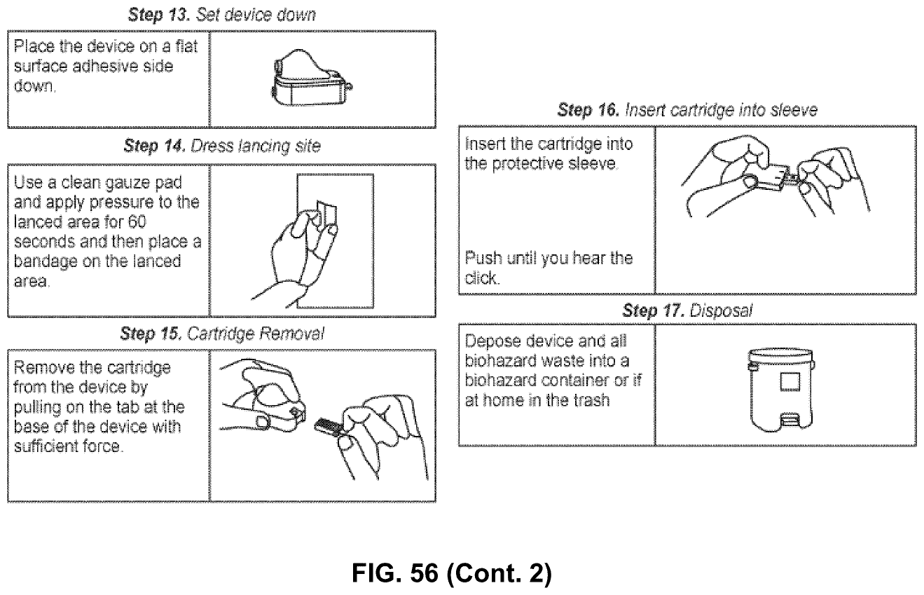

FIG. 53 and FIG. 56 illustrate exemplary procedures to collect and store blood using a device described herein.

Disclosed herein, in certain embodiments, are kits for use with one or methods described herein. A kit can include the device for blood sample collection described herein. The kit can comprise a sample pouch or transportation sleeve, wherein the pouch or sleeve is used to store a cartridge comprising at least one solid matrix strip. A desiccant can be added to the pouch or sleeve. In some embodiments, the desiccant is a silica gel desiccant. The kit can further comprise a sample return envelope, a bandage, an alcohol prep pad, a gauze pad, or a combination thereof.

A kit can include labels listing contents and/or instructions for use, and package inserts with instructions for use. A set of instructions can be included.

In one embodiment, a label is on or associated with the pouch or sleeve. In one embodiment, a label is on a pouch or sleeve when letters, numbers or other characters forming the label are attached, molded or etched into the pouch or sleeve itself; a label can be associated with a pouch or sleeve when it is present within a receptacle or carrier that also holds the pouch or sleeve, e.g., as a package insert. The label can indicate directions for use of the contents, such as in the methods described herein.

The devices, methods, systems and kits disclosed herein can comprise one or more sample separation units. Sample separation units can be used, e.g., to separate plasma from blood, cells from a water sample, or cells from cell free components. The solid matrix can be used to store circulating or cell-free nucleic acids (e.g. DNA or RNA) separated from a sample, e.g., a blood sample, after filtration. The circulating DNA can be tumor circulating DNA. For blood samples one or more components can be used to separate plasma or specific cells from other components of a blood sample. Alternatively, devices, methods and systems can selectively separate any number of sample components including cells, plasma, platelets, specific cell types, DNA, RNA, protein, inorganic materials, drugs, or any other components.

Non-limiting embodiments of the sample stabilization unit can employ sample separation components to separate other non-plasma components as well. Sample separation components can be connected to the sample acquisition component e.g., through channels, including microchannels, wicking of absorbent materials or other means that allow sample to flow through the device. The systems and methods for separating the sample are exemplary and non-limiting.

There are many methods for performing separation, some of which use size, deformability, shape or any combination thereof. Separation can occur through one or more membranes, chambers, filters, polymers, or other materials. Membranes, substrates, filters and other components of the device can be chemically treated to selectively stabilize components, facilitate flow of sample, dry the sample, or any combination thereof. Alternative separation mechanisms can include liquid-liquid extraction, solid-liquid extraction, and selective precipitation of target or non-target elements, charge separation, binding affinity, or any combination thereof. Separation phase can be comprised of one or more steps, with each step relying on different mechanisms to separate the sample. One such mechanism can utilize size, shape or deformation to separate larger components from smaller ones. Cell separation can occur through a sorter that can, for example, rely on one or more filters or other size exclusion methods to separate components of the sample. Separation can also be conducted through selective binding wherein specific components are separated by binding events while the unbound elutant moves into or through alternate chambers.

In some devices, systems, methods, or kits, a single membrane, substrate, or filter can be used for separation and collection of one or more sample components from the bulk sample. Single membrane, substrate, or filter methods can comprise a device wherein samples can be applied to one end of the membrane, substrate, or filter and as the sample flows through a first component of the sample, for example cells, can be separated from a second component of the sample, for example plasma, based on the size of the membrane, substrate, or filter pores. After operation of the device the membrane, substrate, or filter containing the first component of the sample, cells in this example, can be severed from the portion containing the second component of the sample, plasma in this example, necessitating an additional step of severing the membranes, substrates, or filters. In another method, two separate membranes, substrates, or filters can be used for the separation and collection sample components; specifically, a first membrane, substrate, or filter for the separation of one component, for example blood cells, and a second membrane, substrate, or filter for collection of other components, for example plasma. These membranes, substrates, or filters can be arranged such that a distal end of the first membrane, substrate, or filter contacts a proximal end of the second membrane to facilitate the separation of a large component, for example cells, via the first membrane, substrate, or filter and the collection of a second smaller component, for example plasma, via the second membrane, substrate, or filter.

### INTRODUCTION

A first step towards improving the availability of bio-sample testing and diagnostic testing can involve the development of systems and methods that enable quick sample collection, stabilization and preservation of biological components such as DNA, RNA and/or proteins. Once stabilized, such bio components can be transferred to laboratories that can perform one or more biological assays on the bio components. Easier sample collection can be particularly beneficial for diagnosing clinical conditions. There can also be a benefit to having a subject collect, prepare, and optionally analyze their own blood sample without the assistance of a medical practitioner or access to a medical facility or to have an untrained individual collect and stabilize bio samples from a subject. Technological development in this area can make use of at least three components; first, new developments to provide simple tools for collecting biological samples, such as blood; second, new systems, devices and methods to provide simple user-friendly mechanisms for separating, stabilizing and/or storing collected samples or sample components; and, new compatible approaches for analyzing the samples provided through these new methods.

New technologies can be user-friendly and effective enough that acquisition, collection, optional separation and stabilization of the sample can be performed by trained and un-trained end users. Disclosed within are devices, systems and methods that overcome current limitations by addressing some of the aforementioned issues.

Devices, systems and methods disclosed herein, can provide easier sample collection systems, with user friendly sample collection, optional separation, stabilization and storage of samples. Furthermore, devices, systems, and methods can provide approaches and tools for laboratories to more easily receive, prepare and analyze samples. Provided herein are a compatible set of methods, tools, and systems that can enable samples to be easily collected, stored, pre-treated and prepared for analysis so that sample detection can be accomplished with reduced effort on behalf of the user, patient or sample provider. Devices, systems and methods disclosed herein can reduce the burdens of diagnostic testing by simplifying the process of collecting, optionally separating, and stabilizing bio-samples.

Simplifying the process for blood sample collection can involve devices, systems and methods that separate sample components. In some embodiments methods for collecting venous blood can rely on one or more dedicated medical professionals to oversee every step of the blood collection process; from collecting the samples to post-collection procedures which can include separation steps including centrifugation, followed by labeling and cold storage to stabilize samples until they are transferred to a laboratory for testing. In alternate embodiments, the herein disclosed systems and methods can be configured so that an end user can self administer blood sample collection and provide a stabilized sample to a detection facility for analysis.

The herein presented systems, devices and methods can comprise multiple components including (i) a sample acquisition component (SAC) for simple collection of one or more bio-samples (e.g., blood, urine, or environmental samples such as water or soil), (ii) one or more stabilization components for stabilizing bio-analytes from the one or more bio-samples (e.g., DNA, RNA, or protein), and (iii) optionally a separation component for separation of one or more sample components (e.g., plasma, or cells). A further component of the system or method disclosed herein can include one or more kits, devices, methods, and systems for processing samples and analyzing user-provided samples.

Devices, systems, methods, and kits can include a sample acquisition component (SAC) for acquiring the sample, as well as a sample stabilization component (SSC) for transferring, collecting, and stabilizing the sample. In some instances the sample stabilization component can be further equipped to separate one or more components of the sample prior to transferring the sample to a solid substrate for stabilization. SACs can be easy to use, enabling un-trained professionals to collect samples at a variety of locations, from the clinic to a patient's home or office. SACs can even enable a donor to collect their own sample at home, without the need to visit a medical clinic or a dedicated point of collection where sample collection can be done by a trainer or un-trained professional.

Non-limiting embodiments can include integrated and non-integrated combinations of components for sample acquisition, sample separation and sample stabilization. Embodiments can include a single integrated device with distinct internal components for stabilizing components of the sample. Additional integrated devices can include a single unit, or two or more units for separating components of the sample prior to selectively stabilizing sample components. Other embodiments can provide non-integrated components within a system or kit; components can include a sample acquisition component for acquiring the sample, and a separate sample collection component for stabilizing and optionally separating the sample. The sample stabilization component can include a solid stabilization matrix for selectively stabilizing and storing components of the sample, and it can also have a component for separating the sample prior to stabilization. In yet additional non-integrated systems or kits a sample stabilization component can be separate from the sample separation unit. Further embodiments can provide methods, devices, systems and kits for receiving, preparing, and/or treating the stabilized samples after the sample has been acquired, separated, and components of the sample have been selectively stabilized.

The terms “bio-sample” and “biological sample” can be used herein interchangeably throughout the specification. A biological sample can be blood or any excretory liquid. Non-limiting examples of the biological sample can include saliva, blood, serum, cerebrospinal fluid, semen, feces, plasma, urine, a suspension of cells, or a suspension of cells and viruses. In a non-limiting example, the biological samples can include plant or fungal samples. The present systems and methods can be applied to any biological samples from any organism including human. Bio-samples obtained from an organism can be blood, serum, plasma, synovial fluid, urine, tissue or lymph fluids. A biological sample can contain whole cells, lysed cells, plasma, red blood cells, skin cells, non-nucleic acids (e.g. proteins), nucleic acids (e.g. DNA/RNA) including circulating tumor DNA (ctDNA) and cell free DNA (cfDNA). Several embodiments can disclose methods, devices, and systems for collecting blood samples; however, the systems and methods disclosed herein are not intended to be limited to obtaining a bio-sample from an organism. For example, disclosed embodiments can be used on samples obtained from the environment. Non-limiting examples of environmental samples include water, soil and air samples. In some cases, a sample used in the methods, compositions, kits, devices, or systems provided herein is not a biological sample.

For the purposes of describing the devices, methods, systems, and kits disclosed herein, any individual that uses the devices, methods, systems, or kits to collect a sample can be referred to as the “end user”. The individual, organism, or environment from which a sample is derived can be referred to as the “donor”. Once the sample is collected it can be deployed to another facility for testing. At the facility the sample can undergo treatment steps that are selected for based on the devices, systems, methods or kits that were used.

### Sample Acquisition Component (SAC)

Devices, systems, methods and kits described herein can include one or more sample acquisition components (SACs). A sample acquired by a system provided herein can be, e.g., a biological sample from an organism, e.g., blood, serum, urine, saliva, tissue, hair, skin cells, semen, or a sample acquired from the environment, e.g., water sample, oil from well, or from food, e.g., milk. Samples can be liquid, solid or a combination of one or more liquids and one or more solids.

Sample volumes can be fixed by components of a unit, including but not limited to the device collection chamber, materials properties of the collection system, sample settings pre-determined by the user, specifications established during device manufacturing, or any combination thereof.

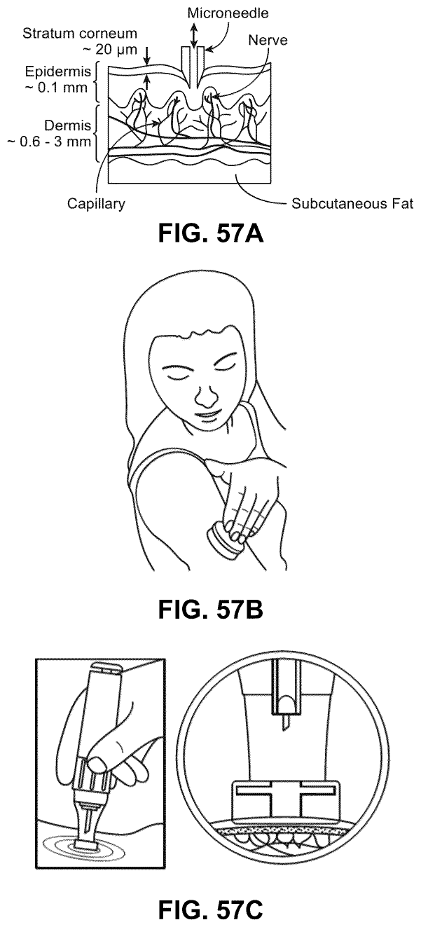

A SAC can include one or more devices for venous blood draw or capillary blood draw. To accomplish venous blood draw or capillary blood draw, the SAC can include one or more piercing elements. The one or more piercing elements can be hollow or solid, and the one or more piercing elements can be configured for pain-free and efficient sample transfer; adaptations can involve use of materials with specific composition, surface microstructure, mechanical properties, structural shapes or combination thereof. The one or more piercing elements can include one or more needles, micro-needles, or lancets (including pressure activated needles or lancets). The SAC can be optionally designed to minimize physical pain or discomfort to the user. AnSAC can include micro-needle technology shown in FIG. 57A, which can allow for shallow penetration of the skin to generate blood flow. Examples for sample, e.g., blood collection, contemplated herein include those described in U.S. Pat. No. 9,033,898. The SAC can be single use. In some cases, a SAC is reusable.

A SAC can collect a volume of, e.g., <1 mL. For example, the SAC can be configured to collect a volume of blood e.g., under 1 mL, 500 μl, 400 μl, 300 μl, 200 μl, 100 μl, 90 μl, 80 μl, 70 μl, 60 μl, 50 μl, 40 μl, 30 μl, 20 μl or 10 μl.

A SACs can be designed to collect a sample volume of from about 1 mL to about 10 mL or from about 1 mL to about 100 mL. Examples of a SAC include a urine cup, a finger stick, or devices, such as those described, e.g., in US. Pub. Nos. 20130211289 and 20150211967.

A SAC can be a component of an integrated device designed for collecting, stabilizing and storing a sample. A SAC can be a separate component that is part of a kit or a system. A SAC can include a vial for collecting the sample.

A kit can include one or more of the following (e.g., when a SAC is non-integrated but is part of a kit): (i) one or more sample separation units, (ii) one or more sample stabilization units, (iii) one or more bio-sample separation and/or stabilization components. A kit used for blood samples can comprise a capillary or transfer tube for collecting a blood drop from a lanced or incised finger and subsequently dispensing the blood onto a device or separate unit for stabilizing or separating and stabilized sample components.

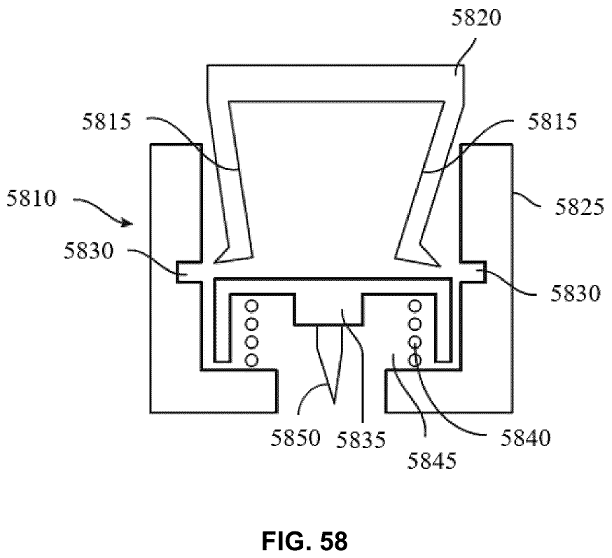

Some embodiments of the sample acquisition component are illustrated in FIG. 57B, FIG. 57C and FIG. 58. As shown in FIG. 57B the SAC can be activated through a mechanical means. Manual activation can be performed by the donor or end user. FIG. 57C illustrates a SAC that can be activated with electrical means. The SAC, as shown, can uses vibration to induce blood flow.

The SAC can use a plunger to create a vacuum that drives the sample into one or more chambers in the device. As shown in FIG. 58, the sample acquisition component (SAC) can have a proximal end, a distal end, an outer surface and a lumen defined with the body 5815. The base attached to the distal end of the body can comprise an outer face, an inner face and comprise one or more apertures 5840. Additionally, the device can have a plunger 5820 comprising a proximal and distal end, with the plunger configured to be user actuated. One or more piercing elements 5850 can be fixed to the face of the plunger, and when the user actuates the SAC from the proximal end to the distal end of the device body, the one or more piercing elements can puncture the skin. A vacuum can be created upon withdrawal of the piercing elements. Blood can pool through the one or more apertures 5840, and the one or more apertures can be configured to draw the sample into the lumen. The sample can move into the lumen and within the device, e.g., through spontaneous capillary-driven flow. Spontaneous capillary-driven flow can also be used to extract fluid from a pool or droplet on a surface, such as human skin, for a reservoir, or from another open microfluidic channel. Spontaneous capillary driven flow can be used to collect the sample from the lance site; it can also be used to manipulate fluids from a reservoir within the device or between sample collection and preparation steps using complex open microfluidic channels. In alternative embodiments, spontaneous capillary-driven flow can be combined with other means of moving sample through the device. The apertures can be optionally adapted for, or optionally contain materials or structures adapted to draw the sample from the penetrated skin into the device. For example in one embodiment, the one or more piercing elements can partially retract into the one or more apertures wherein the properties of the one or more piercing elements itself draw the sample through the aperture and into the sample chamber, either through the one or more piercing elements if the one or more piercing elements is hollow or on the sides of the one or more piercing elements if the one or more piercing elements are solid. Embodiments of these components can be found in US. Pub. No. 20130211289.

Sample collection can occur from sample pooled at or above the skin surface, it can also optionally be collected from one or more reservoirs under the skin. The SAC can, for example, create a lancing motion which cuts into small but plentiful capillaries in the superficial vascular plexus under the epidermis e.g., at depth of 0.3-0.6 mm from the surface of the skin. This disclosure provides a system for mechanically massaging a lance site at other body locations by several different approaches, including oscillating an annular ring surrounding the wound to pump the blood surrounding the wound into the wound for extraction by a needle or capillary tube or oscillating paddles or other members adjacent the wound to achieve the desired blood flow. Further, bringing a drop of blood from the skin in other regions of the body, e.g., the thigh, to a small area on a test device can be difficult. An alternate embodiment of the described herein can work with the needle remaining in the wound and the needle being mechanically manipulated to promote the formation of a sample of body fluid in the wound.

Liquid sample can collect or pool into a collection chamber, after the collection chamber or in lieu of a collection chamber the sample can optionally be absorbed through one or more particles, materials, structures or filters with optimized porosity and absorptivity for drawing the sample into the device. Materials for drawing the sample into the devices herein can consist of any absorptive or adsorptive surfaces, or materials with modified surfaces; optional materials including but not limited to paper-based media, gels, beads, membranes, polymer based matrices or any combination thereof. For example in one embodiment, the SAC can comprise a body that defines a fluid flow path from an inlet opening, wherein the flow path includes a bed of a porous polymer monolith selected to adsorb biological particles or analytes from a matrix drawn or dispensed through the inlet opening and the bed. The porous polymer monolith can absorb biological particles or analytes for later preparation steps. Examples of sample collection on a porous monolith can be found in US Pub. No. US20150211967.

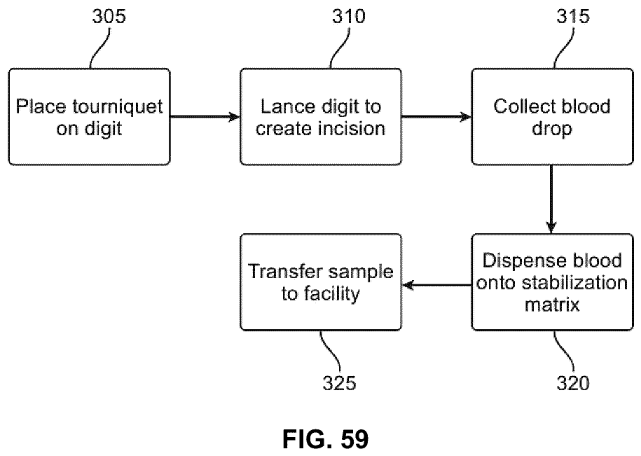

Methods for using the sample acquisition component (SAC) can include piercing the skin with a SAC followed by milking or squeezing of a finger to extract a blood sample. The SAC can be used with a tourniquet or other components for facilitating sample acquisition. A tourniquet, rubber band, or elastic material can be placed around the first, second, or third digit of a subject's hand. Methods can be constructed to improve sample quality. As depicted in FIG. 59, steps can include placing a tourniquet on one of the digits of the donor's finger to apply pressure 305 and lancing the digit to create an incision 310. The method can also include optionally removing the first blood immediately after lancing while still applying pressure. Blood can be collected from the incision 315 by holding a capillary tube against a blood drop formed from the incision site. Blood collected in the capillary tube can be dispersed onto a separate component of the system 320, and the separate part of the component can be deployed to a different location or facility 325 for analysis. In certain embodiments the kit can include components and instructions for facilitating blood collection and efficiency. Methods can further comprise steps for preparing the hand and finger to insure proper blood flow including temperature and position, methods of applying the tourniquet to the finger, methods of sterilization, lancing, and actual blood collection. Methods for collecting blood samples are disclosed, e.g., in U.S. application Ser. No. 14/450,585.

### Sample Stabilization Component

A sample acquired using a sample acquisition component, e.g., a SAC described herein, cancan be transferred to a sample stabilization component (SSC). A SSC can be any device or system that the sample is collected on or transferred to for stabilization and storage. The device or system can comprise channels and compartments for collecting and transferring the samples, and one or more units for separating and stabilizing sample.

In a non-integrated system, the sample acquisition component can be physically separate from the sample stabilization component. In these embodiments, transfer from the sample acquisition component to the sample stabilization component can occur through a variety of means including one or more: needles, capillary tubes, or through direct transfer of the sample from the donor site to the sample stabilization component. In instances where the sample acquisition component is a separate component from the sample stabilization component, application of a sample to a substrate can be achieved using a self-filling capillary for collection from the sample acquisition component, followed by sample transfer to the substrate.

The sample stabilization component can be integrated with the SAC. Integration between the sample stabilization component and the SAC can occur through a shared assembled interface. Sample can move from the sample acquisition component through channels, including microchannels, spontaneous capillary flow, wicking through absorbent materials or other means that allow sample to flow through the sample stabilization component towards or into the substrate with minimal effort on behalf of the end user. Microchannels can be constructed from a variety of materials with properties including adapted shapes with surface microstructures and material properties adapted to facilitate capillary action or other means of sample transfer through the device. Microchannels can comprise any means of transferring sample between chambers, including open microfluidic channels optimized for moving samples using spontaneous capillary flow. Examples of microchannels and devices comprising microchannels can be found in many of the incorporated references, including US Pub. No. 20140038306.

The sample stabilization component can include a structure with multiple layers. It can also have an interface for accepting transfer to one or more layers or to a substrate. The sample stabilization component can be configured for collecting one or more samples, separating components of a sample, stabilizing one or more samples, or any combination thereof. The SSC can further comprise a network of layers and capillary channels configured for transferring sample between multiple layers and into substrate, e.g., as provided for by US Appl. Nos. U.S. Ser. No. 14/340,693 and U.S. Ser. No. 14/341,074.

The SSC can be coupled to the substrate in a variety of ways. The SSC can couple to the substrate in a way that enables contact with a layer for transferring the sample fluid from the integrated device to the substrate. The SSC can be configured such that the device is easily removable from the substrate. The system can further comprise a substrate frame having a region configured to receive the sample on the substrate. The substrate can be attached to the substrate frame in a way that makes it easy to remove the substrate from the system, and the substrate frame can be designed with a barcode to enable automated or semi-automated processing. The system can further be coupled to an external device, wherein the external device comprises a fluidic device, an analytical instrument, or both. In one or more embodiments, the sample stabilization component can be coupled to a substrate, wherein SSC is configured to transfer the sample fluid to the substrate. The substrate can comprise the substrate or the sample separation component. The SSC can either be attached directly to the substrate or to a substrate frame that holds the substrate. In some embodiments, the SSC can further couple to a substrate frame and a substrate cover. The substrate frame and substrate cover can include features to facilitate efficient fluid transfer to the substrate at a region of interest, e.g., at the center of the substrate. In some embodiments, the SSC is packaged with a sample storage substrate, wherein the sample stabilization component is pre-attached to the sample substrate. In some other embodiments, the SSC and substrate are packaged separately, wherein the user can assemble the substrate and the SSC for sample collection, transfer, stabilization and storage. The SSC can be further packaged with a sample acquisition component (SAC).

A SSC can be disposable or re-usable. For example, an SSC can be a single-use disposable device configured to collect the sample and transfer the sample fluid to a substrate and facilitate loading of the fluid sample through desirable areas of the substrate. The SSC can be configured for one time use to reduce or prevent contamination or spreading of infection via the collected sample. The SSC can be configured for reliable and reproducible collection, transfer and storage of biological samples.

After collection and transfer of the biological sample, the substrate can be configured to separate bio-sample components prior to transferring to the stabilization matrix for storage.

### Sample Separation

The SSC can include a sample separation unit comprising one or more substrates, membranes, or filters for separating sample components. The sample separation unit can be integrated within the sample stabilization component, or it can be attached to or separate from the sample stabilization component.

Sample separation can occur at different points in the sample collection process. For example, in an integrated device sample separation can occur within the SSC, for non-integrated devices sample separation can occur outside of the SSC prior to transfer to the sample stabilization component. In other instances the sample can move through the SAC and into the sample separation unit before being transferred to the SSC which can transfer the separated sample to one or more substrates for stabilization and storage.

Sample separation can occur as an intermediate step between sample acquisition and transfer to a sample stabilization matrix. In some instances sample separation and stabilization can occur in one step without the need for user intervention. Sample separation can further occur sequentially or simultaneously with sample stabilization.

The sample acquisition and stabilization can require user action to proceed between one or more phases of the sample collection, optional separation, and stabilization process. An integrated device can require user action to activate sample acquisition, and move sample between separation, stabilization, and storage. Alternatively, user action can be required to initiate sample acquisition as well as one or more additional steps of the sample collection, separation or stabilization process. User action can include any number of actions, including pushing a button, tapping, shaking, rupture of internal parts, turning or rotating components of the device, forcing sample through one or more chambers and any number of other mechanisms. Movement through the phases can occur in tandem with sample collection, or can occur after sample collection. Anytime during or prior to the processing phases the entire sample or components of the sample can be exposed to any number of techniques or treatment strategies for pre-treatment of cells of biological components of the sample; potential treatment includes but is not limited to treatment with reagents, detergents, evaporative techniques, mechanical stress or any combination thereof.

The devices, methods, systems and kits disclosed herein can comprise one or more sample separation units. Sample separation units can be used, e.g., to separate plasma from blood, cells from a water sample, or cells from cell free components. For blood samples one or more components can be used to separate plasma or specific cells from other components of a blood sample. Alternatively, separation devices, methods and systems can selectively separate any number of sample components including cells, plasma, platelets, specific cell types, DNA, RNA, protein, inorganic materials, drugs, or any other components.

Non-limiting embodiments of the sample stabilization unit can employ sample separation components to separate other non-plasma components as well. Sample separation components can be connected to the sample acquisition component e.g., through one or more channels, including one or more microchannels, wicking of absorbent materials or other means that allow sample to flow through the device. The systems and methods for separating the sample are exemplary and non-limiting.

There can be many methods for performing separation, some of which use size, deformability, shape or any combination thereof. Separation can occur through one or more membranes, chambers, filters, polymers, or other materials. Membranes, substrates, filters and other components of the device can be chemically treated to selectively stabilize components, facilitate flow of sample, dry the sample, or any combination thereof. Alternative separation mechanisms can include liquid-liquid extraction, solid-liquid extraction, and selective precipitation of target or non-target elements, charge separation, binding affinity, or any combination thereof. Separation phase can be comprised of one or more steps, with each step relying on different mechanisms to separate the sample. One such mechanism can utilize size, shape or deformation to separate larger components from smaller ones. Cell separation can occur through a sorter that can for example rely on one or more filters or other size exclusion methods to separate components of the sample. Separation can also be conducted through selective binding wherein specific components are separated by binding events while the unbound elutant moves into or through alternate chambers.

In some methods, a single membrane can be used for separation and collection of one or more sample components from the bulk sample. Single membrane methods can use a device wherein samples can be applied to one end of the membrane and as the sample flows through a first component of the sample, for example cells, can be separated from a second component of the sample, for example plasma, based on the size of the membranes pores. After operation of the device the membrane containing the first component of the sample, cells in this example, can be severed from the portion containing the second component of the sample, plasma in this example, necessitating an additional step of severing the membranes. In another method, two separate membranes can be used for the separation and collection sample components; specifically, a first membrane for the separation of one component, for example blood cells, and a second membrane for collection of other components, for example plasma. These membranes can be arranged such that a distal end of the first membrane contacts a proximal end of the second membrane to facilitate the separation of a large component, for example cells, via the first membrane and the collection of a second smaller component, for example plasma, via the second membrane.

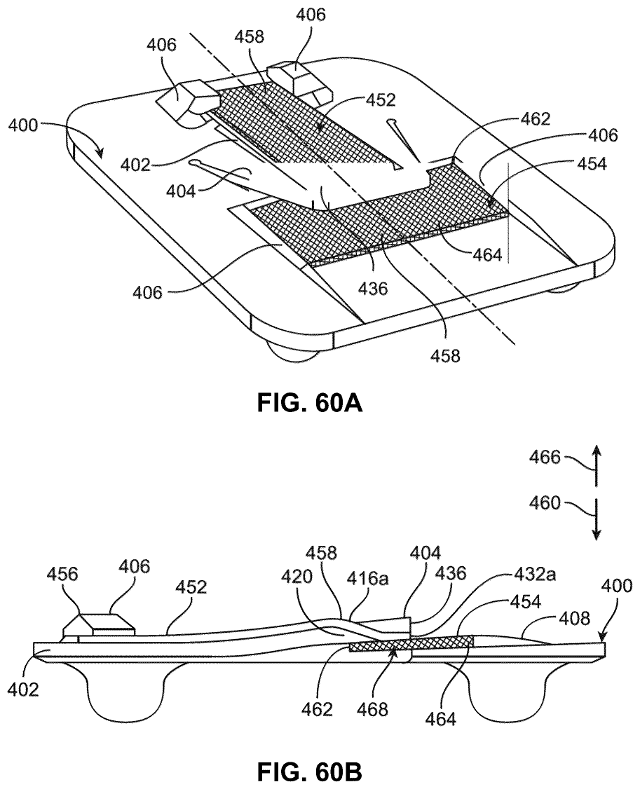

FIGS. 60A and 60B illustrates a sample separation unit that can be used to separate samples prior to stabilization or in tandem with stabilization. Sample separation units can comprise a frame 400, a separation membrane 452, and a collection membrane 454. The frame can include an inner side disposed proximate to a first peripheral portion of the frame. The inner side 402 is formed from a plurality of first slots in the frame 416. The frame further includes an outer side 404 disposed surrounding at least a portion of the plurality of first slots 416. The outer side 404 is formed from a plurality of second slots in the frame 432. A distal end of the separation membrane 420 is disposed under the outer side, and a proximal end of the collection membrane is disposed under at least one of the outer side and inner side such that the proximal end of the collection membrane has an overlapping contact area 428 with the distal end of the separation membrane. Further, the outer side is configured to apply pressure on the separation and collection membranes about the overlapping contact area. The frame 400, separation membrane 452, and collection membrane 454, can be comprised of different materials. The frame 400 can be comprised of a polymer material such as polypropylene, nylon (polyamide), high density polyethylene (HDPE), and polyetheretherketone (PEEK); and it can be manufactured using an injection molding technique and has a uniform thickness. The separation membrane 452 can include suitable materials such as cellulose, a glass fiber, a cellulose acetate, a poly vinyl pyrrolidone, a polysulfone, a polyethersulfone, a polyester or combinations of these materials; and it can be designed to have a geometry compatible with the geometry of the frame 400, specifically, the geometry of the inner side 402 of the frame 400. The collection membrane 454 can include suitable materials such as cellulose, a glass fiber, a cellulose acetate, a poly vinyl pyrrolidone, a polysulfone, a polyethersulfone, polyester, or combinations of these materials; and the collection membrane can be chemically treated. Other embodiments and methods can include the step of displacing, such as by pressing downwards 460, an inner side of the frame 402 to insert a distal end 458 of a separation membrane 452 under the second distal end portion 436 of the outer side of the frame 404 via a first mid-slot 416b of the inner side 402. The method can further include the step of displacing the outer 404 and inner sides 402, for example by applying pressure by pushing upwards 466, to insert a proximal end 462 of a collection membrane 454 under at least one of the outer and the inner sides via a second mid-slot of the outer side 432b, such that the proximal end of the collection membrane has an overlapping contact area 468 with the distal end 458 of the separation membrane 452.

The separation machinery can be optional, for example it can be part of a modular system wherein the user or the manufacturer can insert a cartridge within the path of the sample. In one potential embodiment the sample can be transferred from any of the previously mentioned collection devices into a secondary chamber. The transfer can be facilitated by user action or it can happen spontaneously without user action.

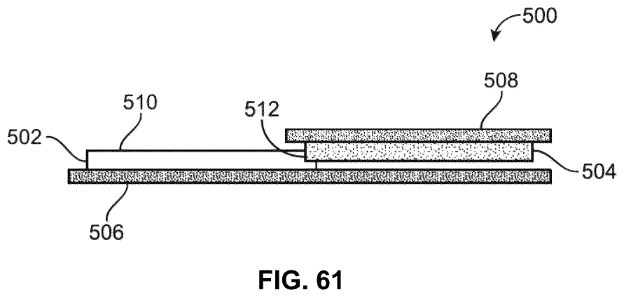

The sample separation unit can use a filtration membrane to separate sample components. FIG. 61 illustrates a sample separation unit with a filtration membrane that separates out the non-cellular fraction of a biological sample. The filtration membrane 502 can have a sample application zone 510 and a transfer zone 512. The filtration membrane can be in direct contact with a solid matrix 504 via the transfer zone 512. A biological sample can be applied to the sample application zone 510 of the filtration membrane, and can be filtered as it moves through the filtration membrane. The filtration membrane can have a plurality of pores. Once the biological sample passes through the filtration membrane, resident intact cells within the biological sample can be retained by the filtration membrane, e.g., mostly at the sample application zone 510 and the non-cellular fraction can be passed through the pores to reach the transfer zone 512 and get transferred and collected onto the dry solid matrix. A filtration membrane can have pore sizes that can range from about 0.01 micron to about 5 micron. The filtration membrane can have a range of pore sizes, for example, between about 1 micron to about 2 micron. The pore size can vary between about 0.22 micron to about 2 microns. When a filtration membrane of 1 micron pore size is used, any other circulating eukaryotic cells and/or pathogenic cells having diameters greater than 1 micron can be retained in the filtration membrane and so will not reach the dry solid matrix upon filtration. Additional separation components are described in US Publ. No. 20150031035.

Filtration can occur at various points in the sample collection process. A non-cellular fraction of a sample can, for example, be filtered out from the biological sample at the point-of-collection itself. Filtration can be performed without any prior pre-treatment of the biological sample. Further filtration can be performed in absence of any stabilizing reagent.

Filtration membrane can be made from a variety of materials. The materials used to form the filtration membrane can be a natural material, a synthetic material, or a naturally occurring material that is synthetically modified. Suitable materials that can be used to make the filtration membrane include, but are not limited to, glass fiber, polyvinlyl alcohol-bound glass fiber, polyethersulfone, polypropylene, polyvinylidene fluoride, polycarbonate, cellulose acetate, nitrocellulose, hydrophilic expanded poly(tetrafluoroethylene), anodic aluminum oxide, track-etched polycarbonate, electrospun nanofibers or polyvinylpyrrolidone. In one example, the filtration membrane is formed from polyvinlyl alcohol-bound glass fiber filter (MF1™ membrane, GE Healthcare). In another example, filtration membrane is formed from asymmetric polyethersulfone (Vivid™, Pall Corporation). In some embodiments, filtration membrane can be formed by a combination of two or more different polymers. For example, filtration membrane can be formed by a combination of polyethersulfone and polyvinylpyrrolidone (Primecare™, iPOC).

After filtration, the separated, non-cellular fraction can be collected onto a dry solid matrix by means of physical interaction. The non-cellular fraction can be collected on to dry solid matrix by means of adsorption or absorption.

### Stabilization Matrix

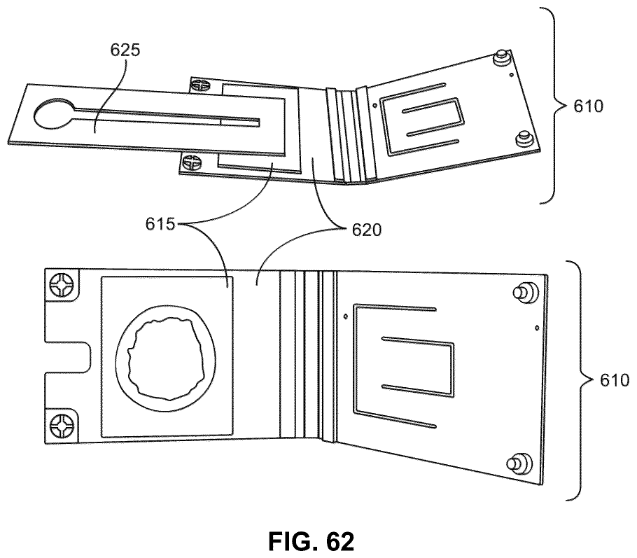

The SSC can be used to transfer, stabilize, and store target components of a bio-sample which can comprise target components such as nucleic acids, proteins, and respective fragments thereof. The SSC can receive, extract and stabilize one or more of these analytes onto a substrate that can be coupled to or housed within the sample stabilization component. FIG. 62 illustrates an example of a sample stabilization component 610 with a sample substrate or matrix 615 for preserving or stabilizing the sample. The substrate 615 can be held in place on one side of the device using a frame 620. The sample can be collected and transferred through a channel 625 to the mounted sample substrate.

The term “target components” as used herein, can refer to one or more molecules, e.g., biological molecules that can be detected or tested for, in a given diagnostic test. The target components for a particular test can need to be adequately preserved and stabilized for good quality diagnostic results.

The nature of the sample can for example depend upon the source of the material, e.g., biological material. For example, the source can be from a range of biological organisms including, but not limited to, virus, bacterium, plant and animal. The source can be a mammalian or a human subject. For mammalian and human sources, the sample can be selected from the group consisting of tissue, cell, blood, plasma, saliva and urine. In another aspect, the bio-sample is selected from the group consisting of biomolecules, synthetically-derived biomolecules, cellular components and biopharmaceutical drug.

The composition, stability and quality of target components can vary depending on their source, therefore to a variety of different substrates can be used to stabilize different components of the sample and prepare the sample for testing. The chemical nature and properties of the target components can vary depending on the origin of the sample, and the degree of required sample stabilization can depend on the diagnostic panel the sample is intended for. Some panels, for example, can require high concentration and low quality sample while others require low concentration and high quality sample. High quality reproducible test results can require high quality samples. The composition of the substrate as well as the substrate and methods for stabilizing sample can differ between assays and between target components or analytes.

Since the nature of target components can differ, the substrate or matrix composition can also be selected for or configured to specific target components. Sample sources and/or the diagnostic tests intended for a particular sample can also be variables that determine the composition of the substrate or matrix. For example, high quality diagnostic test results can require high quality or effective stabilization of target components. In some instances the composition of the substrate or stabilization matrix can be designed to specifically stabilize a particular target component (e.g. DNA, RNA or protein). The matrix can be further configured for use in a particular diagnostic test; for example, if the test requires higher concentration of a particular type of target component then the matrix can be designed to selectively release that target component.

Sample processing can be performed with the different types of tests and substrate compositions in mind. For example, a substrate or matrix designed for a specific target component or diagnostic test can have a color or code that indicates the type of assay it can be used for or with. The substrate can also incorporate one or more tags or labels, including barcodes, RFIDs, or other identifiers that allow the samples or results derived from the substrate to be connected with a particular end user or donor.

The term “substrate” or “stabilization matrix” can refer to any solid matrix including a substrate, or the sample separation component herein described. Substrate can be any solid material including one or more absorbent materials which can absorb a fluidic sample, such as blood.

The substrate or stabilization matrix can comprise one or more different solid components. The solid components can be kept in a substantially dry state of less than 10 wt % hydration. Examples of solid matrix substrate include but are not limited to, a natural material, a synthetic material, or a naturally occurring material that is synthetically modified. The substrate can comprises cellulose, nitrocellulose, modified porous nitrocellulose or cellulose based substrates, polyethyleneglycol-modified nitrocellulose, a cellulose acetate membrane, a nitrocellulose mixed ester membrane, a glass fiber, a polyethersulfone membrane, a nylon membrane, a polyolefin membrane, a polyester membrane, a polycarbonate membrane, a polypropylene membrane, a polyvinylidene difluoride membrane, a polyethylene membrane, a polystyrene membrane, a polyurethane membrane, a polyphenylene oxide membrane, a poly(tetrafluoroethylene-co-hexafluoropropylene) membrane, glass fiber membranes, quartz fiber membranes or combinations thereof. Suitable materials that can act as dry solid matrix include, but are not limited to, cellulose, cellulose acetate, nitrocellulose, carboxymethylcellulose, quartz fiber, hydrophilic polymers, polytetrafluroethylene, fiberglass and porous ceramics. Hydrophilic polymers can be polyester, polyamide or carbohydrate polymers.

The substrate or matrix can comprise one or more dried reagents impregnated therein. The one or more dried reagents can comprise protein stabilizing reagents, nucleic acid stabilizing reagents, cell-lysis reagents or combinations thereof. In one embodiment, the substrate is disposed on a substrate frame. Non-limiting examples of the sample substrate can include a porous sample substrate, Whatman FTA™ card, cellulose card, or combinations thereof. In some embodiments, the substrate can include at least one stabilizing reagent that preserves at least one biological sample analyte for transport or storage. Non-limiting examples of suitable reagents for storage media can include one or more of a weak base, a chelating agent and optionally, uric acid or a urate salt or the addition of a chaotropic salt, alone or in combination with a surfactant.

In some instances, the substrate can comprise a dry solid matrix comprised of cellulose. The cellulose-based dry solid matrix is devoid of any detergent. Cellulose-based dry solid matrix cannot be impregnated with any reagent. Cellulose-based dry solid matrix can be impregnated with a chaotropic salt. Examples of chaotropic salt include, but are not limited to, guanidine thiocyanate, guanidine chloride, guanidine hydrochloride, guanidine isothiocyanate, sodium thiocyanate, and sodium iodide. In some embodiments, the cellulose-based dry solid matrix is FTA™ Elute (GE Healthcare). Examples of sample stabilization components can be found, e.g., in US Pub. Nos. US20130289265 and US20130289257.

Substrate can be comprised of one or more layers of material. The substrate can be arranged into a solid matrix. Layers can be arranged to selectively extract specific bio-sample components. Solid matrix can be a single material, or it can be comprised of multiple materials.

Multiple sample stabilization units can be stored in or attached to a single sample stabilization component. A single sample stabilization unit can be stored in the sample stabilization component. Alternatively, two or more sample stabilization units can be linked together such that one or more components of the bio-sample can move through the different units. The sample stabilization matrix or substrate in any of the mentioned embodiments can have uniform or variable composition. The substrate, sample stabilization unit, or components of either the substrate or the sample stabilization unit can be tagged to indicate the type of target component(s) it is intended for, and/or the diagnostic test(s) for which a sample is intended.

The substrate can be constructed such that sample processing can occur within or adjacent to the sample stabilization component. In some embodiments a sample can move through a sample separation step before being exposed to one or more sample stabilization matrices. In some embodiments the acquisition, optional separation and stabilization steps occur in tandem.

For target components that are present in low concentrations, the substrate can be configured to enhance recovery. In these instances the solid substrate can comprise at least one surface coated with a chemical mixture that enhances the recovery of a biological material from the surface. The chemical mixture can comprise components selected from the group consisting of vinyl polymer and non-ionic detergent, vinyl polymer and protein, non-ionic synthetic polymer and non-ionic detergent, non-ionic synthetic polymer and protein, polyethylenemine (PEI) and non-ionic detergent, non-ionic detergent and protein, and polyethylenemine (PEI) and protein. The solid support can be selected from the group consisting of paper, glass microfiber and membrane. The support can be a paper, for example a cellulose paper including a 903 Neonatal STD card. The solid support can comprise a membrane selected from the group consisting of polyester, polyether sulfone (PES), polyamide (Nylon), polypropylene, polytet-rafluoroethylene (PTFE), polycarbonate, cellulose nitrate, cellulose acetate and aluminium oxide. The vinyl polymer can be polyvinyl pyrrolidone (PVP). The non-ionic detergent can be Tween 20. The protein can be selected from the group consisting of albumin and casein. The non-ionic synthetic polymer can be poly-2-ethyl-2-oxazoline (PEOX). The chemical mixture can comprise any combination of polyvinyl pyrrolidone (PVP), Tween 20, albumin, poly-2-ethyl-2-oxazoline (PEOX), polyethylen-emine (PEI), polyethylenemine (PEI) or casein. Also provided herein are methods for recovering a material, e.g., biological material from a solid support comprising the steps of i) contacting a surface of a solid support, e.g., as described herein with a sample containing a biological material; ii) drying the sample on the surface of the solid support; iii) storing the solid support; and iv) extracting the material, e.g., biological material from the surface. In other aspects, step iii) comprises storing the paper support at a temperature in the range of about 15 to about 40° C. The paper support can be stored at a lower temperature depending on the thermal stability of the biological material. Methods of making the substrate can comprise coating at least one surface of the support with a solution of a chemical mixture that enhances the recovery of a biological material from the surface, wherein the chemical mixture is a mixture selected from the group consisting of polyvinyl pyrrolidone (PVP) and Tween 20, polyvinyl pyrrolidone (PVP) and albumin, Tween 20 and albumin, poly-2-ethyl-2-oxazoline (PEOX) and Tween 20, poly-2-ethyl-2-oxazoline PEOX and albumin, polyethylenemine (PEI) and Tween 20, and polyethylen-emine (PEI) and albumin. The sample stabilization component can provide a use of a solid support as described herein for enhancing the recovery of a material, e.g., biological material from a surface thereof, wherein the disclosed stabilization components and methods are disclosed, e.g., in US Publ. Nos. US20130323723 and US2013330750, the entireties of which are herein incorporated by reference.

The substrate or solid matrix can be configured to selectively preserve nucleic acids including RNA, DNA and fragments thereof. A solid matrix for selectively stabilizing nucleic acids can be comprised of at least one protein denaturant, and at least one acid or acid-titrated buffer reagent impregnated and stored in a dry state. The matrix can be configured to provide an acidic pH upon hydration, extract nucleic acids from a sample and/or preserve the nucleic acids in a substantially dry state at ambient temperature.

A stabilization matrix for extracting and stabilizing nucleic acids can comprise any combination of reagents including a protein denaturant, a reducing agent, buffer, a free-radical trap, a chaotropic agent, a detergent, or an RNase inhibitor in the solid matrix in a dried format. The RNase inhibitor can comprise a triphosphate salt, pyrophosphate salt an acid, or an acid-titrated buffer reagent. The stabilization matrix can further be impregnated with or in the presence of one or more reagents including enzyme inhibitors, free-radical scavengers, or chelating agents. The solid matrix can comprise a protein denaturant, a reducing agent, a buffer, and optionally a free-radical trap or RNase inhibitor.

One or more reagents can be impregnated and stored in a dry state. One or more dried reagents can be optionally rehydrated by the addition of buffer, water or sample. The matrix can further comprise a weak or strong protein denaturant. In certain aspects the solid matrix is a porous cellulose-based paper such as the commercially available 903, 31-ETF, or FTA Elute™. Performance of this method permits the storage of nucleic acids, e.g., RNA which can be an unstable biomolecule to store, in a dry format (e.g., on a solid matrix) under ambient temperatures. The solid matrix can be configured such that hydration of the matrix provides an acidic pH. As noted, in one or more embodiments, RNA quality can be determined by capillary electrophoresis of the extracted RNA through a bioanalyzer. The dry solid matrix can permit prolonged storage of one or more bio-components comprising nucleic acids (e.g., RNA, DNA) in a dry format under ambient conditions. In other aspects, a dry solid matrix for ambient extraction and storage of nucleic acids (e.g., RNA, DNA) from a sample comprises a thiocyanate salt, a reducing agent, a buffer, and optionally a free-radical trap or RNase inhibitor present in a solid matrix in a dried format. A dry solid matrix for extraction and storage of nucleic acids (e.g., RNA, DNA) from a sample comprises at least one metal thiocyanate salt, wherein at least one metal thiocyanate salt is not guanidinium thiocyanate (GuSCN), a reducing agent, a buffer, and optionally a free-radical trap or RNase inhibitor. The solid matrices can comprise nucleic acids (e.g., RNA, DNA) in a dry format can be subjected to a process to release the nucleic acids from the solid matrix in an intact format that is suitable for further analyses of the collected nucleic acid samples.

RNA quality can be quantified as an RIN number, wherein the RIN can be calculated by an algorithmic assessment of the amounts of various RNAs present within the extracted RNA. High-quality cellular RNAcan exhibit a RIN value approaching 10. In one or more embodiments, the RNA extracted from the dry matrix has a RIN value of at least 4. In some embodiments, the matrix provides for ambient extraction and stabilization of a bio-sample and produces intact, high quality RNA with a RIN value in a range from about 4 to about 10, or in some embodiments, the RIN value is in a range from about 5 to about 8. The matrix can be a porous non-dissolvable dry material configured to provide a pH between about 2 and about 7 upon hydration for extracting RNA. The matrix can stabilize the extracted RNA with an RNA Integrity Number (RIN) of at least 4.

Methods for extracting and storing nucleic acids from a sample can comprise steps of providing the sample to a dry solid matrix comprising a protein denaturant and an acid or acid titrated buffer reagent; generating an acidic pH upon hydration for extraction of nucleic acids from the sample; drying the matrix comprising the extracted nucleic acids; and storing the extracted nucleic acids on the matrix in a substantially dry state at ambient temperature. Examples of the aforementioned sample stabilization components can be found, e.g., in US Pub. No. US20130338351.

A sample stabilization unit can include a solid matrix for collection and stabilization of non-nucleic acid components, for example proteins. In these instances, the substrate can be configured to extract and stabilize proteins or peptides from a biological sample for preservation at ambient temperature. In one such embodiment, the solid substrate for collection, stabilization and elution of biomolecules can comprise a trisaccharide under a substantially dry state. The trisaccharide can be selected from melezitose, raffinose, maltotriulose, isomaltotriose, nigerotriose, maltotriose, ketose or combinations thereof. One or more embodiments of a solid substrate can further comprise a melezitose under a substantially dry state. Melezitose can be a non-reducing trisaccharide sugar, having a molecular weight of 504.44 g/mol. In one or more embodiments, the solid substrate can comprise melezitose, wherein a concentration of the melezitose is an amount less than 30%. The melezitose can be impregnated in the substrate; the substrate can also be passively coated or covalently-modified with melezitose. In one or more examples, the substrate is further impregnated with one or more reagents, such as one or more lysis reagents, one or more buffer reagents or one or more reducing agents. In some embodiments, the one or more impregnated reagents comprise cell lytic reagents, biomolecule stabilizing reagents such as protein-stabilizing reagents, protein storage chemicals and combinations thereof impregnated therein under a substantially dry state. Examples of the systems described above and other embodiments are disclosed, e.g., in US20140234942, the entirety of which is incorporated by reference.

Other substrates for stabilizing proteins can comprise a solid paper based matrix comprising cellulose fibers and/or glass fibers and a hydrophilic or water soluble branched carbohydrate polymer. Surface weight of the solid based matrix can be 40-800 g/m². Hydrophilic or water soluble branched carbohydrate polymer can be 4-30 wt % of the matrix. The hydrophilic or water soluble branched carbohydrate polymer can have an average molecular weight of 15-800 kDa, such as 20-500 kDa. The branched carbohydrate polymer can include a dextran. Dextrans can be branched (16)-linked glucans. The branched carbohydrate polymer can comprise a copolymer of a mono- or disaccharide with a bifunctional epoxide reagent. Such polymers can be highly branched due to the multitude of reactive hydroxyl groups on each mono/disaccharide. Depending on the reaction conditions used, the degree of branching can be from about 0.2 up to almost 1. The content of water extractables in said paper can be 0-25 wt %, e.g., 0.1-5 wt % or 3-20 wt %. Very low amounts of extractables can be achieved when the carbohydrate polymer is covalently coupled to the paper fibers and/or crosslinked to itself. In some embodiments, the paper comprises 5-300 micromole/g, e.g., 5-50, 5-100 or 50-300 micromole/g negatively or positively charged groups. Negatively charged groups can be e.g. carboxylate groups, sulfonate groups or sulfate groups, while positively charged groups can be e.g. amine or quaternary ammonium groups. The presence of these groups can improve the protective effect of the branched carbohydrate polymer.

Methods for removing sample can comprise a step of storing the dried paper with the sample, e.g., biological sample for at least one week, such as at least one month or at least one year. In some embodiments the method comprises a step of extracting at least one protein from said paper after storage and analyzing said protein. The extraction can be made e.g. by punching out a small part of the paper with dried sample and immersing these in an aqueous liquid. In some embodiments the protein is analyzed in step by an immunoassay, by mass spectrometry or an enzyme activity assay. An example of the substrate disclosed above is disclosed, e.g., in US Appn. No. US20140302521.

The sample stabilization component can comprise at least one dried sample, e.g., at least one dried biological sample, such as a dried blood sample. Blood and other biological materials, e.g. serum, plasma, urine, cerebrospinal fluid, bone marrow, biopsies etc. can be applied to the sample stabilization component and dried for storage and subsequent analysis or other use. The dried sample, e.g., biological sample can be a pharmaceutical formulation or a diagnostic reagent, comprising at least one protein or other sensitive biomolecule. In another aspect the sample stabilization component can comprise a paper card, with one or more sample application areas printed or otherwise indicated on the card. There can be indicator dyes in these areas to show if a non-colored sample has been applied or not. The device can also include a card holder, to e.g. facilitate automatized handling in racks etc. and it can include various forms of sampling features to facilitate the collection of the sample.

Other examples of stabilization matrix or stabilization components that can be used in the devices herein include, but are not limited to Gentegra-RNA, Gentegra-DNA (Gentegra, Pleasanton CA), as further illustrated in U.S. Pat. No. 8,951,719; DNA Stable Plus, as further illustrated in U.S. Pat. No. 8,519,125; RNAgard Blood System (Biomatria, San Diego, CA).

In some embodiments, the solid matrix can selectively stabilize blood plasma components. Plasma components can include cell-free DNA, cell-free RNA, protein, hormones, and other metabolites, which can be selectively stabilized on the solid matrix. Plasma components can be isolated from whole blood and stabilized on a solid matrix. A solid matrix can be overlapping with or a component of a variety of different devices and techniques. Plasma components can be separated from whole blood samples using a variety of different devices and techniques. Techniques can include lateral flow assays, vertical flow assays, and centrifugation.

A solid matrix can be integrated with or a component of a variety of plasma separation devices or techniques. A solid matrix can be overlapping with or a component of a variety of different devices, such as a plasma separation membrane for example Vivid™ plasma separation membrane. A solid matrix can partially overlap with plasma separation device such as a plasma separation membrane. Examples of devices and techniques for plasma separation are disclosed in patents or patent publications, herein incorporated by reference, including U.S. Pat. Nos. 6,045,899; 5,906,742; 6,565,782; 7,125,493; 6,939,468; EP 0,946,354; EP 0,846,024; U.S. Pat. Nos. 6,440,306; 6,110,369; 5,979,670; 5,846,422; 6,277,281; EP 1,118,377; EP 0,696,935; EP 1,089,077, US20130210078, US20150031035.

In various devices and techniques, a separation membrane can be used. The separation membrane can be comprised of polycarbonate, glass fiber, or others recognized by one having skill in the art. Membranes can comprise a solid matrix. Membranes can have variable pore sizes. Separation membranes can have pore diameters of about 1 μm, about 2 μm, about 4 μm, about 6 μm, about 8 μm, about 10 μm, about 12 μm, about 14 μm, about 16 μm, about 18 μm, about 20 μm. A separation membrane can have pores with diameters of about 2 μm to about 4 μm. A separation membrane can have pores that are about 2 μm in diameter.

Plasma separation can be implemented for a wide variety of sample volumes. Plasma sample volumes can be variable depending on the application for which a solid matrix is used. Sample volumes can be greater than about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, or about 1000 μL. Sample volumes can range from about 250 μL to about 500 μL.

### Preparation of Bio-Components

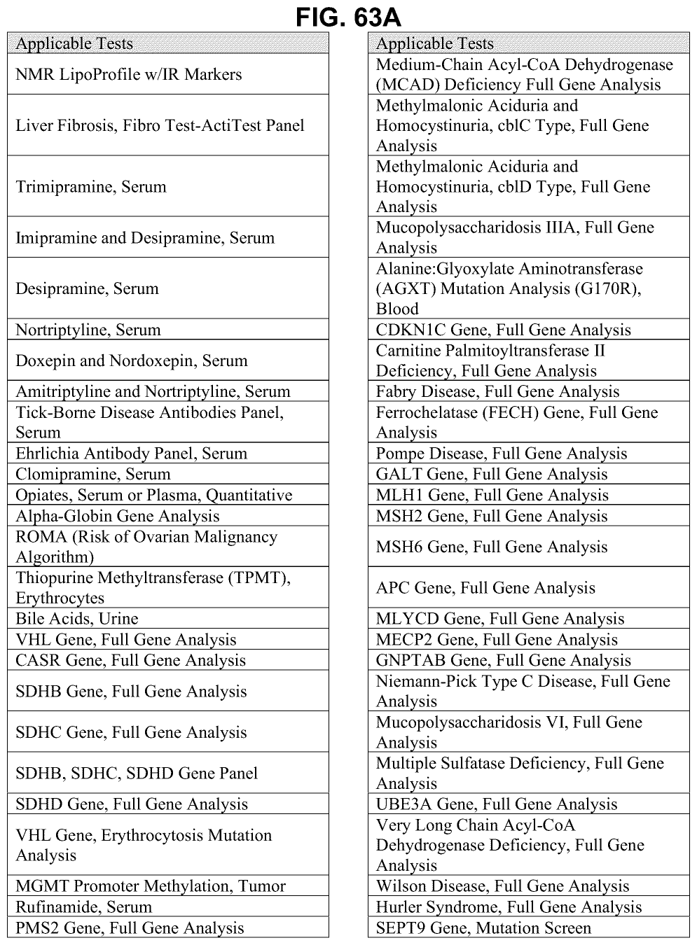

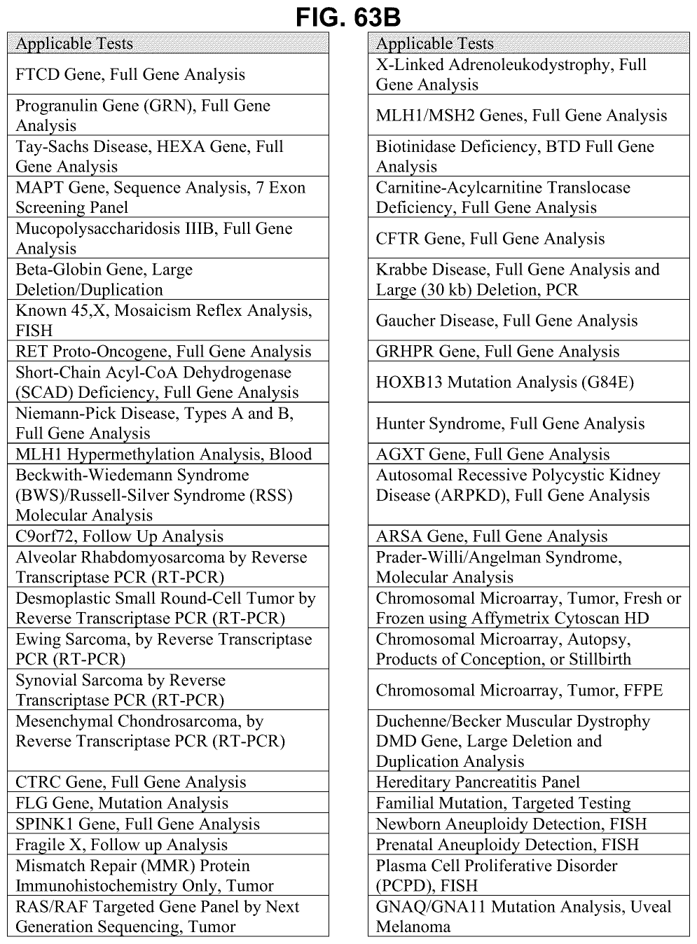

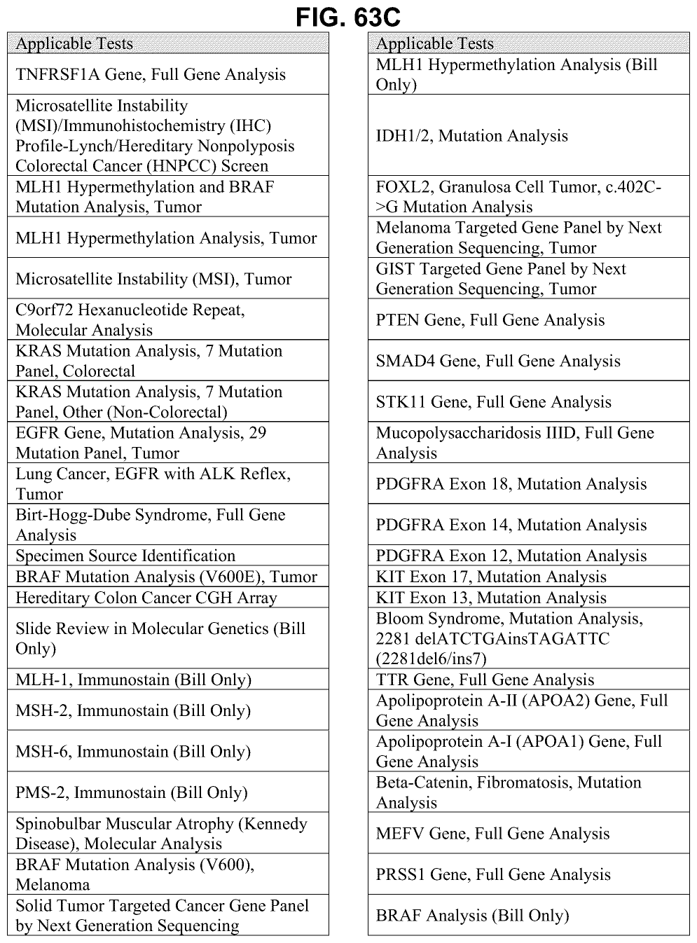

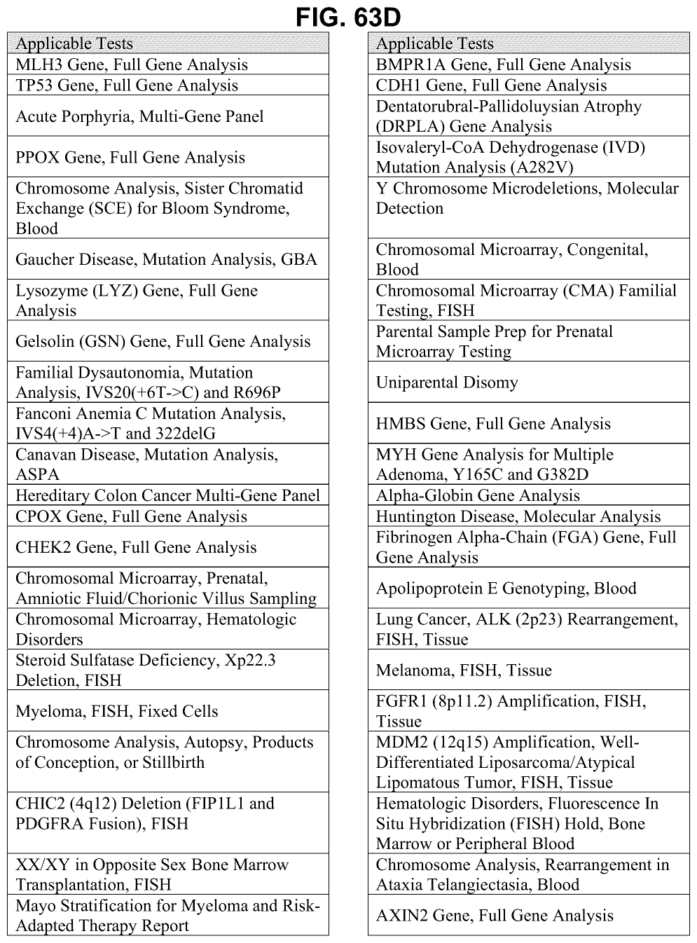

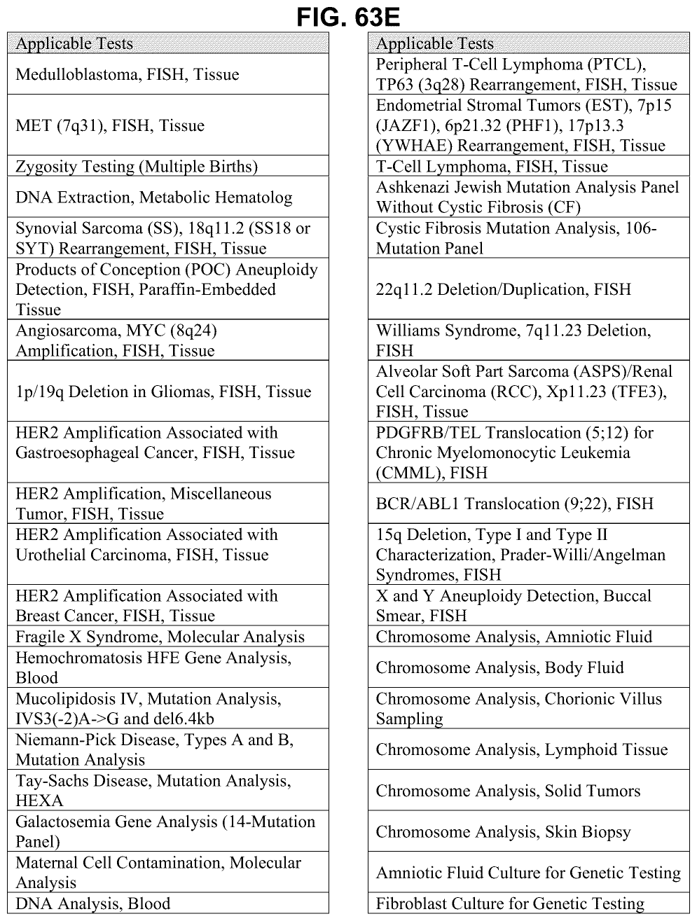

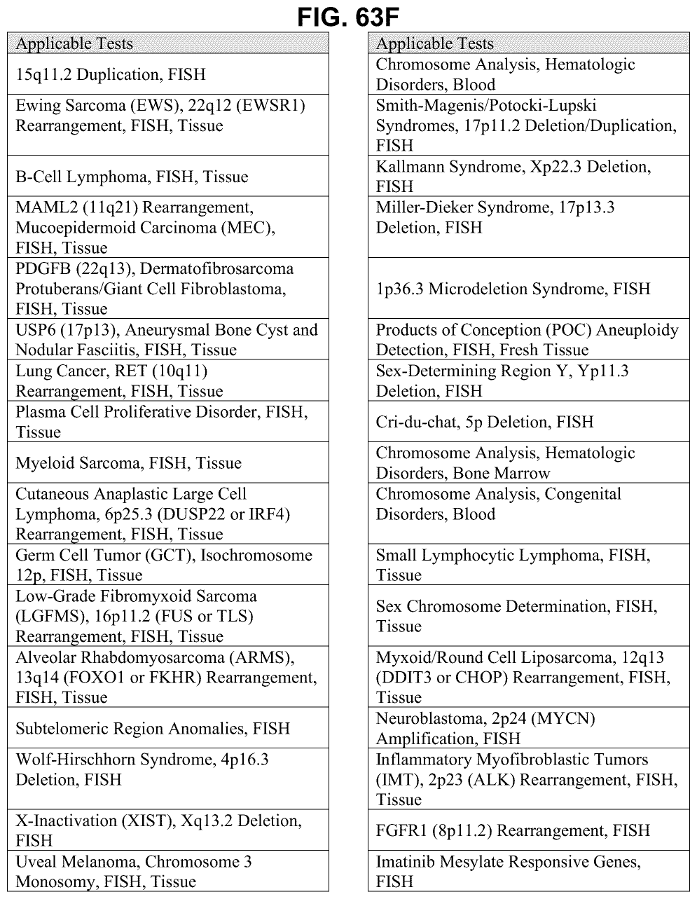

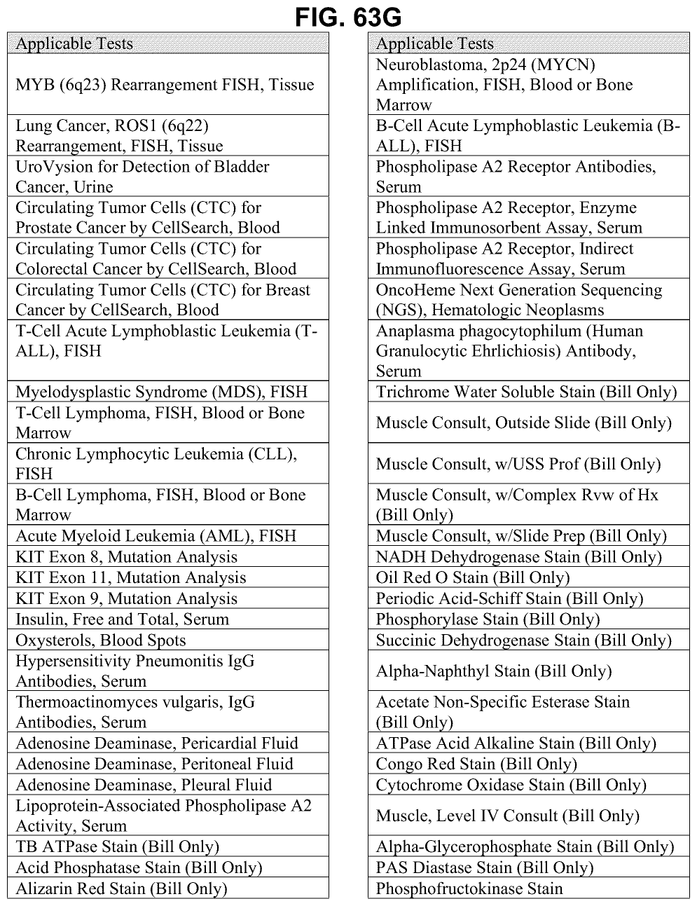

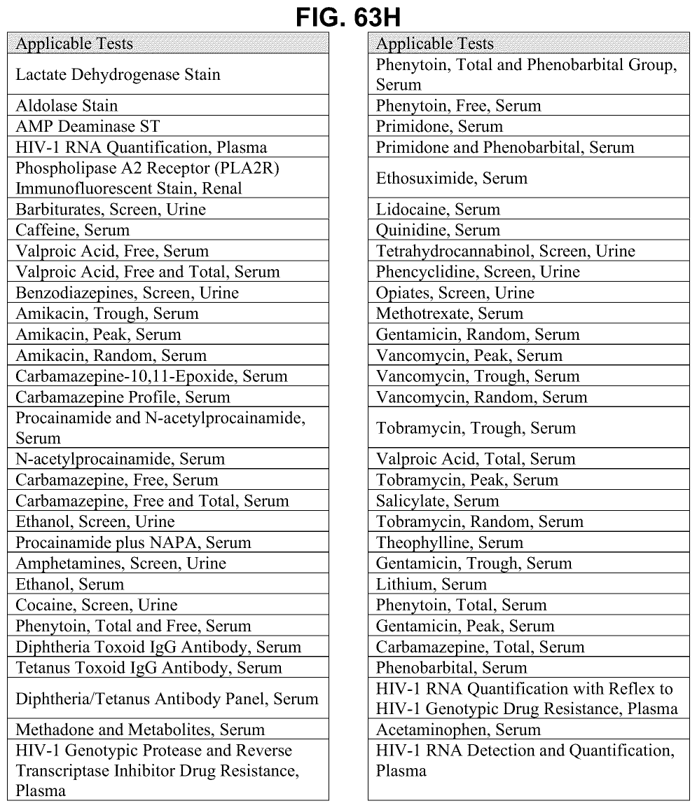

In some embodiments the user or operator can remove components of the system for analysis, for example before sending the sample off to a facility for analysis. The facility can be a CLIA facility, a laboratory, medical office or external dedicated facility. At facility, the samples can be used in any diagnostic tests including but not limited to common panels, thyroid tests, cancer diagnostic tests, tests and screens for cardiovascular disease, genetic diseases/pre-natal testing and infectious disease. A non-limiting list of applicable tests is herein included as FIG. 63A to FIG. 63H with this filing.

The devices and systems for acquiring, collecting, separating and stabilizing samples can be modular; comprising distinct compartments or components. The distinct components can or can not be easily removed or separated. Separable or removable components can include the sample substrate, or a sample separation component.

The devices herein (e.g., sample acquisition components and sample stabilization component) can be transported together to a laboratory for further analysis, e.g., of the bio-components. Alternatively, the stabilization component (e.g., substrate or substrate) can be shipped without a sample acquisition component to a laboratory for further analysis of the bio-components. In some instances, treatment and analysis can be performed on the deployed device, systems or substrate, and either the stabilization component or a component of the device or system can be transported to a healthcare provider or other party interested in the results of the test. Once a bio-sample is received at a location for analysis, the substrate, or components of the sample separation component can be removed. These components can be tagged and/or labeled to indicate the composition or target component that is stabilized on the matrix. Using the tag as an identifier the membranes or substrates can be sorted and prepared for the target test.

Systems and processes for receiving and processing the samples can vary depending on the identity, stability, source or other features of the deployed samples. The quality of a bio-sample component can directly impact the quality, reproducibility and reliability of a diagnostic test result. The aforementioned systems, devices, and methods for sample acquisition, collection, stabilization, and optional separation, can impact the sample composition, stability, concentration, and processing. For example the volume, size, quantity, stability and purity of a bio-sample can vary depending on the sample source and the components used to collect the sample. The quality of the samples and by extension the quality of the diagnostic results can be specific the kits, systems, devices and methods for acquiring the sample; therefore, methods, kits, and systems are also disclosed for receiving, processing, treating and/or preparing components of the bio-sample prior to analysis.

Components processed from the sample can include but are not limited to DNA/RNA including cell-free DNA and circulating-tumor DNA, proteins, antigens, antibodies, lipids including HDL/LDL, and any combination thereof. The bio-sample or target components can be extracted using any of the conventional nucleic acid extraction methods. Non-limiting examples of extraction methods that can include but are not limited to, electroelution, gelatin extraction, silica or glass bead extraction, guanidine-thiocyanate-phenol solution extraction, guanidinium thiocyanate acid-based extraction, centrifugation through sodium iodide or similar gradient, centrifugation with buffer, or phenol-chloroform-based extraction. For nucleic acid analysis the extraction step can help remove impurities such as proteins and concentrate the circulating nucleic acids. Extracted circulating nucleic acids can be inspected using methods such as agarose gel electrophoresis, spectrophotometry, fluorometry, or liquid chromatography.

DNA or nucleotides extracted from the sample can be prepared at a lab facility using various methods for reducing error and producing significant signal with limited sample size. Nucleic acid samples can be treated or subjected to various methods for efficient amplification of the desired target nucleic acid (e.g., “DNA template” or “nucleic acid template”). Modified primers can be designed to minimize or prevent the production of unwanted primer-dimers and chimeric products observed with other nucleic acid amplification methods and kits. Novel primer design methods can avoid the production of spurious nucleic acid amplification products. The methods and kits described used for analyzing the sample can comprise “AT GenomiPhi.” ATGenomiPhi can use modified hexamers are of the general formula: +N+N(atN)(atN)(atN)*N, wherein “+” precedes an LNA base, as described above, and (atN) represents a random mixture of 2-amino-dA, dC, dG, and 2-thio-dT. Other hexamers can comprise the formula (atN)(atN)(atN)(atN)(atN)*N, wherein the notations are consistent between these two hexamer designs. The use of these hexamers in nucleic acid amplification techniques can address, minimize or eliminate the problems associated with the production of primer-dimer formation and chimeric nucleic acids observed in traditional methods by inhibiting the ability of the random hexamers to anneal with one another, by increasing the melting Tₘ of the primers, improving the binding efficiency of the hexamer to the target nucleic acid via the addition of LNAs and 2-amino-dA to the primers, and preventing annealing of the target DNA to itself through the incorporation of 2-thio-dT into the random hexamers. Moreover, the primer modifications can increase their binding strength to the target nucleic acid and permit the utilization of more stringent hybridization buffers that further minimize the likelihood of the production of primer-dimers and chimeric nucleic acid products. These and other methods of DNA amplification methods can be found, e.g., in US Appn. No. US20130210078.

DNA derived from a sample can be analyzed using one or more methods for generating single-stranded DNA circles from a biological sample. The laboratory analyzing the sample can use a method comprising the steps of: treating the biological sample with an extractant to release nucleic acids, thereby forming a sample mixture; neutralizing the extractant; denaturing the released nucleic acids to generate single-stranded nucleic acids; and contacting the single-stranded nucleic acids with a ligase that is capable of template-independent, intramolecular ligation of a single-stranded DNA sequence to generate single-stranded DNA circles. The steps of the method can be performed without any intermediate nucleic acid isolation or nucleic acid purification. In certain embodiments, the steps can be performed in a sequential manner in a single reaction vessel. In certain embodiments, the single-stranded DNA circles can be amplified to enable subsequent analysis of the biological sample. In certain embodiments, the sample mixture can be dried on solid matrix prior to the neutralizing step. In certain embodiments, damage to the DNA can be repaired enzymatically prior to the denaturing step. In other aspects, a method is provided for analyzing a sample, e.g., biological sample. Thus, the single-stranded DNA circles generated according to certain embodiments of the method are amplified, and the amplification product is analyzed. The analysis can be performed by, for example, targeted sequencing of the amplified product. In another aspect, a method is provided for detecting chromosomal rearrangement breakpoints from a biological sample. Thus, the single-stranded DNA circles generated according to certain embodiments described herein are amplified, and the amplification product is analyzed, e.g., by sequencing. Any chromosomal rearrangement breakpoints can be identified by comparing the sequences to a known reference sequence. In yet another aspect, a kit can be provided that comprises an extractant for treating a biological sample to release nucleic acids; a reagent for neutralizing the extractant; and a ligase that is capable of template-independent, intramolecular ligation of a single-stranded DNA sequence. These and other method of DNA amplification methods can be found, e.g., in PCT Appn. No. WO US2015/50760.

Methods for generating a single-stranded DNA circle from a linear DNA can be used on the collected sample. The methods can comprise steps for providing a linear DNA, end-repairing the linear DNA by incubating it with a polynucleotide kinase in the presence of a phosphate donor to generate a ligatable DNA sequence having a phosphate group at a 5′ terminal end and a hydroxyl group at a 3′ terminal end, and performing an intra-molecular ligation of the repaired, ligatable DNA sequence with a ligase in order to generate the single-stranded DNA circle. Steps can be performed in a single reaction vessel without any intervening isolation or purification steps. The phosphate donor can be a guanosine triphosphate (GTP), a cytidine triphosphate (CTP), a uridine triphosphate (UTP), a deoxythymidine triphosphate (dTTP) or a combination thereof. The linear DNA can either be double-stranded or single-stranded DNA. DNA can be a segment of fragmented DNA such as circulating DNA. The ligatable DNA, if in double-stranded form, can need to be denatured prior to intra-molecular ligation reaction. A pre-adenylated ligase that is capable of template-independent, intra-molecular ligation of single-stranded DNA sequences can be employed for the ligation reaction. In other embodiments, the method for generating a single-stranded DNA circle from a linear DNA can employ a DNA pre-adenylation step prior to an intra-molecular ligation step. The linear DNA can optionally be incubated with a polynucleotide kinase in the presence of adenosine triphosphate (ATP) to generate a ligatable DNA sequence that comprises a phosphate group at a 5′ terminal end and a hydroxyl group at a 3′ terminal end. Generation of a ligatable DNA sequence from the linear DNA can be preferred if the linear DNA is in a highly fragmented form. The linear DNA or the ligatable DNA sequence can then be incubated with an adenylating enzyme in presence of ATP to generate a 5′ adenylated DNA sequence. The 5′ adenylated DNA sequence can be incubated with a non-adenylated ligase, which is capable of template-independent intra-molecular ligation of 5′ adenylated DNA sequence to generate the single-stranded DNA circle. All steps of the method can be performed in a single reaction vessel without any intervening isolation or purification steps. ATP can be removed from the reaction mixture (e.g., by treating the reaction mixture with a phosphatase) before the intra-molecular ligation reaction if the non-adenylated ligase is an ATP-dependent ligase. If 5′ adenylated DNA is in double-stranded form, it can need to be denatured prior to the intra-molecular ligation reaction. These and other method of DNA circularization and amplification methods can be found, e.g., in US Appn. No. US20150031086.

Sample treatment can involve steps to prepare RNA for analysis. The methods can involve the production of a nucleic acid structure and its subsequent use in the purification and amplification of nucleic acid. The methods can require a DNA sequence that comprises a double stranded region and a single stranded region. The single stranded region can be complementary to the RNA sequence of interest. The RNA sequence can then hybridized to the single stranded region of the DNA sequence and then the two sequences can be ligated to produce an RNA-DNA molecule. Methods can include steps whereby the 3′ end of RNA is ligated to a double stranded DNA oligonucleotide containing a promoter sequence. This double stranded DNA oligonucleotide can contain a promoter for RNA polymerase within the double stranded region that is followed by a segment of single stranded DNA forming a 3′ overhang. When 3′ overhang contains a string of thymidine residues, the single stranded portion of the double stranded DNA can hybridize to the 3′ end of messenger RNA (mRNA) poly(A) tails. After the addition of ligase mRNA can have one strand of double stranded DNA sequence ligated to 3′ end. When an RNA polymerase is added, the RNA-DNA hybrid molecules can be efficiently transcribed to synthesize cRNA. As transcription reactions using RNA polymerase can transcribe each template multiple times, this method can allow for effective RNA amplification. Another method similar to that described above can involve the ligation of the DNA oligonucleotide to the RNA as described. However, the DNA oligonucleotide can either attach to a solid support or contain an affinity tag. This can allow for very efficient covalent attachment and/or capture of RNA molecules, which can be used for any of a variety of purposes. Additional methods can utilize ligation and subsequent transcription to create complementary RNA containing a user-defined sequence at the 5′ end of the cRNA. This sequence “tag” can be placed between the RNA polymerase promoter and 3′ end of the ligated RNA molecule. The user-defined sequence can be used for purification or identification or other sequence specific manipulations of cRNA. If cRNA product is subsequently ligated and re-amplified according to the described method, the resulting doubly-amplified product can be “sense”, with respect to the original sense template and this new product can have two separate user-defined sequences located at 5′ ends. These sequences can be used for synthesis of cDNA, allowing for full-length synthesis and directional cloning. Those skilled in the art will understand that either with or without the user defined sequences this double amplification method can provide a significant increase in RNA quantity, allowing for analysis of samples previously too small for consideration. These and other methods for amplification of DNA fragments can be found, e.g., in US Appn. No. US20080003602.

Additional methods for analysis of the bio-sample can focus on nucleic acids present in a region of interest in a biological sample, and provide protein expression information for many proteins as well as add to the DNA sequence information. Additional methods for sample analysis can focus on homogeneous subsection of a heterogeneous sample. Specific subpopulations of cells within mixed populations can be accurately identified from predetermined selection criteria and analyzed. Mutations can be identified, which can be useful for diagnosis and/or prognosis or for further investigation of drug targets. These and other methods for DNA amplification can be found, e.g., in PCT Appn. No. US2015/50760.

### Elution of Nucleic Acids

In some cases, nucleic acids, e.g., DNA or RNA, in a sample (e.g., a biological sample), are applied to a stabilization matrix (e.g., nucleic acid stabilization matrix), the sample is optionally dried on the stabilization matrix, and the nucleic acid on the stabilization matrix, e.g., DNA or RNA, are eluted from the stabilization matrix. In some instances, a dried biological sample is stabilized on a stabilization matrix capable of stabilizing a nucleic acid, e.g., as described herein. In some instances, a method comprises the steps of: (a) contacting, e.g., spotting a sample, e.g., a biological sample, comprising a nucleic acid, e.g., DNA or RNA, on a nucleic acid stabilization matrix, (b) optionally drying the sample, e.g., biological sample, on the nucleic acid stabilization matrix, (c) optionally contacting the nucleic acid stabilization matrix comprising nucleic acid with a lysis buffer, (d) optionally contacting the nucleic acid stabilization matrix comprising nucleic acid with a nucleic acid binding buffer (optionally containing an organic solvent), e.g., by submerging the nucleic acid stabilization matrix comprising nucleic acid in the binding buffer; (e) optionally contacting the nucleic acid stabilization matrix comprising nucleic acid with a wash buffer, e.g., by submerging the nucleic acid stabilization matrix in the wash buffer, and (f) contacting the nucleic acid stabilization matrix comprising nucleic acid with an elution buffer, e.g., by submerging the nucleic acid stabilization matrix comprising nucleic acid in the elution buffer, to elute the nucleic acid from the stabilization matrix.

In some cases, nucleic acid, e.g., RNA or DNA, can be extracted from a stabilization matrix, e.g., a paper stabilization matrix, e.g., using an extraction buffer. The extracted nucleic acid, e.g., RNA or DNA, can be bound to a second matrix, e.g., a second solid support, e.g., beads, e.g., magnetic beads. The binding to a second matrix, e.g., a second solid support, e.g., beads, e.g., magnetic beads, can occur in a binding buffer. Nucleic acid, e.g., RNA or DNA, on the second matrix can be washed with one or more wash buffers. Nucleic acid on the second matrix can be treated with an enzyme solution comprising, e.g., a DNase, RNase, or protease, to degrade a specific type of molecule (e.g., RNA, DNA, or protein). Nucleic acid on the solid matrix can be eluted from the solid matrix using, e.g., an elution buffer.

The elution can be performed on at least a portion of the stabilization matrix comprising a sample, e.g., a dried biological sample. In some cases, a portion of the stabilization matrix can be separated from the rest of the stabilization matrix, e.g., a portion of a stabilization matrix can be punched out of the stabilization matrix, and nucleic acids in the separated portion can be eluted. The punches can be about 0.5, 1, 2, 3, 4, 5, 6, 7, 8, 9, 10, 15, 20, 25, 30, 35, 40, 45, 50, 55, or 60 mm in diameter. The punches can be from about 10 to about 60, from about 10 to about 50, from about 10 to about 40, from about 10 to about 30, from about 10 to about 20, from about 1 to about 10, from about 2 to about 9, from about 3 to about 8, from about 4 to about 7, from about 5 to about 6, from about 3 to about 6, from about 1 to about 4, from about 1 to about 3, or from about 1 to about 2 mm in diameter. In some cases, a stabilization matrix comprising nucleic acid is not separated into portions before the nucleic acid is eluted from the stabilization matrix.

In some cases, a nucleic acid stabilization matrix comprising nucleic acid can be contacted with a lysis buffer. In some cases, the binding and retention of a nucleic acid to a stabilization matrix can be enhanced through contacting the stabilization matrix with a binding buffer. In some cases, a portion of the stabilization matrix is contacted with a nucleic acid binding buffer. In some instances, the nucleic acid binding buffer can comprise beads. In some cases, the stabilization matrix is contacted with a wash buffer, e.g., to remove impurities. In some instances, the nucleic acid can be eluted by contacting the stabilization matrix with an elution buffer.

In some instances, the nucleic acid lysis buffer, binding buffer, wash buffer, or elution buffer can comprise a commercially available buffer. For instance, a buffer can comprise TRIzol® manufactured by Thermofisher®, Buffer RLT manufactured by Qiagen®, Buffer RLN manufactured by Qiagen®, RNA Lysis Buffer (RLA) manufactured by Promega, Pure Yield™ Cell Lysis Solution (CLA) manufactured by Promega, PureYield™ Endotoxin Removal Wash manufactured by Promega, PureZOL™ RNA isolation reagent (Bio-Rad™), RNA Lysis Buffer or DNA/RNA Binding Buffer manufactured by Zymo Research Corp, or RNA Capture Buffer manufactured by Pierce™.

In some instances, the nucleic acid lysis buffer, binding buffer, wash buffer, or elution buffer can comprise one or more buffering agents (or pH buffer), one or more salts, one or more reducing agents, one or more chelators, one or more surfactants, one or more enzymes, one or more protein denaturants, one or more blocking reagents, one or more organic solvents, or any combination thereof. For example, the nucleic acid lysis buffer, binding buffer, wash buffer, or elution buffer can comprise one or more protein denaturants and one or more reducing agents. The nucleic acid lysis buffer, binding buffer, wash buffer, or elution buffer can comprise can comprise one or more protein denaturants, one or more reducing agents, and one or more enzymes. The nucleic acid lysis buffer, binding buffer, wash buffer, or elution buffer can comprise one or more buffering agents, one or more protein denaturants, one or more reducing agents, and one or more enzymes. The nucleic acid lysis buffer, binding buffer, wash buffer, or elution buffer can comprise one or more salts and one or more buffers, The nucleic acid lysis buffer, binding buffer, wash buffer, or elution buffer can comprise one or more salts, one or more buffers, and one or more organic solvents. The nucleic acid lysis buffer, binding buffer, wash buffer, or elution buffer can comprise one or more buffers and one or more enzymes. The nucleic acid lysis buffer, binding buffer, wash buffer, or elution buffer can comprise one or more buffers and one or more chelating agents. The nucleic acid lysis buffer, binding buffer, wash buffer, or elution buffer can comprise one or more buffers, one or more chelating agents, and one or more organic solvents.

The one or more buffering agents can be, e.g., saline, citrate, phosphate, phosphate buffered saline, acetate, glycine, tris(hydroxymethyl)aminomethane (tris) hydrochloride, tris buffered saline (TBS), 3-[1,3-dihydroxy-2-(hydroxymethyl) propan-2-yl]amino]propane-1-sulfonic acid (TAPS), bicine, tricine, 3-[1,3-dihydroxy-2-(hydroxymethyl) propan-2-yl]amino]-2-hydroxypropane-1-sulfonic acid (TAPSO), 4-(2-hydroxyethyl)-1-piperazineethanesulfonic acid (HEPES), piperazine-N,N′-bis(2-ethanesulfonic acid)(PIPES), 3-(N-morpholino) propanesulfonic acid (MOPS), 2-(N-morpholino) ethanesulfonic acid (MES), 2-[[1,3-dihydroxy-2-(hydroxymethyl) propan-2-yl]amino]ethanesulfonic acid (TES), cacodylate, glycine, carbonate, or any combination thereof. The one or more buffering agents can be present at a concentration of from about 0.1 mM to about 500, from about 0.1 mM to about 400 mM, from about 0.1 mM to about 300 mM, from about 0.1 mM to about 200 mM, from about 0.1 mM to about 100 mM, from about 0.1 mM to about 50 mM, from about 0.1 mM to about 25 mM, from about 0.1 mM to about 20 mM, from about 0.1 mM to about 15 mM, from about 0.1 mM to about 10 mM, from about 0.1 mM to about 5 mM, from about 0.1 mM to about 4 mM, from about 0.1 mM to about 3 mM, from about 0.1 mM to about 2 mM, from about 0.1 mM to about 1 mM, from about 0.1 mM to about 0.9 mM, from about 0.1 mM to about 0.8 mM, from about 0.1 mM to about 0.7 mM, from about 0.1 mM to about 0.6 mM, from about 0.1 mM to about 0.5 mM, from about 0.1 mM to about 0.4 mM, from about 0.1 mM to about 0.3 mM, or from about 0.1 mM to about 0.2 mM. The buffering agent can be present at a concentration of less than 500 mM, less than 400 mM, less than 300 mM, less than 200 mM, less than 100 mM, less than 50 mM, less than 25 mM, less than 20 mM, less than 15 mM, less than 10 mM, less than 5 mM, less than 4 mM, less than 3 mM, less than 2 mM, less than 1 mM, less than 0.9 mM, less than 0.8 mM, less than 0.7 mM, less than 0.6 mM, less than 0.5 mM, less than 0.4 mM, less than 0.3 mM, less than 0.2 mM, or less than 0.1 mM. The buffering agent can be present at a concentration of more than 500 mM, more than 400 mM, more than 300 mM, more than 200 mM, more than 100 mM, more than 50 mM, more than 25 mM, more than 20 mM, more than 15 mM, more than 10 mM, more than 5 mM, more than 4 mM, more than 3 mM, more than 2 mM, more than 1 mM, more than 0.9 mM, more than 0.8 mM, more than 0.7 mM, more than 0.6 mM, more than 0.5 mM, more than 0.4 mM, more than 0.3 mM, more than 0.2 mM, or more than 0.1 mM.

The one or more salts can be, e.g., sodium chloride, sodium acetate, sodium bicarbonate, sodium bisulfate, sodium bromide, potassium chloride, potassium acetate, potassium bicarbonate, potassium bisulfate, potassium bromate, potassium bromide, or potassium carbonate. The one or more salts can be at a concentration of about 0.1 mM, 5 mM, 10 mM, 25 mM, 50 mM, 100 mM, 250 mM, 500 mM, 750 mM, or 1000 mM in a buffer. The one or more salts can be at a concentration of less than 0.1 mM, 5 mM, 10 mM, 25 mM, 50 mM, 100 mM, 250 mM, 500 mM, 750 mM, or 1000 mM in a buffer. The one or more salts can be at a concentration of at least 0.1 mM, 5 mM, 10 mM, 25 mM, 50 mM, 100 mM, 250 mM, 500 mM, 750 mM, or 1000 mM.

The one or more reducing agents can be, e.g., beta-mercaptoethanol (BME), 2-aminoethanethiol (2MEA-HCl (cysteamine-HCl)), dithiothreitol (DTT), glutathione (GSH), tris(2-carboxyethyl) phosphine (TECP), or any combination thereof. The concentration of the one or more reducing agents can be about 0.1 mM, 0.5 mM, 1 mM, 10 mM, 50 mM, 100 mM, 250 mM, or 500 mM. The concentration of the one or more reducing agents can be less than 0.5 mM, 1 mM, 10 mM, 50 mM, 100 mM, 250 mM, or 500 mM. For example, the concentration of DTT can be from about 0.05 mM to about 100 mM, from about 0.5 mM to about 50 mM, or from about 5 mM to about 10 mM. The concentration of TCEP can be from about 0.05 mM to about 50 mM, from about 0.5 mM to about 50 mM, or from about 0.5 mM to about 5 mM. The concentration of BME can be from about 0.05% to about 10%, from about 0.5% to about 5%, or from about 1% to about 10%. The concentration of GSH can be from about 0.05 mM to about 25 mM, from about 0.5 mM to about 10 mM, or from about 5 mM to about 10 mM. The concentration of the one or more reducing agents can be about 1 mM, 10 mM, 50 mM, 100 mM, 250 mM, or 500 mM.

The one or more chelators can be, e.g., a carbohydrate; a lipid; a steroid; an amino acid or related compound; a phosphate; a nucleotide; a tetrapyrrol; a ferrioxamines; an ionophor; a phenolic; or a synthetic chelator such as 2,2′-bipyridyl, dimercaptopropanol, ethylenediaminotetraacetic acid (EDTA), ethylenedioxy-diethylene-dinitrilo-tetraacetic acid, ethylene glycol-bis-(2-aminoethyl)-N,N,N′, N′-tetraacetic acid (EGTA), a metal nitrilotriacetic acid (NTA), salicylic acid, or triethanolamine (TEA). The concentration of the one or more chelating agents in a buffer can be about 0.1 mM, 1 mM, 5 mM, 10 mM, 20 mM, or 25 mM. The concentration of the one or more chelating agents in a buffer can be less than 0.1 mM, 1 mM, 5 mM, 10 mM, 20 mM, or 25 mM. The concentration of the one or more chelating agents in a buffer can be more than 0.1 mM, 1 mM, 5 mM, 10 mM, 20 mM, or 25 mM.

The one or more surfactants can be, e.g., an anionic, cationic, nonionic or amphoteric type. The one or more surfactants can be polyethoxylated alcohols; polyoxyethylene sorbitan; octoxynol such as Triton X 100™ (polyethylene glycol p-(1,1,3,3-tetramethylbutyl)-phenyl ether); polysorbates such as Tween™ 20 ((e.g., polysorbate 20) or Tween™ 80 (polysorbate 80); sodium dodecyl sulfate; sodium lauryl sulfate; nonylphenol ethoxylate such as Tergitol™; cyclodextrins; or any combination thereof. The one or more surfactants can be present at a concentration of less than 0.001%, less than 0.005%, less than 0.01%, less than 0.015%, less than 0.02%, less than 0.025%, less than 0.03%, less than 0.035%, less than 0.04%, less than 0.045%, less than 0.05%, less than 0.055%, less than 0.06%, less than 0.065%, less than 0.07%, less than 0.075%, less than 0.08%, less than 0.085%, less than 0.09%, less than 0.095%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, or less than 0.1% by volume relative to the total volume of the elution buffer. The one or more surfactants can be at a concentration of about 0.01%, 0.05%, 0.1%, 0.5%, 1%, 5%, or 10%. The one or more surfactants can be at a concentration of less than 0.01%, 0.05%, 0.1%, 0.5%, 1%, 5%, or 10%. The one or more surfactants can be at a concentration of more than 0.01%, 0.05%, 0.1%, 0.5%, 1%, 5%, or 10%.

The one or more protein denaturants can be, e.g., a chaotropic agent, e.g., a chaotropic salt. A chaotropic agent can be butanol, ethanol, guanidine chloride, guanidine hydrochloride, guanidine isothiocyanate, lithium perchlorate, lithium acetate, magnesium chloride, phenol, propanol, sodium iodide, sodium thiocyanate, thiourea, urea, or any combination thereof. The concentration of the chaotropic agent in a buffer can be about 0.1 mM, 1 mM, 10 mM, 100 mM, 1 M, 6 M, or 8 M. The concentration of the chaotropic agent in a buffer can be at least 0.1 mM, 1 mM, 10 mM, 100 mM, 1 M, 6 M, or 8 M. The concentration of the chaotropic agent in a buffer can be less than 0.1 mM, 1 mM, 10 mM, 100 mM, 1 M, 6 M, or 8 M.

The nucleic acid lysis buffer, binding buffer, wash buffer or elution buffer can further comprise one or more enzymes. A lysis buffer, binding buffer, wash buffer or elution buffer can comprise DNase or RNase in amounts sufficient to remove DNA or RNA impurities, respectively, from each other. A lysis buffer, binding buffer, wash buffer or elution buffer can comprise lysis enzymes such as hen egg white lysozyme, T4 lysozyme and the like, as well as enzymes such as carbohydrases, phytases, and proteases such as trypsin, Proteinase K, pepsin, chymotrypsin, papain, bromelain, subtilisin, or elastase. A protease can be a serine protease, a cysteine protease, a threonine protease, an aspartic protease, a glutamic protease, or a metalloprotease, or an asparagine peptide lyase.

The nucleic acid lysis buffer, binding buffer, wash buffer or elution buffer can be an aqueous solution having a pH from about 2 to about 10, from about 2 to about 9, from about 2 to about 8, from about 2 to about 7, from about 2 to about 6, from about 2 to about 5, from about 2 to about 4, or from about 2 to about 3. The nucleic acid lysis buffer, binding buffer, wash buffer or elution buffer can be an aqueous solution having a pH from about 6 to about 8, from about 6 to about 7.9, between about 6 to about 7.8, between about 6 to about 7.7, between about 6 to about 7.6, between about 6 to about 7.5, between about 6 to about 7.4, between about 6 to about 7.3, from about 6 to about 7.2, from about 6 to about 7.1, from about 6 to about 7, from about 6 to about 6.9, from about 6 to about 6.8, from about 6 to about 6.7, from about 6 to about 6.6, from about 6 to about 6.5, from about 6 to about 6.4, from about 6 to about 6.3, from about 6 to about 6.2, or from about 6 to about 6.1. The nucleic acid lysis buffer, binding buffer, wash buffer or elution buffer can be an aqueous solution having a pH of at least about 3, at least about 4, at least about 5, at least about 6, at least about 7, at least about 8, at least about 9, or at least about 10.

The nucleic acid lysis buffer, binding buffer, wash buffer, or elution buffer can further comprise additional ingredients. For example, a lysis buffer, binding buffer, wash buffer, or elution buffer can comprise a protein blocking agent that minimizes non-specific binding such as bovine serum albumin, fetal bovine serum, and the like. A lysis buffer, binding buffer, wash buffer, or elution buffer can comprise additional nucleic acids (to include DNA or RNA) from an organism distinct from the subject such as bacterial DNA, bacterial DNA, yeast DNA, yeast RNA, mammalian nucleic acids including primate nucleic acids such as human or chimpanzee DNA, or non-mammalian nucleic acids including nucleic acids from fish such as herring, mackerel, krill, or salmon DNA and the like.

A lysis buffer, binding buffer, wash buffer, or elution buffer can comprise an amount of one or more organic solvents, e.g., to enhance binding of a nucleic acid to the stabilization matrix. The one or more organic solvents can be methanol, ethanol, DMSO, DMF, dioxane, tetrahydrofuran, propanol, isopropanol, butanol, t-butanol, or pentanol, acetone and the like. In some instances, a binding buffer can comprise less than 0.01%, less than 0.05%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, less than 1%, less than 1.5%, less than 2%, less than 2.5%, less than 3%, less than 3.5%, less than 4%, less than 4.5%, less than 5%, less than 5.5%, less than 6%, less than 6.5%, less than 7%, less than 7.5%, less than 8%, less than 8.5%, less than 9%, less than 9.5%, less than 10%, less than 11%, less than 12%, less than 13%, less than 14%, less than 15%, less than 16%, less than 17%, less than 18%, less than 19%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 99%, or 100% of an organic solvent by volume relative to the total volume of the solution. The organic solvent can be at a concentration of at least 0.1%, 1%, 10%, 50%, 75%, or 100%. The organic solvent can be at a concentration of about 0.1%, 1%, 10%, 50%, 75%, or 100%.

The stabilization matrix, or the portion of the stabilization matrix, can be contacted with a volume of the nucleic acid binding buffer, wash buffer or elution buffer of less than 5 μL, less than 10 μL, less than 15 μL, less than 20 μL, less than 25 μL, less than 30 μL, less than 35 uL, less than 40 μL, less than 45 μL, less than 50 μL, less than 55 μL, less than 60 μL, less than 65 μL, less than 70 μL, less than 75 μL, less than 80 μL, less than 85 μL, less than 90 μL, less than 95 μL, less than 100 μL, less than 110 μL, less than 120 μL, less than 130 μL, less than 140 uL, less than 150 μL, less than 160 μL, less than 170 μL, less than 180 μL, less than 190 μL, less than 200 μL, less than 250 μL, less than 300 μL, less than 350 μL, less than 400 μL, less than 450 μL, less than 500 μL, less than 550 μL, less than 600 μL, less than 650 μL, less than 700 μL, less than 750 μL, less than 800 μL, less than 850 μL, less than 900 μL, less than 950 μL, less than 1,000 μL, less than 1.5 mL, less than 2 mL, less than 2.5 mL, less than 3 mL, less than 3.5 mL, less than 4 mL, less than 4.5 mL, less than 5 mL, less than 5.5 mL, less than 6 mL, less than 6.5 mL, less than 7 mL, less than 7.5 mL, less than 8 mL, less than 8.5 mL, less than 9 mL, less than 9.5 mL, or less than 10 mL. The stabilization matrix, or portion of the stabilization matrix, can be contacted with about 0.1 mL, 0.2 mL, 0.5 mL, 0.7 mL, 1 mL, 2 mL, 5 mL, 7 mL, or 10 mL of buffer. The stabilization matrix, or portion of the stabilization matrix, can be contacted with at least 0.1 mL, 0.2 mL, 0.5 mL, 0.7 mL, 1 mL, 2 mL, 5 mL, 7 mL, or 10 mL of buffer.

The volume of binding buffer, wash buffer, or elution buffer contacted with the stabilization matrix can be dependent on the surface area of the stabilization matrix. The amount of binding buffer, wash buffer, or elution buffer can be less than 1 μL/mm², less than 2 μL/mm², less than 3 μL/mm², less than 4 μL/mm², less than 5 L/mm², less than 6 μL/mm², less than 7 μL/mm², less than 8 μL/mm², less than 9 μL/mm², less than 10 μL/mm², less than 12 μL/mm², less than 14 μL/mm², less than 16 μL/mm², less than 18 μL/mm², less than 20 μL/mm², less than 25 μL/mm², less than 30 μL/mm², less than 35 μL/mm², less than 40 μL/mm², less than 45 μL/mm², less than 50 μL/mm², less than 55 μL/mm², less than 60 μL/mm², less than 65 μL/mm², less than 70 μL/mm², less than 75 μL/mm², less than 80 μL/mm², less than 85 μL/mm², less than 90 μL/mm², less than 95 μL/mm², less than 100 μL/mm², less than 150 μL/mm², less than 200 μL/mm², less than 250 μL/mm², less than 300 μL/mm², less than 350 μL/mm², less than 400 μL/mm², less than 450 μL/mm², less than 500 μL/mm², less than 550 μL/mm², less than 600 μL/mm², less than 650 μL/mm², less than 700 μL/mm², less than 750 μL/mm², less than 800 μL/mm², less than 850 μL/mm², less than 900 μL/mm², less than 950 μL/mm², or less than 1,000 μL/mm². In some cases, the amount of binding buffer, wash buffer, or elution buffer can be from about 10 μL/mm² to about 1,000 μL/mm², from about 10 μL/mm² to about 900 μL/mm², from about 10 μL/mm² to about 800 μL/mm², from about 10 μL/mm² to about 700 μL/mm², from about 10 μL/mm² to about 600 μL/mm², from about 10 μL/mm² to about 500 μL/mm², from about 10 L/mm² to about 400 L/mm², from about 10 μL/mm² to about 300 μL/mm², from about 10 μL/mm² to about 200 L/mm², from about 10 μL/mm² to about 100 μL/mm², from about 10 μL/mm² to about 90 μL/mm², from about 10 μL/mm² to about 80 L/mm², from about 10 μL/mm² to about 70 μL/mm², from about 10 μL/mm² to about 60 μL/mm², from about 10 μL/mm² to about 50 μL/mm², from about 10 μL/mm² to about 40 μL/mm², from about 10 μL/mm² to about 30 μL/mm², or from about 10 μL/mm² to about 20 μL/mm².

The lysis, binding, washing or elution of the nucleic acid can be performed in the presence or absence of agitation from an agitation source. An agitation source can be a rocker, vortexer, mixer, shaker and the like. In some cases, an agitation source can be set to a constant speed. The speed can be less than 1 rotations per minute (rpm), less than 5 rpm, less than 10 rpm, less than 15 rpm, less than 20 rpm, less than 25 rpm, less than 30 rpm, less than 35 rpm, less than 40 rpm, less than 45 rpm, less than 50 rpm, less than 55 rpm, less than 60 rpm, less than 65 rpm, less than 70 rpm, less than 75 rpm, less than 80 rpm, less than 85 rpm, less than 90 rpm, less than 95 rpm, less than 100 rpm, less than 150 rpm, less than 200 rpm, less than 250 rpm, less than 300 rpm, less than 350 rpm, less than 400 rpm, less than 450 rpm, less than 500 rpm, less than 550 rpm, less than 600 rpm, less than 650 rpm, less than 700 rpm, less than 750 rpm, less than 800 rpm, less than 850 rpm, less than 900 rpm, less than 950 rpm, less than 1,000 rpm, less than 1,500 rpm, less than 2,000 rpm, less than 2,500 rpm, less than 3,000 rpm, less than 3,500 rpm, less than 4,000 rpm, less than 4,500 rpm, less than 5,000 rpm, less than 5,500 rpm, less than 6,000 rpm, less than 6,500 rpm, less than 7,000 rpm, less than 7,500 rpm, less than 8,000 rpm, less than 8,500 rpm, less than 9,000 rpm, less than 9,500 rpm, or less than 10,000 rpm. The speed can be about 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm. The speed can be at least 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm.

The binding, washing or elution can be performed at a temperature of about 0° C., less than 1° C., less than 2° C., less than 3° C., less than 4° C., less than 5° C., less than 6° C., less than 7° C., less than 8° C., less than 9° C., less than 10° C., less than 11° C., less than 12° C., less than 13° C., less than 14° C., less than 15° C., less than 16° C., less than 17° C., less than 18° C., less than 19° C., less than 20° C., less than 21° C., less than 22° C., less than 23° C., less than 24° C., less than 25° C., less than 26° C., less than 27° C., less than 28° C., less than 29° C., less than 30° C., less than 31° C., less than 32° C., less than 33° C., less than 34° C., less than 35° C., less than 36° C., less than 37° C., less than 38° C., less than 39° C., less than 40° C., less than 45° C., less than 50° C., less than 55° C., less than 60° C., less than 65° C., less than 70° C., less than 75° C., less than 80° C., less than 85° C., less than 90° C., less than 95° C., or about 100° C. In some cases, the binding, washing or elution can be performed at a temperature of from about 10° C. to about 100° C., from about 10° C. to about 95° C., from about 10° C. to about 90° C., from about 10° C. to about 85° C., from about 10° C. to about 80° C., from about 10° C. to about 75° C., from about 10° C. to about 70° C., from about 10° C. to about 65° C., from about 10° C. to about 60° C., from about 10° C. to about 55° C., or from about 10° C. to about 50° C. In some cases, the lysis, binding, washing or elution can be performed at a temperature of from about 20° C. to about 50° C., from about 20° C. to about 48° C., from about 20° C. to about 46° C., from about 20° C. to about 44° C., from about 20° C. to about 42° C., from about 20° C. to about 40° C., from about 20° C. to about 38° C., from about 20° C. to about 36° C., from about 20° C. to about 34° C., from about 20° C. to about 32° C., from about 20° C. to about 30° C., from about 20° C. to about 28° C., from about 20° C. to about 26° C., from about 20° C. to about 24° C., or from about 20° C. to about 22° C. The temperature can be about 10° C., 20° C., 25° C., 30° C., 37° C., 50° C., or 65° C.

The lysis, binding, washing or elution can be performed for less than 1, less than 5, less than 10, less than 15, less than 20, less than 25, less than 30, less than 35, less than 40, less than 45, less than 50, less than 55, or less than 60 minutes. The binding, washing or elution can be performed for less than 0.5, less than 1, less than 1.5, less than 2, less than 2.5, less than 3, less than 3.5, less than 4, less than 4.5, less than 5, less than 5.5, less than 6, less than 6.5, less than 7, less than 7.5, less than 8, less than 8.5, less than 9, less than 9.5, less than 10, less than 10.5, less than 11, less than 11.5, or less than 12 hours, less than 18 hrs, less than 1 day, less than 1.5 days, less than 2 days, less than 2.5 days, less than 3 days, less than 3.5 days, less than 4 days, less than 4.5 days, less than 5 days, less than 5.5 days, less than 6 days, less than 6.5 days, or less than 7 days. The lysis, binding, washing, or elution can be performed for about 0.25 hr, 0.5 hr, 1 hr, 2 hr, 5 hr, 10 hr, 12 hr, 18 hr, 24 hr, 3 days, or 1 week, or more. The lysis, binding, washing, or elution can be performed for at least 0.25 hr, 0.5 hr, 1 hr, 2 hr, 5 hr, 10 hr, 12 hr, 18 hr, 24 hr, 3 days, or 1 week.

The eluted nucleic acid can be transferred to a container for storage or further processing, or can be transferred to an assay vessel for characterization.

### Elution of Proteins

Protein can be eluted from a matrix, e.g., a matrix described herein. A sample, e.g., a biological sample, can be stabilized on a stabilization matrix capable of stabilizing a protein, e.g., as described herein, prior to elution. The elution can comprise contacting the stabilization matrix with an elution buffer. The contacting can comprise incubating (e.g., with agitation) the stabilization matrix in the elution buffer to elute the protein from the stabilization matrix. Before elution, the stabilization matrix can be contacted with a binding and or wash buffer.

The elution can be performed on at least a portion of the stabilization matrix comprising a sample, e.g., a dried biological sample. In some cases, a portion of the stabilization matrix can be separated from the rest of the stabilization matrix and used for further processing. In some cases, the portion is more than 5%, 15%, 25%, 35%, 45%, 55%, 65%, 75%, 85%, or 95% of the stabilization matrix. In some cases, the portion is less than 5%, 15%, 25%, 35%, 45%, 55%, 65%, 75%, 85%, or 95% of the stabilization matrix. The portion of a stabilization matrix can be punched out of the stabilization matrix, and proteins in the separated portions can be eluted. The portion separated for further processing can comprise 100%, or about 90%, 80%, 70%, 60%, 50%, or less of a sample that was applied to the matrix. The punches can be about 0.5, 1, 2, 3, 4, 5, 6, 7, 8, 9, 10, 15, 20, 25, 30, 35, 40, 45, 50, 55, or 60 mm in diameter. The punches can be at most 0.5, 1, 2, 3, 4, 5, 6, 7, 8, 9,10, 15, 20, 25, 30, 35, 40, 45, 50, 55, or 60 mm in diameter. The punches can be from about 10 to about 60, from about 10 to about 60, from about 10 to about 60, from about 10 to about 30, from about 10 to about 20, from about 1 to about 10, from about 2 to about 9, from about 3 to about 8, from about 4 to about 7, from about 3 to about 6, from about 4 to about 5, from about 1 to about 4, from about 1 to about 3, from about or from about 1 to about 2 mm in diameter. In some cases, a stabilization matrix comprising protein is not separated into portions before the nucleic acid is eluted from the stabilization matrix.

Protein can be eluted from the stabilization matrix, or portion of the stabilization matrix, by contacting the stabilization matrix, or portion of the stabilization matrix, with an appropriate elution buffer. The elution buffer can comprise one or more buffering agents, one or more surfactants, one or more polyols, one or more salts, one or more blocking agents, one or more reducing agents, one or more organic solvents, one or more chelating agents, one or more salts, e.g., described herein, or any combination thereof. For example, the elution buffer can comprise one or more buffers and one or more polyols. The elution buffer can comprise one or more buffers and one or more surfactants. The elution buffer can comprise one or more buffers and one or more salts. The elution buffer can comprise one or more buffers and one or more reducing agents. The elution buffer can comprise one or more buffers, one or more surfactants, and one or more polyols. The elution buffer can comprise one or more buffers, one or more surfactants, and one or more salts. The elution buffer can comprise one or more buffers, one or more salts, and one or more reducing agents. The elution buffer can comprise one or more buffers, one or more surfactants, and one or more chelating agents. The elution buffer can comprise one or more buffers, one or more salts, one or more reducing agents, one or more chelating agents, one or more surfactants, and one or more polyols.

The one or more buffering agents can be, e.g., saline, citrate, phosphate, phosphate buffered saline (PBS), acetate, glycine, tris(hydroxymethyl)aminomethane (tris) hydrochloride, tris buffered saline (TBS), 3-[1,3-dihydroxy-2-(hydroxymethyl) propan-2-yl]amino]propane-1-sulfonic acid (TAPS), bicine, tricine, 3-[1,3-dihydroxy-2-(hydroxymethyl) propan-2-yl]amino]-2-hydroxypropane-1-sulfonic acid (TAPSO), 4-(2-hydroxyethyl)-1-piperazineethanesulfonic acid (HEPES), piperazine-N,N′-bis(2-ethanesulfonic acid)(PIPES), 3-(N-morpholino) propanesulfonic acid (MOPS), 2-(N-morpholino) ethanesulfonic acid (MES), 2-[[1,3-dihydroxy-2-(hydroxymethyl) propan-2-yl]amino]ethanesulfonic acid (TES), cacodylate, glycine, carbonate, or any combination thereof. The buffering agent can be present at a concentration of from about 0.1 mM to about 500, from about 0.1 mM to about 400 mM, from about 0.1 mM to about 300 mM, from about 0.1 mM to about 200 mM, from about 0.1 mM to about 100 mM, from about 0.1 mM to about 50 mM, from about 0.1 mM to about 25 mM, from about 0.1 mM to about 20 mM, from about 0.1 mM to about 15 mM, from about 0.1 mM to about 10 mM, from about 0.1 mM to about 5 mM, from about 0.1 mM to about 4 mM, from about 0.1 mM to about 3 mM, from about 0.1 mM to about 2 mM, from about 0.1 mM to about 1 mM, from about 0.1 mM to about 0.9 mM, from about 0.1 mM to about 0.8 mM, from about 0.1 mM to about 0.7 mM, from about 0.1 mM to about 0.6 mM, from about 0.1 mM to about 0.5 mM, from about 0.1 mM to about 0.4 mM, from about 0.1 mM to about 0.3 mM, or from about 0.1 mM to about 0.2 mM. The buffering agent can be present at a concentration of less than 500 mM, less than 400 mM, less than 300 mM, less than 200 mM, less than 100 mM, less than 50 mM, less than 25 mM, less than 20 mM, less than 15 mM, less than 10 mM, less than 5 mM, less than 4 mM, less than 3 mM, less than 2 mM, less than 1 mM, less than 0.9 mM, less than 0.8 mM, less than 0.7 mM, less than 0.6 mM, less than 0.5 mM, less than 0.4 mM, less than 0.3 mM, less than 0.2 mM, or less than 0.1 mM. The buffering agent can be present at about 0.1 mM, 1 mM, 10 mM, 25 mM, or 50 mM. The buffering agent can be present at at least 0.1 mM, 1 mM, 10 mM, 25 mM, or 50 mM.

A one or more surfactants can be, e.g., an anionic, cationic, nonionic or amphoteric type. The one or more surfactants can be, e.g., polyethoxylated alcohols; polyoxyethylene sorbitan; octoxynol such as Triton X 100™ (polyethylene glycol p-(1,1,3,3-tetramethylbutyl)-phenyl ether); polysorbates such as Tween™ 20 ((e.g., polysorbate 20) or Tween™ 80 (polysorbate 80); sodium dodecyl sulfate; sodium lauryl sulfate; nonylphenol ethoxylate such as Tergitol™; cyclodextrins; or any combination thereof. Each of the one or more surfactants can be present at a concentration of less than 0.001%, less than 0.005%, less than 0.01%, less than 0.015%, less than 0.02%, less than 0.025%, less than 0.03%, less than 0.035%, less than 0.04%, less than 0.045%, less than 0.05%, less than 0.055%, less than 0.06%, less than 0.065%, less than 0.07%, less than 0.075%, less than 0.08%, less than 0.085%, less than 0.09%, less than 0.095%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, less than 0.1%, less than 1%, less than 2%, less than 3% or by volume relative to the total volume of the elution buffer. The one or more surfactants can be present at a concentration of about 0.01%, 0.05%, 0.1%, 0.5%, 1%, 5%, or 10%. The one or more surfactants can be present at a concentration of at least 0.01%, 0.05%, 0.1%, 0.5%, 1%, 5%, or 10%. The one or more surfactants can be present at a concentration of from about 0.01% to 1%, from about 0.05% to 1%, from about 0.1% to 1%, from about 0.15% to 1%, from about 0.2% to 1%, from about 0.25% to 1%, from about 0.3% to 1%, from about 0.35% to 1%, from about 0.4% to 1%, from about 0.45% to 1%, from about 0.5% to 1%, from about 0.55% to 1%, from about 0.6% to 1%, from about 0.65% to 1%, from about 0.7% to 1%, from about 0.75% to 1%, from about 0.8% to 1%, from about 0.85% to 1%, from about 0.9% to 1%, or from about 0.95% to 1%,

The elution buffer can be an aqueous solution having a pH from about 2 to about 10, from about 2 to about 9, from about 2 to about 8, from about 2 to about 7, from about 2 to about 6, from about 2 to about 5, from about 2 to about 4, or from about 2 to about 3. The elution buffer can be an aqueous solution having a pH from about 6 to about 8, from about 6 to about 7.9, from about 6 to about 7.8, from about 6 to about 7.7, from about 6 to about 7.6, from about 6 to about 7.5, from about 6 to about 7.4, from about 6 to about 7.3, from about 6 to about 7.2, from about 6 to about 7.1, from about 6 to about 7, from about 6 to about 6.9, from about 6 to about 6.8, from about 6 to about 6.7, from about 6 to about 6.6, from about 6 to about 6.5, from about 6 to about 6.4, from about 6 to about 6.3, from about 6 to about 6.2, or from about 6 to about 6.1. The elution buffer can be an aqueous solution having a pH of at least about 3, at least about 4, at least about 5, at least about 6, at least about 7, at least about 8, at least about 9, or at least about 10. The pH can be about 6, 6.5, 7, 7.5, 8, or 8.5.

In some instances, the one or more polyols can be a glycol such as ethylene glycol or propylene glycol, or a glycol polymer such as polyethylene glycol (PEG) of various weights such as PEG300, PEG400, PEG600, PEG1000, PEG3000, and PEG6000. In some instances, the one or more polyols can be a sugar. In some cases, the sugar can be sucrose, glucose, fructose, trehalose, maltose, galactose, lactose or any combination thereof. In some instances, the one or more polyols can be a sugar alcohol. In some cases, the sugar alcohol can be glycerol, erythritol, threitol, xylitol, sorbitol and the like.

The one or more salts can be sodium chloride, sodium acetate, sodium bicarbonate, sodium bisulfate, sodium bromide, potassium chloride, potassium acetate, potassium bicarbonate, potassium bisulfate, potassium bromate, potassium bromide, or potassium carbonate. The one or more salts can be at a concentration of about 0.1 mM, 5 mM, 10 mM, 25 mM, 50 mM, 100 mM, 250 mM, 500 mM, or 750 mM. The one or more salts can be at a concentration of less than 0.1 mM, 5 mM, 10 mM, 25 mM, 50 mM, 100 mM, 250 mM, 500 mM, or 750 mM. The one or more salts can be at a concentration of at least 0.1 mM, 5 mM, 10 mM, 25 mM, 50 mM, 100 mM, 250 mM, 500 mM, 750 mM, or 1000 mM.

The one or more blocking agents can be bovine serum albumin, fetal bovine serum, and the like.

The one or more organic solvents can be, e.g., methanol, ethanol, DMSO, DMF, dioxane, tetrahydrofuran, propanol, isopropanol, butanol, t-butanol, or pentanol, acetone and the like. The elution buffer can comprise less than 0.01%, less than 0.05%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, less than 1%, less than 1.5%, less than 2%, less than 2.5%, less than 3%, less than 3.5%, less than 4%, less than 4.5%, less than 5%, less than 5.5%, less than 6%, less than 6.5%, less than 7%, less than 7.5%, less than 8%, less than 8.5%, less than 9%, less than 9.5%, less than 10%, less than 11%, less than 12%, less than 13%, less than 14%, less than 15%, less than 16%, less than 17%, less than 18%, less than 19%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 99%, or 100% of an organic solvent by volume relative to the total volume of the solution. The concentration of the one or more organic solvents in the elution buffer can be at least 1%, 5%, 10%, 50%, 75%, or 100%. The concentration of the one or more organic solvents in the elution buffer can be about 1%, 5%, 10%, 50%, 75%, or 100%

The stabilization matrix, or portion of the stabilization matrix, can be contacted with a volume of the elution buffer of about, or less than 5 μL, less than 10 μL, less than 15 μL, less than 20 μL, less than 25 μL, less than 30 μL, less than 35 μL, less than 40 μL, less than 45 μL, less than 50 μL, less than 55 μL, less than 60 μL, less than 65 μL, less than 70 μL, less than 75 uL, less than 80 μL, less than 85 μL, less than 90 μL, less than 95 μL, less than 100 μL, less than 110 μL, less than 120 μL, less than 130 μL, less than 140 μL, less than 150 μL, less than 160 μL, less than 170 μL, less than 180 μL, less than 190 μL, less than 200 μL, less than 250 μL, less than 300 μL, less than 350 μL, less than 400 μL, less than 450 μL, less than 500 μL, less than 550 μL, less than 600 μL, less than 650 μL, less than 700 μL, less than 750 μL, less than 800 μL, less than 850 μL, less than 900 μL, less than 950 μL, less than 1,000 μLless than 1.5 mL, less than 2 mL, less than 2.5 mL, less than 3 mL, less than 3.5 mL, less than 4 mL, less than 4.5 mL, less than 5 mL, less than 5.5 mL, less than 6 mL, less than 6.5 mL, less than 7 mL, less than 7.5 mL, less than 8 mL, less than 8.5 mL, less than 9 mL, less than 9.5 mL, or less than 10 mL. The stabilization matrix, or portion of the stabilization matrix, can be contacted with about 0.1 mL, 0.2 mL, 0.5 mL, 0.7 mL, 1 mL, 2 mL, 5 mL, 7 mL, or 10 mL, or more of elution buffer. The stabilization matrix, or portion of the stabilization matrix, can be contacted with at least 0.1 mL, 0.2 mL, 0.5 mL, 0.7 mL, 1 mL, 2 mL, 5 mL, 7 mL, or 10 mL of elution buffer.

The volume of elution contacted with the stabilization matrix can be dependent on the surface area of the stabilization matrix. The amount of elution buffer can be less than 1 μL/mm², less than 2 μL/mm², less than 3 μL/mm², less than 4 μL/mm², less than 5 μL/mm², less than 6 μL/mm², less than 7 μL/mm², less than 8 μL/mm², less than 9 μL/mm², less than 10 μL/mm², less than 12 μL/mm², less than 14 μL/mm², less than 16 μL/mm², less than 18 μL/mm², less than 20 μL/mm², less than 25 μL/mm², less than 30 μL/mm², less than 35 μL/mm², less than 40 L/mm², less than 45 μL/mm², less than 50 μL/mm², less than 55 μL/mm², less than 60 μL/mm², less than 65 μL/mm², less than 70 μL/mm², less than 75 μL/mm², less than 80 μL/mm², less than 85 μL/mm², less than 90 μL/mm², less than 95 μL/mm², less than 100 μL/mm², less than 150 μL/mm², less than 200 μL/mm², less than 250 μL/mm², less than 300 μL/mm², less than 350 μL/mm², less than 400 μL/mm², less than 450 μL/mm², less than 500 μL/mm², less than 550 μL/mm², less than 600 μL/mm², less than 650 μL/mm², less than 700 μL/mm², less than 750 μL/mm², less than 800 μL/mm², less than 850 μL/mm², less than 900 μL/mm², less than 950 μL/mm², or less than 1,000 μL/mm². In some cases, the amount of elution buffer can be from about 10 μL/mm² to about 1,000 μL/mm², from about 10 μL/mm² to about 900 μL/mm², from about 10 μL/mm² to about 800 μL/mm², from about 10 μL/mm² to about 700 μL/mm², from about 10 μL/mm² to about 600 μL/mm², from about 10 μL/mm² to about 500 μL/mm², from about 10 μL/mm² to about 400 μL/mm², from about 10 μL/mm² to about 300 μL/mm², from about 10 μL/mm² to about 200 μL/mm², from about 10 μL/mm² to about 100 μL/mm², from about 10 μL/mm² to about 90 μL/mm², from about 10 μL/mm² to about 80 μL/mm², from about 10 μL/mm² to about 70 μL/mm², from about 10 μL/mm² to about 60 μL/mm², from about 10 L/mm² to about 50 μL/mm², from about 10 μL/mm² to about 40 μL/mm², from about 10 μL/mm² to about 30 μL/mm², or from about 10 μL/mm² to about 20 μL/mm².

The protein can be eluted from the stabilization matrix by incubating the stabilization matrix in the elution buffer in the presence or absence of agitation from an agitation source. An agitation source can be a rocker, vortexer, mixer, shaker and the like. In some cases, an agitation source can be set to a constant speed. The speed can be less than 1 rotations per minute (rpm), less than 5 rpm, less than 10 rpm, less than 15 rpm, less than 20 rpm, less than 25 rpm, less than 30 rpm, less than 35 rpm, less than 40 rpm, less than 45 rpm, less than 50 rpm, less than 55 rpm, less than 60 rpm, less than 65 rpm, less than 70 rpm, less than 75 rpm, less than 80 rpm, less than 85 rpm, less than 90 rpm, less than 95 rpm, less than 100 rpm, less than 150 rpm, less than 200 rpm, less than 250 rpm, less than 300 rpm, less than 350 rpm, less than 400 rpm, less than 450 rpm, less than 500 rpm, less than 550 rpm, less than 600 rpm, less than 650 rpm, less than 700 rpm, less than 750 rpm, less than 800 rpm, less than 850 rpm, less than 900 rpm, less than 950 rpm, less than 1,000 rpm, less than 1,500 rpm, less than 2,000 rpm, less than 2,500 rpm, less than 3,000 rpm, less than 3,500 rpm, less than 4,000 rpm, less than 4,500 rpm, less than 5,000 rpm, less than 5,500 rpm, less than 6,000 rpm, less than 6,500 rpm, less than 7,000 rpm, less than 7,500 rpm, less than 8,000 rpm, less than 8,500 rpm, less than 9,000 rpm, less than 9,500 rpm, or less than 10,000 rpm. The speed can be about 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm. The speed can be at least 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm.

The elution can be performed at a temperature of about 0° C., less than 1° C., less than 2° C., less than 3° C., less than 4° C., less than 5° C., less than 6° C., less than 7° C., less than 8° C., less than 9° C., less than 10° C., less than 11° C., less than 12° C., less than 13° C., less than 14° C., less than 15° C., less than 16° C., less than 17° C., less than 18° C., less than 19° C., less than 20° C., less than 21° C., less than 22° C., less than 23° C., less than 24° C., less than 25° C., less than 26° C., less than 27° C., less than 28° C., less than 29° C., less than 30° C., less than 31° C., less than 32° C., less than 33° C., less than 34° C., less than 35° C., less than 36° C., less than 37° C., less than 38° C., less than 39° C., less than 40° C., less than 45° C., less than 50° C., less than 55° C., less than 60° C., less than 65° C., less than 70° C., less than 75° C., less than 80° C., less than 85° C., less than 90° C., less than 95° C., or about 100° C. The elution can be performed at a temperature of from about 10° C. to about 100° C., from about 10° C. to about 95° C., from about 10° C. to about 90° C., from about 10° C. to about 85° C., from about 10° C. to about 80° C., from about 10° C. to about 75° C., from about 10° C. to about 70° C., from about 10° C. to about 65° C., from about 10° C. to about 60° C., from about 10° C. to about 55° C., from about 10° C. to about 50° C. from about 20° C. to about 50° C., from about 20° C. to about 48° C., from about 20° C. to about 46° C., from about 20° C. to about 44° C., from about 20° C. to about 42° C., from about 20° C. to about 40° C., from about 20° C. to about 38° C., from about 20° C. to about 36° C., from about 20° C. to about 34° C., from about 20° C. to about 32° C., from about 20° C. to about 30° C., from about 20° C. to about 28° C., from about 20° C. to about 26° C., from about 20° C. to about 24° C., or from about 20° C. to about 22° C. The elution be performed at about 10° C., 20° C., 25° C., 30° C., 37° C., 50° C., or 65° C.

The elution (e.g., agitation) can be performed for less than 1, less than 5, less than 10, less than 15, less than 20, less than 25, less than 30, less than 35, less than 40, less than 45, less than 50, less than 55, or less than 60 minutes. The elution can be performed for less than 0.5, less than 1, less than 1.5, less than 2, less than 2.5, less than 3, less than 3.5, less than 4, less than 4.5, less than 5, less than 5.5, less than 6, less than 6.5, less than 7, less than 7.5, less than 8, less than 8.5, less than 9, less than 9.5, less than 10, less than 10.5, less than 11, less than 11.5, or less than 12 hours. The elution can be performed for less than 0.1 days, less than 0.2 days, less than 0.3 days, less than 0.4 days, less than 0.5 days, less than 0.6 days, less than 0.7 days, less than 0.8 days, less than 0.9 days, less than 1 days, less than 1.5 days, less than 2 days, less than 2.5 days, less than 3 days, less than 3.5 days, less than 4 days, less than 4.5 days, less than 5 days, less than 5.5 days, less than 6 days, less than 6.5 days, or less than 7 days. The elution (e.g., agitation) can be performed for about 0.25 hr, 0.5 hr, 1 hr, 2 hr, 5 hr, 10 hr, 12 hr, 18 hr, 24 hr, 3 days, or 1 week. The elution (e.g., agitation) can be performed for at least 0.25 hr, 0.5 hr, 1 hr, 2 hr, 5 hr, 10 hr, 12 hr, 18 hr, 24 hr, 3 days, or 1 week. Table 1 lists examples of protein elution buffers.

TABLE 1
Examples of protein elution buffers

| Buffer 1 | TBS | | 0.1% Triton-X | 10 mM DTT | 0.5 mM |
|---|---|---|---|---|---|
| | | | 100 | | EDTA |
| Buffer 2 | TBS | | 0.1% Triton-X | | |
| | | | 100 | | |
| Buffer 3 | TBS | | | | 0.5 mM |
| | | | | | EDTA |
| Buffer 4 | TBS | | | 10 mM DTT | |
| Buffer 5 | 25 mM Tris pH | 100 mM | 0.1% Triton-X | | |
| | 7.0 | KCl | 100 | | |
| Buffer 6 | 25 mM Tris pH | 50 mM | | 10 mM DTT | 0.5 mM |
| | 7.5 | NaCl | | | EDTA |
| Buffer 7 | 25 mM Tris pH | | 0.1% Triton-X | | |
| | 7.6 | | 100 | | |
| Buffer 8 | 25 mM Tris pH | | | 10 mM DTT | |
| | 8.0 | | | | |
| Buffer 9 | 25 mM HEPES | 100 mM | 0.05% Tween | | |
| | | KCl | 20 | | |
| Buffer | 25 mM HEPES | 50 mM | | | |
| 10 | | NaCl | | | |
| Buffer | 10 mM HEPES | | 0.1% Tween 80 | | |
| 11 | | | | | |
| Buffer | 10 mM HEPES | | | | |
| 12 | | | | | |
| Buffer | PBS | | 0.05% Tween | | |
| 13 | | | 20 | | |
| Buffer | PBS | | | 10 mM DTT | |
| 14 | | | | | |
| Buffer | PBS | | | | 0.5 mM |
| 15 | | | | | EDTA |
| Buffer | PBS | | 0.1% Tween 80 | | |
| 16 | | | | | |
| Buffer | 50 mM Tris pH | 25 mM | 0.05% Tween | | |
| 17 | 7.5 | KCl | 20 | | |
| Buffer | 50 mM Tris pH | 25 mM | 0.1% Tween 80 | | |
| 18 | 7.0 | KCl | | | |
| Buffer | 50 mM Tris pH | 100 mM | | | 0.5 mM |
| 19 | 8.0 | NaCl | | | EDTA |
| Buffer | 50 mM Tris pH | 150 mM | | | |
| 20 | 8.5 | NaCl | | | |

The eluted protein can be transferred to a container for storage or further processing, or can be transferred to an assay vessel for characterization.

### Uses of Matrices

A sample can be applied to one matrix (e.g., one layer). A sample can be applied to a top matrix, and at least part of the sample can contact a second matrix, e.g., a second matrix below the top matrix. Matrices can be stacked, e.g., with about, or at least 2, 3, 4, 5, 6, 7, 8, 9, 10, 20, 50, or 100 layers. The stack can comprise the same matrix, e.g., each matrix in the stack can have the same composition. The stack can comprise matrices with different compositions, e.g., a matrix configured for stabilizing a nucleic acid can be on top of a matrix configured to stabilize protein, or vice versa. The types of matrices in the stack an alternate; e.g., a matrix configured to stabilize a protein, a matrix configured to stabilize a nucleic acid, followed by a matrix configured to stabilize a protein, etc. The volume applied to a top matrix can pass through at least 2, 3, 4, 5, 6, 7, 8, 9, or 10 layers of matrices. Using multiple matrices can increase yield of recovery of a desired biomolecule, e.g., nucleic acid or polypeptide.

A sample can pass through a plurality of matrices, but the matrices do not contact each other. For example, a sample can be applied to a matrix configured to stabilize a nucleic acid, the nucleic acid can be eluted from the matrix configured to stabilize the nucleic acid and be applied to a matrix configured to stabilize a protein. The matrix configured to stabilize the protein can be used, e.g., to remove contaminates, e.g., protein contaminates from the nucleic acid. A sample can be applied to a matrix configured to stabilize a protein, and the protein can be eluted from the matrix configured to stabilize the protein and be applied to a matrix configured to stabilize a nucleic acid. The matrix configured to stabilize the nucleic acid can be used, e.g., to remove contaminates, e.g., nucleic acid contaminates, from the protein.

Generally, a sample can contain or is suspected of containing one or more analytes. The term “analyte” as used herein can refer to any substance that can be analyzed using the assays or immunoassay devices. As an example, an immunoassay device can be configured to detect the presence of 1, 2, 3, 4, 5, 6, 7, 8, 9, 10 or more analytes in a sample. Non-limiting examples of analytes can include proteins, haptens, immunoglobulins, hormones, polynucleotides, steroids, drugs, infectious disease agents (e.g., of bacterial or viral origin), drugs of abuse, environmental agents, biological markers, and the like.

As used in the specification and claims, the singular form “a”, “an” and “the” include plural references unless the context clearly dictates otherwise. For example, the term “a cell” includes a plurality of cells, including mixtures thereof.

As used herein, the term “about” a number refers to that number plus or minus 10%, 9%, 8%, 7%, 6%, 5%, 4%, 3%, 2%, 1%, of that number.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

### Examples

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Methods and Kits for Tagged Sample Collection and Preparation

Sample acquisition and stabilization components are deployed to end users. The sample stabilization components are specifically configured for a designated set of diagnostic tests; in particular they are configured for specific target component. For example, DNA, RNA and protein target components each have different substrate composition and identifying color. Tests configured for an RNA target component have a red stabilization matrix, tests configured for DNA have a green stabilization matrix, and tests configured for a protein target component have a blue stabilization matrix. Furthermore, each deployed system has a unique barcode; this barcode is used to sort and separate the samples by type (RNA/DNA/Protein) for batching of the samples, and for connecting the sample results with the identifying information corresponding to the donor from which the sample came. The sample stabilization components have multiple barcode labels, one that remains permanently fixed to the sample stabilization component and several removable adhesive labels for easy processing. The labels display the unique barcode associated with the deployed system. After the sample acquisition and stabilization system has been used to acquire the sample, the system is returned to a facility where it is collected. The fixed barcode is used to mechanically separate the received systems by substrate type. For example, all the sample stabilization components with red stabilization matrix are collected together and assembled into batches of 96. Assays are performed based on the sample color tag. A batch of 96 red samples are collected together, two labels from each of the samples are transferred to two sets 96 RNAse-free tubes each assembled in order based on the order they were scanned in. Each rack of 96 labeled sample tubes are set-up and organized identically. The sample stabilization components for each of the 96 samples is opened in the order they were scanned in and the red colored substrate is removed and placed, like a filter, onto a rim disposed within the RNAse-free sample tubes with the corresponding sample barcode label. This is done until the substrate for each of the 96 samples is in the correctly labeled and corresponding 96 sample tubes. A “red kit” designed for the target components from the red substrate is opened, revealing red-topped containers of RNase-free PCR buffers, reagents and other molecular components, the bottles each have step numbers on them as well, so that they can be easily and quickly used to perform the different treatment steps for the red samples. An aliquot of the “step 1 red reagent” is added to the top of the red colored substrate in each of the 96 labeled tubes, the tubes are closed and the reagents are left for a few minutes to soak into the substrate. After the reagents and buffers have soaked in, the sample is centrifuged-driving the contents of the substrate into the liquid solution that forms at the bottom of the sample tube. Another aliquot of the “step 1 red reagent” is added again to the top of the substrate and the centrifugation is repeated. The tubes are opened and the solid substrate is removed from each sample; then an aliquot of “step 2 red reagent”, comprising a buffered solution containing DNA molecules that are partially double-stranded with a single stranded region that is complementary to target component RNA, is added to the liquid solution. The tubes are closed and placed in a PCR machine at a temperature that encourages Brownian motion without inducing denaturation of the double stranded DNA molecule; at this temperature RNA from the substrate hybridizes with the single stranded region of the DNA molecule. The DNA molecule has a promoter for RNA polymerase within the double stranded region and 3′ overhang of the single stranded region has a string of thymidine residues. The poly(A) tails of 3′ end of messenger RNA (mRNA) from the red substrate hybridize with the thymine residue overhangs of the DNA molecule. The tubes are then opened and an aliquot of the “step 3 red reagent” comprising ligase enzymes and buffer, is added to the tube. The samples are heated and ligation occurs between the mRNA and the double stranded DNA molecule, forming a double stranded RNA-DNA molecule with the entire mRNA incorporated as one strand of the molecule. The tube is opened and an aliquot of “step 4 red reagent” comprising RNA polymerase is added to each of the 96 tubes, the tubes are closed and 32 PCR cycles of repeated denaturation and annealing are run on the sample to amplify the RNA templates and produce a library of anti-sense cRNA. An aliquot of the amplified cRNA PCR product is transferred to each of the corresponding 96 empty labeled sample tubes, and an aliquot of “step 1 red reagent” is added to the second rack of 96 empty labeled tubes. Steps 2-4 are repeated resulting in ligation and polymerization, this time the resulting in sense strands of the mRNA. These sense strands are then sequenced using standard sequencing protocols, the results are analyzed using standard gene expression profile methods and the barcode number is used as an identifier to determine the donor associated with the given results.

Other kits for performing similar batches of analysis also available; a green kit works for green substrate components which selectively stabilize DNA and blue kits work for blue substrate components which selectively stabilize proteins. Different methods are used to treat samples from each of these different substrates.

### Example 2: Kit with Lancet and Tourniquet with Sample Separation Component

A kit is deployed to an end user or donor. The kit comprises a crystalline-activated pouch for warming hands, a lancet, a tourniquet, alcohol pads, gauze, pressure activated lancet, a self-filling capillary and a sample stabilization component with integrated sample separation unit and sample stabilization matrix. The end user warms donor hands to encourage stimulation of blood flow prior to lancing; this is accomplished by activating a crystalline-activated hand-warmer pouch and holding it between digits of the hand. The donor's hands are relaxed and positioned below the heart and muscles, while the donor sits comfortably in a chair with hand and arms loosely positioned on the arm of the chair. A tourniquet is placed on the donor's non-dominant hand and a site is selected on the donor's middle finger. A rubber band tourniquet is wrapped around the last digit of the finger and then twisted to continue to loop around the finger several times creating a tourniquet. A loop is left available for easy removal. Pressure builds at the fingertip and the fingertip appear slightly red and engorged. Sterilization of the sample site is done; first a side of the fingertip is chosen and then an alcohol pad is swiped past the area before the area is dried with a piece of sterile gauze. The donor holds and pulls on the free loop of the tourniquet during lancing. Lancing process can depend on the type and source of lancet provided. The protective cap of the lancet is removed and the lancet is placed toward the side of the sterilized finger. The lancet is placed to avoid the center of the fingertip, which is calloused and contains a higher density of nerve endings. The lancet is pressed down until the spring in the lancet is engaged and a clicking noise is heard indicating that the skin has been pierced. The first evidence of blood is immediately removed after lancing, and mild but constant pressure is applied to the finger. A self-filling capillary is held horizontal to the incision site and touched against the forming blood droplet using the self-filling capillary (e.g. Microsafe®, Safe-Tec Clinical Products, LLC, Ivyland PA), the capillary self-fill to a black line printed on the plastic shaft and then self-stops. A plastic bulb is present, and it is not depressed during the filling step. When the collected blood reaches the black line and stops filling, pressure is be withdrawn from the fingertip, and the free loop of the rubber band is released to reduce the pressure of the finger tourniquet. Blood is dispensed to the sample stabilization component; the sample stabilization component is placed on a flat surface, and the blood on the outside of the capillary is wiped with clean with sterile gauze. The filled capillary is held upright over the bottom of the sample stabilization component and the collected sample is being dispensed slowly and evenly pressing on a plastic bulb of the filled capillary. The capillary is fixed in place over the bottom of the sample stabilization component while dispensing. The capillary is discarded when all blood is dispensed onto the sample stabilization component. Post-procedure, the blood sampling component is left undisturbed while the finger tourniquet is completely removed and the incision site is cleaned. Pressure is applied to the incision using sterile gauze to stop bleeding and the hand is raised above the heart to assist in clotting. The sample stabilization component is left undisturbed for approximately 5-10 minutes post-procedure and observed to determine if the blood drop is still raised on the filter and if filter still appears “wet.” In this case a separation component is used, so the raised “wet” droplet of blood is observed, and then straw-color plasma starts to appear on the top of the sample separation component. The appearance of sample separation is used to indicate that the sample can be placed back into a storage container. The blood sampling component is labeled using a barcode label, and left at room temperature. The storage container is sealed and deposited in the mail.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A method, comprising
obtaining a device, comprising:
(a) one or more piercing elements configured to penetrate skin of a subject to expose blood;
(b) a plunger configured to drive the one or more piercing elements into the skin of the subject; and
(c) a removable collection chamber for collecting the blood;
applying the device to the skin of the subject;
actuating the plunger to cause the one or more piercing elements to pierce the skin of the subject;
creating a vacuum upon withdrawal of the one or more piercing elements from the skin; and
collecting greater than 100 μL of the blood in the removable collection chamber, wherein the removable collection chamber is configured to be removed from the device after the blood is collected in the removable collection chamber.

2. The method of claim 1, further comprising contacting the blood with a stabilizing reagent that preserves the blood for transport or storage.

3. The method of claim 1, further comprising contacting the blood with silica.

4. The method of claim 1, further comprising contacting the blood with one or more salts.

5. The method of claim 1, further comprising contacting the blood with one or more chelating agents.

6. The method of claim 1, wherein the one or more chelating agents comprise ethylenediaminotetraacetic acid (EDTA).

7. The method of claim 1, further comprising contacting the collected blood with a polymer.

8. The method of claim 1, wherein the one or more piercing elements comprise a microneedle.

9. The method of claim 1, wherein the one or more piercing elements are configured to minimize physical pain to the subject.

10. The method of claim 1, wherein the one or more piercing elements are configured to allow for penetration of the skin of the subject.

11. The method of claim 1, wherein the device comprises a body comprising a base comprising at least one aperture.

12. The method of claim 11, wherein the one or more piercing elements are configured to pass through the at least one aperture in the base when the plunger is actuated.

13. The method of claim 1, wherein the collecting comprises collecting greater than 500 μL of the blood.

14. The method of claim 1, wherein the collecting comprises collecting greater than 750 μL of the blood.

15. The method of claim 1, wherein the blood comprises one or more analytes.

16. The method of claim 15, wherein the one or more analytes comprise nucleic acid.

17. The method of claim 15, further comprising performing an assay for the one or more analytes.

18. The method of claim 17, wherein the performing an assay is at a location remote from a site of the collecting.

19. The method of claim 1, further comprising sequencing a nucleic acid from the blood or an amplified product thereof.

20. The method of claim 1, further comprising removing the removable collection chamber from the device following the collecting.
